SEQUENCE LISTING (127 sequences)

SEQ ID NO: 1 - DNA, 912 bp, Nicotiana tabacum
  source 1..912: mol_type = unassigned DNA
atggagaaatacgagaaattggagaaagtaggagaaggaacgtacggcaaagtatataaagcaaaggacaaagcaacgggtcaattggtggcgctgaagaaaactcggctagaaatggacgaagaagggattccacccactgctttaagagaaatatcacttcttcaaatgctttccaattctctctacatcgttcgtctcctctgtgtcgagcaaattgacaaaaatgggaagcctcttctttacctagtttttgagtatttggatactgatctgaagaaattcgtcgattctcatcgtaaaggtcctaatcctagacctctccctccttctctcatccagagtttcttatatcaattgtgcaaaggggtcgctcactgccatagccatggagttctccacagagatttgaagccacagaacctattagtggacaaagagaagggcatacttaagattgctgatttgggtcttggaagggctttcactgtcccaataaagagctacacccatgagattgttactctatggtacagagctcctgaagtcttgttgggatctactcattactcaactgcggttgatatgtggtctgtgggatgtatttttgccgagatggttcgaaggcaggccttatttcctggtgactctgagtttcagcaattgcttcacatattcaggttgttaggaaccccaactgagaagcagtggcctggagtgagttcactccgcgactggcatgtttatccaaaatgggaacctcagaacttggcctctgctgttccagcattgggtcctgatggtgtggatctcctcacgaaaatgctccaatatgatccggcagataggatttcagcaaaagctgcacttgatcatccatactttgatagcttggacaagtcgcaattctga

SEQ ID NO: 2 - DNA, 858 bp, Nicotiana tabacum
  source 1..858: mol_type = unassigned DNA
atggagactgtaaaaaagagtgcatcggcaatggaagcattcgagaagcttgagaaggtaggggaaggtacttacggaaaggtgtacagagcgagagatagggttactggcaaaatcgtagcactgaagaagacgaggcttcacgaggacgaagaaggtgttcctcccactactctccgcgagatctctcttctgcggatgctctctagggatcctcacattgtcaaactgatggatgttaaacaaggccagaacaaagaaggaaagacggttctctacttggtctttgagtacatggatactgatgtcaagaaatttattcgtagtttccgcgcaaatggagaaaacattccccctaaaactgtcaagagcttgatgtaccaactatgcaaaggagttgctttctgccatggtcatggcgtgttacacagggatctgaaaccacacaatcttctgatggaccgtaagacgaatgtgctcaaattagcagattttggacttggcagagcttatactctgcccatcaagaagtacacgcatgagatattaaccctatggtatagagcccctgaggttcttcttggagctactcattactccacagcagttgacatgtggtctgttggttgtatctttgctgaactggtcacaaaacaagccctcttcccaggagactctgagctgcaacaactgcttcacattttcagattgctaggtactcctaatgaagaactctggcccggggtgagcaagctagtaaactggcatgaatacccccaatggaacccccagccactctcaactgctgtccctggtctagatgaagatgggctccaccttctaactgtaagtgtttga

SEQ ID NO: 3 - DNA, 939 bp, Nicotiana tabacum
  source 1..939: mol_type = unassigned DNA
atggagactgtaaaaaagagtgcatcggccatggaggcattcgagaagcttgagaaggtaggggaaggtacttacggaaaggtgtacagagcgagagatagggttactggcaaaatcgtagcactgaagaagacgaggcttcatgaggacgaagaaggtgtccctcctactactctccgcgagatctctcttctgcggatgctttctagggatcctcacattgtcaaactgatggatgttaaacaaggccagaacaaagaaggaaagacggtcctatacttggtgtttgagtacatggatactgatgtcaagaaatttattcgtagtttccgcgcaaatggagaaaacattccccctaaaactgtcaagagcttgatgtaccagctgtgcaaaggagttgctttctgccatggtcatggtgtgttacacagggatctgaaaccacacaatcttctgatggaccgtaagacgaatgtgctcaaattagcagattttggacttggcagagcttatactctgcccatcaagaagtatacgcatgagatattaaccctgtggtatagagcccctgaggttcttcttggagctactcattactccacagcagttgacatgtggtctgttggttgtatatttgctgaactggtcacaaaacaagccctcttcccaggagactctgagctgcaacaactacttcacattttcagattgctaggtactcctaatgaagaactctggcccggggtgagcaagctagttaactggcatgaatacccccaatggaacccccagccactctcaactgctgtccctggtctagatgaagatgggctccaccttctatctgagatgttgcattatgagccagctaagaggatttcagcaaagaaagctatggaacatccctatttcgatgatctggacaaaactcctctctga

SEQ ID NO: 4 - DNA, 912 bp, Nicotiana tabacum
  source 1..912: mol_type = unassigned DNA
atggagaaatacgagaaattggagaaagtaggagaaggaacgtacggcaaagtatataaagcaaaggacaaggcgacgggacaattggtggcgctgaagaaaactcggctagaaatggacgaagaagggatcccacccactgccctaagagaaatctcgcttcttcaaatgctttcccattctctctacatcgttcgtctcctctgtgtcgagcaaattgacaaaaatgggaagccccttctttacctagtttttgagtatttggatactgatctgaagaaattcgtcgattctcatcgtaaaggtcccaatcctagacctctccctccttctctcatccagagtttcttatatcaattgtgcaaaggggtagctcactgccatagccatggagttctccacagagatttgaagccacagaacctattagtggacaaagagaagggcatacttaagattgctgatttgggccttggaagggctttcactgtcccaataaagagctacacccacgagattgttactctatggtacagagctcctgaggttttgttgggatctactcattactcaactgcggttgatatgtggtctgtgggatgtatttttgccgagatggttcgaaggcaagccttatttcctggtgactctgagtttcagcaactgcttcacatattcaggctgttaggaaccccaactgagaagcagtggcctggagtcagttcactccgcgactggcatgtttatccaaaatgggaacctcagaacttggcctctgctgttccagcattgggtcctgatggcgtggacctcctcacgaaaatgctccaatatgatccggcagataggatttcagcaaaagctgcacttgatcatccatacttcgatagcttggacaagtctcagttttga

SEQ ID NO: 5 - DNA, 222 bp, Nicotiana tabacum
  source 1..222: mol_type = unassigned DNA
atggagggggtagatagtgattgcgaggtggtggaagaaggatgcatgactccgaggcgtgacacgtgtaggataatggtgaattctttgtgcccgccgccgccgccgaaaaagaaacgagtttatgtcaaacaacagcgtcctccgcctaaggaaggttattttcaaccacctgatcttgagcttttctttgccattgtaaggagagaggcttgtgcttaa

SEQ ID NO: 6 - DNA, 1602 bp, Nicotiana tabacum
  source 1..1602: mol_type = unassigned DNA
atgcaaagcttgattttctctggcgagaagaacatgattgctgcttatttatttatacctctagaactgcatcggaatgtaatacaggtgtgtcatcgagttcaacgcactttctgctcgatgaggcatgcaaatataaaacatggatcttttcatcttgaagagcacaatatgcgaatcacacgagcacgagcaagagtgtcgggttcatcaggacgattaccacccctacacccatccacaaaacaggataagaagcaggcattgggagcagagtccaaaagatcgaaaagatctgcctcagatgagaacagacctggtacctctagtattgctactggtgttcagcctaagagaagggctgttcttaaagacatgaagaatgtacttcatgagaactctcacatgaattgcatcaacggaagcaaaattcaggttaagaaaggctccgataaaaggaacaataaggcgaaacctgctgtttctctaaaattgtcacagctccaagagaaaggaaaagaggatatagctgataaagtaaagaaagtgaaggttgagggatcacaagaaatcagttcgggggcaaactgcaaggaggatatgttaccacagctaagtagatatgtcactccagcacaatgtggtttagtccatctagtgcctgtgaacagaagttcctgcaaggccttcccacttcagaatgtaatgaaaaaagatgaaagcaaagtttgccagaaacaagaaggctttgctaatctaggaattgctgatattgattcaagacacaaggatccactgatgtgtagtctgtatgctcctgatatttataacaacttgcatgccattgagtttgaccgtaggccttctgttgattacctggaaaagctgcagctggacattaacaagggtatgcgaggtattctgattgattggcttgtggaggtttcagaagaatataggctggttcctgacacactttacctaactgttaatcttattgaccggttcctatctgaaaattacattgaaaaacaaaagctgcaactacttggagttacctgcatgctaatagcttccaaatttgaagagatttgtgcacctcgtgttgaagaattttgcttcattacagataacacttactcgaaggaagaggtaataaaaatggaaagcagagtcttgaaccttttgagctttcaacttgcttctccaaccactaagaaattcctgaggagattcattcaagcagctcaagcttcttacaaggttccctctgtcgaactggaattcatggcaaattatttagcagagttaacacttgttgactatgggtttcttaagtttcttccatctcttactgctgcatcagctgtatttctagctagatggacgcttgatcaatctaaccacccatggaatccaactttggaacactataccagatacaaggtatcagagcttagaactacagtttttgcactgcaagagttacagatgaacaccagtggctgcaccctcaatgccatacgtgaaaaatatagacaaccaaagttcaagtccgtggctactttagcagcttcaaaaccagtccaatcactgttctaa

SEQ ID NO: 7 - DNA, 1245 bp, Nicotiana tabacum
  source 1..1245: mol_type = unassigned DNA
atggatatatctgatgagaatcagttcactaggaaatcactggttggtgaggcaggaatgggaaacagcaagattggagtggagacaagacacaacagaagagcactgagagtgattaatcagaatttattaggacctaatccatatcgttgtgttgttaacaagagaagattatcacatgcaaatggaatcatctacgacaagaatcctacgaggaaattgactgcacaaattgctagctcacaccagcattaccccgaggaaaccaagaaaccaaaactagcagcggaagattttaggatttgggaagaacatgtggcagctaaagaccaacccatgtctatgtctttggaacaagaagcaacattttcaaatgacaagacagaaatggaggttgaaatggaggatatatttgaggaggcactaatagatattgacagtgatgacactaacaatccgcttgcagttgttgactatgtggaagatctatatgccaactacagaaaaatggagggttatagctgtgtttcaccaaactatatgacacaacagtttgacatcaatgaaaggatgagagctacattagtagactggctcattgaggtaaatcacaagtttgagctcagggaagagacgttattcttgactgttaattcgatagacagatttttggagaagcaaatagttgcaagaaagaagttgcagcttgttgggctggttgctatgctattagcatgcaaatatgaagaggtctctgtccctgtggtggatgatttggtgattatttcggacaacgcctatacaaggaaagaggttcttgaaatggaaacattaatgctcgatacactgcagtttaatatgtcagttccaactgcatatgtttttatgagaagatttctcaaggctgctcaagctgataggaagcttgaggtcctgtctttcttcttaatcgagctttgcctcgtggaatatgaaatgcttaagtttccaccatctttcatggctgctgctgcaatttatacagctcagtgcacgctatatggtgtcatgcaatggagtaaaacatgtgaatggcatacaagttactcagaagatcaacttctgaaatgctcgagatctatcgtgatctaccaccaaaaggcagcaacagggaaactaacaggagtacataggaagtatagcacatctaaatttggctatgcagcaaaatttgagccagcacttttcctggtgcagatcaaataa

SEQ ID NO: 8 - DNA, 1311 bp, Nicotiana tabacum
  source 1..1311: mol_type = unassigned DNA
atggccatatctgatgagaacaatcctacaatggttaaacccacaaatgtgcaaggtggggctggaatgggtaccagaaagtttggtggagtggaaacaagaaacaacagaagagcattgggagtgattaatcagaatttagtgggtggtactcatccatttccttgtgttgttaacaagagaggattatctgaagcaaatggaatctgtgacaagaatcttcaaattccagctcatagacctatcacgaggaaatttgcagctcaaattgctagctctcagcaaaatcgctccgaggaaaacaagaaagctaaaatagcagcagaagaattcagtatatgggaggatatccctctgacagatgtggaggaaaatgaggcagctaaagatcaacctgttcctatgtctttggaacaaacagaaacagtgactaatgacaagaatcaaatggaggtagaaatggaggatatatttgaggaaactattatagatattgatggcgacgatgcaaagaacccgcttgcagttgttgaatatgtacaagatttgtttgcctcctatagaaaaatggagggttgtagctgtgtttctccggattatatggcacaacagtttgacatcaatgagaaaatgagatccattctaattgactggctcattgaggtacatcacaagtttgagctcagagaagagactttgtttctgactgttaatttgatagatagatttttggagaagcaaggtgttgttaggaagaagttgcagcttgttgggttggttgccatgctattagcatgcaaatatgaggaagtttctgttccattagtggatgatttcgtgtttatttcggacaaagcctattcaaggaaggaggttcttgaaatggaaagaatgatgctcaacacactgcagtttaacatgtcagttccaactgcatatgtttttatgagaagatatcttaaggctgctcaatctgataggaagctcgagctgctgtctttcttcttggttgagctttgtctagtggaatatgaaatgctcaagtttccaccatcattcatagcagctgcagcaatctatacagctcagaccacactctacggtgtccagcaatggagtaagacatgcgagtggcatactagttactcggaagatcaacttatggagtgttcgagatcgattgtgagctatcaccagaaggcagcaacaggaaaactaacaggagtacataggaagtacagcacatctaaatttggttatgcagcaaagtgtgagcctgcccattttcttgtgcagacacaacaacaatag

SEQ ID NO: 9 - DNA, 432 bp, Nicotiana tabacum
  source 1..432: mol_type = unassigned DNA
atggagaaaaggattcttggccaattggagtggtacttaacagttccaacaccttacgtgttcctcgtccgctacattaaagctgctgtttccaatgcacagatggaaaacatggtttatttcctggctgaattggggttaatgaattatgcaaccaatatatactgcccatcgatgattgctgcctcagcagtctatgttgctcaacacacgctgaattgcactccattttggaacgacacactaaaattgcatactggtttctcagagtctcagctactgggttgtgcaaagttgctcgtaagctatcacatggaagctccagaacacaagctgaaagtgatttacaagaagtattccaaaccagagagaggtgctgttgcactgcaacctccagccaaatccctcttggctgcttcttcatatgaatag

SEQ ID NO: 10 - DNA, 906 bp, Nicotiana tabacum
  source 1..906: mol_type = unassigned DNA
atggcttcaagaaacgttcttcaacagcagaatataggtgaggcagtccccggggcgttaaagcagaaaaatatggcagcagcagcacaagggagaaatcgcaaggcgcttggtgacattgggaataatatggtaactgttcgtggtgtcgagggaaagccacttcctcaacgccccataacgaggggcttttgtgcacaattgctcgctaatgcacaagcagcagctgaaaaccaaaagaaatctatggttgttaatggggatgcaccgatcgttgctaaaggagttctaccggttaaaggtgcagcaaagaaaccagttcaaaagaaagctgctgttaaaccaaagcctgatgttattgaaattagtcctgatactgaagaacaagtgaaggaaaataagcaaaagaagaaggctggtgatgattcttcagtaaagaaagcaactcttacttcaactctcactgctaggagcaaggctgcctgtggactgagtcataaaccaaaggtccagattgtggatattgatgctgcagatgtgaataatgagttggctgtggtggaatatgttgaggatatttacaatttttataagatagctgagaatgagagcagaattcatgactacatggattcacagcctgagataactgcaaggatgagagctattctgattgattggttgattgaagtgcatcacaaatttgagcttagtcaagagactctttaccttacaatcaatatcgtcgatcgttacctcgctgtgacaactacatcaaggagggaattgcagttagtaggcatgagtgctatgctcattgcctctaaatatgaagaaatttgggctcctgaggtacatttcaagtctaattatgccaagttactttcccattttgaaagttgctaa

SEQ ID NO: 11 - DNA, 1422 bp, Nicotiana tabacum
  source 1..1422: mol_type = unassigned DNA
atggcttcaagaatcgttcttcaacagcagaatagaggtgaggcagtccctggggcagtaaagcagaaaaagaatatggcaccagaagggagaaataggaaagcacttggcgatattgggaatgtggctacaggtcgtgggctcgaaggaaaaaagccacttcctcagaaacctgtggctgttaaggtgaaaggagcaaatgttgctaaagtacctgcagcaaggaaaccagctcagaagaaagccacagttaagccaaaccccgaggatattattgaaattagtcctgacacccaagaaaaactgaaggagaagatgcaaaggaagaaggctgataaagactcattaaaacagaaagcaactcttacttctactctcactgctcgaagcaaggctgcatgtggtctgagtaaaaaaccaaaggagcaggtagtggacattgatgctgcagatgtgaacaatgagttggcagttgtggaatatgttgaagacatttacagcttctacaaacttgctgagaatgagacaagagttcatgactacatggactcacagcctgagataaatgataggatgagggcagttctgatagattggttggttgaagtccaccaaaaatttgaacttaatccagagactctttacctcacaatcaacattgtggatcgctaccttgcggtgaagactacatcaagaagggaattgcagttactgggcattagtgccatgctcatagcctccaaatatgaagaaatttgggctcctgaggtcaatgactttgtgtgcatctcagacaaaagttacactcatgatcaggtgttagctatggagaaagaaattcttgggcaattggaatggtacttaacagtcccaacaccttacgtatttctcgcccgtttcattaaagcttctctacctgattcagaaattgagaacatggtatattttctggctgagctggggttgatgaattatgcaaccattatctactgcccctcgatgattgctgcctcagcggtctatgctgctcgacacaccctcaataggacaccattttggaatgagacacttaagctgcacactggtttctcagagtctcagctaatagagtgtgcaaggttgttagtgagctatcaatctgcggctgcgactcacaagctaaaggtgatatacaagaagtactccagtccggagagaggtgttgtttcattactaactccggccaaatccttgttggctgcatcatcatcaagtgtgttgtcggagcaagctgatttacgcaaaagcacagaagcagcagcaacatcatcatccaaaatggtggtggtgggttgtcaaaggtgccacatgtatgtcatggtaactgaagctgatccaagatgcccccaatgcaagagcactactactaggaagatgacttaa

SEQ ID NO: 12 - DNA, 1608 bp, Nicotiana tabacum
  source 1..1608: mol_type = unassigned DNA
atgagtagggcttgttccctggttcaagagtactatgactgttgcttcttatttatacaaagctctagaattgtggtggaacgcaatgcagttgtagcccagataataggcatatcaccacctggatcaatgaggaatgcaaatatgacaattggatcttctaatcttaaagagcccactatgcgaatcacaagatcacgggcaaaagccttgggttcatcaggaggattaccacctcgacacccgtctgtcagacaggataacaaacagggactgggagcacagggaactaagtacaaaagatctgcctcggatgagaataatccagttactaatgctagtacagcctgtcaacagcctaagcgaagggccgttctcagggatgtcaccaatgtgctttgtgaaaattcatacatgaattgcatcaatagaagcaaatttcaggttaagaaattctctgatacgaggaattcaaaggtgacacctgctattttggtaaaaagaccgcataatgaagatagaaaagaaaacacgattgaagaagcaaaaaaagtaaagatcgagaaatcacaagaacactgttcacaagcacgcttcaaggaccttacattaactcacccaagtaaatatatcactccagcacagtgtggttttgttgatcttatgcctgtgaataggagtttacctacagccattgcagtcccgaatacaacagaaaaagatgaaaccaaggtttgccagaaacaagaaggctccgattctcttggtatagcagatatagattcaaagcacaaggatccactaatgtgtagtctatatgctcctgatatatatagcaatttgcatgccatggagcttgaccggcggccttcatttaattacatggaaaagctgcagcgggacgttaacaagggtatgcgaggtattctaattgattggctggtggaggtttctgaagaatataggctggttccggacacactttacctgactgtacatctcattgataggttcctctctgagaattacattgaaaaacaaaagctgcagctgctcggagttacctgcatgctaattgcttccaaatatgaagaaatttgtgcccctcgtgtggaagaattttgctttattacagacaacacttactcaaaagaagaggtagtaagaatggaaagtctagtattgaattttttgggctttcaacttgctgctccgaccactaaaaagttcctgaggagatttgttcaagcagcacaagcttcatatgaggttccctctgttgaactggaattcatggcaaactatttagcagagctaacacttgttgactatagttttcttaagtttcttccgtctatcactgctgcatcggctgtatttctagccaaatggacacttgatcaatctaaccacccatggaatccaactttggagcactacactagttatacagcgctagagctgaaaaccacagttcttttgctgcaagatttacagctgaacaccagcggaagcaccctgaatgccattcgtgaaaagtatagacaaccaaagttcaagtccgtggcaactttatcatctccggaaccagtccaatcactgttctaa

SEQ ID NO: 13 - DNA, 2193 bp, Nicotiana tabacum
  source 1..2193: mol_type = unassigned DNA
atgggtaaattgaactcgcagaaacatatatccaccataaaagatggagttacagaactcaaggtttacgaagaggcagataagataaaaatccaaagccgtgattctctcagcaggcgctgcaagggaatgtctggtgcgcctaacatgagtgatgtgcagaccagtcggaagagctcggagagtgatatcaagcacatagagaggataaaagcaaaatgcagtacttctgttaaggtaaatgtcaaaagaaaagtattgacagatatcagcaatatcaggggcaactcttcgagaaccaaatcatataatagctccaaactgctggtgtcaaatggtaaatgcccaaaaaatgcaagcaattcagcaagaaaatttatcatgggaaatgtgaggccaaacttgaacggagctactggtgacaagcaaatcttgacacgggcaccattcaaagatacaaaagcctcatttgatggccgaaagaccagaattcaaggccgtaaatcagttaccactggaatcaggccaactggaaggaatgatttaccgccatcaaggaggtctttacctatactacagcaggtgaacatcgagggtacagacaataaagagaagggaaaggtgagggcgaacttgaataaagctactgatgacaagcaaatcttgacacaggcaccacgcaaagatatgaaagcctcctttgatggcccaaagaccagaattcaagtccgtaaatcagttaccactggaatcaggagaactggaaggaatgctttaccgccatcaaggcggtctttaccaatactacagcaggtgaacgtagaggacacgaacaataaagagaaggtaaattccaaaaagctggagaaaggcaaaggaataagtggtgtttcagttttggcaaagcctaaggccgcaggagatgttttaccacagttaagcaaccacagcaacatacggagaaatcgagttggtgatgcctcggctagaatggctccccggggtcaagctaaggtggaagttggagcattgagaagaaaatcagtcaggacagttctgaaaattactgctagcagtctcaattcacaaaagtgttcaaagtcgaactccatgtcaggtgtgcacaaatgcacctctcgagtttccattccatgtaaaaggctggtggatgttaggacatcttccctatcaaaatatgcaacatcagagatttcagctgagcaacctcatcaaaaggaagttccttctagtagtagtggcagcttagctacaccggaattgtcaattgccaggaggaaatctgatcgtaggaagtcttttacgtgtttactgatggcaagatcaaagcttatgaaggagctatgtggaaatgtagagctggacaatttgtcaaacatttatgatagttgcaatcatcttgaagttactgaatatgttgatgacatctatcaatattattgggttatagaggcacagaatcagcctatcaaaaactacatggagactcagaaggaaataacaccccaaatgcgtggcatattgatcaactggcttattgaagtccatctgaaatttgatttgatgcaagaaactctatttctcatggttacactcctggactactacttatcattagcaagggtcaagaagaatgatttgcagttagttggccttacttcactgttgttggcatcaaaatatgaggaccttttccatccgagggtcatggacttactaagcatctctgctgagtcatacacaagagatcagatgctggaaatggagaaagatatcttaaggaaattgaagtttcgccttaatgcagcaactccttatgtcttcatgctaaggcttcttaaggctgctcaagcagacacaaggtttgaacatctggcattttacctcatcgagttgtgcttggttgaatacgaagctttgaactacaagccatccatgctttgtgcatcagctatttatgtggcaagatgtacgatgcaaatgacgccagcctggacaccactgctggggattcatggacgttatcaagaatcccaattcagacattgtgcggaaatgatcttgaggtttcacaaagctgcaagcacagcacttttgaaagtcacgcatgagaaatatatgcagtccagtaacagcaaagttgctgctataaaacctttgcagagtctcccttaa

SEQ ID NO: 14 - DNA, 1326 bp, Nicotiana tabacum
  source 1..1326: mol_type = unassigned DNA
atggttggatcagacgagaacttttcaggtgtgatgagggcttcaaatcttcaaggggggttaaggcctggtgttggaggaggaaaattgactgcaggagtgggacaaaataggagggcattaagtacaatcaataggaatgtaattggcgctccccctttaccctatgctgtcaacaagagaaatggcatttctgacaacaaagccaatgctgctaacaaaatcccttctgttccgattcatcgtccaatcacaaggaagttagctgcacaaatcgcaagcaaacagcagcaacctgcagtcgaggtaacaaagccaccagtcccagtggcaccaaatagaaatggatcagaagactgcattattatcgatgctgaagaatacaaggccgctggtgattcttccgtgccaatgtttgtgcaacatactgaagcaatgatggaggaaattgataggatggatgaggagatagagatggaagatgtagaagactggccaattgtggatatagacagtgctgataaaaagaacacgcttgcagttgtggagtacatcgatgacatctatgcttactacaagaagactgaagttcttagctgtgtccctccaaactatatggaacagcaaattgatgttaatgaaaggatgagagccatcctcattgactggctgattgaggtacactacaagtttgaactgatggaggagaccttgtacttaaccgtgaatctcatcgatagattcctggcggttcagtcagtgattaggaagaaacttcagcttgtcggaataacagctatgcttctcgcctgcaaatatgaagaggtttctgtacctgtagtggaggatctaattttgatatctgacaaggcttacaccagaaaagaagtgcttgacatggaaaagttgatggttaataccttacagttcaacgtaacagtgcctacagcatatgtgtttattaggcgatttcttaaagccgctctgtctgataaaaaggtggagctcatgtctttcttcttgatagagctatgtttggttgaatatgagatgcttaaattcccaccatcaatgctagcagctgcttccatctttactgctcaatgcactcttggtgtttctaaggagtggaataaaacctgtgagaagcatagtagctatgctaaaaatcagcttttggaatgctcgagattgatggtttctttccatcagaaggcagcaagtgggaagcttactggcgtgcaccgcaagtacagcacttgtaaatatggctatgctgctagatgtgaaccagcttctttcctgttagaagcagcatggttctag

SEQ ID NO: 15 - DNA, 1452 bp, Nicotiana tabacum
  source 1..1452: mol_type = unassigned DNA
atggcgacgacccagaatagacgttcgtcagtttcgagtgcgacggcaaagaggcaagcgatgacggctaattcgtcgttggagaataataatcatgggaaattggtggccaagaaacgaccagccctcactaatatcagtaatcatacaactgcctccgctcgtaactcgctctctcactcctccaaactcgcaccatgtacatccaaggctgtaagcattaagaagagcaatagtaatgcagcttcctcagttctgccaacatcctcctttgtcaaaccaatcagcaaaactgtgtctattccaagaagtgatgcagctatccctaagatcactgccattcctcttcctgctacttgcagcatggacatttctccatctcactcggacggatcattggtctccatggatgaaactatgtccacttctgactcactgagaagtccggatgttgagtacattgacgacaaccaaacagctgcattcgattccattgagaagaaggcgtttagcaccctctacatctcagaagatgtcaaagcagcagatatatgcaagagagatgtacttgtagacatcgaatcaggggataaaattgccaacattgataacaattttgttgatccacaattatgtgctacaatggcctgtgacatatacaaacacttgagagccacagaggtaaagaaaaggccttccacagatttcatggagaaagttcagaaggacatcaacgctagcatgcgtgctattttgattgactggcttgttgaggttgccgaggaatacaggcttgtcccggacacattgtatctgactgttaactacattgatcgatatctctccggcaatttgatggacaggcaacgactacagttgcttggagtagcttgcatgatgatcgcatccaaatatgaggagatctgtgcgcctcaagtagaagagttctgctacatcacagacaacacttacttcaaggaggaggttttacaaatggagtctaccgttttaaattacttgaagtttgaaatgacagccccaacagccaaatgttttctgaggaggttcgttcgtgctgctcaaggacttaatgaggttctgtcactgcagttggaacacttggccagctacatagcagaactctctcttttagagtacaacatgctttgttatgctccatcagtcattgctgcttctgcaattttcttagccaaatatattcttctcccctcaaagaaaccttggaactctaccttgaggcattatactctgtatcaaccctctgatctacgagactgtgtcgtggcactacatagcctttgttgcaacaacaacaattctagtttaccagcaatcagggaaaaatacagccagcacaagtacaaatttgttgcaaagaagtattgccctccaacaatacctgtagagttcttccagaacataagctgctaa

SEQ ID NO: 16 - DNA, 1239 bp, Nicotiana tabacum
  source 1..1239: mol_type = unassigned DNA
atgggtgtatctaatgagaacaatcctaccatgattaaacccacaaatgtgcaaggtgaggcagaattgggttgcagaaagtttggaatggaaacaaggaacaacagaagagcattaagtgtgattaaccagaattttgttggagctaagccatacccttgtgttgttaataagagagtattatctgaagctaatgggatctgtaacaagaatcctcctgttccagctcatagacctattacaaggaaatttgctgcacaaattgctaactcaaagcagcattatcctgaggaaaacaagaaaccaaaaatagcagctgaaggtttaagtgtgtatgaggatgtaccaataatagatgtggaagaatatgaggcagcagcaaaagaccagccagttccaatgtctttggaacaaactcaaatggaaattgagatggaggatatatttgaggagagtgtgatagatattgacagtaatgatgcgaagaacacgctcgcagttgttgactatgtggaagatctgtatgcttactactcaaaaatggagggctgcaatcgtatcccgccagactatataggacaacagtttgacatcaacgagaggatgagatctatactaattgactggctcattgaggtacaccacaagtttgatctcagggaggagacattattcctgactgttaatttgatagatagatttttggagaaacaatccgttgtgagaaagaagctgcagcttgttggtctcgtcgccatgttactagcgtgcaaatacgaggaagtttctctccctgtggtggatgatttggtggtcatttcggataaagcatacacaaggaaggaggttcttgaaatggaaaaattgatgctcaacacactacagtttaatatgtcacttccaactccatatgtttttatgagaagatttctcaaagctgctcaatcggatagaaagcttgagctactttcgttcttcttgatcgagctttgcctcgtggaatatgaaatgcttaaattcccaccatcgtttatcgctgctgctgcaatctatacagctcagtgcacactttatggtgttaaacaatggagtaagacgtgcgagcttcacacaaaatactcggcagatcaactcctggagtgttcaagattgattgtggaattccaccaaaaggcagcgacagggaaactaacaggggatctatattctaaacaaaatccagaaaataaagaagagtaa

SEQ ID NO: 17 - DNA, 1320 bp, Nicotiana tabacum
  source 1..1320: mol_type = unassigned DNA
atggtaggatcagacgagaattgccaaggtgttatcatggcttcaaatgttcaaggggctggaggaggaaaagtgacaatggggcataatagaagggctctaagtacaataaatgggaatatagttgaagctccagcatacccttgcaaagtacacaagagaaatggcatcactgacaagagcgcgaatggtgttaagaatcctcccattccaattcatcgaccggtaacgagtatgggggattcttgctgcttcaggaaatttgctgcacaaatggctaccaagctgcagcaaccaacggtaacaaagcagccagtccaaacagcaactgatagaaatgaatcagaagaccgcatcattattgatgtagaagattacaaggccacaagtgactatgatcctgtgccaatgtttgtgcaacatacagaagcaatgatggaggaaattgataggatggacgcagagacggagatggaagatgtagaggagacactaattgtggacatagacagtgctgataaaaagaacccacttgcagttgtggagtacattgatgacatgcatgcttactacaagaagaccgagagttctagctgtgcccctccaaattacatggaacaacaatttgatattaatgagaggatgagagctattctcattgactggctgattgaggtacattacaagtttgatctgatggaagagactttgtatttgaccgtaaatcttatcgatagattcttggcagtccaacaagtgattaggaaaaaactccagcttgttggggtaacagctatgcttctagcctgcaagtatgaagaagtttcagttcctgtcgttgaggatcttattttgatctctgataaggcttacaccagaaaagaagtgcttgagatggagaagttgatgatcaataccttacagttcaacctatcagtgcctacagcatatgtgtttatgatgcgatttcttaaagctgctcagtctgataagaaggtggagctcctgtctttctttatgactgaactatgcctcgttgagtatgaaatgcttaggttcccaccttcaatgctagctgctgcggcaatatttactgcccaatgtgctctaagtgcgcctaacgagttgagtaaaacttgtgagaagtatagccactacactcaagatcagcttttggagtgctcaagactgatggtttctttccatcagaaggctgcaattgggaagcttactggtgtatacagaaagtatagcatctctaaatatggatttgttgcaaaatgcccacctgcttcttttcttttagaggcatgcttttag

SEQ ID NO: 18 - DNA, 1104 bp, Nicotiana tabacum
  source 1..1104: mol_type = unassigned DNA
atggcagaccaagaaaattgtgttagagtgactcgattagccaagaaaagagcagcagaagcaatggttcagcacctgcaacagcccaacaagaagagagtggtgttgggtgagattcggaatttgtccaatcagattcagatgtttgattctgagcctcttaagcccaaatgtaataagcagactactaagaggaaggtgaaaaggtctgttagtgtgaaagagagagaatttagggaggaggatgttgattctaaattggatgatgatccccagatgtgcagtgcttatgtttctgatatatatgagtatcttcatcaaatggagattgagaaaaagagaagaccattgtctgattaccttgagaaagttcagaaggatgtaactgcaaacatgagaggggttttagtggattggctagtggaagttgcagaggaatacaagcttctttcagacactttatatctcgctgtagcctacatcgacagatacttatcgataaaggtcatccctagacaaagacttcagctattgggtgtttcttcaatgctcattgcctcgaagtatgaggagattaagcctccacgtgttgaggatttttgttacattacagacaatacatatacaaagaaagatgtggtaaagatggaggctgatgtgctacaatcccttaaatttgaaatgggcaatcctacaaccaaaacatttctcagaagatttactagggttgctcaagaagattgtaaaaactccaatttgaagttagagttcctggggtgttacctagcagagttaagtttattggattacaactgtgtgaagttcttaccttctttggtagcagctgctgtgatattcctttcaagattcacattgcagccaaagttacatccttggagtgtgggcctcgaacaaaactcgggatatagagcagcagatctaaaggaatgtgttcttatcatacatgacttgcaattaagtagaagaggaggctctttggtagctgcgaggaacaaatacaagcaacataagttcaaatatgtgtcaacattgtcttctcctctggaaataccagattcattctttgaagatacaagacaatga

SEQ ID NO: 19 - DNA, 1449 bp, Nicotiana tabacum
  source 1..1449: mol_type = unassigned DNA
atggcgacgactcagaatagacgtaattcagtttcgagtgcggtggcaaagcggcaagcgatggcggagaataatcacgggaaattgccggcgggtggggccaagaaaagaccagccctcactaatatcagtaatcatacaactgcttccgctcgtaactcgctctctcactcctccaaactcgcaccatgtacatctaaggttgtaagcattaagaagaacaatagtaatgcagcttcctcagttctgccaacatcctcctcctttgtcaaaccaatcagcaaaactgtgtctcttccaagaagtgatgcagctgtccctaagatcactgcaattcctcctcttccttccacttgcagcatggacatttctccgtctcactcggacggatcattggtctccatggatgaaactatgtccacttctaactcactgagaagtccggatgttgagtacattgacgacaaccaaacagctgcatttgattccattgagaagaaggcttttagcaccctctacatctcagaagatgttaaagcagcagatatatgcaagagagatgtactcgtggatatggaatcaggggataaaattgccaacattgataacaatcttgttgatccacaattatgtgctacaatggcctgtgacatatacaaacacttgagagccacagaggtaaagaaaaggccttccacagatttcatggagaaagttcagaaagacataaatgctagcatgcgtgctatcttgattgactggcttgttgaggttgccgaggaatacaggcttgtcccagacacattgtatctgactgttaactacattgatcgatatctctctggcaatttgatggacaggcaacgactacagttgcttggagtagcttgcatgatgatcgcatccaaatatgaggagatctgtgcgcctcaagtagaagaattctgctacataacagacaacacttacttcaaggaggaggtcttgcaaatggagtctaccgttttaaattacttgaagtttgaaatgacagccccaacagccaaatgttttctgaggaggttcgttcgcgctgctcaaggacttaatgaggttctgtcactgcagttggaacacttggccagctacatagcagaactctctcttttagagtacaacatgctttgttatgctccatcagtcattgctgcttctgcaattttcttagccaaatatattcttctcccctcaaagaaaccttggaactctaccttgaggcattatactctgtatcaaccctctgatttacgagactgtgtcatggcactacatagcctttgctgcaacaacaacaattctagtttaccagcaatcagggaaaaatacagccagcacaagtacaaatttgttgcaaagaagtattgtcctccaacaatacctgtagagttcttccagaacataagctgctaa

SEQ ID NO: 20 - DNA, 1155 bp, Nicotiana tabacum
  source 1..1155: mol_type = unassigned DNA
atgaagaagcaagagaaagaggcaataatggcggatttagaaaattgtggcagagttacacggctagccaagaaaagggcagcagaggctatggcctcacatcaacaacaacaccctagtaagaaacgggtggttttgggcgagattcagaatttttctaatctgggtgtgagtcaaattaagggtttgaacactgagcctaaaaaacaacccaaatcgaagcagcagcaatctaagaggaaactgaaaagggctgtgactagtaaaattgataaggaggagttgaatgtggataatgttgatgctaattacgatgaccctcagatgtgtagtgcttatgtttctgatatatatgattatcttcgtaaaatggagattgaggaaaagaggagacctttgcctgattacttagagaaagttcagaaggatttgagcccaaacatgagaggggtattagtcgattggctagtggaagttgcagaggaatacaagctactttcagacactttatatcttgctgtttcctacatcgatagattcttatcaacaaatgtcatcaccaggcaaaaacttcagcttttgggtgtttcgtcaatgctcatttctgcgaagtatgaggagattagtccaccacatgttgaagatttttgttacataacggataacacatatactaaggaagaggtggtgaaaatggaagctgatgtgcttaaaacactcaactttgagatgggaaatcccacagtgaaaacatttctcagaagatttactggggttgctcaagaagattataaaacccccaatttgcagttggagtttttgggctattacctagcggagttaagcatattggattatagctgtgtgaaatacgtaccttctttgctggctgctgctgtggtattcctttcgaggtttacactacaacctaatacacatccttggagtttggctcttcaacaatactcgggatataaagcagcggatttgaaggaatgtattcttatcttacacgacttgcaattaagtagaagaggaggctctttagcggctgtgagggacaaatataagcagcataagtttaagtgtgtgtcatcgttgacctctccagtggaaataccagcttcattctttgaagatatgagacaattgtaa

SEQ ID NO: 21 - DNA, 1326 bp, Nicotiana tabacum
  source 1..1326: mol_type = unassigned DNA
atggttggatcagacgagaacttttcaggtgtgatgagggcttcaaatcttcaaggaggattaaggcctgttgttggaggaggaaaattgactgcaggagttggacaaaatagaagggcacttagtacaatcaataggaatgtaattggagctccccctttaccctatgctgtcaacaagagaaatggcatttctgacaacaaagccaatgctgctaacaaaatccctcctgttccgattcatcgtccaatcacaaggaagttagctgcacaaatcgcaagcaaacagcagcaacctgcagtcgaggtaacaaagccaccagtcccattggcaccaaatagaaatgaatcagaagactgcattattatcgatgctgaagaatacaaggctactggtgattcttccgtgccaatgtttgtgcaacatactgaagcaatgatggaggaaattgataggatggacgaggagatagagatggaagatgtagaagactgtccaattgtggatatagacagcactgataaaaagaacacgcttgcagttgtggagtacattgacgacatctatgcttactacaagaagactgaggttcttagctgtgtccctccaaactacatggaacagcaaattgatgttaatgaaaggatgagagccatcctaattgactggctgattgaggtacactacaagtttgaactgatggaggagaccctgtatttgactgtgaatcttatcgatagattcctagcagttcagtcagtgattaggaaaaaacttcaacttgtcggaataactgcattgcttcttgcctgcaaatatgaagaggtttctgtacctgtagtggaggatctaattttaatttctgacaaggcttacaccagaaatgaagtgcttgtgatggaaaagttgatggttaataccttgcaattcaatgtaacagtgcctacagcatatgtgtttatgaggcgatttcttaaagccgctcagtctgataaaaaggtggagctcatgtctttcttcttgatagagctatgtttggttgaatatgagatgcttaaattcccaccatcaatgctagcagctgctgctatctttactgctcaatgcactcttggtgtttctaaggagtggaataaaacctgtgagaagcatagcagctatgctaaagatcagctttcggaatgctcgagattgatggtttctttccatcagaaggcagcaagtgggaagcttactggcgtgcaccgaaagtacagcacttctaaatatggctatgctgctagatgtgaaccagcttctttcctgttagaagcagcatggttctag

SEQ ID NO: 22 - DNA, 1215 bp, Nicotiana tabacum
  source 1..1215: mol_type = unassigned DNA
atgaaaggtggtgaggcagaaatgggaaacaacaagtttgactttggagtggagacaagacacaacagaagagcactgagagtgattaatcagaatttgttaggacctaatccatatcgttgtgttgttaacaagagaggattatcacatgcaaatggaatcatctacgataagaatcctacgaggaaattgactgcaccaattgctagctcacaccagcattaccccgaggaaacaaagaaaccaaaactagcagctgaagattttaggatttgggaggaacatgtggcagctaaagaccaactcatgcctatgtctttggaacaagaagcaacattttcaaatgacaagacagaaatggagattcaaatggaggatatatttgaggaggcactaatagatattgacagtgatgatgcaaagaacccacttgcagttgttgactatgtgaatgatctgtaccccaactacagaaaaatggagggttatagctgtgtttcaccaaactatatgacacaacagtttgatatcaatgaaaggatgagagctatattagtagactggctcattgaggtacatcacaagtttgagctcagggaagagacgttattcttgaccgttaattcgatagacagatttttggagaagcaaacagttgcaagaaagaagttgcagcttgttgggctggttgctatgctattagcatgcaaatatgaagaggtctctgtcccagtagtggatgatttggtgattatttcggacaacgcctataggaggaaagaggttcttgaaatggaaacattaatgctcaatacactgcagtttaatatgtcagttccaactgcgtatgtttttatgagaagatttctcaaggctgctcaagctgataaaaagcttgaggtcctgtcctttttcttaatcgagctttgcctcgtggaatatgaaatgcttaagtttccaccatctttcatggctgctgcggtagtctatacagctcagtgcacgctttatggtgtcaagcaatggaataaaacctgtgaatggcatacaagttactcagaagatcaacttctggagtgctcaagatcgatcgtgagctaccaccgaaaggcagcaacagggaaactaacaggagtacataggaagtatagcacatctaaatatggctacgcagcaaaatatgagccagcactttttctggtgcagatccaataa

SEQ ID NO: 23 - DNA, 1314 bp, Nicotiana tabacum
  source 1..1314: mol_type = unassigned DNA
atggccatatctgatgagaacaatcctacaatgattaaacctacaaatgtgcaaggtggggctggaatgggtaccagaaagtttggtggagtggaaacaagaaacaacagaagagcattgggagtgattaatcagaatttagtgggtggtgctcatccttttccttgtgttgttaacaaaagaggattatctgaagcaaatggaagatgtgacaagaatctgcctattccagctcatagacctatcacgaggaaatttgcagcacaaattgctagctctcatcaacatcgctccgaggaaaacaagaaagctaaaatagcatcagaagaattcagtatatgggaggatatccctctcacagatgtggaggaaaatgaagcagctaaagatcaacctgttcctatgtctttggaactaacagaaacagtcccaaatgacaacaagaatcaaatggaagtagaaatggaggatatattagaggaaaatataatagatatcgatggcgatgatgcaaagaacccccttgcagttgttgaatatgtacaagatttgtttgcctcctatagaaaaatggagggttgtagctgtgtttctccggactatatggcacaacagtttgacatcaacgagagaatgagatccattctaatcgactggctcattgaggtacatcacaagttcgagctcagggaagagacgttgttcctgactgttaatttgatagatagatttttggagaagcaaggtgttgtcagaaagaagctgcagcttgttgggttggttgccatgctattagcatgcaaatatgaggaagtttctgttccattggtggaagatttggtgtttatttctgacaaagcctattcaaggaaggagattcttgaaatggaaagaatgatgcttaacacactgcagtttaacatgtcagttccaactgcatatgtttttatgagaagatatcttaaggctgctcaatctgataggaagcttgagctgctgtctttcttcttggttgagctttgtctagtggaatatgcaatgctcaagtttccaccatcattcatagctgctgcagcaatctatacagctcagaccacactctacggtgtccagcagtggagtaagacatgcgagtggcatactagttactcggaagatcaacttatggagtgctcgagatcgattgtgagctatcaccagaaggcagcaacaggaaaactaacaggggtgcataggaagtacagtacatctaaatttggttatgcagcaaaatgtgagcctgcccattttcttgtgcagacacaacaacaatag

SEQ ID NO: 24 - DNA, 2181 bp, Nicotiana tabacum
  source 1..2181: mol_type = unassigned DNA
atgggtaaattgaactcgcagaaacatatatccaccataaaagatggagttacagaactcaaggtttacgaagaggtagataagataaaaatccaaagccgtgattctctcagcaggcgctgcaagggaaagtctggtgcgcctaacatgagtgatgtgcagaccagttggaagagctcggagaggggtatcaagcacatagagaggataaaagcaaaatgcaggacttgtgttaaggtaaatgtcaaaagaaaagtattgacggatatcagcaatatcaggggcaactcttcgagaaccaaatcatataatagctccaaactgctggtgtcagatggtaaatgcccaaaaaatgcaagcaattcagcaagaagatttatcatgggaaatgtgaggacaaacttgaacggagctactggtgacaagcaaatcttgacacgggatatgaaagcctcctttgatggcccaaagaccagaattcaaggccgtaaatcagttaccactggaatcaggccaactggaaggaatgatctaccgccatcaaggaggtctttacctatactacagcaggtgaacatcgagggtacaaacaataaagagaagggaaaggtgagggcgaacttgaataaagctactgatgacaagcaaatcttgacacaggcaccacgcaaagatatgaaagcctcctttgatggcccaaagaccagaattcaagtccgtaaaccagttaccactggaatcaggagaactggaaggaatgctctaccgccatcaaggaggtctttaccaatactacagcaggtgaacgtcgaggacacgaacaataaagagaaggaaaattccaaaaagttggagaaaggcaaaggaataagtggtgtttcagttttggcaaagcctaaggccgcaggagatgttttaccacagttaagcaaccacagcaacatccggagaaatcgagttggtgatgcctcggctagaatggctccccggggtcaagctaaggtggaagttggagcattgagaagaaaatcagtcaggacagttctgaaaattactgctagtggtctcaattcacaaaagagttcaaagtcgaactccatgtcaggtgtgcacaaatgcacctctcgatttgccagtccatgtaaaaggctggtggatgttaggacatcttccctatcaaaatctgcaacatcagagatttcagctgagcaacctcatcaaaaggaagttccttctagtagtagtggcagcttagctacaccggaattgtcaattgccaggaagaaatctgaccgtaggaagtcttttacgtgtttactgatggcaagatcaaagcttatgaaggagctatgtggaactgtagagctggacaatttgtcaaacatttatgatagttgcaatcatcttgaagttacagaatatgttgatgacatctatcagtattattgggttatagaggcacagaatcagcctatcaaaaactacatggagactcagaaggaaataacaccccaaatgcgtggcatattgatcaactggcttattgaagtccatttgaaatttgatttgatgcaagaaactctatttctcatggttacactcctggactactacttaacattagcaagggtcaagaagaatgatttgcagttagttggccttacttcactgttgttggcatcaaaatatgaggaccttttccatccgagggtcatggacttactaagcatctctgcagagtcatacacaagagatcagatgctggaaatggaaaaagatatcttaaggaaattgaagtttcgccttaatgcagcaactccttatgtcttcatgctaaggcttcttaaggctgctcaagcagacacaaggattgaacatctggcattttacctcatcgagttgtgcttggttgaatatgaagctttgaactacaagccatccatgctttgtgcatcagctatttatgtggcaagatgtacgatgcaaatgacgccagcctggacaccactgctggggatgcatgcacgttatcaagaatcccaactcagacattgcgcggaaatgatcttgaggtttcacaaagctgcaagcacagcacttttgaaagtcacgcatgagaaatatatgcagtccagtaacagcaaagttgctgctataaaacctttgcagagtctcccttaa

SEQ ID NO: 25 - DNA, 1278 bp, Nicotiana tabacum
  source 1..1278: mol_type = unassigned DNA
atggataacaatagtgttggtgttcctcacaatttacccagaggagaaatgggaggaaaacagaaaaatgcacaagccgatggaagaaataggcgtgctctcggagacattggcaaccttgtgcctgctcctgctgcagaaggaaagcctaaagcagctcagatttctcgccctgtgactaggagcttttgtgcacagttgctagctaatgcacaagaagagaagaacaagaaaccactagcagaagttgtcaataaagatgtaccagccaagaagaaggcatcagataaggaaatgaagactgttgggggaagtccattgagcaaaagaaaagcaaagaagtctggaaagactctcacttctactctcactgctagaagcaaggctgcttgtggactttccaatagaccaaagtatgagattgaggacatcgatgtcgctgatgctgataatcatttggctgctgtagagtatgttgaggatatctacaacttctacaagctcactgagggtgaaagtcgagtggatgacgactacatgaactttcaaccagacctgaatcataagatgagagccattttagtggactggttaatagaagttcacaggaaatttgagcttatgcctgagagcctttaccttacaattaccatactggaccgtttcctctcgctgaagacggttccaaggaaggaacttcagttagttggcattagctcaatgctaattgcttgcaagtatgaagagatttgggcaccagaggtgaatgatttcattcatatatcagacaatgcatatgccagagagcaaatacttcagatggagaaagcaattcttgggaagttggaatggtatttgacagttccaacaccatatgtttttctggttaggtacattaaagctgcaacaccatctgataatcaggagatggagaacatgacattcttttttgctgaacttggtcttatgaactacaagatcacaatatcataccgcccatcaatgctagcagcatcgtccgtttatgctgctcgtagcactctcaacaaaactccactatggactcaaactctgcagcaccatactggctactcagaagatcagttgatggaatgtgcaaagatattggttagttatcacttggatgctgcagaaagtaagctgaaagcaatttacaggaagttttcgagtccagatagaggtgctgttgcattcttcccaccagcaagaaatctcctacctactactactactgatgctgcttcctcctcttcttga

SEQ ID NO: 26 - DNA, 1311 bp, Nicotiana tabacum
  source 1..1311: mol_type = unassigned DNA
atggttggatcaaacgagaattgccaaggtgttataatggcttcaaatgttcaagggggattaggggctggaggaggaaaggtgacaatggggcctaatagaagggctctaagcacaataaatgggaatatagttgaagctccagcatacccttgtaaagtacacaagagaaatggcatcactgacaagagcgcgaatggtgttaagaatcctcccattccaattcatcgaccaatcacaaggaaatttgctgcacaaatggctaccaagcagcagcaaccaacggttgaggtaacaaagcagccagtccaaacagcacctgctaaaaatgaatcagaagactgcatcattattgatgcagaagattacaaggccacaagcgactatgatcctgtgccaatgtttgtgcaacatacagaagcaatgatggaggaaattgatcggatggacgctgagatggagatggaagatgtagaagagacactaattgtggacatagatagtgctgataaaaagaacccacttgcagtagcggagtacattgatgacatgcatgcttactacaagaagaccgagagttctagctgtgcccctccaaattatatggaacaacaatttgatattaatgagaggatgagagctattctcattgactggctgattgaggtacattacaagtttgatctgatggaagagactttgtatttgaccgtaaatcttatcgatagattcttagcagtccaacaagtgattaggaaaaaactccagcttgttggggtaacagctatgcttctagcctgcaagtatgaagaagtttcagttcctgtcgttgaggatcttattttgatctctgataaggcttacaccagaaaagaggtgcttgagatggagaagttgatgatcaataccttacagttcaacctaccagtgcctacagcatatgtgtttatgatgcgatttcttaaagctgctcagtctgataagaaggtggagctcctgtcttttttcatgactgaactatgcctcgttgagtatgaaatgcttaggttcccaccttcaatgctagctgctgcagcaatatttacagcccaatgtactctaggtgtgcttaacgagtggagtaaaacttgtgagaagtatagccactatactcgagatcagcttttggagtgctcaagactgatggtttctttccatcagaatgctgcaactgggaagcttgctggtgtgcatagaaagtatagcatctctaaatatggctttgttgcaaaatgcccacctgcttcttttcttttagaggcaagcttttag

SEQ ID NO: 27 - DNA, 1269 bp, Nicotiana tabacum
  source 1..1269: mol_type = unassigned DNA
atgggtgtatctaatgagaacaatcctagcatgattaaacccagaaatgtgcaaggtggggcagaattaggttacagaaagtttggagtggagacaaggaataacagaagagcattaagtgtgattaatcagaattttgttggagctaagccatacccttgtgttgttaataagagaggattatctgatactaacaagaatcctcctgttccagctcatagacctatcacaaggaaatttgctgcacaaattgccaactcaaagcagcattatcctgaggaaaacaagaaaccaaagatagcagctgaaggtttaagtgtatatgaggatgtagctatagtagatgtggaagaatatgaggcagcagcaaaagaccagccagttccaatgtctttggaacaaactcaaatggagattgagatggaggatacatttgaggagagtgtgatagatattgacagtaacgatgcgaagaacccgctcgcagttgttgactatgtggaagatctctatgcctactactcaaaaatggagggctgcagtcgtatctcgccagactatataggacaacagtttgacatcaacgagaggatgagatctatactaatcgactggctcattgaggtacaccacaagtttgatctcaaggaagagacattattcctaactgttaatttgatagatagatttttggagaaacaatctgttgtgagaaagaaactgcagcttgttggtctcgtcgccatgttactagcgtgcaaatatgaggaagtttctctccctgtggtggatgatttggtggtcatttcggataaagcatacacaaggaaggaggttcttgaaatggaaaaattgatgctcaatacgctgcagtttaatatgtcggttccaactccatatgtttttatgagaagatttctcaaagctgctcaatcggataaaaagcttgagctactttcgttcttcttgatcgaactttgcctcgtggaatatgaaatgcttaaatttccaccatcatttatcgctgctgctgcaatctacacagctcagtgcacattttatggtgttaaacaatggagtaagacgtgcgagctgcacactaaatactcggaagatcaacttctggagtgttccagattgattacgggattccaccaaaaggcagcaacagggaaattaaccggggtacatagaaagtacaatacatctaaatttggttatgtggcaaaatgtgagcctgctcattttcttcttgtgcagacccgataa

SEQ ID NO: 28 - DNA, 1479 bp, Nicotiana tabacum
  source 1..1479: mol_type = unassigned DNA
atgaggaatgcaaatatgacaattggatcttctaatcttaaagagcccactatgcgaatcacaagatcacgggcaaaagccttgggttcatcaggaggattaccacctcgacacccatctgtcagacaggataacaaacagggactgggagcaaagggaactaagtacaaaagatctgcctcggatgagaataatccagttactaatgctagtacagcctgtcaacagcctaagcgaagggctgttctcagggatgtcaccaatgtgctttgtgaaaattcatatatgaattgcatcaatagaagcaaatttcaggttaagaaattctctgataagaggaattcaaaggtgacacctgctattttggcaaaaagaccgcatcatgaagatacaaaagagaacacgattgaagaagcaaaaaaagtaaagatcgagaaatcacaagaacactgttcacaagcacgcttcaaggaccatacattaactcagccaagtaaatatatcactccagcacagtgtggttttgttgatcttatgcctgtgaataggagtttacctacagccattgcagtcctgaatacaacagaaaaagatgaaaccaaggtttgccagaaacaagaaggctccgattctcttggtatagcagatatagattcaaagcacaaggatccactaatgtgtagtctatatgctcctgatatatatagcaatttgcatgccatggagcttgaccggcggccttcatttaattacatggaaaagctgcagcgggacgttaacaagggtatgcgaggtattctaattgattggcttgtggaggtttctgaagaatataggctggttccggacacactttacctgactgtacatctcattgatcggttcctctctgagaattacattgaaaaacaaaagctgcagctgctcggagttacctgcatgctaattgcttccaaatatgaagaaatttgtgcccctcgtgtggaagaattttgctttattacagacaacacttactcaaaagaagaggtagtaagaatggaaagtctagtattgaattttttgggctttcaacttgctgctccgaccactaaaaagttcctgaggagatttgttcaagcagcacaagcttcatatgaggttccctctgttgaactggaattcatggcaaactatttagcagagctaacacttgttgactatagttttcttaagtttcttccgtctattactgctgcatcggctgtatttctagccaaatggacacttgatcaatctaaccacccatggaatccaactttggagcactacactaggtatacagcgctagagctgaaaaccatagttcttttgctgcaagatttacagctgaacaccagcggaagcaccctgaatgccattcgtgaaaagtatagacaaccaaagttcaagtccgtggcaactttatcgtctccgcagccagtccaatcactgttctaa

SEQ ID NO: 29 - DNA, 1269 bp, Nicotiana tabacum
  source 1..1269: mol_type = unassigned DNA
atggataacaaaactgttgttgttcctcacaatttacccaaaggagaaatgggaggaaaacagaaaaatggacaagccgatggaagaaataggcgtgctctcggtgacattggcaaccttgtgcctgctcctgctgtagaaggaaagcctaaagcagctcagatttctcgccctgtgactcggagcttttgtgcacagttgctagctaatgcacaagcagagaagaacaagaaagcactagcagaaattgtcaataaagatgcaccagccaagaagaaggcatcagataaggaaataaagactgttgagggaagttcattgagcaaaagaaaagcaaagaagagcggaaagactctcacttctactctcactgctagaagcaaggctgcttgtggactttccaatagaccaaagtatgagattgatgacatcgatgtcgtggatgctgataatcatttggctgctgtagagtatgttgaggatatctacaacttctacaagctcactgagggtgaaagtcgagtggatgattacatgaactttcagccagacctgaatcacaagatgagagccattttagtggactggttgatagaagttcacaggaaatttgagcttatgcctgagagcctttaccttgcaattaacatactggaccggttcctctcgctgaagacggttccaaggaaggaacttcagttagttggcattagctcaatgctaattgcttgcaaatatgaagagatttgggcaccagaggtgaatgatttcattcatatatcagacaatgcatatgccagagagcaaatacttcagatggagaaagctattcttgggaaattggaatggtatttgactgttccaacaccatatgtttttctggttagatacattaaagctgcaacaccatctgataatcaggagatggagaacatgacattcttttttgctgaacttggtcttatgaactacaagaccacaatatcatactgcccatcaatgctagcagcatcgtccgtttatgctgctcgtagcactctcaacaaaactccattatggactcaaactctgcagcaccatactggctactcagaagatcagttgatggaatgtgcaaagcaattggttagttatcacttgggtgctgcagaaagtaagctgaaagcaatttacaggaagttttcgagtccggatagaggtgctgttgccttcttcccgccagcaagaaatctcctacctactactactgatgctgcttcctgttcttga

SEQ ID NO: 30 - DNA, 1488 bp, Nicotiana tabacum
  source 1..1488: mol_type = unassigned DNA
atgaggcatgcaaatataaaacatggatcttttcatcttgaagagcacaatatgcgaatcacacgagcacgagcaagagcaagtgtgttgggttcatcaggacgattaccacccctacacccatccacaaaacaggataagaagcatgtattgggagcagagtccaaaagatcgaaaagatctgcctcagatgagaacagacctggtacctctagtactgctactggtgttcagcctaagagaagggctgttcttaaagacatgacgaatgtacttcatgagaactctcacatgaattgcatcaacggaagcaaaattcaggttaaaaaaggctccgataagaggaacaataaggcgaaacctgctgtttcggtaaaattgtcacagctccaagagaaaggaaaagaggatatagctgataaagtaaagaaagtgaaggttgagggatcacaagaaataagttcgggggcaaactgcaaggaggatatgttacctcagctaagtagatatgtcactccagcacaatgtggtttagtccatctagtgcctgtgaacagaagttcctgtaaggccatcccacttcaggatataatgaaaaaagatgaaagcaaagtttgccggaaacaagaaggctttgctaatctaggagttgctgatattgattcaagacacaaggatccactgatgtgtagtctgtatgctcctgatatttataacaatttgcatgccattgagtttgaccgtaggccttctgttgattacctggaaaagctgcagctggacattaacaagggtatgcgaggtattctgattgattggcttgtggaggtttcagaagaatataggctggttccagacacactttacctaactgttaatcttattgaccgtttcctatctgaaaattacattgaaaaacaaaagctgcaactacttggagttacctgcatgctaatagcttccaaatttgaagagatttgtgcacctcgtgttgaagaattttgcttcattacagataacacttactcgaaggaagaggtaataaaaatggaaagcagagtcttgaaccttttgagctttcaacttgcttctccaaccactaagaaattcctgaggagattcattcaagcagctcaagcttcttacaaggttccctctgtcgaactggaattcatggcaaattatttagctgagttaacacttgttgactatgggtttcttaagtttcttccatctcttactgctgcatcagctgtatttctagctagatggacgcttgatcaatctaaccacccatggaatccaactttggagcactataccagatacaaggtatcagagcttagaactacagtttttgcactgcaagagttacagatgaacaccagtggctgcaccctcaatgccatacgtgaaaaatatagacaaccaaagttcaagtccgtggctactttagcagcttcaaaaccagtccaatcactgttctaa

SEQ ID NO: 31 - DNA, 1341 bp, Nicotiana tabacum
  source 1..1341: mol_type = unassigned DNA
atggcttcaagaaacgttcttcaacagcagaatataggtgaggcagtacccggggcactaaaacagaagaatatggcagcagcagcacaaggtagaaaccgaaaggcgcttggtgatattgggaataatgtggtaactgttcgtggcgtcgagggaaagccacttcctcaacgccccataacaaggagcttttgtgcacaattgctcgctaatgcacaagcagcagctgaaaaccaaaagaaatctttggtggttaatggggatgcaccgatcgttgctaaaggagctctagcggttaaagctgcagccaaaaaaccagctcataagaaagttgctgtaaaaccaaagcctgatgtgattgaaattagtcctggtactgaagaacaagtgaaggaaaataagcaaaagaagaaggctggtgatgactcttcactaaagaaagcaactcttacttcaactctcactgctaggagcaaggctgcttgtggactgagtcataaaccaaaggtccagattgtggacattgatgctgttgatgtgaataatgagttggctgtggtggaatatgttgaggatatttacaatttttataagatagctgagaatgagagcagaattcatgattacatggattcacagcttgagataactgaaagaatgagagctattctgattgattggttgattgaagtgcatcacaaatttgagcttagtcaagagactctttacctcacaatcaatatcgtcgatcgttatctcgcggtcacaactacatcaaggagggaattgcagttagtaggcatgagtgctatgctcattgcctctaaatatgaagaaatctgggctcccgaggtgaatgactttgtgtgcatctcagataaagcttacagtcatgagcaggtgttgggaatggagaaaaggattcttggccaattggagtggtacttaacagttccaacaccttatgtgttcctcgttcgctttatcaaagctgctgtttctaatgcacaaatggaaaacatggtttatttcctggctgaattggggttaatgaattatgcaacaaatatctactgcccatcgatgattgctgcctcagcagtctatgttgctcaacacacactgaattgcactccgttttggaacgacacactaaaattgcatactggtttctcagagtctcagctactgggttgtgcaaagttgctggtaagctatcacatggaagctccagaacacaagctgaaagtgatttacaagaagtattcgagacctgagagaggtgctgttgcactgcaacctccagccaaatccctgttggctgcttctttatatgaataa

SEQ ID NO: 32 - DNA, 1422 bp, Nicotiana tabacum
  source 1..1422: mol_type = unassigned DNA
atggcttcaagaatcgtccttcaacagcagaatagaggtgaggcagtccctggggcattaaagcagaaaaagaatgtggcagcagaaggaagaaataggaaagcgcttggtgatattgggaatgtggctacaggtcgtggggtcgaaggaaaaaagccacttcctcagaaacctgttgctgttattgtaaaaggagcaaatgttgctaaagtacctgcagtaaggaaaccagctcagaagaaagccacagttaaaccaaaacctgaggagattattgaaattagtccggacactcaagaaaaactgaaggagaagatgcaaaggaagaaggctgataaagactcattaaaacagaaagcaactcttacttcaactctcactgctcgaagcaaggctgcatgtggtctgagtaaaaaaccaaaggagcagatagtggatattgatgctgcagatgtgaacaatgagttggcagttgtggaatatgttgaagacatttacagcttctacaaacttgctgagaatgagacaagagtccatgactacatggattcacagcctgaaataaatgataggatgagagcagttctgattgattggttggttgaagtccaccaaaaatttgaacttaatccagagactctttacctcacaatcaacattgtcgatcgctaccttgctgtgaagagtacatcaagaagggacttgcagctagtgggtgtcagtgccatgctcatagcctccaaatatgaagaaatttgggctcctgaggtcaatgactttgtgtgcatctcagacaaaagttacactcatgatcaggtgttaactatggagaaagaaattcttgggcaattggaatggtacttaacagttccaacaccttatgtgttcctagcacgtttcattaaagcttctccacctgattcagaaactgagaacatggtatattttctggctgagctggggttgatgaattatccaaccattatctactgcccttcaatgattgctgcctcagcggtctatgctgctcgacacaccctcaataggacaccattttggaatgagacacttaagctgcacactggtttctcagaatctcagctaatagagtgtgcaaggttgttagtgagctatcaatctgcggctgcaactcacaagctaaaggtgatatacaagaagtactccagtccggaaagaggtgttgttgcattgctaactccagccaaatccctgttggctgcatcatcattacgtgtgtcgtcggagcaagctgatttaggcaaaagcacagaagcagcagcaacatcatcatcccctatggtggtggtgggttgtcaaaggtgccacatgtatgtcatggtaactgaagctgatccaagatgcccccaatgcaagaacactactactaggaagatgacttaa

SEQ ID NO: 33 - DNA, 1767 bp, Nicotiana tabacum
  source 1..1767: mol_type = unassigned DNA
atgatacaagttaaggaggaatctcagactctggactttggtggatttgcttcttgttcatctttctctgatagcagttacgaggctagcactccaagatactcctccgaacctggttctagttatcgaaggagctcaggtccaactaaacgttcttcccaggcaggctggacagaagaagaggataatctgttgactgacgtggtgaaaaggttcaaagggagaaactggaaaaaaatagctgagtgcatgaatggaaggactgatgtgcagtgcttgcatcgctggcagaaagttctgaatcctgagcttgtaaagggtccttggtcaaaggaggaggatgacctgattattgagttagttgagaaatatggctgtaagaagtggtcttttattgccaagtctttgcctggtcgcattggtaagcaatgtagggaaaggtggcacaaccatcttgacccaaccataaaaagagatgcttggactgaacaggaagaatcagtcctatgccactatcaccaaatatatgggaacaagtgggcagaaattgcgaggtttctgcctggaaggactgataatgcaattaaaaatcattggaattcctcagttaagaaaagatcgaacttgaatttgccaagtgggttagtgctggataccgaaagtgaggaatctcctaatttctctagtgacaagaaaaaactagagatccagaagcatccattacaagctcaaaatgcagaacaaacaatctttttaggcgagcagacaggattggataatgctgctgttgctttgtcaactgatctgagaattggatatgcttattctgctggaaatgctttgcataaggatacttctttatttggagcctgtatatcagcagaagaaaatgtgagggatctgataaagccacttggtggaataccatttggcaaggcagatgttcttccaattggtgagacagataaaccatgtcaatccaatttaagtcgcactaaaatatcatatccactctcagcctcttcttcagattttcctttggatcagttgcaccacacaagatggagtacttctcaagttgaggctgttcatcctactacttttgggagcatgtatgaatctcccaagaggtctaggcacgacactgttaacgatcctgaccatgattttttgagtttgtcattggctagctttactgaggttcgttcccaaagtaacaagaagaataaagcatatgacacacaatcttctttgggtctcaagcagcagggctccttgtattatgaaccaccacagttaaaggacatgctgattcctttaacagatgaaaaccttagtagagacaaccttatcacggaaaaaaatggtcatccattttgctctactcctcctagtcttaaattaacagtctctgctaatggtagcagtccagaatctgtcttaaggaattctgcaatgagttacacaggaactccttcaatcataagaaagaagaattccagatttcccgaagctgcgacgcattctagttgcacaggcaccaccactcccacacataatttcccaagagcttctgacagggaagacacctcaaacctgaaggacagattttctgggtgtaaatcatcagtttcgggaaaatctcttggaagacggttggaatatgcctttgatatggaatgggatgcctctagatgttgcacaccagtttctgcagcttcaccttgtgcacttagacttggtgctaatacgatgctgacaccataa

SEQ ID NO: 34 - DNA, 3132 bp, Nicotiana tabacum
  source 1..3132: mol_type = unassigned DNA
atggaaagtgatagaataagcactccttcagatggcactagcagtagtctccaaagagttcggcctttgcatgggagaactagtggtcctacgagacgttcgacaaaggggcagtggactactgaagaagacgagatcctacgcaaagctgtccaacgttttaagggcaaaaactggaaaaaaatagcggaatgttttaaagaccggactgatgtacaatgcttgcaccggtggcagaaagttcttaatcctgaacttgtcaaaggtccatggtctaaagaggaggatgaagtaatagttgaattagttaagaaatatggccccaaaaagtggtccaccatcgctcaacatttgccgggacgtattggaaaacaatgtcgagaaaggtggcacaatcatctgaatcctggaataaacaaggaagcttggacgcaggaggaggagttgactctgattcgcgcccaccaaatttacgggaataaatgggcagagttaacaaagtatttgcctggaaggacagataatgcaataaaaaatcactggaatagttctgtcaaaaagaaattggactcgtatttggcatccggtttacttgcacagttcccttctctgcctaatgtcaaccgtcagaaccaatcaatcccttcttcgacgaagttgcaacagagtagtgaagatgacagtgttcgtaaagaaggaatcgaaatggaggaagcttcagaatgcagtcaagggtcaaatcttgcaggctgttcccagtctacaagtgacatgggcaatacatttgtacatacaagagaggagggcaagttgctggaggattcaaattataggaaggacccaagctccagttcagcaccatgctctgaatactataccccagcctttgaagatattaccttttcaatggcagaagtgcctagtgaacttgacgaatccaagctcctggagcataacttctcacatgactgggcagcatccatgggaaaagaatggcagtttaatccagatgacatacctaatatttctccgctggagttgatgcaggattcttcagggctcttcatgcagtgtttaactggtaatgggaatcacgaaatggttacctttccacagcaaaatgcagtgaagtatgaaatgactaatgtcggaagcatggttgtgggtttagataagcccaatgagatgtttacctctgtggagggttgcgggatggtataccctgaggcaggaattccacaatacattccctctgaaactggtatgaacggtgctgatgaaactgcagattctttgatttgccaatcgtcgaactatcagatctctgaaggtggaaatatgtctatagagaattgctgcaaccctctgtgttcacatgttatgggaacttcatccggtcaaccattttctattccttcacagttttcttcagagcaaagctcactcatgtttggtactgccgcaaatcactttcataatccatcgcagggaaacccagcacaggagtcccacacaagtaactctgatggttttctatatccctttgaatctggtactccttgtgacaacataatggacgatcctctcctggaagagcaactggatcaaactaaagattctctacagctagtttctgtcaatgattttcgctcaactccttcaaatactattcaaacatgtccattggtgaacgaaaattcgagcgtaccagaagagcagaaggatggaggagccttatactatgagcctcctcgttttccgagcttggacattccatttttcagttgtgatcttatacaatctggtgcagatgcacagcaagagtatagccctcttggcatccgccagttgatgatgacttctgtgaactgtcttactccatttaggctgtgggattcaccatctagagatggaagtccagatgctgtcctgagaagtgctgccaaaactttcaccagcacaccttccatattaaagaagcgacaccgtgatttggtgtcacctttgtcagaaaagagatgtgaaaagaagcttggaagtgatctccgtcaagaatcattctctgatctgtctaaggatttttctcgactagatgttatgtttgacgaggctgcaaatgaaaaagcaacaaagtcttctctaactatggatcagacgttggaacttcaagcttcatctgaagataaagaaaacataaatccaactgaagatggaagtaaggaggaggacaaggtacgcaatggactttccagcgagagacagttagatggaggtgaagttcactataaagagaaagtaacaaggaagggcacaaagggtggagccaatagtgcaattggaaagataaaacaaccttctggagttctggttgaactgaacgcaagtgacctgttcttctctcctgatcgttttggagccaagtctggtagagctacaaatctctgcagtaaagctctaggaaatcagtatgctagacgactcgaagctgcatcaaatcaaggttctgtttcatcttcatttgagacttcatgtttttctgttatttgctctcctcgtatacgtggaaagaaagacggaagtagttttgtcatcactacatcaatgcaatctgctccagcaccaacagccttggacaactcagctgaaacttcaggaaatggagttggcgctgagactgtaagcatatctggagaaacgccttataaaaggagtattgaatctccttcagcttggaaatctccatggttcatcaactcttttctgtcaagcccaagacttgataatgaacttaattttgaggatcttgcgctgtttatgagcccaggtgacagaagctatgatgctattggattaatgaagcaattgagtgagcagactgcaggggcatttgcagatgcccaggaggtcttgggaggtgaaactccagagtcgatcctacgggggaggaactccaaaaaccagaaagcagatgaaaatcattctcttttgtcagcaaatgttatgagtgagaggcgtactcttgatttcagtgaatgtggatcccctggaaagggaaaggaaactgaaattttttgcaccagcaacaacagttttgcaagtccttcctcctacctattgaaaggctgcaggtag

SEQ ID NO: 35 - DNA, 474 bp, Nicotiana tabacum
  source 1..474: mol_type = unassigned DNA
atgggcatgaaaaatgatgccttactgaagtctaaggcaagacgcaaaagggtggagaaaccccagaaaagtgttgggaagataaatgcagaaaaagctcaagaaagacccgtattatttctggctattaataatctaacgataggaaaatccagcaacaacagcttgacatcacctgatgtttcttcatcctgcagcagcagcattattacattcggggaaaaccaaaagatgaatattgaaatggaaaacacggttatcttggagagcaatcctgaaatttatcagtctgattgtttgagtattgagtcattggatcagttcgatacgagttcattctggtttatctacttaatgatgcccatcgtcttattttatgaaagattaattagaatctgcgacctccagagacagatccaaaatttgatctttatgggttcgagttcacgactttactgcagcctattgttttag

SEQ ID NO: 36 - DNA, 879 bp, Nicotiana tabacum
  source 1..879: mol_type = unassigned DNA
atggaaacagacatgtctttcttgtctaaaagttcaagtagctctagtgatgaagaaattggattgaggagaggtccatggactgttgaagaagacagtctcctcgtaaattacatttcccaacatggcgaaggccgatggaacatgcttgcacaccgtgctggattgaagagaacaggcaagagttgcagattgagatggctgaattacctgaaaccggacgttaaaagagggaatctcactcctcaggaacaactcctgattcttgaacttcatttcaagttgggtaacagatggtcaaagattgcgcaatatctaccaggaagaacagataatgaaatcaagaattactggagaacaagggtgcagaaacaagctaagcatttgaagattgactccaatagtgcagcatttcaacacatgattcgatgtatttggatacctcggcttctacaaaagatacaaggttcatctgctattccatcaattcagacttcacaatcaacttcattattggattcacaatatggtcctttaaatatcacagaaattacacaaaccccacaagttttaagcttagagagaaacagcatcagcagcagcagatgctgcagttcgagatcaccttcgtcagaatccatgagtatctataaatctcccaacattatttcggaatgtcctaaaattccacctcgtgagatgggtgattctgttgtcaatgttcattttccatttgacgacaacagttatgatatggatactttcagcccagcaactggcaactttttgacaaattatgatcaaatggtgggtggagaaaacaatatgatgaatggtgatattttagctgacagcttctggagcatggatcaattttag

SEQ ID NO: 37 - DNA, 738 bp, Nicotiana tabacum
  source 1..738: mol_type = unassigned DNA
atggcaaggacacgttgctatgacaaaagtggattgaagaagggaacttggacacctgatgaagataggaaattagcagcttatgttagtaaatatggttgctggaattggcgtcaacttcccaagtttgctggattagctaggtgtgggaagagctgcagactgagatggctgaattatctccaaccaaatatcaaaagaggaaactatactaaagaagaagatgaaattatcatgaagctccacgcagaaattggaaacaaatggtcggtaattgctgctcacttacctggaagatcagacaatgacataaagaatcattggcacacctctctcaaaaagagatcaactcgggaatattcaacttcaactgactcaataaagagatcatctaacaacagttatcaagccaacagtcaaaaaaagagacgtgaaaatgaaactcaactaaatgcaaatgagagttttcaattgtcaccgatgcaatcatgcagtactgaggtttcttcttgtgctacaattgatcaaaatgtggaaaatatacatggcgaacgcgaggtttttcaagaagaaatatttgaagtatctagcggaagtttttggacagaaccatttttagtagatagttttaacactgctagtgattgttttgtaccatcattcgatgatcatggagtatttgtatctccattttcccctgttatgagctatggtgaattactatgctcatattattaa

SEQ ID NO: 38 - DNA, 708 bp, Nicotiana tabacum
  source 1..708: mol_type = unassigned DNA
atggtgagaactccttctgttgacaaaaatggaataaagagaggtgcatggagtgaagaagaagacaacaaactaaaagcttttgttgaaagattcggtcatccgaattggaggcaattgcctaaacatgctggtttaatgagatgtggaaagagctgcagactgagatggatgaattacttgaggcctggtttgaagaaagggaattatagccttgaagaagaggaactcattattaaactacacaaggaacatggaaacagatggtcagtcattgctgcaagattacccggaagatccgacaacgatgtcaaaaaccagtggcatgctcatctcaagaaacgtgccaaaacaaatactaataacaattcaccaattatggagcaattttctgagtcttcgcagtctggatctcaaagtgagcaatattctcataaagtatcagaacaggaagctggctgtgatacggctagtgtaaatgccgttgatacttcagtagaggtttcatcaactgatttatattcgagtttttcccttttaaatggaatggattggattgaagaagatcatatcaggtcaatggaacaacttccagcagatttctttaacttttgttggacaaatcccatcgacaattttcagacagaaccctttgacaattttcagacggaacccttagacaatttctggagacaaccattcttttaa

SEQ ID NO: 39 - DNA, 846 bp, Nicotiana tabacum
  source 1..846: mol_type = unassigned DNA
atggtgagagctccttgttgtgagaagatggggttgaaaaaaggaccatggactcctgaagaagatcaaattcttgtttcttatattcaaacaaatggccatggcaattggagagccctccctaaactagctggacttttgagatgtgggaagagttgcagattgcgttggactaactatttgcgtccagatataaagaggggaaactttactagagaagaagaagactccattattcagttacatgaaatgcttggcaatagatggtctgcaatagcagcgagattaccgggacgaacggacaatgaaattaaaaatgtatggcacacccacttgaaaaagaggcttaaaaattaccagcctcctcaaaactccaaaagacactccaaaaacaaccttgattccaaagctcctagtacttctcaaaccttcaataattcagacaattttagcaatatccaagaagatattaatgggcccgtgaccggcccgaactcgccacaacgatcgtctagtgagatgtcgactgtcacggttgattcaacagccatgacgaccatcacaatcgatgatcagaatatgtttaagcaattagatgagatggactcgtctgaaaattttattccagagattgatgagagtttttggacggatgatttatccacaagcgataactcgacttttggtatggagggtaccggtggagaattacaagtccaatttccattttcttcggtgaagcaagaaagtatggacatggttggagcaaaattagaggacgacatggacttttggtacaatgttttcataaagtccggggacttactagatttaccggaattttga

SEQ ID NO: 40 - DNA, 1002 bp, Nicotiana tabacum
  source 1..1002: mol_type = unassigned DNA
atgggaaggcctccttgttgtgataaagcaaatgtgaaaagaggcccttggactgctgaggaagatgcaaaaattcttgcttatgtagccagtcatggaattggcaactggaccttggtccctcagaaagcaggtctgaataggtgtggaaaaagctgtaggctcagatggaccaattatctacgtcctgatcttaagcatgacaacttcacacctcaagaagaagagtgcatcattgagcttcacaaaaccattggtagcaggtggtctttaatagcaaagcaattacctgggagaacagacaatgatgtcaaaaattattggaacacaaaactgaagaaaaagcttgtgaacatgggaattgaccctgtaacacataaaccatttgctcaagtctttgctgagtatggaaaaatcagtggtctcccaattcaaaatgcaagaaatcatatctgtttgcccaacaatactactgaaatatcaaagcaacttccattctcattacgagaaaactactccactcaaaaatacacatgggatcctaaggctcagtatcaagtcatccatgaggagactcttcaaacacacagttttagtgaagtctcccccttgatttcatcagcaacttattttaacccaacagtattcagctcatcgtcttcttacgcttccgtgcaatctcaggttcataccacggcatcttcttcgtcaacatctacttggaacgagtttgtatttggagatctgtgtacatccacagatacagaacaaaaacaggaataccaactccaagctgggatatatttgtcaaaggatctgtcaaattcagttcacaaggacaatcctacttgtggggaagtgactgaagttgaggaaaaccaatctgttgaagaagccacttgttcctctgctgtggattcgttcgtggacaccatcttggctcgcgacaagcaaatgctaatggattttcctccacttttagatgtataccttgattattga

SEQ ID NO: 41 - DNA, 2997 bp, Nicotiana tabacum
  source 1..2997: mol_type = unassigned DNA
atggaaagtgataaaaccagcacgactccttcagatgatatcagtagtctgcaaagagttcagcctttgaacgggaggacgagtggtcctaagagacgttccagtcagtggactcctgaggaggatgaaatcttgcgtcaagctgtccaactgtttaaggggaaaagctggaaaaggattgcggaatgttttaaggaccggacagatgtgcaatgcttgcacaggtggcagaaagttcttgatcccgaacttgtcaaaggctcatggactaaggaggaggatgataaactaatcgaattagtgaacagatatggccccaaaaaatggtccaccattgcacaagagttagcaggacgtattggaaagcaatgtcgggaaaggtggcacaatcatctgaatccttcaataaacaaagaaccttggacacaagaggaggaattgactctaattcgtgcccatcaagtttatggaaacaagtgggcagagttagcaaaagttttgcatggaaggtctgacaatgcaataaagaatcattggcatagttctgttaaaaagaaactggactcgtatttggcatcaggtttacttgcacagttccctgctctacctaatgtcaaccatcagaaccaatcagtcccttcttcttctatgacgttgcaacaaaatagtgaagatgaaagcgttcacaaacaaggaacagaagcggaggatagttttgttaaaaagaaactggactcatatttggcatcaggtctacttggacagttccctgctctgcctaatgtcaaccatcagaaccaatcagtcccttcttcttctatgacgttgcaacaaaatagtgaagatgaaagcgttcacaaagaaggaacggaagcagaggaagtgcctgaatgcagtcaaggctctacttttgctggctgttctcagtctacaagtgacttgggcaacacatttgtgcatgtaagagagaatggtgggatgtcggaggaatcaatttgtaaaaaggatgcaacctccagcactgctccatgttgtaggaactataacccggtttttcaagatgtttcttgttcaatgctaaaagttcctagtgaacttgtggattccaagttccttgagcataatttatcacatgactggggcaattccatggaagaagattggcagtttaatagggatgacatacctaatatttctcctcccgagctaattcaggaatcttcagggatttccgtgcactgtttaaatggcaatgaaaaccatgacatggaagcaactactaatgtaggaaacgtggttgagggtccatataatcccaacgaaatgtttgtttgtgtggacggttgcatgatggtataccctgaggaaggaattcctcaatgctcctctgaaactggggttaatggctgtggtcaacctgcatattctttattttaccgatcatcaaactatcagatccctgaagtaggagatatggttccacaaaactgcaatgctttaagttttgatgattttgaagcttcatcccatcagcctttttctgttcctttacaattttcttcagaggatagatcgcctgtgtttgaccttgttttaaatcagttccataatcctccgcttgaaagcccagatcatatgaaagattcctcaaggatagttcctgtgaatgatcttggctcaactacatcaaacactgttcaaacatgtctgctgaatgaaaaatcatttgtacaagaaaagcagaaagatggaggaggtttatgctacgaccctcctcgttttccaagctcagatgttcctttcttttgttgtgatcttatgcaatctggttcagatacacaggaagagtatagcccttttggcatccggcagttgatgatgacttcagccaactgccttactccattaaggttgtgggattcaccatcaagagatgatagtccagatgctatcttgaaaagtgctgccaaaacttttacagggacaccttctatactaaagaagcgacatcgtcatttactgtcgcctttgtcagaaaagagatgtgagaaaaggcttgaaagcgatctcaatcaggaatcattctctaatatgacttcaaacttttcccgactagacgatatgtttgatgagtcagcaaatgaaaaagcgtctatggaagacggagaaaatctaccatcctcagaagatggaagaaaagaggagggtgaaatttctggagccaatgatgcaatgggaaaggtaaaacagcctcctggagttctggttgagcttagctcaaatgacctgttcttatctcctgatagtttcttgatcaagtgtgatagagctacaagtctaagtaataaagctctgggtaagcagtatgctagacgacttgaagctgcatcaaatcaagttactgtttcgtcctctttcgagacttcatgcttttctgttgtttgctctcctgacatacgtgggaagcgtagaagcagtgttgtcctagctacatcagctgcattggggaatacagctgaagattctgaaaatagatttggtactgagactttaagcatatctggagagacaccttataaaaggagttttgaatctccttcagcatggaaatctccatggttcatgaattcttttccgccaagcacaagatatgatatagaacttgcatttgaggatcttgcgcgttttatgagccctggtgacagaagctatgatgctattgggttaatgaagcaattaagtgagcagacagcagcttcaattgcagatgcccatcagatcttgggaagtgaaactccagaaacaaatttgtcgaaaaggaattccaaaaaacagaaagcagatgaaatttgtaaggcttcaaatgcaacgagtgagagacgcacactcgatttcaatgaatgtggaacaccaggaaagggaaaggaaactaccaaatttggcagcaacaacagtttttcaagtccttcctcctacctgttgaaatattgcagataa

SEQ ID NO: 42 - DNA, 3048 bp, Nicotiana tabacum
  source 1..3048: mol_type = unassigned DNA
atgttggattatccagatttgcctttgtctttgaccatggaaagtgataaaaccagcacgacgccttcagatgatatcagtagtctgcaaagagttcagccttcgcacgggaggacgagtggtcctaagagacgttccagtcagtggactcccgaggaggatgaaatcttgcgccaagctgtccaacagtttaaggggaaaagctggaaaaggattgcggaatgttttaaggaccggacagatgtgcaatgcttgcacaggtggcagaaagttcttgatcctgaacttgtcaaaggctcatggactaaggaggaggatgataaactaatcgaattagtgaacagatatggccccaaaaaatggtccaccattgcacaagagttagcaggacgtattggaaagcaatgccgggaaaggtggcacaatcatctgaatcctgcaataaacaaagaaccttggacacaagaggaggaattgactctaattcgtgcccaccaagtttatggaaacaagtgggcagagttagcaaaagttttgcatggaaggagtgacaatgcaataaagaatcattggcatagttctgttaaaaagaaactggactcatatttggcatcaggtttacttgcacagttccctgctctacctaatgtcaaccatcagaaccaatcagtcccttcttcttctatgacgttgcaacaaaatagtgaagatgaaagcgttcacaaagaaggaacagaagcggaggatagttctgttaaaaagaaactggactcatattcggcatcaggtctacttggacagttctctgctctgcctaatgtcaaccatcagaaccaatcagtcccttcttcttctatgacgttgcaacaaaatagtgaagatgaaagcgttcacaaagaaggaatggaagcggaggaagtgcctgaatgcagtcaaggctcgaattttgctggctgttctcagtctacaagtgacttgggcaacacatttgtgcatataagagagaacggtgggatgtcggaggaatcaatttgtaaaaaggatgcaacctccagcactgctccatgttgtaggaactatagcccagtttttcaagatgtttcttgttcaatgttaaaagttcctagtgaacttgcggattccaagttccttgagcataatttatcacatgactggggcaattccatggaagaagattggcagtttaatagggatgacatacctaatatttctcctccggagtttattcaggaatcttcagggatttccgtgcactgtttaactggcaacgacaaccatgacatggtagcaactgctaatgtaggaaacgtggttgaggatccatataagcccaacgaaatgtttgtttctgtggacggttccatgatggtataccccgaggaaggaattcctcaatgctctccgtctgaaactggggttaatggctgtggtcaaccttcatattctttattttaccaatcatcaaactatcagatccctgaagcaggagatatggttccacaaaactgcaatgctttaaattttgatgattttgaagcttcattccatcagccattttctgttccttcacaattttcttcagaggatagatcgtctgtgtttgacattgttttaaatcagttccataatcctccgcttgaaggcccagatcatatgaaagattcctcaaggatagttcccgtgaatgatattggctcaactacatcaaacactgttcaaacatgtctgctgaatgaaaattcatttgtacaagaagagcagaaagatggaggagctttatgctatgaccctcctcgttttccaagctcggatgttcctttcttttgttgtgatcttatacaatctggttcagatacacaggaagagtatagcccttttggcatccggcagttgatgatgacttcggcgaactgccttactccattaaggttgtgggattcaccatcaagagatgatagtccagatgctatcttgaaaagtgctgccaaaacttttacagggacaccttctatactaaagaagcgacatcgtcatttactgtcgcctttgtcagaaaagagatgtgagaaaaagcttgaaagcaatctcaatcaggaatcattctataatatgtctacaaacttttcccgaccagacgatatgtttgatgagtcagcaaatgaaaaagcatctatggaagacaaagaaaatctacatccatcctcagaagatggaagaaaagaggagggcgaaatttctggagccaatgatgcaacgggaatggtaaaacagcatcctggagtgctggttgagcttagctcaaatgacttgttcttttctcctgatcgtttcttaatcaagtgtgatagagctacaagtctaagtaataaagctctgggtaggcagtatgctagacgacttgaagctgcatcaaatcaagttactgtttcgtcctcttttgaaacttcatgcttgtctgttgtatgctctcctgacatatgtgggaagcatagaggcagtgttgtcatagctacatcaactgctttggagaatacagctgaagattctgaaaatggatttggtgctgagactttaagcatatttggagagacaccttttaaaaggagttttgaatctccttcagcatggaaatctccatggttcatgagttcttttccgccaagcacaagatatgatacagaacttgaatttgaggatcttgccctttttatgagcccgggtgacagaagctatgatgctattgggttaatgaagcaattaagtgagcagacagcaccttcaattgcagatgcccatcagatcttgggaagtgaaactccagaaacaaacttgtcgaaaaggaattccaaaaaaccgaaagcagatgaaaattgtacccttctggcttcaaatgctacgagtgagagacgaacactcgattttaatgaatgtggaattccaggaaagggaaaggaaactaccaaatttggcagcaacaacaacagcttttcaagtccttcctcctacctgttgaaatattgcagataa

SEQ ID NO: 43 - DNA, 3129 bp, Nicotiana tabacum
  source 1..3129: mol_type = unassigned DNA
atggaaagtgatagaataagcactccttcagatggcactagcagtagtctccaaagagttcggcctttgcatgggagaactagtggtcctacgagacgttcaacaaaggggcagtggactactgaagaagacgagatcctacgcaaagctgtccaacgttttaagggcaaaaactggaaaaaaatagcggaatgttttaaagaccggactgatgtacaatgcttgcaccggtggcagaaagttcttaatcctgaacttgtcaaaggtccatggtctaaagaggaggatgaagtaatagttgaattagttaagaaatatggccccaaaaagtggtccaccatcgctcaacatttgccgggacgtattggaaaacaatgtcgagaaaggtggcacaatcatctgaatcctggaataaacaaggaagcttggacgcaggaggaggagttgactctgattcgtgcccatcaaatttacgggaataaatgggcagagttaacgaagtatttgcctggaaggacagataatgcaataaaaaatcactggaatagttccgtcaaaaagaaattggactcgtatttggcatcaggtttacttgcacagttccctgctttgcctaatgtcaaccgtcagaaccaatcaatcccttcttcggcgaagttgcaacagagtagtgaagatgatagtgttcgtaaagaaggaaccgaaatggaggaagcttcagaatgcagtcaagggtcaaatcttgctggctgttcccagtctacaagtgacatgggcaacaaatttgtacatacaagagaggagggcaagttgctggaggattcaaattataggaaggacccaagctccagttcagcaccatgctctgaatactataccccagcctttgaagatattaccttttcaatggcagaagtgcctagtgaacttgacgaatccaagctcctggagcataccttctcacatgactgggcagcatccattggaaaagaatggcagtttaatccagatgacatacctaatatttctccgctggagttgatgcaggattcttcagggctcttcatgcagtgtttaactggtaatgggaatcacgatatggttacctttccacagcaaaatgcagtgaagtttgaaacgactaatgtcgggagcatggttgtgggttttgataagcccaatgagatgtttacctctgtggagggttgcaggatggtataccctgaggcaggaattccacaatacattccctctgaagctggtacgaacggtgctgatgaaactgcagattctttgatttgccaatcatcgaactatcagatctctgaaggtggaaatatgtctatagagaattgcaaccctctctgttcagatgttatgggaacttcatccggccaaccattttccattccttcacagttttcttcagagcaaagctcactcatgtttggtactgccgcaaatcagtttcataatccattgcagggaaacccagcacaggagtcccacacaagtaactctgatggttttctatatccctttgaatctggtactccttgtgacaacataatggacgatcctctcctggaagagcaactggatcaaactaaagattctctacagctagtttctgtcaatgattttcgcacaactccttcaaatactattcaaacatgtccattggtgaacgaaaattcgagcataccagtagagcagaaggatggaggagccttatactatgagcctcctcgttttccgagcttggacattccatttttcagttgtgatcttatacaatctggtacagatgcacagcaagagtacagccctcttggcatccgccagttgatgatgacttctgtgaactgtcttactccatttaggctgtgggattcaccatctagagatggaagtacagatgccgtcctgagaagtgctgccaaaactttcaccagcacaccttctatattaaagaagcgacaccgtgatttggtgtcacctttgtcagaaaagagatgtgaaaagaagcttggaagtgatttccgtcaagaatcattctctgatctgtctaaggatttttctcgactagatgttatgtttgacgaggctgcaaatgaaaaagcaacaaagtcttctctaactacggatcaaacattggaacttgaagcttcatctgaagataaagaaaacataaatccaactgaagatggaagtaaggaggaggacaaggtacgtaatggactttccaacgagagacagttagatggaggtgaagttcactataaagagaaaggaacaagggagggcacaaagggtggagccaatagtgcaattggaaagataaaacaaccttctggagttctggttgaactgaacgcaagtgacctgttcttctctcctgatcgttttggagccaagtctggtagagctacatatctcagcagtaaagctctaggaaatcagtacgctagacgactcgaagctgcatcaaatcaaggttctgtttcatcttcatttgagacttcatgtttttctgttatttgctctcctcgtatacgtggaaagaaagacggaagtagttttatcatcactacatcaatgcaatctgctccagcaccaacagccttggacaactcagctgaaacttcaggaaatggagttggcgcggagactgtaagcatatctggagaaacgccttataaaaggagtattgaatctccttcagcttggaaatctccatggttcatcaactctcttctgtcaagcccaagacttgataatgaacttaattttgaggatcttgcactgtttatgagtccaggtgacagaagctatgatgctattggattgatgaagcaattgagtgagcagactgcaggggcatttgcagacgcacaggaggtcttgggaggtgaaactccagagtcaatcctacgggggaggaactccaaaaaccagaaagcagatgaaaatcattcacttttgtctgcaaatgttatgagtgagaggcgtactcttgatttcagtgaatgtggatcacctggaaagggaaaggaaactgaaaatttttgcacgagcaacaacagcttttcaagtccttcctcctacctattgaaaggctgcaggtag

SEQ ID NO: 44 - DNA, 954 bp, Nicotiana tabacum
  source 1..954: mol_type = unassigned DNA
atgggaagagctccttgctgtgacaaaaacaacgttaagagagggccatggtcgcctgaagaagattctaagttgaagtcatatattgaacagaatgggacaggtggcaactggattgctttgcctccaaaaattggcctaaatagatgtggaaagagctgtaggcttagatggttaaattatctgcgtccaaatattaagcatggagggttctcagaagaagaagatagaatcatttgcagcctctacataagtattggaagcaggtggtcaataattgcagcacaactccctggaagaacagataatgatataaagaactactggaacactaggctgaagaagaagctatttggaaagcagcgccaaaagcaaggatcaagaaaaggaaaagaaatcaactccaatatggtaatttccaataacaataacaacaaccaattcccatgttggcctgagcttcccatcttgcagccaataccatactctaatgatgaaccaagatttaatgaccattcttccataagaaaactgttgataaaacttggaggtaaattttcagacgaagatcaaccgataaatgaagcaacaaatcctcaatatcctatggataattcattattgatgcagccgatatatcagaatattcctatcaatatgatctcttctgctccaatagataatgtcttgggaaatgctcaatacaacatggatagggcagctagcagttttacagctgagcttgagcatatgattcaaaataatcaacaaaaattggacggtcttgaatttttatatgaggattatatgcttattgataaatctgcgtctacttctggaggaaacttggactgggaatcgatgaatccttttgtacttcctcttcctcctataaatgatgaaggttttcaacaaggtgttatatttcaagagaataatactatggcacaataa

SEQ ID NO: 45 - protein, 303 aa, Nicotiana tabacum
MEKYEKLEKVGEGTYGKVYKAKDKATGQLVALKKTRLEMDEEGIPPTALREISLLQMLSNSLYIVRLLCVEQIDKNGKPLLYLVFEYLDTDLKKFVDSHRKGPNPRPLPPSLIQSFLYQLCKGVAHCHSHGVLHRDLKPQNLLVDKEKGILKIADLGLGRAFTVPIKSYTHEIVTLWYRAPEVLLGSTHYSTAVDMWSVGCIFAEMVRRQALFPGDSEFQQLLHIFRLLGTPTEKQWPGVSSLRDWHVYPKWEPQNLASAVPALGPDGVDLLTKMLQYDPADRISAKAALDHPYFDSLDKSQF

SEQ ID NO: 46 - protein, 285 aa, Nicotiana tabacum
METVKKSASAMEAFEKLEKVGEGTYGKVYRARDRVTGKIVALKKTRLHEDEEGVPPTTLREISLLRMLSRDPHIVKLMDVKQGQNKEGKTVLYLVFEYMDTDVKKFIRSFRANGENIPPKTVKSLMYQLCKGVAFCHGHGVLHRDLKPHNLLMDRKTNVLKLADFGLGRAYTLPIKKYTHEILTLWYRAPEVLLGATHYSTAVDMWSVGCIFAELVTKQALFPGDSELQQLLHIFRLLGTPNEELWPGVSKLVNWHEYPQWNPQPLSTAVPGLDEDGLHLLTVSV

SEQ ID NO: 47 - protein, 312 aa, Nicotiana tabacum
METVKKSASAMEAFEKLEKVGEGTYGKVYRARDRVTGKIVALKKTRLHEDEEGVPPTTLREISLLRMLSRDPHIVKLMDVKQGQNKEGKTVLYLVFEYMDTDVKKFIRSFRANGENIPPKTVKSLMYQLCKGVAFCHGHGVLHRDLKPHNLLMDRKTNVLKLADFGLGRAYTLPIKKYTHEILTLWYRAPEVLLGATHYSTAVDMWSVGCIFAELVTKQALFPGDSELQQLLHIFRLLGTPNEELWPGVSKLVNWHEYPQWNPQPLSTAVPGLDEDGLHLLSEMLHYEPAKRISAKKAMEHPYFDDLDKTPL

SEQ ID NO: 48 - protein, 303 aa, Nicotiana tabacum
MEKYEKLEKVGEGTYGKVYKAKDKATGQLVALKKTRLEMDEEGIPPTALREISLLQMLSHSLYIVRLLCVEQIDKNGKPLLYLVFEYLDTDLKKFVDSHRKGPNPRPLPPSLIQSFLYQLCKGVAHCHSHGVLHRDLKPQNLLVDKEKGILKIADLGLGRAFTVPIKSYTHEIVTLWYRAPEVLLGSTHYSTAVDMWSVGCIFAEMVRRQALFPGDSEFQQLLHIFRLLGTPTEKQWPGVSSLRDWHVYPKWEPQNLASAVPALGPDGVDLLTKMLQYDPADRISAKAALDHPYFDSLDKSQF

SEQ ID NO: 49 - protein, 73 aa, Nicotiana tabacum
MEGVDSDCEVVEEGCMTPRRDTCRIMVNSLCPPPPPKKKRVYVKQQRPPPKEGYFQPPDLELFFAIVRREACA

SEQ ID NO: 50 - protein, 533 aa, Nicotiana tabacum
MQSLIFSGEKNMIAAYLFIPLELHRNVIQVCHRVQRTFCSMRHANIKHGSFHLEEHNMRITRARARVSGSSGRLPPLHPSTKQDKKQALGAESKRSKRSASDENRPGTSSIATGVQPKRRAVLKDMKNVLHENSHMNCINGSKIQVKKGSDKRNNKAKPAVSLKLSQLQEKGKEDIADKVKKVKVEGSQEISSGANCKEDMLPQLSRYVTPAQCGLVHLVPVNRSSCKAFPLQNVMKKDESKVCQKQEGFANLGIADIDSRHKDPLMCSLYAPDIYNNLHAIEFDRRPSVDYLEKLQLDINKGMRGILIDWLVEVSEEYRLVPDTLYLTVNLIDRFLSENYIEKQKLQLLGVTCMLIASKFEEICAPRVEEFCFITDNTYSKEEVIKMESRVLNLLSFQLASPTTKKFLRRFIQAAQASYKVPSVELEFMANYLAELTLVDYGFLKFLPSLTAASAVFLARWTLDQSNHPWNPTLEHYTRYKVSELRTTVFALQELQMNTSGCTLNAIREKYRQPKFKSVATLAASKPVQSLF

SEQ ID NO: 51 - protein, 414 aa, Nicotiana tabacum
MDISDENQFTRKSLVGEAGMGNSKIGVETRHNRRALRVINQNLLGPNPYRCVVNKRRLSHANGIIYDKNPTRKLTAQIASSHQHYPEETKKPKLAAEDFRIWEEHVAAKDQPMSMSLEQEATFSNDKTEMEVEMEDIFEEALIDIDSDDTNNPLAVVDYVEDLYANYRKMEGYSCVSPNYMTQQFDINERMRATLVDWLIEVNHKFELREETLFLTVNSIDRFLEKQIVARKKLQLVGLVAMLLACKYEEVSVPVVDDLVIISDNAYTRKEVLEMETLMLDTLQFNMSVPTAYVFMRRFLKAAQADRKLEVLSFFLIELCLVEYEMLKFPPSFMAAAAIYTAQCTLYGVMQWSKTCEWHTSYSEDQLLKCSRSIVIYHQKAATGKLTGVHRKYSTSKFGYAAKFEPALFLVQIK

SEQ ID NO: 52 - protein, 436 aa, Nicotiana tabacum
MAISDENNPTMVKPTNVQGGAGMGTRKFGGVETRNNRRALGVINQNLVGGTHPFPCVVNKRGLSEANGICDKNLQIPAHRPITRKFAAQIASSQQNRSEENKKAKIAAEEFSIWEDIPLTDVEENEAAKDQPVPMSLEQTETVTNDKNQMEVEMEDIFEETIIDIDGDDAKNPLAVVEYVQDLFASYRKMEGCSCVSPDYMAQQFDINEKMRSILIDWLIEVHHKFELREETLFLTVNLIDRFLEKQGVVRKKLQLVGLVAMLLACKYEEVSVPLVDDFVFISDKAYSRKEVLEMERMMLNTLQFNMSVPTAYVFMRRYLKAAQSDRKLELLSFFLVELCLVEYEMLKFPPSFIAAAAIYTAQTTLYGVQQWSKTCEWHTSYSEDQLMECSRSIVSYHQKAATGKLTGVHRKYSTSKFGYAAKCEPAHFLVQTQQQ

SEQ ID NO: 53 - protein, 143 aa, Nicotiana tabacum
MEKRILGQLEWYLTVPTPYVFLVRYIKAAVSNAQMENMVYFLAELGLMNYATNIYCPSMIAASAVYVAQHTLNCTPFWNDTLKLHTGFSESQLLGCAKLLVSYHMEAPEHKLKVIYKKYSKPERGAVALQPPAKSLLAASSYE

SEQ ID NO: 54 - protein, 301 aa, Nicotiana tabacum
MASRNVLQQQNIGEAVPGALKQKNMAAAAQGRNRKALGDIGNNMVTVRGVEGKPLPQRPITRGFCAQLLANAQAAAENQKKSMVVNGDAPIVAKGVLPVKGAAKKPVQKKAAVKPKPDVIEISPDTEEQVKENKQKKKAGDDSSVKKATLTSTLTARSKAACGLSHKPKVQIVDIDAADVNNELAVVEYVEDIYNFYKIAENESRIHDYMDSQPEITARMRAILIDWLIEVHHKFELSQETLYLTINIVDRYLAVTTTSRRELQLVGMSAMLIASKYEEIWAPEVHFKSNYAKLLSHFESC

SEQ ID NO: 55 - protein, 473 aa, Nicotiana tabacum
MASRIVLQQQNRGEAVPGAVKQKKNMAPEGRNRKALGDIGNVATGRGLEGKKPLPQKPVAVKVKGANVAKVPAARKPAQKKATVKPNPEDIIEISPDTQEKLKEKMQRKKADKDSLKQKATLTSTLTARSKAACGLSKKPKEQVVDIDAADVNNELAVVEYVEDIYSFYKLAENETRVHDYMDSQPEINDRMRAVLIDWLVEVHQKFELNPETLYLTINIVDRYLAVKTTSRRELQLLGISAMLIASKYEEIWAPEVNDFVCISDKSYTHDQVLAMEKEILGQLEWYLTVPTPYVFLARFIKASLPDSEIENMVYFLAELGLMNYATIIYCPSMIAASAVYAARHTLNRTPFWNETLKLHTGFSESQLIECARLLVSYQSAAATHKLKVIYKKYSSPERGVVSLLTPAKSLLAASSSSVLSEQADLRKSTEAAATSSSKMVVVGCQRCHMYVMVTEADPRCPQCKSTTTRKMT

SEQ ID NO: 56 - protein, 535 aa, Nicotiana tabacum
MSRACSLVQEYYDCCFLFIQSSRIVVERNAVVAQIIGISPPGSMRNANMTIGSSNLKEPTMRITRSRAKALGSSGGLPPRHPSVRQDNKQGLGAQGTKYKRSASDENNPVTNASTACQQPKRRAVLRDVTNVLCENSYMNCINRSKFQVKKFSDTRNSKVTPAILVKRPHNEDRKENTIEEAKKVKIEKSQEHCSQARFKDLTLTHPSKYITPAQCGFVDLMPVNRSLPTAIAVPNTTEKDETKVCQKQEGSDSLGIADIDSKHKDPLMCSLYAPDIYSNLHAMELDRRPSFNYMEKLQRDVNKGMRGILIDWLVEVSEEYRLVPDTLYLTVHLIDRFLSENYIEKQKLQLLGVTCMLIASKYEEICAPRVEEFCFITDNTYSKEEVVRMESLVLNFLGFQLAAPTTKKFLRRFVQAAQASYEVPSVELEFMANYLAELTLVDYSFLKFLPSITAASAVFLAKWTLDQSNHPWNPTLEHYTSYTALELKTTVLLLQDLQLNTSGSTLNAIREKYRQPKFKSVATLSSPEPVQSLF

SEQ ID NO: 57 - protein, 730 aa, Nicotiana tabacum
MGKLNSQKHISTIKDGVTELKVYEEADKIKIQSRDSLSRRCKGMSGAPNMSDVQTSRKSSESDIKHIERIKAKCSTSVKVNVKRKVLTDISNIRGNSSRTKSYNSSKLLVSNGKCPKNASNSARKFIMGNVRPNLNGATGDKQILTRAPFKDTKASFDGRKTRIQGRKSVTTGIRPTGRNDLPPSRRSLPILQQVNIEGTDNKEKGKVRANLNKATDDKQILTQAPRKDMKASFDGPKTRIQVRKSVTTGIRRTGRNALPPSRRSLPILQQVNVEDTNNKEKVNSKKLEKGKGISGVSVLAKPKAAGDVLPQLSNHSNIRRNRVGDASARMAPRGQAKVEVGALRRKSVRTVLKITASSLNSQKCSKSNSMSGVHKCTSRVSIPCKRLVDVRTSSLSKYATSEISAEQPHQKEVPSSSSGSLATPELSIARRKSDRRKSFTCLLMARSKLMKELCGNVELDNLSNIYDSCNHLEVTEYVDDIYQYYWVIEAQNQPIKNYMETQKEITPQMRGILINWLIEVHLKFDLMQETLFLMVTLLDYYLSLARVKKNDLQLVGLTSLLLASKYEDLFHPRVMDLLSISAESYTRDQMLEMEKDILRKLKFRLNAATPYVFMLRLLKAAQADTRFEHLAFYLIELCLVEYEALNYKPSMLCASAIYVARCTMQMTPAWTPLLGIHGRYQESQFRHCAEMILRFHKAASTALLKVTHEKYMQSSNSKVAAIKPLQSLP

SEQ ID NO: 58 - protein, 441 aa, Nicotiana tabacum
MVGSDENFSGVMRASNLQGGLRPGVGGGKLTAGVGQNRRALSTINRNVIGAPPLPYAVNKRNGISDNKANAANKIPSVPIHRPITRKLAAQIASKQQQPAVEVTKPPVPVAPNRNGSEDCIIIDAEEYKAAGDSSVPMFVQHTEAMMEEIDRMDEEIEMEDVEDWPIVDIDSADKKNTLAVVEYIDDIYAYYKKTEVLSCVPPNYMEQQIDVNERMRAILIDWLIEVHYKFELMEETLYLTVNLIDRFLAVQSVIRKKLQLVGITAMLLACKYEEVSVPVVEDLILISDKAYTRKEVLDMEKLMVNTLQFNVTVPTAYVFIRRFLKAALSDKKVELMSFFLIELCLVEYEMLKFPPSMLAAASIFTAQCTLGVSKEWNKTCEKHSSYAKNQLLECSRLMVSFHQKAASGKLTGVHRKYSTCKYGYAARCEPASFLLEAAWF

SEQ ID NO: 59 - protein, 483 aa, Nicotiana tabacum
MATTQNRRSSVSSATAKRQAMTANSSLENNNHGKLVAKKRPALTNISNHTTASARNSLSHSSKLAPCTSKAVSIKKSNSNAASSVLPTSSFVKPISKTVSIPRSDAAIPKITAIPLPATCSMDISPSHSDGSLVSMDETMSTSDSLRSPDVEYIDDNQTAAFDSIEKKAFSTLYISEDVKAADICKRDVLVDIESGDKIANIDNNFVDPQLCATMACDIYKHLRATEVKKRPSTDFMEKVQKDINASMRAILIDWLVEVAEEYRLVPDTLYLTVNYIDRYLSGNLMDRQRLQLLGVACMMIASKYEEICAPQVEEFCYITDNTYFKEEVLQMESTVLNYLKFEMTAPTAKCFLRRFVRAAQGLNEVLSLQLEHLASYIAELSLLEYNMLCYAPSVIAASAIFLAKYILLPSKKPWNSTLRHYTLYQPSDLRDCVVALHSLCCNNNNSSLPAIREKYSQHKYKFVAKKYCPPTIPVEFFQNISC

SEQ ID NO: 60 - protein, 412 aa, Nicotiana tabacum
MGVSNENNPTMIKPTNVQGEAELGCRKFGMETRNNRRALSVINQNFVGAKPYPCVVNKRVLSEANGICNKNPPVPAHRPITRKFAAQIANSKQHYPEENKKPKIAAEGLSVYEDVPIIDVEEYEAAAKDQPVPMSLEQTQMEIEMEDIFEESVIDIDSNDAKNTLAVVDYVEDLYAYYSKMEGCNRIPPDYIGQQFDINERMRSILIDWLIEVHHKFDLREETLFLTVNLIDRFLEKQSVVRKKLQLVGLVAMLLACKYEEVSLPVVDDLVVISDKAYTRKEVLEMEKLMLNTLQFNMSLPTPYVFMRRFLKAAQSDRKLELLSFFLIELCLVEYEMLKFPPSFIAAAAIYTAQCTLYGVKQWSKTCELHTKYSADQLLECSRLIVEFHQKAATGKLTGDLYSKQNPENKEE

SEQ ID NO: 61 - protein, 439 aa, Nicotiana tabacum
MVGSDENCQGVIMASNVQGAGGGKVTMGHNRRALSTINGNIVEAPAYPCKVHKRNGITDKSANGVKNPPIPIHRPVTSMGDSCCFRKFAAQMATKLQQPTVTKQPVQTATDRNESEDRIIIDVEDYKATSDYDPVPMFVQHTEAMMEEIDRMDAETEMEDVEETLIVDIDSADKKNPLAVVEYIDDMHAYYKKTESSSCAPPNYMEQQFDINERMRAILIDWLIEVHYKFDLMEETLYLTVNLIDRFLAVQQVIRKKLQLVGVTAMLLACKYEEVSVPVVEDLILISDKAYTRKEVLEMEKLMINTLQFNLSVPTAYVFMMRFLKAAQSDKKVELLSFFMTELCLVEYEMLRFPPSMLAAAAIFTAQCALSAPNELSKTCEKYSHYTQDQLLECSRLMVSFHQKAAIGKLTGVYRKYSISKYGFVAKCPPASFLLEACF

SEQ ID NO: 62 - protein, 367 aa, Nicotiana tabacum
MADQENCVRVTRLAKKRAAEAMVQHLQQPNKKRVVLGEIRNLSNQIQMFDSEPLKPKCNKQTTKRKVKRSVSVKEREFREEDVDSKLDDDPQMCSAYVSDIYEYLHQMEIEKKRRPLSDYLEKVQKDVTANMRGVLVDWLVEVAEEYKLLSDTLYLAVAYIDRYLSIKVIPRQRLQLLGVSSMLIASKYEEIKPPRVEDFCYITDNTYTKKDVVKMEADVLQSLKFEMGNPTTKTFLRRFTRVAQEDCKNSNLKLEFLGCYLAELSLLDYNCVKFLPSLVAAAVIFLSRFTLQPKLHPWSVGLEQNSGYRAADLKECVLIIHDLQLSRRGGSLVAARNKYKQHKFKYVSTLSSPLEIPDSFFEDTRQ

SEQ ID NO: 63 - protein, 482 aa, Nicotiana tabacum
MATTQNRRNSVSSAVAKRQAMAENNHGKLPAGGAKKRPALTNISNHTTASARNSLSHSSKLAPCTSKVVSIKKNNSNAASSVLPTSSSFVKPISKTVSLPRSDAAVPKITAIPPLPSTCSMDISPSHSDGSLVSMDETMSTSNSLRSPDVEYIDDNQTAAFDSIEKKAFSTLYISEDVKAADICKRDVLVDMESGDKIANIDNNLVDPQLCATMACDIYKHLRATEVKKRPSTDFMEKVQKDINASMRAILIDWLVEVAEEYRLVPDTLYLTVNYIDRYLSGNLMDRQRLQLLGVACMMIASKYEEICAPQVEEFCYITDNTYFKEEVLQMESTVLNYLKFEMTAPTAKCFLRRFVRAAQGLNEVLSLQLEHLASYIAELSLLEYNMLCYAPSVIAASAIFLAKYILLPSKKPWNSTLRHYTLYQPSDLRDCVMALHSLCCNNNNSSLPAIREKYSQHKYKFVAKKYCPPTIPVEFFQNISC

SEQ ID NO: 64 - protein, 384 aa, Nicotiana tabacum
MKKQEKEAIMADLENCGRVTRLAKKRAAEAMASHQQQHPSKKRVVLGEIQNFSNLGVSQIKGLNTEPKKQPKSKQQQSKRKLKRAVTSKIDKEELNVDNVDANYDDPQMCSAYVSDIYDYLRKMEIEEKRRPLPDYLEKVQKDLSPNMRGVLVDWLVEVAEEYKLLSDTLYLAVSYIDRFLSTNVITRQKLQLLGVSSMLISAKYEEISPPHVEDFCYITDNTYTKEEVVKMEADVLKTLNFEMGNPTVKTFLRRFTGVAQEDYKTPNLQLEFLGYYLAELSILDYSCVKYVPSLLAAAVVFLSRFTLQPNTHPWSLALQQYSGYKAADLKECILILHDLQLSRRGGSLAAVRDKYKQHKFKCVSSLTSPVEIPASFFEDMRQL

SEQ ID NO: 65 - protein, 441 aa, Nicotiana tabacum
MVGSDENFSGVMRASNLQGGLRPVVGGGKLTAGVGQNRRALSTINRNVIGAPPLPYAVNKRNGISDNKANAANKIPPVPIHRPITRKLAAQIASKQQQPAVEVTKPPVPLAPNRNESEDCIIIDAEEYKATGDSSVPMFVQHTEAMMEEIDRMDEEIEMEDVEDCPIVDIDSTDKKNTLAVVEYIDDIYAYYKKTEVLSCVPPNYMEQQIDVNERMRAILIDWLIEVHYKFELMEETLYLTVNLIDRFLAVQSVIRKKLQLVGITALLLACKYEEVSVPVVEDLILISDKAYTRNEVLVMEKLMVNTLQFNVTVPTAYVFMRRFLKAAQSDKKVELMSFFLIELCLVEYEMLKFPPSMLAAAAIFTAQCTLGVSKEWNKTCEKHSSYAKDQLSECSRLMVSFHQKAASGKLTGVHRKYSTSKYGYAARCEPASFLLEAAWF

SEQ ID NO: 66 - protein, 404 aa, Nicotiana tabacum
MKGGEAEMGNNKFDFGVETRHNRRALRVINQNLLGPNPYRCVVNKRGLSHANGIIYDKNPTRKLTAPIASSHQHYPEETKKPKLAAEDFRIWEEHVAAKDQLMPMSLEQEATFSNDKTEMEIQMEDIFEEALIDIDSDDAKNPLAVVDYVNDLYPNYRKMEGYSCVSPNYMTQQFDINERMRAILVDWLIEVHHKFELREETLFLTVNSIDRFLEKQTVARKKLQLVGLVAMLLACKYEEVSVPVVDDLVIISDNAYRRKEVLEMETLMLNTLQFNMSVPTAYVFMRRFLKAAQADKKLEVLSFFLIELCLVEYEMLKFPPSFMAAAVVYTAQCTLYGVKQWNKTCEWHTSYSEDQLLECSRSIVSYHRKAATGKLTGVHRKYSTSKYGYAAKYEPALFLVQIQ

SEQ ID NO: 67 - protein, 437 aa, Nicotiana tabacum
MAISDENNPTMIKPTNVQGGAGMGTRKFGGVETRNNRRALGVINQNLVGGAHPFPCVVNKRGLSEANGRCDKNLPIPAHRPITRKFAAQIASSHQHRSEENKKAKIASEEFSIWEDIPLTDVEENEAAKDQPVPMSLELTETVPNDNKNQMEVEMEDILEENIIDIDGDDAKNPLAVVEYVQDLFASYRKMEGCSCVSPDYMAQQFDINERMRSILIDWLIEVHHKFELREETLFLTVNLIDRFLEKQGVVRKKLQLVGLVAMLLACKYEEVSVPLVEDLVFISDKAYSRKEILEMERMMLNTLQFNMSVPTAYVFMRRYLKAAQSDRKLELLSFFLVELCLVEYAMLKFPPSFIAAAAIYTAQTTLYGVQQWSKTCEWHTSYSEDQLMECSRSIVSYHQKAATGKLTGVHRKYSTSKFGYAAKCEPAHFLVQTQQQ

SEQ ID NO: 68 - protein, 726 aa, Nicotiana tabacum
MGKLNSQKHISTIKDGVTELKVYEEVDKIKIQSRDSLSRRCKGKSGAPNMSDVQTSWKSSERGIKHIERIKAKCRTCVKVNVKRKVLTDISNIRGNSSRTKSYNSSKLLVSDGKCPKNASNSARRFIMGNVRTNLNGATGDKQILTRDMKASFDGPKTRIQGRKSVTTGIRPTGRNDLPPSRRSLPILQQVNIEGTNNKEKGKVRANLNKATDDKQILTQAPRKDMKASFDGPKTRIQVRKPVTTGIRRTGRNALPPSRRSLPILQQVNVEDTNNKEKENSKKLEKGKGISGVSVLAKPKAAGDVLPQLSNHSNIRRNRVGDASARMAPRGQAKVEVGALRRKSVRTVLKITASGLNSQKSSKSNSMSGVHKCTSRFASPCKRLVDVRTSSLSKSATSEISAEQPHQKEVPSSSSGSLATPELSIARKKSDRRKSFTCLLMARSKLMKELCGTVELDNLSNIYDSCNHLEVTEYVDDIYQYYWVIEAQNQPIKNYMETQKEITPQMRGILINWLIEVHLKFDLMQETLFLMVTLLDYYLTLARVKKNDLQLVGLTSLLLASKYEDLFHPRVMDLLSISAESYTRDQMLEMEKDILRKLKFRLNAATPYVFMLRLLKAAQADTRIEHLAFYLIELCLVEYEALNYKPSMLCASAIYVARCTMQMTPAWTPLLGMHARYQESQLRHCAEMILRFHKAASTALLKVTHEKYMQSSNSKVAAIKPLQSLP

SEQ ID NO: 69 - protein, 425 aa, Nicotiana tabacum
MDNNSVGVPHNLPRGEMGGKQKNAQADGRNRRALGDIGNLVPAPAAEGKPKAAQISRPVTRSFCAQLLANAQEEKNKKPLAEVVNKDVPAKKKASDKEMKTVGGSPLSKRKAKKSGKTLTSTLTARSKAACGLSNRPKYEIEDIDVADADNHLAAVEYVEDIYNFYKLTEGESRVDDDYMNFQPDLNHKMRAILVDWLIEVHRKFELMPESLYLTITILDRFLSLKTVPRKELQLVGISSMLIACKYEEIWAPEVNDFIHISDNAYAREQILQMEKAILGKLEWYLTVPTPYVFLVRYIKAATPSDNQEMENMTFFFAELGLMNYKITISYRPSMLAASSVYAARSTLNKTPLWTQTLQHHTGYSEDQLMECAKILVSYHLDAAESKLKAIYRKFSSPDRGAVAFFPPARNLLPTTTTDAASSSS

SEQ ID NO: 70 - protein, 436 aa, Nicotiana tabacum
MVGSNENCQGVIMASNVQGGLGAGGGKVTMGPNRRALSTINGNIVEAPAYPCKVHKRNGITDKSANGVKNPPIPIHRPITRKFAAQMATKQQQPTVEVTKQPVQTAPAKNESEDCIIIDAEDYKATSDYDPVPMFVQHTEAMMEEIDRMDAEMEMEDVEETLIVDIDSADKKNPLAVAEYIDDMHAYYKKTESSSCAPPNYMEQQFDINERMRAILIDWLIEVHYKFDLMEETLYLTVNLIDRFLAVQQVIRKKLQLVGVTAMLLACKYEEVSVPVVEDLILISDKAYTRKEVLEMEKLMINTLQFNLPVPTAYVFMMRFLKAAQSDKKVELLSFFMTELCLVEYEMLRFPPSMLAAAAIFTAQCTLGVLNEWSKTCEKYSHYTRDQLLECSRLMVSFHQNAATGKLAGVHRKYSISKYGFVAKCPPASFLLEASF

SEQ ID NO: 71 - protein, 422 aa, Nicotiana tabacum
MGVSNENNPSMIKPRNVQGGAELGYRKFGVETRNNRRALSVINQNFVGAKPYPCVVNKRGLSDTNKNPPVPAHRPITRKFAAQIANSKQHYPEENKKPKIAAEGLSVYEDVAIVDVEEYEAAAKDQPVPMSLEQTQMEIEMEDTFEESVIDIDSNDAKNPLAVVDYVEDLYAYYSKMEGCSRISPDYIGQQFDINERMRSILIDWLIEVHHKFDLKEETLFLTVNLIDRFLEKQSVVRKKLQLVGLVAMLLACKYEEVSLPVVDDLVVISDKAYTRKEVLEMEKLMLNTLQFNMSVPTPYVFMRRFLKAAQSDKKLELLSFFLIELCLVEYEMLKFPPSFIAAAAIYTAQCTFYGVKQWSKTCELHTKYSEDQLLECSRLITGFHQKAATGKLTGVHRKYNTSKFGYVAKCEPAHFLLVQTR

SEQ ID NO: 72 - protein, 492 aa, Nicotiana tabacum
MRNANMTIGSSNLKEPTMRITRSRAKALGSSGGLPPRHPSVRQDNKQGLGAKGTKYKRSASDENNPVTNASTACQQPKRRAVLRDVTNVLCENSYMNCINRSKFQVKKFSDKRNSKVTPAILAKRPHHEDTKENTIEEAKKVKIEKSQEHCSQARFKDHTLTQPSKYITPAQCGFVDLMPVNRSLPTAIAVLNTTEKDETKVCQKQEGSDSLGIADIDSKHKDPLMCSLYAPDIYSNLHAMELDRRPSFNYMEKLQRDVNKGMRGILIDWLVEVSEEYRLVPDTLYLTVHLIDRFLSENYIEKQKLQLLGVTCMLIASKYEEICAPRVEEFCFITDNTYSKEEVVRMESLVLNFLGFQLAAPTTKKFLRRFVQAAQASYEVPSVELEFMANYLAELTLVDYSFLKFLPSITAASAVFLAKWTLDQSNHPWNPTLEHYTRYTALELKTIVLLLQDLQLNTSGSTLNAIREKYRQPKFKSVATLSSPQPVQSLF

SEQ ID NO: 73 - protein, 422 aa, Nicotiana tabacum
MDNKTVVVPHNLPKGEMGGKQKNGQADGRNRRALGDIGNLVPAPAVEGKPKAAQISRPVTRSFCAQLLANAQAEKNKKALAEIVNKDAPAKKKASDKEIKTVEGSSLSKRKAKKSGKTLTSTLTARSKAACGLSNRPKYEIDDIDVVDADNHLAAVEYVEDIYNFYKLTEGESRVDDYMNFQPDLNHKMRAILVDWLIEVHRKFELMPESLYLAINILDRFLSLKTVPRKELQLVGISSMLIACKYEEIWAPEVNDFIHISDNAYAREQILQMEKAILGKLEWYLTVPTPYVFLVRYIKAATPSDNQEMENMTFFFAELGLMNYKTTISYCPSMLAASSVYAARSTLNKTPLWTQTLQHHTGYSEDQLMECAKQLVSYHLGAAESKLKAIYRKFSSPDRGAVAFFPPARNLLPTTTDAASCS

SEQ ID NO: 74 - protein, 495 aa, Nicotiana tabacum
MRHANIKHGSFHLEEHNMRITRARARASVLGSSGRLPPLHPSTKQDKKHVLGAESKRSKRSASDENRPGTSSTATGVQPKRRAVLKDMTNVLHENSHMNCINGSKIQVKKGSDKRNNKAKPAVSVKLSQLQEKGKEDIADKVKKVKVEGSQEISSGANCKEDMLPQLSRYVTPAQCGLVHLVPVNRSSCKAIPLQDIMKKDESKVCRKQEGFANLGVADIDSRHKDPLMCSLYAPDIYNNLHAIEFDRRPSVDYLEKLQLDINKGMRGILIDWLVEVSEEYRLVPDTLYLTVNLIDRFLSENYIEKQKLQLLGVTCMLIASKFEEICAPRVEEFCFITDNTYSKEEVIKMESRVLNLLSFQLASPTTKKFLRRFIQAAQASYKVPSVELEFMANYLAELTLVDYGFLKFLPSLTAASAVFLARWTLDQSNHPWNPTLEHYTRYKVSELRTTVFALQELQMNTSGCTLNAIREKYRQPKFKSVATLAASKPVQSLF

SEQ ID NO: 75 - protein, 446 aa, Nicotiana tabacum
MASRNVLQQQNIGEAVPGALKQKNMAAAAQGRNRKALGDIGNNVVTVRGVEGKPLPQRPITRSFCAQLLANAQAAAENQKKSLVVNGDAPIVAKGALAVKAAAKKPAHKKVAVKPKPDVIEISPGTEEQVKENKQKKKAGDDSSLKKATLTSTLTARSKAACGLSHKPKVQIVDIDAVDVNNELAVVEYVEDIYNFYKIAENESRIHDYMDSQLEITERMRAILIDWLIEVHHKFELSQETLYLTINIVDRYLAVTTTSRRELQLVGMSAMLIASKYEEIWAPEVNDFVCISDKAYSHEQVLGMEKRILGQLEWYLTVPTPYVFLVRFIKAAVSNAQMENMVYFLAELGLMNYATNIYCPSMIAASAVYVAQHTLNCTPFWNDTLKLHTGFSESQLLGCAKLLVSYHMEAPEHKLKVIYKKYSRPERGAVALQPPAKSLLAASLYE

SEQ ID NO: 76 - protein, 473 aa, Nicotiana tabacum
MASRIVLQQQNRGEAVPGALKQKKNVAAEGRNRKALGDIGNVATGRGVEGKKPLPQKPVAVIVKGANVAKVPAVRKPAQKKATVKPKPEEIIEISPDTQEKLKEKMQRKKADKDSLKQKATLTSTLTARSKAACGLSKKPKEQIVDIDAADVNNELAVVEYVEDIYSFYKLAENETRVHDYMDSQPEINDRMRAVLIDWLVEVHQKFELNPETLYLTINIVDRYLAVKSTSRRDLQLVGVSAMLIASKYEEIWAPEVNDFVCISDKSYTHDQVLTMEKEILGQLEWYLTVPTPYVFLARFIKASPPDSETENMVYFLAELGLMNYPTIIYCPSMIAASAVYAARHTLNRTPFWNETLKLHTGFSESQLIECARLLVSYQSAAATHKLKVIYKKYSSPERGVVALLTPAKSLLAASSLRVSSEQADLGKSTEAAATSSSPMVVVGCQRCHMYVMVTEADPRCPQCKNTTTRKMT

SEQ ID NO: 77 - protein, 588 aa, Nicotiana tabacum
MIQVKEESQTLDFGGFASCSSFSDSSYEASTPRYSSEPGSSYRRSSGPTKRSSQAGWTEEEDNLLTDVVKRFKGRNWKKIAECMNGRTDVQCLHRWQKVLNPELVKGPWSKEEDDLIIELVEKYGCKKWSFIAKSLPGRIGKQCRERWHNHLDPTIKRDAWTEQEESVLCHYHQIYGNKWAEIARFLPGRTDNAIKNHWNSSVKKRSNLNLPSGLVLDTESEESPNFSSDKKKLEIQKHPLQAQNAEQTIFLGEQTGLDNAAVALSTDLRIGYAYSAGNALHKDTSLFGACISAEENVRDLIKPLGGIPFGKADVLPIGETDKPCQSNLSRTKISYPLSASSSDFPLDQLHHTRWSTSQVEAVHPTTFGSMYESPKRSRHDTVNDPDHDFLSLSLASFTEVRSQSNKKNKAYDTQSSLGLKQQGSLYYEPPQLKDMLIPLTDENLSRDNLITEKNGHPFCSTPPSLKLTVSANGSSPESVLRNSAMSYTGTPSIIRKKNSRFPEAATHSSCTGTTTPTHNFPRASDREDTSNLKDRFSGCKSSVSGKSLGRRLEYAFDMEWDASRCCTPVSAASPCALRLGANTMLTP

SEQ ID NO: 78 - protein, 1043 aa, Nicotiana tabacum
MESDRISTPSDGTSSSLQRVRPLHGRTSGPTRRSTKGQWTTEEDEILRKAVQRFKGKNWKKIAECFKDRTDVQCLHRWQKVLNPELVKGPWSKEEDEVIVELVKKYGPKKWSTIAQHLPGRIGKQCRERWHNHLNPGINKEAWTQEEELTLIRAHQIYGNKWAELTKYLPGRTDNAIKNHWNSSVKKKLDSYLASGLLAQFPSLPNVNRQNQSIPSSTKLQQSSEDDSVRKEGIEMEEASECSQGSNLAGCSQSTSDMGNTFVHTREEGKLLEDSNYRKDPSSSSAPCSEYYTPAFEDITFSMAEVPSELDESKLLEHNFSHDWAASMGKEWQFNPDDIPNISPLELMQDSSGLFMQCLTGNGNHEMVTFPQQNAVKYEMTNVGSMVVGLDKPNEMFTSVEGCGMVYPEAGIPQYIPSETGMNGADETADSLICQSSNYQISEGGNMSIENCCNPLCSHVMGTSSGQPFSIPSQFSSEQSSLMFGTAANHFHNPSQGNPAQESHTSNSDGFLYPFESGTPCDNIMDDPLLEEQLDQTKDSLQLVSVNDFRSTPSNTIQTCPLVNENSSVPEEQKDGGALYYEPPRFPSLDIPFFSCDLIQSGADAQQEYSPLGIRQLMMTSVNCLTPFRLWDSPSRDGSPDAVLRSAAKTFTSTPSILKKRHRDLVSPLSEKRCEKKLGSDLRQESFSDLSKDFSRLDVMFDEAANEKATKSSLTMDQTLELQASSEDKENINPTEDGSKEEDKVRNGLSSERQLDGGEVHYKEKVTRKGTKGGANSAIGKIKQPSGVLVELNASDLFFSPDRFGAKSGRATNLCSKALGNQYARRLEAASNQGSVSSSFETSCFSVICSPRIRGKKDGSSFVITTSMQSAPAPTALDNSAETSGNGVGAETVSISGETPYKRSIESPSAWKSPWFINSFLSSPRLDNELNFEDLALFMSPGDRSYDAIGLMKQLSEQTAGAFADAQEVLGGETPESILRGRNSKNQKADENHSLLSANVMSERRTLDFSECGSPGKGKETEIFCTSNNSFASPSSYLLKGCR

SEQ ID NO: 79 - protein, 157 aa, Nicotiana tabacum
MGMKNDALLKSKARRKRVEKPQKSVGKINAEKAQERPVLFLAINNLTIGKSSNNSLTSPDVSSSCSSSIITFGENQKMNIEMENTVILESNPEIYQSDCLSIESLDQFDTSSFWFIYLMMPIVLFYERLIRICDLQRQIQNLIFMGSSSRLYCSLLF

SEQ ID NO: 80 - protein, 292 aa, Nicotiana tabacum
METDMSFLSKSSSSSSDEEIGLRRGPWTVEEDSLLVNYISQHGEGRWNMLAHRAGLKRTGKSCRLRWLNYLKPDVKRGNLTPQEQLLILELHFKLGNRWSKIAQYLPGRTDNEIKNYWRTRVQKQAKHLKIDSNSAAFQHMIRCIWIPRLLQKIQGSSAIPSIQTSQSTSLLDSQYGPLNITEITQTPQVLSLERNSISSSRCCSSRSPSSESMSIYKSPNIISECPKIPPREMGDSVVNVHFPFDDNSYDMDTFSPATGNFLTNYDQMVGGENNMMNGDILADSFWSMDQF

SEQ ID NO: 81 - protein, 245 aa, Nicotiana tabacum
MARTRCYDKSGLKKGTWTPDEDRKLAAYVSKYGCWNWRQLPKFAGLARCGKSCRLRWLNYLQPNIKRGNYTKEEDEIIMKLHAEIGNKWSVIAAHLPGRSDNDIKNHWHTSLKKRSTREYSTSTDSIKRSSNNSYQANSQKKRRENETQLNANESFQLSPMQSCSTEVSSCATIDQNVENIHGEREVFQEEIFEVSSGSFWTEPFLVDSFNTASDCFVPSFDDHGVFVSPFSPVMSYGELLCSYY

SEQ ID NO: 82 - protein, 235 aa, Nicotiana tabacum
MVRTPSVDKNGIKRGAWSEEEDNKLKAFVERFGHPNWRQLPKHAGLMRCGKSCRLRWMNYLRPGLKKGNYSLEEEELIIKLHKEHGNRWSVIAARLPGRSDNDVKNQWHAHLKKRAKTNTNNNSPIMEQFSESSQSGSQSEQYSHKVSEQEAGCDTASVNAVDTSVEVSSTDLYSSFSLLNGMDWIEEDHIRSMEQLPADFFNFCWTNPIDNFQTEPFDNFQTEPLDNFWRQPFF

SEQ ID NO: 83 - protein, 281 aa, Nicotiana tabacum
MVRAPCCEKMGLKKGPWTPEEDQILVSYIQTNGHGNWRALPKLAGLLRCGKSCRLRWTNYLRPDIKRGNFTREEEDSIIQLHEMLGNRWSAIAARLPGRTDNEIKNVWHTHLKKRLKNYQPPQNSKRHSKNNLDSKAPSTSQTFNNSDNFSNIQEDINGPVTGPNSPQRSSSEMSTVTVDSTAMTTITIDDQNMFKQLDEMDSSENFIPEIDESFWTDDLSTSDNSTFGMEGTGGELQVQFPFSSVKQESMDMVGAKLEDDMDFWYNVFIKSGDLLDLPEF

SEQ ID NO: 84 - protein, 333 aa, Nicotiana tabacum
MGRPPCCDKANVKRGPWTAEEDAKILAYVASHGIGNWTLVPQKAGLNRCGKSCRLRWTNYLRPDLKHDNFTPQEEECIIELHKTIGSRWSLIAKQLPGRTDNDVKNYWNTKLKKKLVNMGIDPVTHKPFAQVFAEYGKISGLPIQNARNHICLPNNTTEISKQLPFSLRENYSTQKYTWDPKAQYQVIHEETLQTHSFSEVSPLISSATYFNPTVFSSSSSYASVQSQVHTTASSSSTSTWNEFVFGDLCTSTDTEQKQEYQLQAGIYLSKDLSNSVHKDNPTCGEVTEVEENQSVEEATCSSAVDSFVDTILARDKQMLMDFPPLLDVYLDY

SEQ ID NO: 85 - protein, 998 aa, Nicotiana tabacum
MESDKTSTTPSDDISSLQRVQPLNGRTSGPKRRSSQWTPEEDEILRQAVQLFKGKSWKRIAECFKDRTDVQCLHRWQKVLDPELVKGSWTKEEDDKLIELVNRYGPKKWSTIAQELAGRIGKQCRERWHNHLNPSINKEPWTQEEELTLIRAHQVYGNKWAELAKVLHGRSDNAIKNHWHSSVKKKLDSYLASGLLAQFPALPNVNHQNQSVPSSSMTLQQNSEDESVHKQGTEAEDSFVKKKLDSYLASGLLGQFPALPNVNHQNQSVPSSSMTLQQNSEDESVHKEGTEAEEVPECSQGSTFAGCSQSTSDLGNTFVHVRENGGMSEESICKKDATSSTAPCCRNYNPVFQDVSCSMLKVPSELVDSKFLEHNLSHDWGNSMEEDWQFNRDDIPNISPPELIQESSGISVHCLNGNENHDMEATTNVGNVVEGPYNPNEMFVCVDGCMMVYPEEGIPQCSSETGVNGCGQPAYSLFYRSSNYQIPEVGDMVPQNCNALSFDDFEASSHQPFSVPLQFSSEDRSPVFDLVLNQFHNPPLESPDHMKDSSRIVPVNDLGSTTSNTVQTCLLNEKSFVQEKQKDGGGLCYDPPRFPSSDVPFFCCDLMQSGSDTQEEYSPFGIRQLMMTSANCLTPLRLWDSPSRDDSPDAILKSAAKTFTGTPSILKKRHRHLLSPLSEKRCEKRLESDLNQESFSNMTSNFSRLDDMFDESANEKASMEDGENLPSSEDGRKEEGEISGANDAMGKVKQPPGVLVELSSNDLFLSPDSFLIKCDRATSLSNKALGKQYARRLEAASNQVTVSSSFETSCFSVVCSPDIRGKRRSSVVLATSAALGNTAEDSENRFGTETLSISGETPYKRSFESPSAWKSPWFMNSFPPSTRYDIELAFEDLARFMSPGDRSYDAIGLMKQLSEQTAASIADAHQILGSETPETNLSKRNSKKQKADEICKASNATSERRTLDFNECGTPGKGKETTKFGSNNSFSSPSSYLLKYCR

SEQ ID NO: 86 - protein, 1015 aa, Nicotiana tabacum
MLDYPDLPLSLTMESDKTSTTPSDDISSLQRVQPSHGRTSGPKRRSSQWTPEEDEILRQAVQQFKGKSWKRIAECFKDRTDVQCLHRWQKVLDPELVKGSWTKEEDDKLIELVNRYGPKKWSTIAQELAGRIGKQCRERWHNHLNPAINKEPWTQEEELTLIRAHQVYGNKWAELAKVLHGRSDNAIKNHWHSSVKKKLDSYLASGLLAQFPALPNVNHQNQSVPSSSMTLQQNSEDESVHKEGTEAEDSSVKKKLDSYSASGLLGQFSALPNVNHQNQSVPSSSMTLQQNSEDESVHKEGMEAEEVPECSQGSNFAGCSQSTSDLGNTFVHIRENGGMSEESICKKDATSSTAPCCRNYSPVFQDVSCSMLKVPSELADSKFLEHNLSHDWGNSMEEDWQFNRDDIPNISPPEFIQESSGISVHCLTGNDNHDMVATANVGNVVEDPYKPNEMFVSVDGSMMVYPEEGIPQCSPSETGVNGCGQPSYSLFYQSSNYQIPEAGDMVPQNCNALNFDDFEASFHQPFSVPSQFSSEDRSSVFDIVLNQFHNPPLEGPDHMKDSSRIVPVNDIGSTTSNTVQTCLLNENSFVQEEQKDGGALCYDPPRFPSSDVPFFCCDLIQSGSDTQEEYSPFGIRQLMMTSANCLTPLRLWDSPSRDDSPDAILKSAAKTFTGTPSILKKRHRHLLSPLSEKRCEKKLESNLNQESFYNMSTNFSRPDDMFDESANEKASMEDKENLHPSSEDGRKEEGEISGANDATGMVKQHPGVLVELSSNDLFFSPDRFLIKCDRATSLSNKALGRQYARRLEAASNQVTVSSSFETSCLSVVCSPDICGKHRGSVVIATSTALENTAEDSENGFGAETLSIFGETPFKRSFESPSAWKSPWFMSSFPPSTRYDTELEFEDLALFMSPGDRSYDAIGLMKQLSEQTAPSIADAHQILGSETPETNLSKRNSKKPKADENCTLLASNATSERRTLDFNECGIPGKGKETTKFGSNNNSFSSPSSYLLKYCR

SEQ ID NO: 87 - protein, 1042 aa, Nicotiana tabacum
MESDRISTPSDGTSSSLQRVRPLHGRTSGPTRRSTKGQWTTEEDEILRKAVQRFKGKNWKKIAECFKDRTDVQCLHRWQKVLNPELVKGPWSKEEDEVIVELVKKYGPKKWSTIAQHLPGRIGKQCRERWHNHLNPGINKEAWTQEEELTLIRAHQIYGNKWAELTKYLPGRTDNAIKNHWNSSVKKKLDSYLASGLLAQFPALPNVNRQNQSIPSSAKLQQSSEDDSVRKEGTEMEEASECSQGSNLAGCSQSTSDMGNKFVHTREEGKLLEDSNYRKDPSSSSAPCSEYYTPAFEDITFSMAEVPSELDESKLLEHTFSHDWAASIGKEWQFNPDDIPNISPLELMQDSSGLFMQCLTGNGNHDMVTFPQQNAVKFETTNVGSMVVGFDKPNEMFTSVEGCRMVYPEAGIPQYIPSEAGTNGADETADSLICQSSNYQISEGGNMSIENCNPLCSDVMGTSSGQPFSIPSQFSSEQSSLMFGTAANQFHNPLQGNPAQESHTSNSDGFLYPFESGTPCDNIMDDPLLEEQLDQTKDSLQLVSVNDFRTTPSNTIQTCPLVNENSSIPVEQKDGGALYYEPPRFPSLDIPFFSCDLIQSGTDAQQEYSPLGIRQLMMTSVNCLTPFRLWDSPSRDGSTDAVLRSAAKTFTSTPSILKKRHRDLVSPLSEKRCEKKLGSDFRQESFSDLSKDFSRLDVMFDEAANEKATKSSLTTDQTLELEASSEDKENINPTEDGSKEEDKVRNGLSNERQLDGGEVHYKEKGTREGTKGGANSAIGKIKQPSGVLVELNASDLFFSPDRFGAKSGRATYLSSKALGNQYARRLEAASNQGSVSSSFETSCFSVICSPRIRGKKDGSSFIITTSMQSAPAPTALDNSAETSGNGVGAETVSISGETPYKRSIESPSAWKSPWFINSLLSSPRLDNELNFEDLALFMSPGDRSYDAIGLMKQLSEQTAGAFADAQEVLGGETPESILRGRNSKNQKADENHSLLSANVMSERRTLDFSECGSPGKGKETENFCTSNNSFSSPSSYLLKGCR

SEQ ID NO: 88 - protein, 317 aa, Nicotiana tabacum
MGRAPCCDKNNVKRGPWSPEEDSKLKSYIEQNGTGGNWIALPPKIGLNRCGKSCRLRWLNYLRPNIKHGGFSEEEDRIICSLYISIGSRWSIIAAQLPGRTDNDIKNYWNTRLKKKLFGKQRQKQGSRKGKEINSNMVISNNNNNNQFPCWPELPILQPIPYSNDEPRFNDHSSIRKLLIKLGGKFSDEDQPINEATNPQYPMDNSLLMQPIYQNIPINMISSAPIDNVLGNAQYNMDRAASSFTAELEHMIQNNQQKLDGLEFLYEDYMLIDKSASTSGGNLDWESMNPFVLPLPPINDEGFQQGVIFQENNTMAQ

SEQ ID NO: 89 - DNA, 2499 bp, Nicotiana tabacum
  source 1..2499: mol_type = unassigned DNA
tctaggagcaaaaaaaaaaaaaaaaaaaaaaacaagtagtagtagtagtagtaaatggaaaaatagagaggcaattttttttagtatcttactttatccattagcacttaaaaaacataggtttacatgctctcattgtcacgccaggccgctaattaaatggtacatttcatcatccccattttttggcctcatagttaattatcacaatttctgaaagcaacatcagaacaaccccacatttcttgtggtcctataattactgctagatagtccaaacccatctgcctatttagggcttgtgaatgaggattgaaaatggtagagaatatttgcaaaggcatcatgcatatatggaaaagactaaagagagagttagtgccttgcaaagcggattctcacagttgtagaaaaggactcaccattttcaagaatactcccggcatgaagatggacaatttaatataaaaaacaaagaaaatagtaattaagtgcgttgagatgaatgaaatttatccgctcttaattgaatatggggaattgaaagaaatttctgataataaattaatgagtcttacatgacgtggatccgacttaatctagtctatttaaactaagaatagataagaatagagaatatagacaaaagagaggctcattggctagggtttcaagggagttccttgaacataagtggcaagtacaagcacaaagccaatttccatggactaaagatgaataagatgtgtcgtgtggtatggtgggaaggtgaggaggtatggggtaattggagatgctaaacctctctaaaagctcttttgctccaaatatctaaatccatctctatcacttttggcgactgccccaaaatttgcaacttatgaattaaagttttaatatttttaagttaataaattctgaattaataatttaacatattcaataaactttttaaaacaaattacgtatataccatcaaactggctgcaccatgatcactttctaaactcacaatgacatatggatttaatcaggcacaaagtcatgttgatagaaagagatagtacggagaatgaagaaaaaaggtaggggagagagatggggtgagtggggaaaagatagggttctctttttagtgaaagcgacagggtctgagaaccctaggtcaaaagttgcataaacctctatacaggcttcttcactcccttactactaatatactctcattaaggcttgaggtttaattcattaaaattgtggtttaattattgtatcccctcaaacgaaataattgtccttgtcgaggttagacaatgttgcgtactattttcaaacgcagtcagccattattctcctatcctttacagtcgagattcaaagacagaaagtagcatgcaagctgttattaatttactttgattaggactttgccaagaaaatgaagaaccttttcttttttcttttaatttagttatcttacaacatgtaatttttcctagcaagcaaatacggtaacttttttttttattctcatttaatttgttggagctattgctactttgatgacttcaaccaaatcctggttggtaggcggagggtgctgacgatggaaactacccctcttgtccaaatacgataacctaaaaaatagaataatagcttattgtactgtgctgcaaaaattgcattgtcagtatacataattaaaatctattttgaatgtgtggagggcaaagaggggtgactggtctagggttgtagaaatcaggtgggagagagaatggtatttgtctctgtgtcagctgatatcacgtgaagaggcacaataagaagtccttcgtatccattcacttcccaaaaataccggcattactacaaatatagtactagcacttgctttctctatccccatctttgctatttcctttccctttccaactttttggctttagaattgcaaagatggagggaattgtggttctttgtatctgtaaaatttttcctccaagctccagttgtagctagcttaatgcgtggacgcgcgcgcacacactagaaatctgcaatctatatatatattcacaaggcactcacatatcaaaaaccacatagacattgtatagagagagctgtcgttctcaagcagaaaaaatgatatgatttcatcagcatgtggtcaaccaaatagttcaattctagtctttgcttcctctttctaattactgtataaatagagccacaaggacatagaattgagaaaataaaagacaataaaaacaaatctagctacttaagcgaatgatgatgactctctctcagtagtcttaactcttaatacccttgttttccttcttgtgctgcagtttgattggttaattaacctaatcaaaagatgttttaactgtgttttatccgtctttctcaagatctatcttagtcccaccacatagctccctcaagctacagctgcaaaatatatactatatatatatataacaa

SEQ ID NO: 90 - DNA, 2500 bp, Nicotiana tabacum
  source 1..2500: mol_type = unassigned DNA
tgacattgctttggtggtttaagttctcagccagtatatgcattgtcctaataggtctcacatggaagcagcactgagagttgtaagatacataaaagaagctcctggcttaggtctcttcttgcctgtaaaatcttcagatcaactaagtgctttttgtgactcagattggggagcatgtatacaaattagaaggttagttacagggtacttggtaaagtttgaaagtgctcttatatcctggaagtctaagaagcagagtactgtgtctaggagctctaatgaagctgagtttataagtatggcttcatgtgcagcagaagttacctggccggtaggactgttcagtgaacttggtgtcaaggttaaacttcctataaacttggtatgtgatagtaaagctgcaatccaaattgcagcaaatccaatcttccatgaaagaacaaaacatattgatatagactgtcactttgtaaaggaaaagctaagtctaaggatgctaaaaactgagtatgtcaacatgaggatcaactggcagatatacttacaaaaggattgtgaagagctcaacatgtacatttgctgaacaagctagggttgaagaatctgtatcaaccatcagcttgagagggagtgttaatcaacatggttaccactagtttatttataaagtgtaaatgctaaaccatagctagtgagttagttaatagttagttgagtttgttataaatattagtcagctgtacagtttaacatagcttctctttcagaaatgaaaattgctcttctctcatttcctctcttctagattcttcttctccctccttctcttagctcagatctctcttatgacagctaacaataaatacgaatatttcttgtaacggttgctcattgaatgttgtctttctcaaccgatatctttctttcaagttttccccccgattcgagtatttttgaaactcactcagcaccggtcacatattcgtaatcggtgccagctatttgcttactcatatcttatttgacttcattgtcacgtgtcagacagaagtatgtgcgcatataccatcaagtctcaatttgaaataaaatcaacttaagcagttaaaagtcaaatctcttttagttcggtctttaaaataataatttaaataatgaacctataaaacacgcaactcacactgaatataggggcagacataaaagccgaaagactgaattccgaaccggaccgaattatttcggtatttcgatatcggtttattcagtatttcggtactatttcggtataggatttttagttattcggtatttcggtacgatcctcggtattgaaatttcgatatttcggtataccgaaataccgaataatttaagtacaccttccttcactgcccagcccgttatcaattttcagcccaagtttctaacttgttatttctttcccttagccagtagcctactaagattaagcccaacgccccaacctaacattagaaattattataattagaaaagtataaagaaagtactcacattctactgctatgctcatgtagtgatttctattagaaattattagaagtgaaggtactgcccacattttcttgttgctatactcattatcacgcaattagaaattttctaatgaattagaattcagtagttcagcacagaggcggatgtagcgtattacctacgggttcaactgaacctataactttcgacacagagtaaaaatttatatgtaaaaattctttaaaattgtaaaaatcgtagatatgaacccataactttaaaaatataatgggtaacattaaaattgaacccatagaatttaaatcctggattcgcctctggttcagcattgtttagttcacaaaaatatggtacgatgccgaaccgtatcgaaaccataccgaaccaaacaagaagatatcgaacaataccgaactactttggtacagtatttggtatgcacacttgatatatcgaataccgaaataccgaaccgtaattttcgaataccgtaccgaaataccgaacactcacccataactaaacattaaaaagctagaactcaggtgtttaatgactaaacggaagtaagatctagataatccgtcactctgttgatttgtaaggctatcgacatgcaaaagtggaagcaaaatggagccgaaattttaacaaaaatgctgaaccaataccatgaaattgatgaatggtgggaccctatttcactcttttagaatttgcgtaagaccagaaaataacttcaatcgaaatcaaaataaataccaacccttttaggccccaaatcactacgtgtgatttgcaaacgtcattagccttatgtaaacagtgacctcatgccaacatattatcgcagcctataaatcttagtttacatttcattttctttcaaacacacacacctcacaatagaactaagttgtaagagtttcattttctttgttctttctcacaaaccaaa

SEQ ID NO: 91 - DNA, 2499 bp, Nicotiana tabacum
  source 1..2499: mol_type = unassigned DNA
tgccagacagtctaaaattcaaaaataggacagcccacatcccctccaccccactagatctcactacttcttaattaggacttgtgggggggagtagagggatttgcaaaggcggccgcggcatgcatatacggaaaagagaagttagtggtttgcaaagatgattgtggaaaagggcttacctaacaatgaagaagaggaagagggtagatggataataataactcaaaaatagaaagaagagggacaagtgggggctcaatggctaaggttttaagggaggtcttggaaacatcattagcaagtacatgcagtattaccaccctacatcagactgtggggtctgatggaatattcttctactatactcttttaaatagagggaattaatgagcctttataatgttgaaatttataagaaaatagtataactttttaaatctcttaattatgtatcaaatcaaatctcatatttaccctttagtggatctattcactgaagtctgaataaatcgtaccagtaattcataccgaatgtgtaaataatttgaaagaaagatagacaaggcctcaatgctagggatttgttagcatcaatagcaagtataagcagaaatatataccaccaaggagtgaggtggaagaattaagatttactctaatgaaatatatatatcaagattgagtcatgtgaatgaagaatttcttagtaccttaaattaagcgaatatcacataacagaggtgtaaaaaaacgaaagatggacaagctaagtggctctcaatggctggggttttaagggatcggaggtcttgttaacatcagacggaagtacaattagagtatatatgctactctaataatacttggctacaaacataaaaaaatatctctatcactatctctcaactcgccatatagacttaattggcacaaagtcatgctgatggaaagagatataaggagaatgggaagacaaaaaaaaaagtggggatagaaagagtgtccaagtagctagaagggggtgggggtggggggtgggggagttgttgttttagttgtggaagagataggtttctctttttagtgaaagtgacatatatagctagactgagaaccctggtcaaaagttgctttgccttaacgtttctaaatgcctgacctctgagaggctatcttctcctctcattctctcgacccttactgttcatatacccccaaatttgaggtctaatttatccacactatggtttcttactgttgtcttcttctctgaaacaatattgcttgtcgatcttggacttggccacgtcaacgtgtaacttcagcaactagggtgactccaagtcatagacagatctaggtcgacttctgtgaatttaactaaacaaattatttaatttcgactcaaaatagatatgtactatatatatatatagtaaacttattgtgaacttactaacttaaaattttcaagtttgcagtattttttggaataagaaaggggaaaaagaggcagaaaaccccatatttcttcctctttggagttgacgctaaagggataaagctaacatgcaagctcttaacaaataacatactcagtataatctcacaaatggggtatagagaggataaaacgtacacaaatcttaacaatatacacagtgtaattccataagtgagttctgtggacggtagtagcttaccccttgcctttaacatgcaagctcttaagctttgtatttttattttgttctttctttttggagaggaagaagtggtggttgaagactagagataaggaagaaaagagaaggatttttgtcagttgctatcacgtgaactgaaggggcacaattagagagaagtctatatgcttcacttcccataaaatcagttgtaactacaacaagtactaagagtgtcccctccattttctttctttccctcaattccctttcatacttttaaagcttaattccacagctagaaaaagaagcctttctttttctctagaggtatttagcaaagatggaaggacaatattacagctctctttgtctctacaggtaacaaaccattgcctgtctttctcaatctccagtatttccagctatcttataatgctttgagtactcccacaaaacacatgcattatagccactagctacatatatatatatatttgtaaaaccacacattaatttagctgtcattctcaaccaaaaagctatgttatcatcaacatattgacaaattacctataattccttcccctctagctatatgatctatctcactttattatgcacttaaaaagttatgttgtccctctcaaaagtcttaattaattaaccttgttttgcatcttgctgcagctagctagcttattaaattgacaaactcagaagatgttgtggttctttcaacttcaataaaaagctaagagtagtacttgtgcttgtatatccgtccttctcaagctcaagtcccacttca

SEQ ID NO: 92 - DNA, 3425 bp, Nicotiana tabacum
  source 1..3425: mol_type = unassigned DNA
gttggagctaaaaataagagaaatggacaagtgtcaaattctagcgtccatggtagcgcgacgcactattgtggcgctagaaatagaagaccaaaatttccaatgttggcgtcgaccttgcgttgcggtattttttctatttcgctagggacaagattatttcaacaaaaggggttctaaacctaatttggaggatatctaacatactttgaaggcgaattcacgtaagggaatacaaaccacgcttggaaggggctttcaactagtttttcttctcttcttttctcttcctttcatctcattatgtattagttctagggttgttggtacttacattaacgttatagtttgaagcttggattatcttattattttatcatattggtttatttattcaatcttgcgcttgataatttaattttaattgattgatcaccaattaaatactatctacgaatttaggattgaaatcgggagagaaaattttagattgcatataggattgagtagagtaagatcttgaacctgaattacgagggaacgaatttgcgattaggatataaggatatacctaatcgtcttgcttggttactatacgggaattattaatacgttcttattaatcctaatccactggaatataggcgttgagttagcttgaacaggcgagtagtacttcgggagaatactacgagtaatattaaattgtcaatcaataaactagataaatttataagatagtttaagtaaaaaactcaatgagattgttagttgacccataactctgaaatattttctcccattagattgtctttaagcttgccggcatagtttttctagttttctagtttacaactctagattagttatagttaacaatcacactttagaaaatcgcttgagtagattaattgttaatttagttgatagttaatcataagtcatcgagggaacgatactctacttatcactttattacttatcgaccacgtatacttgagtgcgtttgggagcaacaaatttttgacgccgttgccgggacgttagttgatgttttcaactagtgaacaaaatgcaaatatgttatgcaattcggtgccatttacacgtctatgaggagttatattaaagaactttatgtaggatgttggttggatcctacaagctaaaattatggctaagaaaattgtgaattactaataaacttgtaataagataaaaaataatttcctagaacgtaatagaattgaagtgagattagaccgacacctcttcgtcggaaaactatgttatatatgtaggttattttatatatatatatatatatatatataattttcttggcgtttaattttttatattttgattcttcctactaataattctaactctatcactgaattaaacgtacaggattcatttcttatacaaaagaggttgatcattattaggtctgggcactgtcagaggctgaccgatatgagtagttcttcacatgcttggcagcaatttgaactgtgattgcttgaggggcgaaaaagagagtagaatctaagttcggtaatttttatctgaattctgtatttgtcttaaaaatttattgagtatgcataaaattattattttaaactcagtaatttaaaaaatttagaattcgaactcataaatttcaaattgggactccacctctgattgtttgtaagtggagtttaggggcagaactagctcaaaaagttcgggttcgattgaactcagtaaatttgattcaaagtctatatatttattgaaaaatcaactaaatatgtatatatacaataaatttcaaattcataaaaatttaaatcctgaattaacctaatagtaaaaccgcagactctaactagtggtctagtttagagagtcaaattatggtttttaacaaccttaaacaagcacaaatacttttccactattggttcaattttggttgttaacaaccttgattggtaattacgtacttgcatgggcatttgaaaattaagttacgtacgtgtaaaacgttttagagtagtccgtactaattaagaacacaaacactgcttgagattttgtggcggaagtttgttttgacttagcatgggtaggcccacgaattccccattttgaataaaagacaacctgtgctagtcgattagctattatttaattactagaatattacttactccctcctttttaatttagacgatttagtttgacttggcacaaagtttaaagaaaaaaaaaagacttttgaaatatgtggtgttaaaatcttaatgggcaaaagctaagtggagtcatgatatttgtgtgactataaaaacttctcattaagaataaagtgagtaaaataaaaaattaaagtcaaattatttctaaatatagaaatatatcattcttttttgaacggactaatacggaaagtgtgtcatttaaattaaaataaataaagtaatatttatcatatgattttaacatgtaaatatcatacaagtaatctaatcgtcaagcgcggatctaataaataagggacgggtattttgtttaggctgtgtatatataattttttaaaatctactaaaaaagaacaaataatagatttgtaaattagagggatatggtagaatctaactataaacccttaaagttcaaatcttgtatctgcttgtggtaatagtgtatatatattttttacacgtttttgttgtatagaactcaaactaaaaagggcattccagtgcacaaagcatctcctattcacacacaattcggtgaagggccgcactgtatgcaaggggtgtgatatcggcagtctatcctgatgcaagcatcaatggttgattccacggctcgaatccgttacctataggtcatacggagataactttaccgttactccaagtcccccttctacataaaacttgcatcaatagctgatttcacgactcgaacccataacctagttgatacgaagataactttaccgttgcttcaaggtccgtctacacaaaactgatcaaattattttcataaataaagaagctatcatttctctataaatagaactagagtccttgcatattccaacataagtatcagttccaggaaaatcaagacataatctgttagcttttctctttgccattctcatggattccttaccagtctcctccattgaatctctagtcattgagatcaagaaagagatgttctcaaaccaagaatttaacacttttgtcaccccaatatctgcctatgacactgcttggttggccatgatttcttataataatcaagaagaagccattaatggtcattctttttctggccctatgtttaagagttgtttaaattggattctcaacaaccaaaatgagcaaggattttggggagaatccaatggt

SEQ ID NO: 93 - DNA, 2512 bp, Nicotiana tabacum
  source 1..2512: mol_type = unassigned DNA
aagctggtacccttatgtttagtccaagaaaaataaccatacccaaatataagggtttgttgaagacggaaatatataaacaaataaacaaatatcatcatatctccgatagtttaaaattttagattggatatttcacacaatttatcaaaattttcaaaaaaaaattctactttttcttgcttggaacttggaaggggaaggggtggtggtggagatagggcggggcatcttctatctagtctatgtgattaatataacaaaacaaaaagggcgaggcaaaaacatggatgaatggtggtccttttctatatttatatggattgttacgatacgtcgatttcactttgcaaaataccaattagattcatttagttatctttttgatcactctgcttttactatcatatatatataggagtccttccacgtttcgcatgtgtcattgtttatattttccatggtcttccttccaaatggctaaaaaaatttgacacagtggtcccaaaagtttatagaaatagaattcaacagtgaggcatatacctatgaattctattttacatcttcatcgtataaaatagaatgtgttataaactttacctcgtgatgcttacaaggggtgaaaatataaaagcactttatagatttacaagagtcacaccttgatttatcctaagattttatttttttacatgccaaacaatgaagtatgggagatccaattggaataacatcaaatttaataaaattcgaaatagtcagagagctgtctactgaggtatattgaaacttatttttttttaatagaaaatatcaaatacttagcaatatattaaaatgtttcataaattacattgtttaaaccaagcgttgaaacatatgctgatacgaggtaggcttattgatgaatttataagggcctcattggaaaagacgatccaaagcaatgggctaaaaaattggcccattttctgccacccagtgtatggttattactagtttcacccacacagatttgcacttcattagaggacaatgttgctgaatttgaaacataagtccatttatctccactgtacagtccttcctggagtccaatcctgaccatatcttcatgattttatgtaatgtggtgaataagcaaagtttcatgttatgctttgtctcattttatagcaaattcatttcctcataaaatttacttcaaaaaagtttcgtttgattttcagaaatcaaaatatgcttttcggtaaccaaatggttttcaattttgtttacgaagaacttaaaactttccaacaccctacatctatgattgcaagttaaaattgcagaaatatgacactttttggagtggtctttatcgtttaacttcacttgcactttaagggcaaaagttaaaagtgtttccatgaagcaagcgagggataacacttattaaacttgaaattctactcatagaccaaaacaaggacaaaaattcaagactatctatgtgggtaaacgtacgaaaattgggcttctccagattagagccggaccttgtggaaagacagagaaattcgaggcccacttccagtttctaaggagattaagcctatcaaacgatggtccagaacgaaatatgtctttctttattctctactatatagctgactcagaatcgttagaatttgcaatttcctcataataaaatgtgaggcagtatagattcgaaaacctttgttgaagattattgactcagctacgcgaaacaaactgtagtatccaatgtaccgattaacaagcgactggttaactatgaatttgttagctcgacaaaatcaccggttaataatgagtttgtgagttcgataaaatctaattttctgatagaaattttatatattatgcagaaatttaataaaagtagacttaacttatatattttagcattgactcttttgaagtaaaatccattccatctaaattatgacttccctacatcgagtaagtaagttgcgtctgtatcctcattttacccacttttcgctatgcaattattcaaggatctttacacaaatagcaagccaatattaattatttattttttttagtcatatatataaattatacatatattatatacccattaattatttttaatttaagtgatagattggacgactatttggattaattcttcgttattcaagataatagatgtcgtctctaatacatgagctagaagataataaggattactaggccgaaaggctgatggaaatgaacaagaagataagctcctaaatggaaacagtacggaaaaagtcaaagagcagtgcatgggaggaatcatcagtcagaaaaggaagccacgtgtcaagtagaaacaagcacgtgtccatgcaaaagccacgtaactcccttccatcacatcttccttcttcaaaacctcgtgttttactctctcttttctcactgccagtgatcgtcaggactgtgcatgtttgtttaaaaactaaaggca

SEQ ID NO: 94 - DNA, 941 bp, Nicotiana tabacum
  source 1..941: mol_type = unassigned DNA
tttaaaacgccaaaaaataagggacggccatggcgtagcaatgactattgttccgtaggtcatttgtgaaggaccgacaaaattttaggcgatccgggtccgatttttcggttctgctcaagaaaactcgatctgcacgaatagcttataatcaaacccttttttttttttcaagaatgtctaattgatcctaaaaagacaaccttatatgttccaccatggcaggatcggccttattactaagggtttgccaatagacacgtttttaacattcaagtaaaaaaaacatttattcttaataacccacctactatagtacgttatttggcagtagactacgttgtacatttgggaccatttggaacactcgtctttcacgagtcactaatttttgtgttgaatgcataaaatttgtttttttctttttcgaaattgaacaattttatcttcgatcacacctatagtatattattaccttattgttagaaaatattttattttattattgactcctaataaaaagtggggtaaatttgggtcttttttttaaagaatgtgaactactcatttcactttggttagaacaaatatgagaagatttgctaatgacagcaaaatgaatagaccaaaagcgtaacgaatattaaaaataaacaattccgaataactggttactgaaaattgtggaactctacatagccgttgtgagtatggtattgtttgttcttgtgggcagaataactagttacggaaaatttatgaatttgcttcacattatttttttcattttcttttttgcttcaaaagataagtacaagtttttatactcttatttcattgcctataaatacctctattgagttactgctcattcacaactctaaatagcaatctttcttattattaaaattcctatccttttttactcattcagagaaacg

SEQ ID NO: 95 - DNA, 5000 bp, Nicotiana tabacum
  source 1..5000: mol_type = unassigned DNA
aaaaatatgagaatcgtgcggaatttcagttttatggaactaaaacgccaaaaatggaggaacggtcatggagaggcgataactaatgttccttaggtttttttagatgggccgaaaaaattttaggcgatacgggtaaggcgtccgaaacacacgaaaatttgaaaatcgggcgaattcttgttttatgacactacaacaccaaaaagtatggaagattatggcgatgcgatgcgatgaataatgttccttaggttatttttgatgggtgacaacattttaggttatacgagaccgggttcccgggcccacaccagtggcgtgggctatattgcacacgaaaatatgggaatcgagcgaatttctacatttatggcactaaaatgcaaaacaaaaaggaacagtcatggcgaggcaatgactaatgttcctagggttatttagatgggccggaaaagttttaggtgatcctggtcgtggcgctcgagcccacgatggtagtgtgggcacacgaaaatctgggaaccgtgctgaattctagttttatggccttaaaacaccaaaaccaacaaagaggaattctcatggaggggcgatgaataatgttccttaggttgttttagatattccagaaagattttaggtgatccgggttgcggtgcctgggcccacacccagtggggtgggctatagcgcatgcaaatgtgggaatcgagcggaattctaattttatagccctaaaacaccaaataatgttgaacggtcatgacacgatgatgactaatgttccttaggttatttttgatgggctagcaaaatgttagaagattcgggtccggctgcccgggcccatgccagcacaagaaaatctgggaatcaggcaaattccatattatggcgattatggcgaggcgatgaataatattacttagattgttttttacaggctggcagaattttaggttatccaggtgcgggcgtctgggcctctgccggtggcgtgggctgtagtacacaaaaattagggaatcaggtagaattttagttttatggccctaaaataccaaaaaaggaggaaaagtcatggcgaggtgatgactaatgtgccttaggtaatattttatgttccgaaaaaaatttatgtgattcaggtccgggagctcgtgcccatgcagatggcgtgggctatagcacccgaaaatctcaaaatcatgtggaattccatttttatggccataaaatgctaaacataaggaacggtcatggagaggcgatggctaatgttcattgggtcgtatttgataggacggcaaaattataggcgattcaggtccggcgccgggcccatgcagatggcgtgggctaataatagcacacgaaaatctgagaatcgggtggaattcatgttttatggccctaaaacgcaaaaaacaaatgagaaacggtcatggcgaggcgatgacaaatgttccatgagtcatttttgatgggccagcaaaagtttaggcgattcggtttcgggggcccgggcccatgcagatggcctgggctattgcacacgaacaactaggaatctggtggaattccacttttacagcaataaaataccaaacaatgagaaacggttatggcgaggcgacgactagtgttccttaggtcggtttagatgtccggaaaaataataggcgatcgggtccaggcgtccgggcctgcgccaattgcgtgggccatagcatatgaaaatatgggaatcgggtgaaattcgagttttatggccctaaaatactaaaaaggaggacggtcatggcgaggcgatgattaatgttccttaggtcactttttatgggcagacataatttttggcgatctgtgtctgggagaccgaggccatgtaggtagcgtaagctatagcacacgacataattccaggtttatggacctaaaacatcaaaaatggaggaacggtcatggtgagccgattactaatattccttaggtcgttttggatgggccgaaaaaatttagggcgattcgggtgcggacgcccgagcacatacaaatggtgtgggctataccacacgaaaatctgattacctgacagaattccagttttatggccctaaaatgccaaaatcgaagaacggtcatggtgaggcgatgactaatgttcctaaggtcgttcttgatgggccgacaaaattttaggcgattcgggtgcggacgcccaagcacatgtagatggcgtgggctataccacacgtaaagcagggaatcgggcggaattcaagttttatggccctaaaacgccataaacgaagaacggttatggcgaggcgatgactaatgttccttaaatcatttttgatgggccgtcaaaattttaagcgattcgggtcagggcgccctggtccatgcagatggcgtagcacacgaaaatctaagaattgagcagaattctagttttatggccttaaaacgccaaggaacgaggaacggtcatgtgaaggctatgattaatgttccttaggtcgtttttaagggcaggcaaaactttaggctattcagttctggacgcccagacccatgtagatggcgtggctatagaacacgaaaatctaggaatcaggcggaattccagttttatggtcataaaatgctaaaaatgaggaactgtcatggagaggcgacaactaatattcgttgggtcatttttgatggtccagcaatattttagatgattcgggtttgggcgcccgggcccatgcagatggcatgtgctattgcatacacaaatctgtgaaccgggtggaattcaagttttatggccctaaaatacaaaaaaatagagaaacggtcatggcgatgcgatggatatgttccataaggcatttttgatgggatagcaaacttttaggtgattctggtccgggagctagggtctatgcagatggcgtgggctattgcacacgaaaatttaggtatcggtggaatttcagttttatggccataaaatgcataaaatgagaaacggttatagcgaggctatgaccagtgtgtccttaggtcgttttagatgggctggcaaaatgttaggcgatttgagtccgggtgcccgggcctgcgccaattgcgtgggtcatagcatacgaaaatataggaatcggacgaaattctagttttatggccctaaaatactaaaaaggaggaactgtcatgacgaggcgatgactaatgtttcttaggtcgtgtttgttaggccggcataattttaggcgatctgggtctggacgcccagggtcatgcaggtagcataggctgtagcacacgaaaattttgaaatcgggcgaaattctagttttatggccttaaaacgcgaaaaacaaaatgaatggtcatggagatgcgatgactaatgttccttaggtcgttcttgatgggacggcaaaatgttaggcgattcgggtgtgggcgcctgggcacatgcatatggcgtgggccatcatagcatatgaaaatatgggaatcgggcagaatttcagttttatggccctaaaacgccaaaaacgaaaaacggccatggcgaggtgatgactaatattccttaggtcatttctgataggccggcaaaattttagacgattcgggtaaggacgccctggcccatgcagatggcgtagcacacgaaaatctgggaatcgagaagaattccagttttatggccctaaaatgcgaaataacatggaacggtcatgtcaaggcgatgattaatttttcttaggttttttttgatgggcaggaaaaatattaggttatgcccatgcagatggcgtggctatagcacacgaaaatctgggaatcgggcggaattctagttttatggccctaaaacgccaaaatgtgaggaacggtcatgtcaaagcaatgattattattccttagttcatttttgattgtcggacaaaatgttaggcaattcgggtccgttcacccggatggatgcatatggtgtgggctagtacacgaaaatcttggaatcggacggaattccagttttatggctttaaaacgccaaaaaataagggacggccatggcgtagcaatgactattgttccgtaggtcatttgtgaaggaccgacaaaattttaggcgatccgggtccgatttttcggttctgctcaagaaaactcgatctgcacgaatagcttataatcaaacccttttttttttttcaagaatgtctaattgatcctaaaaagacaaccttatatgttccaccatggcaggatcggccttattactaagggtttgccaatagacacgtttttaacattcaagtaaaaaaaacatttattcttaataacccacctactatagtacgttatttggcagtagactacgttgtacatttgggaccatttggaacactcgtctttcacgagtcactaatttttgtgttgaatgcataaaatttgtttttttctttttcgaaattgaacaattttatcttcgatcacacctatagtatattattaccttattgttagaaaatattttattttattattgactcctaataaaaagtggggtaaatttgggtcttttttttaaagaatgtgaactactcatttcactttggttagaacaaatatgagaagatttgctaatgacagcaaaatgaatagaccaaaagcgtaacgaatattaaaaataaacaattccgaataactggttactgaaaattgtggaactctacatagccgttgtgagtatggtattgtttgttcttgtgggcagaataactagttacggaaaatttatgaatttgcttcacattatttttttcattttcttttttgcttcaaaagataagtacaagtttttatactcttatttcattgcctataaatacctctattgagttactgctcattcacaactctaaatagcaatctttcttattattaaaattcctatccttttttactcattcagagaaacg

SEQ ID NO: 96 - DNA, 5000 bp, Nicotiana tabacum
  source 1..5000: mol_type = unassigned DNA
gctgatctacaaagggcttagaaagaagtaagaaaagaaatggataaaggaaagggaaagattgcagaatcctcagagctgttgaggaagaggagatggaactggtccatcaagaaaggggtacatcagtggaggttcctaccccaagccaaaaagcccaagacttcctccaagaagtcttcctctgtgtctgaggctgttgaacctacactagccaagaggacaagatctgcagtgaaaacaacaatcaaaaatttctgaagatgatgactggagtggagaagaagaagaaaatgattctgagaaggaacaggatagcttgccagtttggcaaaagaaagattttaaagggtagactgctgaaagacctggtggaaccaggaatgatgagatgtggatgctttagctgctcaggatggaaggacatggtccttcagatggatggtaggctagccagaaatgagctaattgaatttatggcaatgtctggttaaggatggcatgttagtagcctggtgaaaggagttcaagtgcagtttgatgcaaagaaactgggagagatcctggacataccctctgaagggtttgatgactatacaaggcaaaggtggccctgcctggactccctccctactgctcttgtaattaccaggaggttttgtgattctgaagatgtgaatgaagccagggctgtgcagaaaagtgaaatgaggcctgagcacaaggacttgtttgaatttgtcaacaagtgtttattgcccagacaggagagaagtcacattgccaattacatggatctagttcttatagagtgcctggagagaggaaagcaaatcaattgtctgccttcattatcaagctgctcgacagggttataaatggctccaaggctcatgctactctttatggctttattctaaccacagttctggatagtctcaatgtgcctctaaagaaatgggaaatgatctcgagaaaggatcactttggcatcaatactcttcttgcttgtgactatgcagtcaatgacatcccaaatgaacctggtacatcctagaagacacccatcaacagcaaagtcaggactctggttcaggaatatgtagccaaggatgctgaaatagctaggcttttggctcgtgtgattgaagtgaaatttgagagggatggtctcagaactgagcttgacaaagaaaaggagaaaaatgatggaattcttcataacatgctgaaccttctccaagcccaaacccaaccatatagttcttccaagccttaggactcctagcttttgtctcctgaacttgtttagtacctcagtgacccagattagggatttttctatcttttatttttgctcatgatttggatgtttttctttctttttgtggattgttggtggcaacatatctctgtcaatgataactactattttgctcttgtttaatgttaatttgtccttgatattttaaatatattttcttgattactgatgattactccatgattacatttgcagttgccgcagtggccatgggtacttattaaaatctgggaatcacactttgtatgtaacatttcgatgatgccaaaagggagaagagagttgtgctttacacacattctgaaataagtaatatttataacctaattaacctggtccttgatgttaagtgaattttctaagtttagtattgatggttaagctgagttcttacaggtcccaaataagtaaaaagcacagagtttgtcatcatcaaaaaggggaatttgttggcccaagaacaggtgaagttttgaagatgacaaaagaactcagacatggaccaggtccatcttgtgaagcacagtcatgatcaacctatacatgtgagatgcacgtgaaagagataagtcttactgattaagcaacaatatctcttgatctgatcgaaaaggatgaagatagtgttagagtttgagatcatcatgaactcttccacgatagaagagcagcaattgagtcacaatcaaactctgattactaacctattaaatgtcagtttgttctcttttacaggaaatacacatacgcaaaagttaaactaaattgagagcaaaagagcaaggcgattttgcaagcaatttatgtggatttgagtgtgcactcctgaagctacttgaacgagatagaagaaccagttccatcgtgtctatctttttctagttcaattgtagtaggtggtttaaattatacctttcagctttcatagaagcaattgtattagatacctagagtgttcaagttatagctaacttgaagttgtcgcaacagttgaggttgtgtgccacaacgggattagagttaatccttaggtttataaagagtttttgtaaaagctattttggctcagtgattttagtggaagtttgggaaaatcctactgagttgtaggtcatggttttttcaccttttgagccaggtgttttccacgtaaaaatctccgtgttctttatttctgtatttattattccgcaattagtagtagttggaacacctagaaaaccaagttcttctatagagtagttaagcgaaaattgggtgccacacaaacacccctctagtgtggtattgacgcttaaacatcaattagttaatttctggagcaactagctactagttgttattaaaatagttagtttctttgttagctaatatgttgttttggttgtaaattagttccggtgtgtagtttggactttggaagaagacttgtaccaattgtgtttgttattttctttggtcttatgaatgctcacactaaacatcaagttggtatttgattttgcatttgaattagaagtagtattgagacgtgttgttgctatgcataagtagtaaaagattggtttgagcttagttggtttcgtgataagattggaaataaaagaaatgtgtcaaagttatgtaaaaatcagtaaaataggctgctaccttttaatattaccacaagtccacatttattttagttttaaacaatataaatttgttaaggtaatcttcttacaatagtctcaacttttaatttagtaacagataattgcaaagtcaatccaactaatcatacactacccatatgtaaaaaaaaaaaaaaaaaaaaaaaaaaaaaaaagtgaacaatcaatgcacaaaaagaaaaaaaaatatttttcttctttaattaattccataacatagtccttaaaattagttaattcttgttttagaaaaattgtaacagtctagttaattctccaaaatgaagcaaaagattttttttcttaagtattacgtcactttttttattaatccaccaaataaattaaattagatttagttcactaaataaactcaataagatcagatgttttatttattttaaaaataactcaattacttaatcacaaatgatcatgactaactcaaaagtaatttgttttaacaaaaataattaatttcgtcttaaccgatgtcgggacgactcattttggaataaatacataaataaaatggccaggtcgcgaggacacgtcatattccaattctttcaaataagcttgttatttattaacttgagtaggctccaattttaggtgcggcgcacgaactaaggtcggagatattcatcttggttagcgtaagctagggttggggatattcgtcctagtttgagattaattaagtcatcaacagtaaaagtggacataggcaaaacatgaaaaccgaataaagcacaatttatccataattaattcatgccaaatttaagttaataaagcaactgtgctagaaccacggactcggagaatgctttacacttctccccgatcaacaaaaatctttattcggactttatttttgcagaccgataataatagagtcaaatcttcctttgactagggattcaaataaaaagtgacttggaacatgcaaaaatcaattccaagcgggcgaatctgtaaacaaaaaatccttattcaaatttgtcactttaattgaaaaactctttaacccactattcataacatatatatttttggggtagaaaaggggttgacagttatgacctactttatgcatcagtgttcgaatttattttgatcaacacccttttggaagagcgtttgatagaaatggttggcttaataaacaatcatattatcatcacctgcggaatcatatcattaacttttgaaaattaaaatggttttcaaagacgttttgataaaagaattcctattgtcgcagttggaatctacaagaccaagatgttgatctagtgctatatttggagaaagtgccttaattaaaaaaaattgttcattagttgtcttaagattttttattatttaaaaaaaaattaagacacaaagaaacacatttacgagtatatgtcggccgactaatgtgaagttcccacggacaacccacacatattgtggtcaagatggattctatcataatcaaaagtcatcatcaattcaattctcatatttggcatctcaagtacatgcacaaaagcaacttaggatgtaagtttatatgcacattcttgaaatagaacctatttatacgtagtacttaattagttacagtagtattatttattctctgctacagagctatggtttatcaaatatatcagattatcatttgttgtgtaggccatttccttatttgtacttggtattaattctggcaaaagcacaaaactgggaaatgaggttcttcttccttaatatgagtcacagattagtaccactactatagccaagaaaatgtgaaatcatatagtactaaatattaatttcagatgccaaaaccataaatttcccctcctccatcattgaaaacccctctgtcctttcccctagagagacccctttttcctctctctctccttctctttttattagacgcatatattctctcttctttctctttctagggttttcacctgaaatagttttatttcggtgat

SEQ ID NO: 97 - DNA, 5000 bp, Nicotiana tabacum
  source 1..5000: mol_type = unassigned DNA
atttaaaaccctaaaaaacaagatttttatctatctgcatggtgaaggacaaagaggtcttcaatctcaggttcttttttttttttttttttatatatatatcttgtttgggtttaaggttattgggctgatgaatgttttaattttaacataggtctacttacgtagtagttataggttgataatgagatataattaactaagtctttgtataatgcagatcctgaacttaatctttatttgtattatttttttttgttactgaaagattctgttaccaaattttatcagtctatttaattagaggccaacgattgttaggtatgtggcacttcgagtgggaaatgatatattcccattaaaggtgttaattaaccaccaaattgttctttaggtctgtttgtcattttgtattaaggtggatggtttattatatatatcttctctttaatgctaatcatgcttaactttttcatttagtaccagcaagcatatttgtttactttattggttattccttatcaaagtcttcatcttgttgcttttttttattgtactttacaaaagatttctagtattaatggaaagtgctcatatttggaaaaagacatggccaacaagaaatgtctatataccccatttcttcttcttcttctttttttccgaaaatttcttatttttgtttttatttctgtttcttgttgagtgctttcatggtagaagaagaagtaggagattcttggacatggctgcatgagaattgttaaataaccccgtatacatacacaagtagtgttggctgtctttgatatcaaaccatttattgccctaatttctgccttttgtcccctcaacaaaaccatcaaagttctcaaagagggtttattcttgtttcccactttgccccccacctattagggccaccccaccaaagggatctctctcgtgtctagtgttttttcccaaggaccaccactcctttttttttctctaccataacttcgtccacaccatcttattgtgatattttcgtttaatgaatttgcagccatgccttcattcatcatcagaactcagtcataagcacagattctgagagagtaattaatgaatgaatcagtggtgatttgacgtaaagtatacatgattatggtttttagctgaataagcagagggagaaaatatatacatatataaacaagtagagtaaaagaatgacgcaagattagtaccaaaagagtgaaggaagagatttaatattatagggaaaagggaagtagtaggtgatacttgacaggttgataagatggttattactacaagttgatgtattgacgctaactcacgcaagagagacctactcactgtacaatatttttacaagaataagcgattctttctctctttacttgcaagaattgtgtgttgtgtgagttgtatggcgcattttctaggagcctgtggtagtgatggatgtattcatataatacaatacaatacatatggaatagatagatagataagatggtgcacgcatgagaggcaattatgcaacttacgtcaactacttccatccatccatcttttcttccttctgtttctgtctgatatagtgagtatatgcttgtgctggtgttgtgtgcttttctggcctgggattttcctaacactttagataatttaggttcccatcaataataatgtctttttagaggagcatcatcgatagatattcaaatattaaacctggcctagctactatctagggcgtctgctaggtttttccattaccctttgtatatctcttatgtgggaccttttgtttatggaagaatatggagtacttttattcatctcgtagggtcttgaatacaagattttatatatatcactctttaaaaatgaccatcctaaaattcttcctctttcatttgcatttaccagaattgatattagtacctaaactagtactcttcactgaggccttttgtatttagtcctattatatttgaatttggcactatttaaattaaaaaaataatctacaataaaaaattcttccctaaacattacccatcaaatactcaccacctaaggtaactctaccatgtattaattttttggatcaaatctagtgaggattaattctccacttatgttctttcggaactggctaagtaatcttcaaaagctagggcatctccgcagtcatatcgtgccctcccaagtatagcgaccgcttctatattttccctgaatttcatctgtgctagggcttgttttcacgttgatttcaaacaaaggctaacaatttcattgagtaactttttctcatttcaggactcgaaccttaaacctctgttcaaataacttctaagaagtatatatgtatacaatgtttgtattcattgtgacaaagtattatgagttgtacaactttcttgtgaagatagagcataatgttaaacaaggatctatatagagcataatgtcaacaaaacaacaacatcaacccagtaatcatcctactaataggatttggggagggtagagtgtacgtaaaccttacccatacaggggagggggtaaagaggttattttcgggagaccctcggctcagagacaaaaaaatctataataacaacagaaaccagacaaataatatcagcatcataagagacaacaaataagtggaatgacaataattatgccaataataacattgaaaaaaataaaattaaaaattaaaaataaaaatagtgtgatgaacaaaaatcgctagcagtcttagacaaaacactatcagactagctggaacaacgaggaaaaacgctgaagtaccccctaacctacaaccctaatgctcgacatccacacctccctatccagggtcatgtcctcggaaatctcaaatcgcgtcatgtcctgcctgatcacctcgccccattacttcttaggtcgccctctacctcttctcatacctgtcaaagccaaccgtcacacctcctaactggggcatctaggcttctcggggccggccagcccgtagatctaaaaggatccatccatagcgcccgaaccatccacgcctcgctttccgcatcttgtcgtccatgggagccacgcccaccttatccagccttatccagcctagtgtgcccccacatcatctcaacatcctcatttcgactactttcatcttctggatacaagaattcttgactggccaacattcagctccatacaacatggtcgatctaaccaccactctataaaacttgcctttaaatttcagtggcatgttagtatgtttactttagatacaatgttttttagagtcttatagtcttgttagaatactatatattataaaatatggagacttctgggcacttttgttttattttatataagataggattggaatgaattcaattggagggacatgcatgataaatgaatattcatgtagccgatatatgtttgggactgaaacgacattattattgtgaaatattttacaattgcatttcacactcactgaagtgaaactttgattccacgtcggtcaatacttaggtgttacggtttggctgcgaggggaatcgaagagagcaaattaattaaagtatttaatgaggaatcatgagttagttggtggaattataatagtcaaatgaatgagttattcgcctgataatatagttgatagtagtatatactatatatgttgatactagttattggtggtgacctaattaagtaaagagaagagaagagtggttatgtaaaggaatctaggtatagtgggggatggggggaggcaaggttaaaagaaaggtggaaaatccaagaatcctgcttcctctagtaacatagcatatcctgcaattcgtgcttttgtttcctctcacaagataactacttttttgattaattattacatttgacacatacatacaaacctataaaattagacatccttatggaatcttacgactccgaacttgtcatatatcctttaattatgcttagctttttgctaaaaacaaaaaggatatccttattccaaaatgcaactaggagcatcttcccacatttcttttttatgcctctgcatcatcaaatcccataatgccgcacaacaatttctttttacttaagtatattctagcttagctatttcatacgaataatgggtatacaaatttgcttattttaggttttaaataccgatttaaatatattggatgggttcaacttttaaaattcttacactgatatacatgcatagaatatgtggaaaactttaatattaattacaactgctaaacatttgaatggattcttcatgccgtgtgctcctttgttgaagaacacgtactccctccgttcgcatttatttggcacgttttgacttttcacgccccttaagaaataataaataaaatgcataatttatcatgataaacatatcaatttatgcatattttattagatttgagaaaataatttgaaatgagtaataaatactgtgagtataacaggaaatttttttttgtcttctcttgatatgcataaaatagcaagtaaaaataaaaatctatttttaatataaatgtcaagtaaaagtgaatggaggaagtatttggaagggcgatatcgaataaaaaagttatactaataacaaacagcaacaattacaagaaactgtagagttggccagtaccaggtatatatgtagaattttttttatgaggaatttagtgaaacgctagctatttcaacacttcagacatatcaataccaattttatggtttctcttaggtgttgatagattctcttttgtcagcaaacattatttatgaaatttataataaaatgctgctcttttcgagtttacattctccgtcccaatacaatatatacttgatttgacgaaagaaatgtaacaatagaatcttaagtcggaaaaagtcaaatcaaatttgaaaaaaat

SEQ ID NO: 98 - DNA, 5000 bp, Nicotiana tabacum
  source 1..5000: mol_type = unassigned DNA
gaaaactaaaaaaagaactaaggcatttacactaggtcttcaccggggctcgtcccatcaccagaacccgttggcctcaggtctcttggcttctttaatcttggccgacaggtcaaaacctcgggaatggatatcctcgagggtctctctctggtatagccgctttacgtactcggtcttcatctttaagcgggcctcagctatctccacatcagccttatactgggccagcatttcctcgacgtcggaggaatcaatagaagttgcctccaacttggatctcagcgcgttatattcttggtcgagggcatcccattccgtgacggctgagttcagctgtgctcggagttcatcatttagccgagatcacttgtcggccttgtcctttgccacccggagttggtcctttatcgatgtcaactcctcttttgcagcctctttttccgaagccagtaggtccatcctgcccttctacgcctcggtcgtggccttgacctcatccatctcagcccgaagccggtcgatcaggtctaccttctcttggacctgggaagttgcgttgttagtcaccacgagtagcttctcatttttagcctcaaagatccttaccttctcgaccaagtcagcgtgctcccacttcatggacaaggcctccttctgagccttttccagctcggctcagagaattgggaggtacttgagggcctcgtcttattgttcgctgagagacctgtaaatgtccttctcccggatcagttctttaagctcgaactcaagctgactgatttgcaagagagaccgaaggaagctttcatgatgaagcattcaaatctgaaatataataaggtcattagaatatgcggagcaaatattcgcaaaggatacaaaggatagacgtcttacccggttcaatgcctattgcacctcgttgaagaggctctacgtgcctacttcattcatctttgcctaatccttatcggtcaccaggcaacgaaggtagctggctatgccgaccggagcggacagggctcgggcgtccatcgggatagtaagaatgaccattctcttacggtcggggtcaacacccggggggagggaacttcttcactagtttcgggctcaagctcgagctcggcttgctcgctcccgatgacacatccttttttgggacctccaggtgaccaagcctggagaagtcctccaatgcgaccatgtccatgccatcaaagaacgtgttgagggcattatctggacccgacgctatctcatttaatttctctttattagcttgaacctcttcgagcatggactcagtataggacggcgtctctgcgatgtcaatgatactagctacatccttcgggacaggagcggcttctcgataggccatggactccgtctctccctctggttctcgagctagagggggatcgacctcttggtcacctttcctggttgccaccttctccctctcgttgagggtcgactcatgagcaatgaactcaaggtcgtcatcctcaggataatccctaagccggtagagggaatcagatttcggtaccctcgacttggtgctctttttgtttttctggacctggacgaccaccctcttcttcttcacctcgacatccggggagcctgtggatttccttttattcttctttttctcttcccttacggcggcctcgggctgtcccgaagcggagggttcagcgagcgaggacggatcctcgtcctcatccaatggcttgagctcggtggtcatgggtgaacctgtgagaagaaagaagacttagcgatatattcatcaagtatatgaatgtaatcattcgggagagagactcactatgggaacgagcctcccatttgcccttcgagagcttgcgccatacgctcgaagtaggacatctatttgcacatcccctcgattcactccttgaagcaggggaatgtattaggaacctgggcaactgctacataatggcaaatacaggcaattagaaaaaagaataaaaggaaatgccagatactcgagagggaaaagacttacgggatgcgtttcactttttgaggaatggtagaaattcggagaggatgaggtcttcggttttcacccgaacgaacctcccctaccaacctcgatctcggtcctcgtcgatgctcgagaacggagccttgcttgctcggcgaacgatcttaatcaacccctctcggaagattcgaggactgtatagatgaagtagatgttcgagggtgaactgaggtgcttcagttttgtttacaaagtgttggaggaggattacgatcctccaaaaggacaggtgaaggcagaccttgcaccttttatagatgtcgaggactatggggtccaccgggacgagcgtgaaggagtaagtgtaaacacttaggtacccctccacgtgagtggtgatgtctttgttgggcccggggacgaccacctcctttccctcccagttacactctacccgaactatgggtagtgtctcctcagtaacagagcatatgtacctccatgctccctcgtctcggtcctgttgactggaggctttctcgacgttgaagtcattctcgatagagcagcccccgagtacgaagctcttcaagggaggctcatgggctacctcaggaatggccacctcggcatttatggccagattggaaaataaaggagcggtttgttgaggaacgaatttgaaagtttttgctatcgggttctgaaaaatatgaaggtttgaagaaaaaatatgaagatttgaagatagaatggaaatatgaaggtctgggttgaagattgaaagagaatgtatgaagattgaggatgaaggtatgaaaatctaaggagcaatctatgaagatttgaaggagttaaaggtatgtaaagaattcaagggtaaatcaaggagctctagaatcgaaaagtggagaagtgaaaaaggggtcggagcttttatagaggaaggacaatcaatgcatgacgtttcacattcgaggacagtcaatcaacggccgatacgtgtccgatgttagaacgatgcgactaatgggacgtttcattgatccgtcatctcggttgtaacgtacgaagaaaggaatcggggttcatttatcgcttcccgtcgtttcgataaatctatcctccgaaaaacaaggggactatctgtatacgggtaaaatcaggcaatgtctaccctgattctcctataagagaataaagggggagcgcggatccgcgagattgtaatcgaggacagagacccatcgtatcaagatccaagaagagtgaacgatatatctaacatcagacacggcaaagcgatgtaccccggaccgaatataactcctagacctcgggagaagcgggggacggttatgcatgacagataggagactgtatactcgccctcaatcggatattacgacgcgaatctcgtcagtaacaattatggatcaataattactggaaaaagaagatttttaccttttttagacttatactaggactgaaattctcgtactatataaaggtaaagtttttctttgatctgacacattgtaacacgcaattcaaagaaataaaaatttgtttttgccttctaactaatgttaaaaattttgctcacttgttctgttcttcattcacgactggactcgaaccgagggtccaatcgagtacgaggtcactgttcaatctaagatcatgcttggtcataacattgcgattggtttgatcatttatttcgtctttaattcatttatctgttatttttaattattcgtgttgaattaaatcacgtatcatttaaaccgcgtacaaatttaattgttacccatttttaaggtaaacaactatagacgaaaaaaaaaatataaatattaaatattatgtttcgaaagatacacaatagacaagaaaagaaaagaaaaatcccttataaaatttggatttagcccaccagttttattgagacgtctttgtgtgttagttacccggcaaaggttatgaacctactttatgcgtcaatgtccgaatttatttttatcaacatccttttggaagagcttttgatagaaatggttggcttaattagcaatcatattatcatcacctgcgctttggtgttatatcattcggaatcatatcattaccttttgaaatttaaaatggttttcaaagacgtttcgataaaaaaattcctattgtcgcagttggaatctacaagacgaagatgttgatctagtgctatatttggagaaagtgccttaattaaaaataaaaaattgttgatcagttgtcttaagattttttattattaaaaaaaaaaaattaagatacaaagaaacacatttacgagtatatgtcggccgactaattaatgtgaagttccacacggtcaacccacacatattgtggtcaagatagattctatcataatcaaaagtcattatcgactcaattttcatatttggcatcttaagtacatgcacaaaagctacttaggatgtaagtttataatcattcattcttgaaatagaacctatttaatagtacttaattagttacagtagtataatttattctctgctaaagagctattgttcatcaaatatatcagattatcctttgtggtgtagaccatttccttatttgtacttagtattaattctggcaaaagcacaaaactgggaaatgaggttcttcttcattaatgttgagtcaagattagtactactactatagccaagaaaatgtgaaatcatatagtactaactttcccttctccctagctactgataactctaattaatttcagatgccaaaaccataaatttcccctcctccatcattgaaaacccctttgtcctttccccccagacccccttttcctctctctctctctcctttctctttttattagacgcatattctctcttctttctctttctagggttttcacctgaaatagttttatttcgttgat

SEQ ID NO: 99 - DNA, 5000 bp, Nicotiana tabacum
  source 1..5000: mol_type = unassigned DNA
attataagggaaattaaaaccctaaaaacaagattttatctatctgcatggtgaaggacaaagaggtcttcaatctcaggttctttttgtttttttaacttgtttggatatgaggttattgagctgatgaatgttttaattttaacataggcctacttacgtagtagttataggttgataatgatatatatttaactaagtctttgtataatgcagatcctgaacttaatttttatttttattattttgttgttaatgaaagattctgttaccaaattttatcagtctatttaattagaggccaaagattgttaggtatgtggcacttggagtgggaaatgatatattcccattaaaggtgttaattaaccaccaaattgttctttaggtctgtttgtcattttgtattaaggtggatggttcattatcttctctttaatgctaatcatgcttcaccttttcatttagtactagcaagcatatttgtttactttattggttattccttatcaaagtctttatcttgttgcttttttttttattgtactttacaaaagatttctggtattaatggaaagtgctcatatttggaaaaagacatggccaacaagaaaggtgtataccccatttctttttctttttctccaaattttttttttttttttttctgtttcttgttgagttctttcatggaagaagaagaagagtaggagattcttggacatggctgcatgagaattgttattgttttgtgcacttaaataaccccgtatacatacacaagtagtgttggctgtctttgatattgcaccatttattgccctaatttctgccttttgtcccctcaacaaaaccatcaaagttctcaaatagggtttattcttgtttcccactttgccccccacccattagggccaccccaccaaagggatctctctcgtgtctagtgttttttcccaaggaccaccactacttttttttttttctctaccataacttccacaccatcttgtgatctttcgtttaataatgattttgcagccatgccttcgttcatcagaactcggtcataagcacagattctgagagagtaattaatgaatgaatcagtggtgatttgacttatacatgattatggtttttagctcaataagcagagggagaaaatatatataaacaagtaaatctagtagaagaagtagaagttttatagctagagtagtgggaaagaatgacgcaagattagtaccaaaagagtgaaggaagagctttaatatagggaaaagggaagtagtaggtgatacttgacaggttgataagatggttactactacaagttgatgtattgacgctaactcacgcaagagacctactcactgtgcaatatttacaagaagcgattctttctctctttacttgcaagagttgtgtgttccgagttgtatggcgcatatgaaccttttttcatacaatacaatacatatggaatagatagataagacggtgcacgcatgaggcaattatgcaacttaacatcaactacttccatcatcttttcttccctctgtttctgtttctgtttctgtttctgtctgatatactatatgcttctctggcctggattttcctaactcttttgataatttaggttcccatcaataatgtctttttagaggagcatcatatcgatagatattcaaatattaaacctggcctagggctagggcgtctgctaggtttttgcattactctttgtatatctcatctgtgggaccttttgtttatggaagaatatacttttattcatcttgttgggtcttaaattcaagatttaatattactctttaaaaattaatgactatcctaaaattcttcctcttttatttgcatttacaagaattgatattagtacctaaaactcttctctggggccttttgtatttagtccttttatgtttgaaattgacactatttaaataaaacataatctacaataagatgttcttcacccttcgggttgcccggttggtttggatgggatcgattcccccgatatcttctgggttgagcatatcgcacagggcttgtctagtgcggtttgcattccctatgtggtttgcattccctatgtggtttgcatgctattatacatgggtttacccagtggacacaaagtattcaatacagagtgttcacccgaagaacagaggctgtggcaaagattgtaacggccgcgggtttcccctcttacaaaaaaaaaaatgttcttccttaaacattacccatcaaagactcaccaaagatagctctaccaagtattatttttggatcaaatggcatttccacggtcatatctcctcccccccccctcacccccccccccaaagctagtgatcacttccatattttttcctgatttcatcggtgctcaaatacttgttcattcatcttcattccaaacaaaggcgaaaaacttcactattgagtgcttttttcctattccaagtgtcaaaccctaaacttctagtcaaataatatctaagatgtatactcttatactatgtttgtattcattatgacaaagtatgatgagttgtacaattttcttgcggacttagtgaaaatagagcataatgttaaaaaaatatttacatgatattaattagccggattaagtttataacgttagtatatatgtctactttaggtacaatacaagtcttatactcttgtcagaatttatatgtcacaaaatatagaaacttctagctacttttttttaattttatataatataatattggaatgaatttaagtggagcaaaagtgaatattcatgtagtcgatatattctaatctgtttggggctgagatgacatgattgtagtgaaatattgtaccattgcatttcacactcactgaactgaaactttggttccacgtcggtgatcatttgcatgtttcattagtcaatactgtggctgttatgatttggctgcgagggggatcgaagagagcacattaaagtatttaatcaggatttatgagttgaatgctgttagttggcggaattaatagtcaaataatgaatgagttactcgctgatatagttaatagtactccgtatatatgttgattctagttattggtggtgacctaagtaaagagaatagatgagaggagtggtggtatgtaaaggaatctaggtaaaggggtgggggtggggggaggcaaagttgaaaagaaaggtggaaaatccaagaatcctgcttcctctagtaacatagcatatcctgctattcgtgcttttgtttcctctcacaagataactactttttgattaattattacatttgacgcatacaaacctataaaattaaactaatcaacgacatccttatggaatcttacgagtccgaacttgtcatatatataactttaaagtactttgtcacttcttaatatgctcctttaattgtgcttagctttttgctaaaaaacaaaaaggatatccttattccaaaatgcaactgggagcatcttctcacttttcttttttatgcctctgcatcatcaaatcccacaatgccgcacaacaaactcttgttacttaagtatatattctacttcataagaataatgggtataaaaatttgcttattttatgttttaaataccaccgaaaattcataagcaaattcaggatttaaatatattaaatgaattcaacttttaaaattgttgcacttatatatatatatatatatatatatgcatatccaagttgagggatacgggttcacatgaactcatattactttctctaaaccatgtataacaatgttatattttttcaaaattatttaaatatatgtgtgtgaacccattctcaaaatctcttatggtgcaattattattgggtgcacatctacaagtgaaatttgcagctcaaaacctcatctgggcggtcttgttttccgcatggagtataactatatatgtgaaaattactagaatttcaaaatgaatataattttgaaatgttgtgggttcctggtaagagactaaagttaaactcgtcaaatataaattctagatccacctcttcacaatagtgcacccattcttttgaaattctggatctgcctctgttaataatatatatatatatatatatatatatatatatatatatatatatatatatatataaacacaaaaaaatatgtggaaaactttactattaattaccactgctaaacatttgaatggattcttcatgccgtgtgctcctttgttgaagaacacgtacttgggagggcgagatttcgaataaaaaagttatactaataacaaacagcaacaattataagaaaatgaaaataaaagggaaagagcactcacataaactagaaactgtagagttggcaagtaccaggtatatatgtccttgaatgttttttacgaggaattgagtaaaacgctagctatttcaacacatatataaaaagcatcaataccaattttatggtttctcttaggtgttgatagattctcttttgtcagcaaagttcttgcattaactatatgaaatttataataaaaatgctgctcttttaattgagtatacatgcagtctcctaacatatacattctccgtcccgatatatacttgatttgatgcatttcaaaaattaaatgtttgagtgttttggtgaattgtgcttgatatagaagtatttaaaataagaaagaaatgtaacggcagaatcttaagtcgaaagtcaaattaaatttgaaaaataaaaaataatactcttgatacttactagtactagtcaatgggcagctctttcgggactaaccaaaagcattattcttattgtttccggcatagtattaaaatgtaacaatgcttaattatgttacaaaattaatgtttttgtggacttcggaataatttatttctgaattcgccggtgttatcgaaaacatgggga

SEQ ID NO: 100 - DNA, 5007 bp, Nicotiana tabacum
  source 1..5007: mol_type = unassigned DNA
tgggggccctccgcacaagggaggagctaggggaggcaagaaaattacatgtgtatataagattaaattttcttcttcttttttgtgtataggataaatatttaattttcttaatttttttgcgtagttactcttttaattatttttaactcctctgaacgcaagtcttacctccgccgctgactcctctagagaggaggggtctaaatgcatgcaactcaaacgacgaaagggtctttgcctaactaaaggttccttattctaatatatccttctagaaaaggtaatagaagttgattcgaatatcaaagagagatacatgtcggttacaaaaataattctcaagataatcaatagacatctctagattctcggacttaaaagatccatttagacacataatcatattttttttccaaacaatcgtcgaaggcagcttgttgcacaaaatatttcgtatttacataaggtttaaggaaaaatcgtacttcaaagagtatgatgtagataatacattctaattagggtcggctctataggtgtaaggcaagtgtcttaggccccaattttgggaggccccattttttagtaatactatatattttagaggtttataatttttttttaaataattaaataatatatatagtatttttttacttttcatatataaaaaagaaaaacttttaatatattaataaagtaaagattaaaatagtactaaaaaatagttgtcactttgatttgatttgacaactttactttttgctcttcttcccaaataattcctagacccaatccaacatttcaattttcatatagaaggatgagacatagtaacataccttcttcctttcttttgattcgtgctcattttgtttctactagatttctttctttctccaaaactccaacaagccaacaactacatattttgaaatcaaagtcttcaaattttttgaatcagttagtcgggtaatatcattctaactttttagtattatttttatttattatttattattttattaattttaccatgtacatattgtttaatttttttagaatataaatatgtctacaagaaaatatgcatccggatattcaaaaatccaaaagaaaagaaaagttgaaaatttaataaaatctcaaaaaggagttctcgagaatttttgacaaataataaaaaaactaaatcgcaaaatgtaggagaatattctgtagatgaacaagtcactaactttgagtcagatgataacaaaattcaaattgaagaagatgtttatgagaaatctgacgaagaaacaaactttagccttaggccctatattaaactttggccctaggcctcatatgagcttgagtcgcccctaattctaatacaagtattagtggctactttcacggcttgtgacttatggattacaaacaactttacagttacgtcaaggctccacgtagttctcaatttatggagcatatattagatgattaacgcagggaaagattctgctctcctctgatacatggctattattcctcgtttagttcaaaaaggaaaaagagggtagtcttgttatattattggggaatgaattatggtttcaaacttttcaaacttaaaggattttgtacatggtaaaacctaaattgacacgtaacttggtactttcaaagacacgatcttttacgcgatattttaaataaagaaaagatcaagtcaaaacatgggccaaaaagaaaaaccccatgattttttctgataaaaagctgctaacttttagtttgttttatccaataaaacatctttaacggtctgcctgctttagtttaatcctctttttaagatgtaattaagcataaaatagaaaagggaaaaaaaaggtccattggattttggaagaaattttaagaaagtacaagaactagtaaagtcattttgtatagagtatgttaaaaaggtgagtgacaattcgaaaaagaaagcattgataagtcaatcactaaataaaaaagcacacctaataatcattcattcaaaaaaacaaatttctatgaaagataatcattatcataagtcactgcagaaatcccatatacagtagagtaccaggattttacgataaggtgttagcaaactatctattcattttttgacaagcattttatgtttggtcatttgttgggaaaaattagggagaaatttaaaaatagttagatttacaactggtcattaaaaatagcccaatttcaaaagtaatcgaaatttagccacttttcatgtaaagataaatctgagcgaaaatattgttcaaaacccggaaaatacgcccgtatattatactggagttccagcataagtatgcttgaactccagcatattatacgggagttctaggataactatgttggaactccagcataatatgttggagttccagcataagtacactagaactccagcatattatacgggagttccaacaagtataactgtcccgtataatatattggagtttggagcaccggtgctccagtctcccgtatattatacaggagtcagcaaagtataccggtccagcataatatgctggagttcgtacacagatgcaccgaactcacgtatattatgcggaaccggtctctgttgcagcaaaatagtggctatttttcattgacttcgtaaacggtggctatttttgaatgaccagtccgaaaactggctataccgtgctattttgacgaaaaattatccccccacccacccacccacccaaacgcaccttacacacattagtgcacatcttttaactagtttttggttatttttttatttgatgcccgatattcgtatatggatttcgattaattagaattcacaccgaaacattctttcttaggattttgtacatacttaatatgcgaatacaaaacctatgcggaaaggtaagggaacctattcatccctctacagtacttgtgataatgttatacttttttgaatttaatttgggagacatgtcaatctttattttgaaaaaaaaatagaataaaaccatagggaaatgaacaatttatctttcactcctatctcattttatttgtcttgaatttttcaaaattttgaattatattttgaaacttcttcaatttattttcttggaatcttcagaattcaatttaaaattccaaaattccaaggatttagctcccgtttggccacagattttggcttcatttttttaaaaaaaattttgaaaacattctttgtttatgcaatatgatcatgttttaggggaaaaaaattaaaaaaaataaaaaaaaatcaaattcccaaaaactggttaggcaatttttggatgatattttttcttccactcacaaaactttaacatgtccaaacacaacttcaacttcaaaaattattttcaacacaattttaaaaactcttttttcaagtttcaatcaaatctatatccaaatgttagcttagtatcaaataagtgattgaaatcaaattaaaatcgagtggtaaataaaatagaggagagctcggtaaattacaagagtgcggtaaatcttttctcctttactctcactgtagcctattctatctgttgtaactaataagtaactgagctacggaaaaagtgcctagacttttaacttcacaagtataataaatagaagtcaattctttcataatattgtttccatcctatcaaacagactttgtctcactgaccttccttctgagtgtgtcttttatatgtcatttttagtgaatccatatgatttagagactctaatattccacatgcgggtcttaatttggtgtatatgtatatggtaataatttttgttaggtagctgtagtattctattattgttatgtattgactcatcatgtaaataaagccggttagataaggctagaaaaatatgagtatacctagaaattattagcatattgtttggaacatgtcaaaaatttcaatgacctagctagagctgtcaattagtcaaataactttattaatatttacttatgaaaacactttgaaattcttggagtttaagggaaagactactgactaaaaaacaaagcaaaagtctatgcattactatactatacacagcacagcattttccaatagtatttgagatgaatctccaatcagctactgttgttcttttcttttctttatttagtttaagttttatgtgttgatggtatacaaattatttgcacaatcaaatggcttatctggataatataggtaaacctcttgtaatcactaattggtaatctggtaaaaataacactatttctattccaatttatgtgatcaatttcactagacaaaaatttaagaaagaaataaattttttagaacttgtagtcataaacaagttgtaacatttgtatggctataatttttttaacttgtgatgttaaacatgtcagattgtttgtgtagctataaaagtttttcattaggcgtaaaattaaaaatttagattaaattattattaaatttagaaagaggtcattttttttagcgaagtaaaaaagaaatcggttcacataaaccgaaacatagagtaagtaatctgttatgacaaattaaaaattacttgtagtgtaaaaaaatctttacaacattcgtgtatatacttaaatcttttttattttttggcaagagatagttgttcagcaaaagtaagttagaaataggtctgtccttctgactttgtaactctgaaatgaaaatttcaaaatcccttctatttttcttttccccccccccccccctcacaaaccccaactcactcttatttaataaaaagctctacttagaaaagacacccttgtccatctgtctatataggtagaatgagagtaaaggagaaaacatatcctcctctccatttctgtagacaaagattctcaaagagaaacaaattaaacactagagagtgagagagtgctataagaaaaagaat

SEQ ID NO: 101 - DNA, 5000 bp, Nicotiana tabacum
  source 1..5000: mol_type = unassigned DNA
agaagaaatgactgttgaaaagaaaacaaatgcaagtaccattaggaagatttgaaagggcgtttgggtatgggggttgccaagaagattcagactttttttggggttttgtgtagttgtggtagaattattattgaatgaaaaaaaaaaacttcctgtactttaattcgtcagtacatactacatactactacaaagtagttaaaagcctattctatttgtgctttttttttcactcgatgttcaataattatattggtttttgattaaatttgaatttgagcaaggaagatcaacattggagggataaattgtttcctaacgaaggcgattacatacttagaacttgaactcaatatctctaattaaaaatgaagtaatacttataataactccaccacaattcttattgttgtgcatttctttataaaatatgtaaataatgggtcatatatattgtttaccttttctattcatatacatagattttaaattaattatacacatatatataatacattaattattcatatattatatttttgctagctatttttagtttaagcgatttggtaggcgactacttgggttaattctttttttttaatatatatatcaaaataatgaagctgtataatacacttaaaaatcatatttgaaaggtattaaatacgacttaggagagttcttaaaccattttggaaccttgtctacgtacttttatgcaatagctgtttttgtttgtctctgctaaaacctatgctccccaaccgtgcaccaatcaacttagaagttagaactcagaaataaatgtaactatactccacagaaagttaaaaagttttactgttaccattcactcaaggatcagaaactgaaagacaaatgaatcagtgcttcactgttcttcactaaaagaaatactgtttacattagtttcaaaagagtttaatcataaaaacaaatgtaccataaaaaggggagattatcaacctgaaaatgaaacagaacatacgttatatatcaatctatatacggtcgagatcggactcgtctattacacgacagatcgggattgaaacgtaacagttttgaagatcaaccccgggttccgtcggaccgaggtacaaggtcagaatgcccgttctcgagaacatcgagtccatgaccccagaatcaaccctgaccccaaatgagctcgaggaaacatccggataacggaaggcgaaatatccgtaaccggtcgggtatcacggcatgaatttcggcacgtaacaatgagaaaccggctaattagcaaatcatggaattttttaccttttatagaattgtaactaaagtgggattcccctactatgtaaagggggtctgactatttgtacgggacattcattaaacgcatcccaaagtaatataatattattttctttttgtaagctattgttctcctgtatctgatactatttgaattgcatcaagttcaagtgagactcattttttcaaggctataattgttcaagtcgcacggtttgaatttattcgatcattgttcgctttaattacaattcaattcatcgctttatgtcaaattaatccacatatccttaaaaccacttacaaatttaattgttatcaaattttaagggtaaacagtttggcgctcaccgtggagctaaggataatagtggttgtttgatatagattttcataacacacactattttacaattgttcttcgaagtgtctctcatttcaggtttaagctcaaaatgtcaaactcacaattggcacccctacctgcacacaatgagtctggtcaccatggtgaaaataacaacatagcacctggtaacgaggtaccgcccgctgatcccatcagaatttcaatcgcggacccgttggacgctaactcgcatgtggctatcgacatgttacagtctcaacaggcgacgatagctcagttacaaaaccaaagccgcacaccgagcagagttgaactcgatccgtcccggaaaatcacctgcagggaagaaccgtccgcggagaggtcaaatggagatgagtcggggactaaccccgagatcataaaaatgcttgaggaaccgatgatacggattgaatcaggggaaaagaaaatcgaggcaaatgacaagaaggtaaaaacttacaatttcacggtcaaccaaatcccgggagcaccgccggtactgaaaagcttggattccaagaagttcgtgcaaaaacatttccctccgagtgtggccccgaaatcgatcccaaaaacatttatatgcccgagattcttaagtataatgggacaaccgacccaaacgagtatgtcacttcttacacatgccctatcaaagggaacaacttagaggttgatgagatcgagtctgttttgttgaagaaattcggagagaccctgtcaaatggagctatgatatggtatcacttacctcctaattctattgactcatttgcaatgcttgcaaactctttcgtgaaagcacacgccagggctatcaaggtcgagacccagaagtcggacctcttcaaagtaagacagaaggataatgagatgctcaaagagtccgtgtcctagtttcaaatgaaacagaaggacctaccaccggtcgctgatgattgggccgttcaagctttcacccaaggactcaatgttcgaagctcggtggcttcacagcagttgaagcaaaatctgataaagtacccaactgttatttgggccaatgtgcataaccgctatcaatcaaaaatcaaagtcgaagatgatcaacttgaggctctttccgggtcggtttaccctgtcagactcgtcgacagaatcaagagagatatcgaccgtgaaccaaggtcaaacgtagatcattactagccatatgatggagattggaaaagcaataggtctgggtgaagttctacacagaatgaaaagagaaatgatccaggtcagagcactcgaggactcgcaagcaagaacgacttcgacaggcctatcaggcctaaagaagcaccaaggttatcgaaatataactttaatattgatgcggctgccatcgtatcagctatcagacgcatcaaagataccaaatggcctcgacctttacaatccgatccagcccaaagggatcctaaccaaatgtgcaaatatcatggcacttctggccacagaataaaggattgtcgacggttaagagaggaagtagcccggttgttcaataacgggcaccttcaagaatttctgagcgaccgagccaagaatcattttagaaatagggattctaacaaatagaccgaaccagaagaacctcaacacgtcattaacatgatcatcggtggagtcgatgcccctcaagtgctgatgttgaagcgcaccaaagtgtccattacaagggaaaaacggactcgagattacatattagaaggaaccttgtctttcaacgacgaggatgcagaagggatcgtgcagcctcacaatgatgcattggtaatatctgtactcataaataaatctcgagttaagcgtgtgttaattgatccaggtagctcaaccaacatcatccgattgagggtcctagaatggcttggcctacaagatcaaatcatgcctgcagtccgagttctaaatggattcaacatagcatgcaaaaccactaagggagaaataacattgccggtgaataccaccagaaccatccaggaaaccaagttttatgtgatcgaaggagacatgaggtacaacgctctgttcgggaggctaaggatctacagcatgagggcagcaccctcgactcttcaccaagtgttaaagttcccaacgtcgggagggatcaaaacaatctacggggagcaaccggccgcaaaagaaatatttgcagtcgaagaagagatcccggtatagacactagcaacatcaaaggaaccgagttcggataagaaataataggctaaatagcaattatcgacaccagccacgacccaatcggataaaaaggggactgatgaagatgatgattatggggttcccagatcttttatagtccctgatgattctgacgccaccaaatcaatggtcgaggagctggaacaggtcacattaatcgaacgtctacccaatcagaaggtatacctgggcacgaggttaacccccgagcttaggaaaaactcattcaatttcttatagctaacatagactattttgcttggtcccatattgatatgacagggatcccaccggagataatcattcaaaagctgagcctggacttgaaattctacccagtcaagcagaaaaggagaccccagtcaaaaatcaaacatgctttcatcaaggacgagtatttgcacaaaactttcaacatattgaagaagtacaatatgaagctaaacccggagaaatgtgcattcagagtcggatcaggtaaattcctcggattcatggtatccaatcggggaattgagatcaaccccgacaagatcaaatccatcaaagatatcacgatcgtggacaacgtgaaggccgtgcaaagattaatcggccgcatagccgccttggggcaatttatctcgagattctcagataaaagtcaccggttcatttcgctactaaagaagaagcacaacttttcgtggaccccggagtgtcaccgggacttggaggaactcaagcggagatagctgcttcacacaacaaaggcaaacgaacaactatacctatacttggcagtatcggagatagcggtaagtggagtcttggtccgggaagaacaaggtacacaatttccaatttactatgttagcaggaccctaggtgaggccgaaactaggtaccctcacctagaaatattggcattcactttgctaagcgcgtctaggaaactgaaaccatatttctagtgacatcccatatgtgttgtgattaccaaccaattgtggaatataatgtataaacggctactctcgggatgattggccaaatg

SEQ ID NO: 102 - DNA, 5002 bp, Nicotiana tabacum
  source 1..5002: mol_type = unassigned DNA
aatgcagcctcagtcttcaacataaaaaatcacatgagtatataagattaatttttttttttttgtatatatgttagatgttgaatttcttagttgttttgcgtgtgttttttttttttttttttgacctccctcggtaaaagtcttacctccggcaactgtctccgcgagagagagggggtctaaacgcatgtaattattcaatgattgaaggatggtctttgcctaaatactaaaggttccttattgtataggtcataggtgttgattcgaatatcaaagagagattcctgtgggttataaaagtaattctaagataataaatactctttttagaggtgttgttttactgtaaaatacgtatttggttaaagtgatagacatatctagattctcagacataaaagatccatttagacacataatcatatttttgccagatagttgttgcagctgaaaattggagcatatatacttagaagtagcataacactttacttccatatagtcggatggaaaattggagtatcatatcttcttttaattcacagttaaatctttaaacatatttaataaattttttaatacaaatataaagtctatgcaaaagttattgggttggtccaagcccttaatttcgatctctagctccgtccccgcatataattctaaaacttctgcttctcctatgatacatggctaatgtttatcgtttagttcaacaagaaaaagagggtagtcttattatattatgaacggcaaaggtccaaatatgcccttgtactatacgaaattgagtacatttgccatttgttaatactttagctcaaatatacccttaccgtcacgtagttggtccatatacactactagaaatccggtaaaaccgaccaaaaaaaccgaccaactttggtcggtaatggccaataaccgtccaaaacacgaccattacgtgtggacggtattttaggggtcggaaaggcataccgaccaaagttggtgtaaattaccgaccaattttggtcggtcaattaaattaaaaaaaaaccgaccaaagttgatcggtattttaattatgtaatcaaaagattgaccatctgggaatcgaaccggggtttgtattgtggcaggatactattctaccactaggccattagtgcattttgttttaagactgttttttatttgatttatactctttaattgtattttcgcacgaaaataaccgatcaaagttagtcgattttattaaaaaataaaattaccgaccaaagttggtcggttttttaaaatgaccggccgaattaaccgaccaattttggtcggttttttaatattaatttttatttattttaattaaaactgaccaaaattggtcggtttcttgaaaaaaatttccgggactcgaaaatagtttttcgcatttttctccaaagaaaaccgaccaaagttggtcgatttcgtaaaaaaaaattaaaaataaaatattttaaaaaaccgaccaacttttgtcggttttttggtcggtgttttgaccgaccaaagttggtcggtcgaccttggtcggtttttgccgaatttctagtagtgatatacccttagagttacacaattggcacatatatgcccttctcaaaacgaaattcacccaaaaattatggtttaaactttaaaataataaaaacatctcaaactttaacaatactcaaaagaccaaaatatttaaattatttctaaaaagataatttaatgattaaaagcctagagttcaagttgtagtgttataaatttgagttgttagtctttttcatctttttcagctggacattttctattttttttattaactatgtaaattaggggtgtacatggaacgggttggatcgatttttatcaaaactaaaccaaaccgattatatcggtttgaattgttcggttttattggttttttcagattttttgttacataaatattatttcaatcttgctttgttaaattttttagaactaaatatatgttcagtaaaacttaaaaaattgacaaacatatgatctatcttgattaccttatgggagaattttcttagtaattggaattcatgagttttgtcaagtgaaattggtgacgaaaatagagaagacatcagtaattgaggaaatcggataagggagaaagaaaaagaaaaaaagaaaaaaagaagaaagaaaagagaaaggtaaagaaaaaagcactaataaaaaggaaatagtatttgtaatatactttaatacaattaacgtaagagctaattagtttgagtggattccgttttgaaaagggcatacatgtgccaattatataactctaagggcatatatggaccaactatctgacggtaagggcatatttgagttaatatattaacgaatgacaaatgtgctcaatttcgtataatacaaggacatattacattttccctattatgaaatggttcaaacttaaggattttgtacatggtaaaacctaaattgacatgtaacttggtactttccattgggcaaagacacgatcttttacgtgatattttaaatcaagtaaagatcaagtcgggccaaaaagaaaaaaacccatgattttttaagataaaaagctgctaacttttagtttgtttcatccaataaaacatctttaacgatctgtctgctttagtttaatcctctttttaagatgtaactaagcatgaaatagaaaaggggaaaaaaaaggaccattggattttggaagaagttttaagaaagtacaagaactagtaaagtcattttgtatagagtatgttaaaaaggtgagtgacaattcgaaaaagagagagcattgataagtcaatcaataaaataaaagcacacctgataatcattcattcagaaaacaaatttctatgaatgataatcattatcataagtcactgcagaaatcccatatacagtagagtaccaggattttacgataaggtgttagcagactatctattcattttttgacaaccattttacgtttggtcattttttgggaaacgaactctcccaacattcttccaaattaccccacgcaccttactgtgcacatcttttaaccaacttctggttattttttcttttgatgtccgatattcgtatatgaattcccattaattctaagttgcaccgaaatggtttttatcaagattttgtatatatttaatattcgaattcaaaactaatggtcgaaggtggaagatcgtatccatcccatcataatatttggttggtaatatcacacctttttgaatttgggagacttgtcaatttttattttgaaaaaagaaaaaaaaaagaaatagaaactaaaaccatagggaaatgaacaattttattttcactcctacctcattttatttgtcttgaatttttcaattttgttttgaaacttcttcagtttattttcttggaatcttcagaatttaatttgaaattccaaaattccaaggatttagtgtcaaatcagtgcttgaaattaaatttaaaacgagtggtaaataaaatagaggagaactcggtaaattacaggagtgcggtaaatcttttctccttttctctctttggagcctactctattctattgtaactaagtaacttaactacgaaaaacgtgcctagacttttaacttcacaagtataataaatagaagtcaaattctttcataatattgtttccatcctatcaaacagactttgcctcactgactctccttctgagtgtgtcttttttatgtcatttttagtgaatccaattgatttagagactcaaatattccacatgcgtgtcttaatttggtgtatatatggtaataatttttgttaggtagctgtagtattctattattgttatgtattaactcatgtaaataaaagccggttagataagactagaaaaaatagagtctacttagaaattattagcctattgtttggaacatgtcaaaaattcagtgactcagctagagctgtcaattagtcaaataactttattaatattaacttatgaaaacacttggggattcttgtagtttaagggaaagactactgactgaaaaacaaagcaaaagtctatgcattactatattatacacaatacagcattttccaatagtattttagataaatctccaatcagctactgttgttcttttcttttcttttttagtttaagttgtatgtgttgacggtatacaaattatttgcacaattagatggcttatctagataatacgtgtaaatctattgataatcattaattagtaatctggtaaaaataatattgcttttgttctaatataatgtgatatatttgactgggtacgaaatttaaaaaaaaataagacatatagaacttgttgtcttaaacaattcataacatttgtgtggctataattcttttgaaacttatggtgttaaacatgtctaattgtttgtgtatgtataaaagattctcattaagcgtaggaaaatttgaattaaattatttttttaatttaaaaagagatcactccttttagagctgacttaaaaagaaattgattcacataaactcgcacggagggaataagtaatatactatcaaaaattaaaaatcacttgtagtgtaaaaaaatctttacaccaatcgtgtatattctcaattttttttttttttttggcgagaggtagttgttcagcaaaagtaagttagaaataggtctgtacttttgactttgtaactctgaaatgaaaaattcaaaatctcttcttttttactgttttaaaaactccaactcactcttattaatataaagctctagttagcaaagacacccttgtccacttgtctatatagcaagaaagagagtaaaggagaaaacatattctcctctccatttctgtagacaagattctcaaaaagaaacaaattaaacactagagagtgagagagaactataagaaaaagaat

SEQ ID NO: 103 - DNA, 5000 bp, Nicotiana tabacum
  source 1..5000: mol_type = unassigned DNA
agaagaaatgactagctgttgaaaagagaaaacaaatgtaagtacaccattaggaagatttgaaagggcgtttgggtatgggggttggcaagaagattcaaactttttctggggttttgtgtaattgtggtggaattattattattgaaacttctttacttcaatttaaatcgtcggtacatattacgtagttgtagtaaaagccttttcctttttgtgcttttttttttttttcgtgttcgtattaagacttcattaaatccaaatttgcatagggacggtcaacattagaggaataaattgcttcctaacaaagacgattttatactcaagagttcgagcccgaaaaacgacctctggttaagggtaaaaatagtaattacaataactccaccacaatccttattggtgtgcatttcttcattaaatactccctccaatccactttaattgatttgtttttggctatttttatatatattaaggaattatcttttagcattaatcaataatgaaattgaccatattaaccttttagttcattggaaatataacaaatactcctaggctttttaattcaagagcaacttttaaatccgaatttgggctaagaatacaagcttgttcttttttatctgtttttcactcggtgtacgaggactcaattaaatccgaatttgagctaagaatacagacattagaggtaatatgctttctaacaaatgtgactcaatgttcagactcagaactcgatatctctggtaaggatgacatagtacttacaataactccatcataatctttataggtatgtatttctttataaaatatgtaaatagtgttatgattttttgtatcaaaaatgatgaagtataatactcttaaaaatcatactccatccgtttcaatttatgtgaacgtattttctttttagtctgtgccaaaaagaatgacctatttccttatttggaaataatttacctttatgcaatgatttatagtcacacaaaatatatgtgtctcatttttaaccacaagttcaaaagtcttctatctttttttaaactctgtgcccagtcaaatgagttcacataaattaaaacggagggaataataaaaatgtattaaagactacttaggagagttcttaaaaaaccattttggaaccttgtctacgtacttttatgcaataactgcttaagtttgtctctgctaaaacctatgctccccaaccgtgcaccaatcagcttagaaatttgaactcaggaataaatgtaactacactccacagaaacttaaaaagttttactgttaccattcactcaaggatcagaactgaaaaacaaaagaatcagtgcttcactaaaagaaatactgtttacattattttcaaaagagtttaatcattaaaatagatgtaccatcagattagctaaaagataaataatcgttaaaaaaaggagattatcaacttgaaaatgaaacaaattatatgttataatatgtcaaaatatactgacagtataaaaactcgttaaatgtgttaaatcctatgaaaaaactgcccaaataaatatttgagcttaggtgtcaaatgttgtactcaacaacaataacaacaacgcattaggatcctactagtggggtgtccaatgttgtactattgaacattattcaactaacttttgttaggtgttcctgtagtttagtgaaattaaagtccactgttcccctatatattaatcccaaattaattaatcaagtgcagataaaaatttctcattttctattaatttattaagtgtaacaaactaaagaaattcaagaatcttgaatgatgagaaagagtcatgcatgtagaaaaatagataataatacatggaaatatatatgtatttggggatttgcatggtagctcaaagattattggaaagtgacaggaagataaatcaaaatctcagtgttatttcaaaaataaaaggcacagattatttaaataattgacagccagttttataatactatgtgggaggggacagagatcaatccatgtacgtgcatggctaatattaaagtaagggagaaaaaaatattaagttaattgatgattaaaaatagtaaaatttcagacgtatatcacggcaatgaagagtttgatctttaatatctgtataatggtccataatatgatggataggcgttgtttatgatatgattgattgatcattgatcattgactattgtttcttgaataattaatcagtatgggaaagggtcccattaaagttgaccatttgcttagcaatattatcttaggtaagctccatattagtttaatccacttgcgaatatattccgtcctcgcaaatcaatatttacaattcttttttttcagttttctatccggtatctgatacttgcattggtgttcgacaaaatctgtattcgcgtcaaaaaatttcatattatggggcaaaatgctccataataaaagcgactcaatattagggctcgaaccaatggcggaaacaagatttttactaagggaattcaaaaaataaaaacgaaaacacatgaagaacctcagggaattcaacatctaatataaatatatgaaataaaaatttgattctattgtaatttgatatacagtgtaatttacaccgtaggggatttggctaaacctccttccgcgtacctagctccgtccctgactcgaatccgaggtatttggttaaaaatgaaagagtacttctcataacctcgtcggtttttgtttctaatcaatctttatattgttaaaacataaaacgtttacttcctttcttcttcttttaagttttgaaaatgataactacttttgtttgactaatattttgtagtttttgatgctaatcaattttgtaaaaattactgtacttcaactagcgtttactaccccacctcactttaaaaaattccctaaagagataactttttgattaattcataaactaaattgaagaacttttcaaatgagagtaagttgaaaatgcatattatattgtagtatataattgcaattttgcataacttaccgtaaaatgttcttccttttaatgatttgttaatatgggaaatttgaacttttctttctttgaaattgtattcttgtcccatggtttctatgcaatctcaatcatcaaattgcaattatttttttttgttttttgttggcaaattcaggagagcttaggtcagtgatatatgaaaaactattttttactcttatttattttaccctttacttattaaagaataaagtccaagacgaatagacgatgtacaacgcaaatgtaaaaatacaaaaaaatgtttacgacttcttctctatttattttctacttaatttacttattaaacaagtacttacttgttaaactagctaatctgaccaacaatgtgaaaatgtttgacattatacatcttgactttttatttctctattattttctcgatggttacttcaaatcatagatttgctaatctgaccaatatcgtttaacttcaagtagaacgaaatgaacatttcaaggttttagaaaacagttgaaattggaccctaaaataaataaaatgaagttattaataggtttacaccccaatcttatctaatgcttaaaacacatagtgtggagcggaattcattgtcttcacattattgtacattaatcatattttcttaactaattctgacgattatttgtgttcatctaatagaaaatgcaaaagtcattttccttaatacttggcatctttatagtcaaaatataactcatatccaatgcaaaagtacagtcatgcacaacaatttaaagtattaggagcatttattagtttcacttgtttattaatgtaaaagtacgtagtaaaatgaaaggtaaactcaaaaatatcacatacatattttaatttgatcgatcaagtcagcttgagttcttgaactttgtgaaagcaatatatataatatgaatgaaacacttatgcattgcgacattgagagttaatttaagaaaattttaccccacaagttctagctttgattgacaggcctagccacaaagtaagatatgcacaatttatcttagtgcttctatgttgtgtatcaaaactcaacaagttatgtttagtactcctatgatgtttatcctaatttaaaagtcaatattaagtaaataaaaataaaaataaattaaagtctatgtatgtattctcttaccaatgcctatagtttaggcccagaacctaccatctccctgccaacccactactcttactggttttgcagaaatagttgctgatcaaatcatttatccaaagatctagtttcaaccttaaagatggaaggttcgagtcacttgattttgaagtattgacttaatgatgtactttctttaacataaggtgaaattagttgtggacttcatcgataatctgtcgttaaatcgtttgtagctaactaaaaatctatcgcgaaataggattaacgacgaatttttctgtttaactagagaattttttcgtcgctaattaattttttttttttgtagtgagtacatattagaaagaaaaaaaaaaaaaaaaaggaaagtgttgaagtcgtaatgtgtacaacatatgaagtccataacctgccaggtacaattctttaaagaattaaaatggaagaagaaaggtaaaagcaaagattgacaacaatttttttgtggctcgatgaaaattattagtgtcaaagaaagaaataaaggtaaaaaatggcagaggaaagaatcacctttgggaaatagccccttcacaaagactagagtccaaaattacaaacatcaaaagatctttggtcggttctactgtttgcatctcttgttgttgctttcgtcttgtgaaaaatcattgaggtactatgtaaatttataatcagttttttaatcttattgaaagtttcatagtgagaaggaatttat

SEQ ID NO: 104 - DNA, 5000 bp, Nicotiana tabacum
  source 1..5000: mol_type = unassigned DNA
gtactatcagaaaagatttaaatctaaccttcgttataatttgagtccaaatatacccctgatgctatactattggttcaaatatattcttttccattaagtttgtccaaagtggacatccaatcctacgttgcactgacatttgatgatgtgggtgtcacatggcttgccacctcagcgccccaaatccatatattagagactaaaatttgaaatacaataatttttcgtaatggtggcaatagtgatggaagggaagaaaattttagtggtggaaaaaaaaaatagaaggaaggggtaaaataggttatgagcgctgaggtggcatgtcacctcatcaaacgtcagtttttcgtaggattggatgtccaccttggacaaacttaacagaagatgggtatatttaaaccaatagtattacggcaatgatatattttgacccaaagtataacgaaagatatttactcatagtacaacgatacatttcgtcatttaccgaatttcgaatttacattctcaaagaactcataactttgatgttaactaattaagtttaaactttagatcaccctacaattgttgggagacatatcaaacccataccattgatatatataagaactttgtaaaactctttttaatatgttagggtatgtactagctagcaccatggatagtgcgtgtcaccattttacagcaaataatatgcctcatgaattggtagggaaagggtctttaaagttgaagtgtttttttctttttcttggtaggtagtgggggaggggaaaataaggggacaaaacacaaaatcaatttgagtacaatttacacaagtctccatatattgatatagtgctgaaaagaaatataatgttctaaggcaaaggggataacacttagaaattagccaacctcatgtttcttacaagtggatgagggttcgactttgcatttagcatcaccttattctctctaattttcaacaggacatgttaataaaaggatgacaatccagattgacaaagttatgaaagtgaaagcactgtaacatttcattcaatcatgcaagcatctcccgttaatttttctatcttttggaatattacgaaaatcaaaccaacctttttacgtataagtttctaatgacatcagggggactcctgagaaatattctcactcaaggtcgaaaaggaccctttaataatgatcaatataaattttttttataatggtcctttaatattcgaatcagctcgtacacatctcaatttttgtaatgagtacatgttatccccgacaatatatatataagataactttgccctcaagaatttatatcgacagataagagatcacataatatttttgtctttgttggaatgtaaactctacttttatgaaattttattaactggtaggtcacatcctacagagcttttagtcaatgttactattattaagaaggaaaaaaagaagaagcaaaaggaagatattaacaatagtataacaacattcataaacgtgatttaggaaataaagtatggaaaaatgaatagtgaaaaaaagtcaagtacttaggagttaaggttcgtaagttgatgggacagatcgcggtggaaaaggaagataagcttcaaagatacaaaagcaaaggggggttgggttgagattctgagttgtgattaataccactcatgaacaaaaagttatgtcatggcaccattcatgagattaagacaacggtggaattaagaattttattaaagggagtcaaattttaaaaaaataaattcatcaaaaagttaaagagtatcaatatatatcaatatatatatatatatatatatatatatatatatatatatatttatattaaattatctaattacacagtataatttttaacgaaagggtgtcgatcgacacccttgaatgcatgtggccccgccactgcattcagagccaactctcccccaaaagaaaaggcgcaaaacaaaaggatttgaaacagtagttgtggaggtgattcagccatgctttcatgactcatcaatacctactttttatgtttttctttttcctttttattttcaggatataaattttgttgtaaactgatataaagaaataaatttactctcttcaacctattaagcttttaggtgaaattagtaagtcgtacaatttaacatggtgtagaattgatagaagtcgtagacactcataggtgcactttgtagtgaaaattaggagaaattagggttcaaaatccaactaagataaaagatgcttggtaatttcttcacatctgcttagaaataatctgcttaaatttggatgagcaaagttatccaatacaagtgttgctgagaaataatcggtatctaatagaatagtcgagatgcacgcaaataagttttgactctactttaacaaaaaaaacaataggcagatgtatccaaaattcatatcttatggtcactcgtcaacaaaaatattccttatgtttagtccaagaaaaataaccatacccaaatataagggtttgttgaagacggaaatatataaacaaataaacaaatatcatcatatctccgatagtttaaaattttagattggatatttcacacaatttatcaaaattttcaaaaaaaaattctactttttcttgcttggaacttggaaggggaaggggtggtggtggagatagggcggggcatcttctatctagtctatgtgattaatataacaaaacaaaaagggcgaggcaaaaacatggatgaatggtggtccttttctatatttatatggattgttacgatacgtcgatttcactttgcaaaataccaattagattcatttagttatctttttgatcactctgcttttactatcatatatatataggagtccttccacgtttcgcatgtgtcattgtttatattttccatggtcttccttccaaatggctaaaaaaatttgacacagtggtcccaaaagtttatagaaatagaattcaacagtgaggcatatacctatgaattctattttacatcttcatcgtataaaatagaatgtgttataaactttacctcgtgatgcttacaaggggtgaaaatataaaagcactttatagatttacaagagtcacaccttgatttatcctaagattttatttttttacatgccaaacaatgaagtatgggagatccaattggaataacatcaaatttaataaaattcgaaatagtcagagagctgtctactgaggtatattgaaacttatttttttttaatagaaaatatcaaatacttagcaatatattaaaatgtttcataaattacattgtttaaaccaagcgttgaaacatatgctgatacgaggtaggcttattgatgaatttataagggcctcattggaaaagacgatccaaagcaatgggctaaaaattggcccattttctgccacccagtgtatggttattactagtttcacccacacagatttgcacttcattagaggacaatgttgctgaatttgaaacataagtccatttatctccactgtacagtccttcctggagtccaatcctgaccatatcttcatgattttatgtaatgtggtgaataagcaaagtttcatgttatgctttgtctcattttatagcaaattcatttcctcataaaatttacttcaaaaaagtttcgtttgattttcagaaatcaaaatatgcttttcggtaaccaaatggttttcaattttgtttacgaagaacttaaaactttccaacaccctacatctatgattgcaagttaaaattgcagaaatatgacactttttggagtggtctttatcgtttaacttcacttgcactttaagggcaaaagttaaaagtgtttccatgaagcaagcgagggataacacttattaaacttgaaattctactcatagaccaaaacaaggacaaaaattcaagactatctatgtgggtaaacgtacgaaaattgggcttctccagattagagccggaccttgtggaaagacagagaaattcgaggcccacttccagtttctaaggagattaagcctatcaaacgatggtccagaacgaaatatgtctttctttattctctactatatagctgactcagaatcgttagaatttgcaatttcctcataataaaatgtgaggcagtatagattcgaaaacctttgttgaagattattgactcagctacgcgaaacaaactgtagtatccaatgtaccgattaacaagcgactggttaactatgaatttgttagctcgacaaaatcaccggttaataatgagtttgtgagttcgataaaatctaattttctgatagaaattttatatattatgcagaaatttaataaaagtagacttaacttatatattttagcattgactcttttgaagtaaaatccattccatctaaattatgacttccctacatcgagtaagtaagttgcgtctgtatcctcattttacccacttttcgctatgcaattattcaaggatctttacacaaatagcaagccaatattaattatttattttttttagtcatatatataaattatacatatattatatacccattaattatttttaatttaagtgatagattggacgactatttggattaattcttcgttattcaagataatagatgtcgtctctaatacatgagctagaagataataaggattactaggccgaaaggctgatggaaatgaacaagaagataagctcctaaatggaaacagtacggaaaaagtcaaagagcagtgcatgggaggaatcatcagtcagaaaaggaagccacgtgtcaagtagaaacaagcacgtgtccatgcaaaagccacgtaactcccttccatcacatcttccttcttcaaaacctcgtgttttactctctcttttctcactgccagtgatcgtcaggactgtgcatgtttgtttaaaaactaaaggca

SEQ ID NO: 105 - DNA, 5000 bp, Nicotiana tabacum
  source 1..5000: mol_type = unassigned DNA
acatgacccggttgtctcctttgtggtgtctttcagaacatcgccagtagttcgttgatgtggtgttgagtgaatactttttcaatatataagcaatttcgtgccttttttggttcattttgaggcttatgtaatatatttgatcatgtaatcgtgaatacttcaataaaaggtaactcttcctcagataatgtaaccaagaataatttcattgaagggaagcggtataagcataaaggtaactttccctgtgatgaagaatgatgatatcaagtaggcgttgttgatcaactttccaaatgggtggagggtaagctacttcactgccttacttcctctaagttcaagttgggggaggtatgatcggggattctctatcttcttgttgaacaagaaaacagtactactacttagcatatatagtttcaatcaataaataggaactgtactaacaatcagagcccttattttcacttagcatagttgtataccagttagaactcatactgtggaaacagagactcgtggactagcaatgcccttattaaaaactatcatggatgctctactcattgtctctccaaacaacagcactctttcataaaaaggtacttcatgcgacatccattaatacaagaagttcaagatacataacatgcagtaacagaactcgactcaacttagacttatcccacatctagcttgattttccatcaaaccagtgtgcgtaaaagtaaaacataactatagaattgatcccatggttaatcacattcacataacaagcggttgggataaaaagatggtcagttctaaggcttcaaaattaatatagccaattagtcgaacggacggtgtgcaatcctagatgacttctatgaaagtgagacgaatcaatctcccaaaagtttagtacaaagaaataacagcaaccgttcaaacaatctaatacggctacacatattaatcttcacaaattaaacattacaacaaaagcatactaaattctgaactcagtaaagattcaaatcaacagcaccatattatagacataacaacactagtgaattggtcatcaaagcaacgaagagcacagaaaagttcgaaatttaaccaatcacgaacctgatcgaagctcttgtggaacatctacacgcttcctgacttgtggagacaagagtctcgagtagattcggaaactgcccagtctaattttgatacgtattctacaatttccttactaaagagaaaaagagaaagtgtgtgagaatataaaattggcgccttctttcttttggcgctatttatctagttttagcgaccagttatcatgatttttaaagatatttttagattgccaattttcgctgagaaaaaaaggaggtaaaaggggtggtagttaaaagtgataatcaactttatataagactagtcttaatatacgtgcgttgcgcgtgtcataaaaaatattttaaaaaatatatttgtgttataatataaaaattaatacaaatttgcaagcttcaaaataatgaattaatcatatttaggagtttgatccactctagtggtttttggttcatcttgcttcattgtacaaaaaatttgagtatacttccaattctaagtcgttctattgttcaaaggaaaaaaagtgtcctaattgctcttcaacttctaaaatattgcctatctcttaccttaaatatgaattatcatttctgcgtccatattttttttttaaatcaaaattgtctttttcgtttttttttttttagaaaactatactctatagcccacaaaattttaatagctcatgtcttttctcacttacacgaaatggccctttggcccaaacataatagaccgaaatgtctattttgtatattttttgtatagtgacagtctattttgaatatttttttatataatgacagtctattttgtatatatattgtatagtgacggtggggagtgggttgctctagtggtaagcaccctccacttccaaccaagaggttgtgagttcgagtcaccccaagagcaaggtggggagttcttggagggagggagccgagggtctatcagaaaagcctctctaccccagggtagaggtaaggtctgcgtacacactaccctccacataccctactagtgggattatactggatcgttgttgttgttgttgttgtattgtatagtgacggtctatttagtatataattgtatagtgtttgtatattttttgtatagtgacagtctattgtatataaattgtacagtgacagactattttatatattttttgtatactgacagtctattttgtatatatgttgtatagtgacaatctatttattactccctccgttccaatttatgtgaacatgtttgactgggcatgaaatttaagaaaaaataaagacttttggaatttgtggtcctaaacaagtcaaaaggaggtccagagtatttgtggggttataaaagcttctcattaagggtacaattgtaagtttaagctaaattattatcaaatttagaaaagggtcattctttttggaacagaccaaaaaggaaataggttcacataaactggaacaaatggagtatattttttgtatagtgagagtctatttttcatacttctatgctaagtattgactttaaacactgttcaaacttaaacgtgtctctttcgcgtgaaattactctatattgaactactacaactatttgtgccggattttgtcaaaaaattcaattttccggccaagttcgttctgcttcctcctcccatccttccaatcttattctttctgccacttttcagcaatagtgatacaactaagatatgttttaaggttttgaactatttgtgtggaagagttttatggcatatactttttctttggtcgttgagaagagaggtcgacgtcgctcatgaagacattctgccgccgacgaaacagatcttatagctagccgctagaattattttaggctataaaatgtatattataattttggactactggatgatatatgttttgggccgatgggctataaatgaattttgctcatttttttaacgtgttctttattagcataaaaattgcaggatgagtttgttactttaatttcattaatttactctttttttgcagaatatacatacatgaaaagtaacatgtagtaaatattattatgttacatatatagtttaaataaaggaaaaagttaatgtaaggtagaattttaattgactttaaattcctaaattttaggacttctcacctagttcaaataagtaaatatttaataatcagcatcttagttaatttcaaagtactaaatattatgataataattaaatgactattttgtctagtcgaaatctattttttaagggtaaaaaaagcgaacgatatttcgctaagaaccttcgtgcttttaatataatactagtttctatgtacgtgcgttgcacgtaaatctttagtttatcatttatatgaaaaaacaatacattaaatttactggaaaatcatatacaaaaagaccgaataaagccattaatgtgggaacaatgcaatagtatcgtcaatgggaacatgcattgcacatatattccgtgtcaatagttatctttgtgaaataaaaaatgatacatgtaattttatactatcattcatattatgaataaacttgtttgatttttaaaatagaaaataataaagagcacgaaaagttactgctagtcattaaaatttgttatgaacattgactctattatagaataactcatcaagaagaattttaagtgcaatattgaaggatttgctttagtttctaatagtataaatcgatgtctcttatcggatcataaaaccaaaagaactataatgtccatacaactccctatgtatgtttacaatttgctttgtgtgtgtaaacattagaacttttcacctaaatatctcatacctcttaagaaagaatccttgctggatacttttcttttggagcttcacaattataagttaccaattaactaatctctccaagcctctatttaacatatacacgtttatctctggacgaacaaacaatagatggcgtcgaagatccactcttcatcttcctgattttcatcaacttccttatgcctatttatttcttgttttcagttaattcgacaaattaaattagaacaaaatccattactgctggttatcagacaaagaaaaactaaagtaactccttttgccaacaccaatgtagaacataaataagcttaaaggtaatttcttttaaattttaggcaaaacctttatgtaaacgaacatttaagccgtggctttgccattggagttctaatacaaataggacctttagttttcaatgacttgaattctacaactaatgaactgtttttacgtttttggatattaatgttgaagacatgatttaaatatgaagggtaaaaaagtcggtcaatatttcagggacatttttgtcgttcaatatttagcgtgtaacattcgtgtttttataataatatagatatagatatagatttctctccccaaccccgagattttttaaaagtattttaaaatagggttactgtgcacatataagaatcagaaatttccaggaccatagcaatgagcattattgaacagtagtagtacgtatgtcctttctggtataggatatgtagcttcattaaaagatagaaaatgaaaagcgtataaagtttgtgatacatttactaataaatccaacgaataaaagaaatactcgtatataaaagtagaaaagtaagtgtttgtactttttataaaaacacaataggtggagttgagagggatatggaaattgccttggatattatgtaggcatcatgaaccaattaatgggacctacaagattaatgttttggctatccttatcttttattgacaggcccttcaatttaaatcgttgctgcccaa

SEQ ID NO: 106 - DNA, 5000 bp, Nicotiana tabacum
  source 1..5000: mol_type = unassigned DNA
ccgactaacaaaggagtcaaacaatggcgaaactagaaatttacccaagggtattcaaatttaaaaaaagtgaaaacaaattcctacaaagggtgtttaatatgtgttatatacttctaaaacgtaatatttatctataaacacaatgcaatttttcaacgaagagtgatcaactgaccaccttataaccgccactggagtcaaaccctagttttcttttttagcttctttgtattattggtagaggtgaaccattttatacaaacatacatacatatatgtgtgtgtgagagagaaactttataaaaatagtactatgaatattgaatatcgaatttccattctcaaataactcataactttaatgttaactcattgtaaaaccttttttttacatgttagggtctgtactagctagcaccatggatagtgcgtgtcaccattttacagcaaataatatgcctcatgaattggtaggggaagggtctttaaagttagtgtgtttttttttctttttcttggtaggtggtgggggaggggaaaatgaggggacaaaaaaaatcaattggagtacaatttacacaagactccattgtatagtgctgaaaagaaatataatgttgtaaggcaaagaggataacactttagaaattatagccaacctcatgtttcttacaagtggatgagggttcgactttgcatttagcatcaccttattccctctaattttcaacaggacatgttaataaaaggatgacaatccagattgacaaagttatgaaagtgaaagcactgtaacatttcattcaatcatgaaaagcatctcccattaatttttctatcttttggaatattacgaaaatcaaaccaacttttttacgtataaatttctaatgatgtcaggggaactcctgagaaatattctcactcaagttcgaaaaggaccctttaataatgatcaataattttttttaatggtcgtgtaatattcgaatcagctcgtacacatttcaattattgtaacaagtaccttttatcccccaacaatacatatataaggtaacttatcacgtaatatttttgtctttgttggaatgtaaatcctgtttttataatttttatccactttattgaccgctaggtcacatcttatggagcttttttagtcatgataaatattactattattaagaagggagaaaaaagcaaaaggaagatattaacattagtataacaccattcataaacgtgatttaggaaataaagtatgaagaaatgaatagtgaaaaaagtcaagtacttaggagttaaggttcgtaagtagatgggacagatcgcggtggaaaaggaagagaagcttgaaagatacaaaagcaaaggggggttgggttgagattctgagttgtgattaataccactcatgaagaaaaagatatgtcatggcaccattcaggacatacagagccaactctccccccaaaagaaaaggcgcaaaacaaaaggtttgaaacagtacttgtggaggtgattcagccatgctttcatgactcatcaatgcctacttttttttctgtttttgtttttcctttttattttcaggatataaagtttgttgttaacttaaataaagaaataaaattactctcttcaacctattaagctttaggtgaaattagtaactcataaaatttaacatggtatgaattgatagaagtcgtagacacccataggtgcgctttgtagtgaaaattagggttcaaataagataaaacatgcttgatgatttcttctcatcggtttaagtttggatgagcaaagttatccaatacaagtattgccggtacctaataaaatactcgagatgcacgcaaacaaattttgacactactgttaacaataaacaatatgcagctgtccaaaattaatactatatcttattgtcactcagtcaacaaaaatattccttatgtttagtccaagaaaaataaccatacccaattataagggtttgttgaagacagaaatatataaataaatgaatcatcatatctccaatagtttaaaatttcaaatgggggtatttcacacaatttatcaaaattttaaataatttttatactttttcttgcttggaaggggaaggggtggtggtggagatagggcggggcatcttctatctagtctatgtgattaatataactaaacaaaaagggcaaggcaaaaacatggatgaatggtggtccttttctatatttatatggattgttacgatacgtcgatttcactttgcaaaatacccactagattcatttagttttctttttgatcactctgctttttctatcatatatataggaagtccttccacgtttcgcatgtgtcattgtttatattttccatggtcttccttccaaatggctaaataattttgacacagtggtcccgaaagtttatagaaatagaattcaacagtgaggcatatacgtatgaattatattttacatcttcatcgtataaaatagaatgtgctataaactttacctcgtgatgcttacaaggggtgaaaatataaaagcactttatagatttacaagagtcacaccttcttgatttatcctaagattgtatttttttacatgccaagcaatgaagtatgggagatccaattggaataacatcaaaattaataagattcgaaattgtcagagagctgtctactgagatacattgaaacttatttcttttgtacaacagaaaatatcaaatatttagcaacatattaaaatcttcataaattacattgtttaaatcttacgctgtaacatatgttgataggaggtaggcctattgatgaatttataaggtgaaaatacatggactagccagttttcgaactagtaattgaaaaaaggtatattatcacttttagcccgcgccagaaattatttatatttggtagttgaaaaagtgtataaatttgtaattttttgtatataacacacataatgtgtgtgtgagtgtatatatatacacacacaaaaactatatatattttttctattattttgagagtggttatacagtgtcatctttctaatcgaaaatagccagcgtttgcaatgttattaaaaaaaaatagccactattttagctgaaacacggaaagttccagcataatatatcggatttcagagctcctgcatataaacttccagcacattatgaaattccatcacattatgctgaaatttttccggattcttaaggtgttttcattcagattttatctttacataaaaaaatggctaaatttcgattacttttgaaattatagctctttttcaattactaattataaatctgactatttctgattttttgcctctggtcaaaatggcatggcatagtcatttttaggggtggttattgaaaaatagctagtattcacaaagttattgaaaatagtcattatttacggcgaagattaaatcttaacaaatgtacctgagttaaaacacaaaaagttacaacataatatgttggatcatggaactccttcatgtatgcttctagcatattatgttgaaactccagcacattatgaattccaacacattatgtcgaaatctcatatgtaaaaaattcgaactctggcatattatgctggaattttttcgtattttttatcagattttattttcacataacaaagtgactagatttcaataacttttgaaactatgaccaattttcaattaattgtaaatcaaattatttctgatttttttcctccagggcaataatgggactcaactttaggcaacaaaaaactgtttatgtaggtaaacgcacgaaaattgggcttctccagattagagccgggccttgtggaaagacaagaaattcgaggcccacttctagtttctaaggagattaagcgtaaaaatagcactggctagccagttttcggactgatcattcaaaaatagccagtatttgcaaagtcattgaaaaatagccattagtattttgctgcaacacgaaaagttccaacataatatattggagatcagtgcacctatgtatgaacttccagcatattatgctggaactccaacacgcggaaagttccactataatatactggagattggagcaccggtgtcaacaaatctatcatttaataggatttatggctatttttaaatgaccacttgtaaatctgattattttttaatttctcccgcttaagcctaaccaacgatggtcatgaacgaaatgtctgtttttactctctactatatagctgacccagaatcattacaatttgcaatttcctcataataaaatgtgatgtagtatagatttgaaaacccttgaagattattaatttatctttcgcgaaacaaactgtagtatccaaggtaccgaatagtaagcgattggctcacaatgagtctgtttgttcgataaaatcattggttaataatgagtctctgagttccatacaatctaatgctctgatagaaattttatatatatgcagaaagtaataaaaatagactatgacttactatattttaacattcactcttttgaagtaaaatcccttccgtcaaaattatgacttcattacaccaaattgcgcctgtatactcattttaccttctcttcactatgcaattattgagataaaaaatttcgtctctaagacatagctagaggataaggatcttaggccgaaacactgatggaactgaacaagaagataatcacctaaatgggaacagtacggaaaaagtcaaagagcagtgcatgggaggaatcatcagtcagagaaggtagccacgtgtcaagtagaaacaagcacgtgtccatgcaaaagccacgtaactccactccctcatatcttccttcttcaaaacctcgtgttttactccccctttcctcactgccggtgatcgtcaggactgtgcatgtttgtttaaaaattaaaggcg

SEQ ID NO: 107 - DNA, 5000 bp, Nicotiana tabacum
  source 1..5000: mol_type = unassigned DNA
acatgacccggttgtctccttggtggtgtcattcaaaaaatcgccagtagttcgttgatgtggtgttgagtgcatactttttcaatataagcaatttcgtgccttttgtttttccattttgaggcttatgtagtatatttgatcatgtaatcgtgaatatttcaataaaaggtaacttttccttagataatgtgaccaatgatgatctcattgaagggtatacttttcctgtgaagaagaatgatgatactcctatcatgtaggcgttgttgatcaactttcccaatgggtgaagggtaagctacttcactgccttactttttctaagttcaagttggtggaggtgtgattggggatcctttatcttcttgttgaacaagaaaacgctactaattagcaatgaataggaactgtactaacaatcagagcccttattttcacttagtatagttgtatgtataccagttagaacctatactctggaaacagggactcgtcaacaataaccagaagggtggattagaaactatcatgaattccctactcattgtctctccaaacaacaacactcttttataaaaagtgtactgcaagaatcatgttatgcttttaaggaatgtgcatcaagattgaagtatcatgcgacatccattaacacgagaagttcaagatgcacaacgtgcactaactgaagtcgactcaacgtagacttattccacatctaacttgatctttcataaaaccggtgtgcgtaaaacattactatagaattgatcccacggttactcatattcacacaacaagcaattgggacaaaaagatggtcagttctaaggcttcaaaattcatacagccaattagtcgaacatgcggtgtgcaatcctagatgacttctatgaaaatgagaccaatcaatcttccaaaagttcagtacaaagaaataacagcaacccttcaaacaatctaatacaacgacacatattaatcttcacaaattaaacacaacatcaaaagcgtactagattttgaactcggtaaagaatccaaatcaacagcaccgaattatcgacataataccactagtgaattagtcatcaaagcaacgaagagcacagaaaaagttaagaaatttaaccaataacaaacctgatcgaagatctcttagaacatctacacgcttcccgacttgtggcgacaagagtctcaagtagactcggaagctgctcagtctaattttaaatatatattctacactttcctcgcaaaagagaaaaagagagtgtatgagaatattaaattggcgcctttttcttttggcggaattgttctagttttagcgaccagttatcatgattttactactatgtaaatcgcaagcattaaagttatagcaagaaatgagaccgagtgacctaaaaacaatcacatttgtatgtttcggttccacgagcagctctgggcggacctagtaggagctcctttgcactgttgcaggagtgctcaagtacctttactctcttggtgactcagacactactgccatatcaatgagaaaaaaggaggtaaagtggtagttaaaagtgataatccattttatataaaatccctccccccgcccccatacccccggaattgtaaaaatatttttaaatagtgttactatgcgcatatactagtcagaaatttgtaggattacgggaggtagtacgtgcgggtgtacaaaccaaatcggaaaatcgcaccaaaccgaaaagtcaaatcaaaaccgattaaaagattcgactagatttggtttggtattgagtaaaacaacccgaattaaaccgacatataaatataaatttttatgtatacttttaagatttttatatagaattttctttaagaaaatatctaaaaatatttgggattctcttacgggatataatatttaataaaatatgaagtgctccatatttattaaccttaaacaatgggtcgtatgatcactttcttatcaagtgttactgaaatgcgtcaatctctttgttcttccatagtcaagatctattaaattcttatatctttttcgaatttgaagtggttattattatttaagtatcatattgacttttacgtttaattactaaattcggttaaccttgaaagtgtatatcaacaaaaattattgtcggacgactaaaagactaactatcatgtgttattaagtaaattcacgcataagaatatttaatagataatatatttttctaatttttaaaatttttactaaatatatttacttataaaaaatttaacaaagtaagattgaaataatatttaagtaacgaaaaaccagccaaaaccgatatagtttgtgtagtttgatttaaataaaagtcgaacccaacccgatccatgtacacccttaatagtacgtatgtcctttctggcatactggtaggatttgtagctgcattaaagatagaaagcgaaaatcgtacaaagtttgtaatactagtacattactaataaatccaacggataaaagaaatatataaaagtagaaaagtaattggtctagattttatgaaacacaataggtggagttgagaggggtatggaaattggcttggatattatgtaggcatcatgaaccattaatgggacctacaagattaatgttttggatatccatatcttttgttgacaggcccttcaattgaaatctttgttgcccaaaatgattccactgtcaaccaattataagttttttgttaaaaggtttattgcaccattgctccactaacttcatgtgcatttgcaacttaccacacaagagagaagaaaagtttccagtacacaggattaacatttgctaagttgattcggagttttagttagcaaagttgaaattatcaaagacagattttgaaactataagccagtggatcgctaaggggttcctcgttgataaatacttattttcatctggtgtttatactaatccaataaatatataatatatgtctttacaaatatggttataattaaatcataattaattagtacacattgttacctcacgttatcactttaccatgataagagtttggtataaatactggtgggactaagttttttaccttcctcgtttagtgtctacaagcctaattaggtgaagatggtgctggatttttttttttttttgttacaagtgtagactacagaaattaaattactggtcctattgtgcaaaatttgaattcaaatcttcgtgcatttcagtatgttatttggtacaaaaaacatggcatttatggcttcgcattgaggaagaacttgtatatagcttgaatgctcttgttacatccaccctccttaaatacccctagttgatcaacaggcaccaaaaatgtctagacacaaacatgaaccagttttatttctattctctttattgtaggtaattgtttgctgtcatcatataatataagctaggaagaaaataagtaaaaatcatcagatgtgataggtcttgaatagagaagcaaacctaatgtagcataataagaaaggaagattaggtgggagaccattatttatgacctcatcccaattttaatcatatttcattattccaggttactactaccaaacaaaaataaacgacaagaataatgcataaacttccacaatagctcattatttatttatcagccggagtgtattaaagtaatgatataattagaagacaattgagttgctacttaaactgatgctaacctgacgcgttttcgttttccaggtacagtcttccaacatcagatgtcccaattggagttccaagtcaaagtggaattggaaaatggtaagaaaactctcagccttagctgtgacatagacatcaataaagtagaaaaaaatcataagatttcaatgtttgaatgctaagcgacacaaaaatcactaaataatttttttcatttctgactaagtctttatgagcaaaactatttattacttacaattttgtgatgataggagatatcaaatactcgtggaatacttgaggtacccacaaagtgattgagaagttgctgttaattaaaaaatccttgcatcagatcccttcagtttgaggtcaaaagcacaatcatgaaaagccctctcacagcacattcacttgatccattctattttacgtcttttctctaactacgacttaagttcttctaacttttaatcattaccaccctattaaaacttatctaaagactgtttttctctgcttttctccatatttacctcaacgtctccttccatatccaagctttggtactttttccccttgtggtattgattcaggcatggtcccttctccaatcttatcaatcaaacatatactaataaagtataatataccaccgactttggtgtcaaccacgagttcgggagcttgtgaaatatggtggtatttagccaatttttttctttttaataaaattgacaattaggaaccaacttgccggcttcataataaagtatatagttgaaatggttcagaaatattaaattcaattctataaattacttggattcggtccatcctaaaggtggtcacttctaggatcaagccccattgttggctaaattttggttttaaatagattaattagcttggagtcgaacagcaagcattaagtttaccaggtctatgatttagttaggcatgaattgtaatacaaagatgatttatgcagtgattaatgatatcaggatatatatatataagtattgttatactataaataacaacaacaacaacgacccagtaaagtcccactaagtggggtttggggaaggtagtgtgtacgcagaccttatccttaccctgatagggcagagaggttgtttccgatagatcctcagctcaggaagatgaaaataaaacaagaaaacaagaaaagacagtaaccatagaaataatgacagcatcctaaaaaccataaaatagatga

SEQ ID NO: 108 - DNA, 5000 bp, Nicotiana tabacum
  source 1..5000: mol_type = unassigned DNA
aagctagcaccatagatagtgggtgtcaccattttacagcaaataatatgcctcaattggtggagaaagggtctttaaagttgaagtgtttttctttttttctctttctttttcttggtaggtggtggggaaggggaaaatgaggggacaaaacacgaaaaatcaattttgatgtataatttacacaagactcaagctaagtgatagagttgataaaaggataacaataatatgggacacataaattcatgacgtctccgtttatcggatattttctcaaattgatgtaacatatgtttcaagaagttcaaataatgtgtatatagatttatttctataaaatattatttcgaattataaatcttcgactcaagaaaaaataaaaatgttagatatggcaaaggggatagcacttagacaattaggcaacctcatatttgttacaagtgggggtgagggttcaactttgcattatttagcatcactttgttatttattttttttatataaaatttatagaacaggagatgttaatataaggatgacaatccagattgacaaagttgtgatagtgatactactgtaacatcttattcaatcatgcaaagcatctcccattaatttgtccatcttttcgaatattacgaaaatcagacctactttttttacatataattttctctaatgacatcacaggaactactcctgagagaaatattctcactcaagtttgaaaacgactcttaaattatgataaagtgataactacaatgaccggctaactcttgcgtgcattatgagtgtatatagttatatgttatttactctcgtcgtaaatatcaagtgcgaataatatgtatgaaatttagagaaatgagagtaaattagtcgatgtttttaatttacgttgtgatttattcgttactttttgctttctctaaagtcaatttcagtagttcgtggagatacattagattaaattaatctaatctgttaaatactacacggtaaaaaataaaaaatatttaaaacgacataaaattttattatcaaaataagtaaatcacaaattgggggggggtttggctttttttttttttgtccgaaaattattatcatcggtatatataactatgcttcttatattatatttttttagtgaaaattctaattttcgtaggcatacatttaattaagataagtaaatatgatgaataagaacagattgataaacgtgatttaagaaaaataaagaaaggagaaatgaatagtagttccaatttataaaaaaaagtcaagtacttaggagttaaggttcgtgagtagatgggacatatcacggtggaaaaggtagaagaagcttcaaagatacaaaagaaaaagaggggttgagattctgtgttgtgattaatacaattcaatcaaagaaaaagatgtgtcattttggacatacaactctccctaaaataaaatgccaataaacacagtacttgtggacatcatccacccatccttttatgactcatcacacaaaaaaaacacttttttccttcttatttttgaagatattctatgaagtttgttgtaaacttaaacaaaataaatcaacaaaattgtcctatctctaaatagaggagtttttaaaagttaaaaatcaatagaatagatacatgaagtagtcgaaggagatttgacattttattatgtatatatataaaattattttaatcatatataaaaaatataattttctatcaaaaagtgaatcctttcatatttgtatctccgccctatctccaaatattaagctcacacatgaaaagagcgagagacgaatgtagcctattggctatgggttccctcactttcgatatgatgtgtataaaaaaattaattgaaatctcaataaatattagattttaacaataagttaaaaattcaaatcattaagtggggtcttgatttattttcttttgttttctttctcaaactgatgtttttattaaggatttattagaaaaatatttctatctctcggggtaggaattaaatttgctatgtgtatatcctaccttttgttaaatttagtttacagaattatcggttaaatatgttagatatgttattattgttgttacgtccgaatagacacccataggtgtagtgtagtcaaattaataaaaccatggaatacgatgggatatcaaagttgataatttttaactaaaaaaaaatgtcactagatgatttttttttctctcaattgtctaatcttgacagaaagatgtacctaatgaaataataaaagtgcaacaaaaaagaatagacaaatttccaaaaattgaatcttttgttggtactcgtcaacaagttattcttcatgtctaaatcctaaaaaaaatatctcctcgtgtaaagactggtatattgaaaatttaaaataaattaatacatctaaacgtatcatatcttctatataatataaaatttttagtgactcgtttcttacaattcaataatttagtataaataatttgtcgattgcgcggatgaaagaaaggggtggtggtggagatataaagtgtgatcgatagggtatcttgtatgtgattaatatagctaaacaaaaaggtgaaaggcaaaaacatatatgaatggtggtcctattccatatttaaatggattattaggatacgtcgatttcactttgcaaaataccctctatagattcatatattttcttttctcatcaacttttttttctatcatataactagccttactctggcttctcacgtttcacatgtctcaatgtttatttgttttttttccactcttgggcgtctaaagggtttagaaaaatttgacaatgtggaccacccaaagtttataaaattagaattaaacagtcatgaaggcatatacctaggagttccatttacatcttcacgtataaaatgtgtactttatattaattttatttatctcatgattcttattagagatgataaatgtaacattttttttttgtttttattaaattttagaagagataaccttcttgattaattctatgattttccttttttattacgaaactgaaacttaacaattaactgagacataacaaacatgaatgtagttcatatctaaattaacatcaaagccatcatatctaactcaactaagttagtcatggtactcttgtgattgacataaattttacaataaactttcttagctagaacggactcaccaacaaatataaaaccacagaaaaaattatatcgaactcaatactaaacaaagaagtatggaagactaaatatgttgatacgaggtaggcttattggtgattttacaagggcttatatagcaaaagttgatacaaacaaatgggctaaaaagtaaatggcccatttattttatttattttttccattatcaatacacagatttgcacttcgttagcggacaattttgggtaaattatgaaaattgggcctcttcacaatttagatcccgaccgtgtggatggatggacaaggaaattttaggtccagttccagttatattggggagataaaaggaaaaattacgtggttaagtaatatatatattagttaattagttattataataattattttagttaattattattcgcgattaacattagtgataattatgtaggctgagattttgagtttgtataatttgaaattttaagatataatttctataacttttatatagtacaattttataaaatattatttgtataattgataaaatttagatgtttgtgtttgtataaatttatattttagatttatcaaaatataactcaattattcaaactaaccataatacgtacgaattcactcacaaattatacaaacaatacaatttgaaccatagctgcaacccctaataatgcgagctacgactataaagtataattaaatttatttactataacggatatttgcaaaaattctccttaggcataaccaataatggcccctgaacgagttgctcgaaaaagctaattaaagatcattcatttttggaaaactatataaatcagaagactaaaatatagcttttagcttttcacttgaaataagttattttttatttaaataaattattttgataattattaaacactctaataaattaaaaagatgatttttaagtcagattgatcagcttttaagtccatctaaagatgctttaattacatcttattttttaaaaaaaattaattattaaaatttcaaagttttcattgctctcgaagacatttttttaaatgtccggtacattgtaataatatattatttaatggaaggaaagtatagaaggattttttaaaaaaaataattaagtgataaacttttttagaaattataatatgttgtatatttacgttaaatttcttaattttttaatttaacatttcccaaaaattttgtgttatccaaatgtccgaataacaaccaattaactgacactgaatttgtgaattttacgagacaaatgttcttgttaaaatttcatattaggtttcaactgtgagttatttttggaaatttaaattatgtgtgttgatcaaaatactttaaatatttttttttcattatatcatgaattttaaatagatatcatatatttttaaaagatttagatttaaattttactattaatatactaaaccgacatagccttaagttgaaaaaatatcaaattcatcgagggaaaacgaaaattgcaagtatatttggccaaacgaagcacctgtccatgaaatgccacgtaaaaattctaagcaaaaatactacaataagtgcatggaatgagacgagcacgtgtcaagttgaaagaagcacctgtccatgaaatgccacgtaactcacaccttcaaaacctctccattgctacaaatctccatatttttgttttttttttttaagaaaaagataaaagtactagca

SEQ ID NO: 109 - DNA, 2400 bp, Nicotiana tabacum
  source 1..2400: mol_type = unassigned DNA
ttagcacttaaaaaacataggtttacatgctctcattgtcacgccaggccgctaattaaatggtacatttcatcatccccattttttggcctcatagttaattatcacaatttctgaaagcaacatcagaacaaccccacatttcttgtggtcctataattactgctagatagtccaaacccatctgcctatttagggcttgtgaatgaggattgaaaatggtagagaatatttgcaaaggcatcatgcatatatggaaaagactaaagagagagttagtgccttgcaaagcggattctcacagttgtagaaaaggactcaccattttcaagaatactcccggcatgaagatggacaatttaatataaaaaacaaagaaaatagtaattaagtgcgttgagatgaatgaaatttatccgctcttaattgaatatggggaattgaaagaaatttctgataataaattaatgagtcttacatgacgtggatccgacttaatctagtctatttaaactaagaatagataagaatagagaatatagacaaaagagaggctcattggctagggtttcaagggagttccttgaacataagtggcaagtacaagcacaaagccaatttccatggactaaagatgaataagatgtgtcgtgtggtatggtgggaaggtgaggaggtatggggtaattggagatgctaaacctctctaaaagctcttttgctccaaatatctaaatccatctctatcacttttggcgactgccccaaaatttgcaacttatgaattaaagttttaatatttttaagttaataaattctgaattaataatttaacatattcaataaactttttaaaacaaattacgtatataccatcaaactggctgcaccatgatcactttctaaactcacaatgacatatggatttaatcaggcacaaagtcatgttgatagaaagagatagtacggagaatgaagaaaaaggtaggggagagagatggggtgagtggggaaaagatagggttctctttttagtgaaagcgacagggtctgagaaccctaggtcaaaagttgcataaacctctatacaggcttcttcactcccttactactaatatactctcattaaggcttgaggtttaattcattaaaattgtggtttaattattgtatcccctcaaacgaaataattgtccttgtcgaggttagacaatgttgcgtactattttcaaacgcagtcagccattattctcctatcctttacagtcgagattcaaagacagaaagtagcatgcaagctgttattaatttactttgattaggactttgccaagaaaatgaagaaccttttcttttttcttttaatttagttatcttacaacatgtaatttttcctagcaagcaaatacggtaacttttttttttattctcatttaatttgttggagctattgctactttgatgacttcaaccaaatcctggttggtaggcggagggtgctgacgatggaaactacccctcttgtccaaatacgataacctaaaaaatagaataatagcttattgtactgtgctgcaaaaattgcattgtcagtatacataattaaaatctattttgaatgtgtggagggcaaagaggggtgactggtctagggttgtagaaatcaggtgggagagagaatggtatttgtctctgtgtcagctgatatcacgtgaagaggcacaataagaagtccttcgtatccattcacttcccaaaaataccggcattactacaaatatagtactagcacttgctttctctatccccatctttgctatttcctttccctttccaactttttggctttagaattgcaaagatggagggaattgtggttctttgtatctgtaaaatttttcctccaagctccagttgtagctagcttaatgcgtggacgcgcgcgcgcacacactagaaatctgcaatctatatatatattcacaaggcactcacatatcaaaaaccacatagacattgtatagagagagctgtcgttctcaagcagaaaaaatgatatgatttcatcagcatgtggtcaaccaaatagttcaattctagtctttgcttcctctttctaattactgtataaatagagccacaaggacatagaattgagaaaataaaagacaataaaaacaaatctagctacttaagcgaatgatgatgactctctctcagtagtcttaactcttaatacccttgttttccttcttgtgctgcagtttgattggttaattaacctaatcaaaagatgttttaactgtgttttatccgtctttctcaagatctatcttagtcccaccacatagctccctcaagctacagctgcaaaatatatactatatatatatataacaa

SEQ ID NO: 110 - protein, 7 aa, unidentified
  REGION 1..7: note = Description of Unknown:WRKY transcription factor core domain sequence
WRKYGQK

SEQ ID NO: 111 - protein, 7 aa, unidentified
  REGION 1..7: note = Description of Unknown:WRKY transcription factor core domain sequence
WRKYGKK

SEQ ID NO: 112 - protein, 5 aa, unidentified
  REGION 1..5: note = Description of Unknown:WRKY transcription factor "PRSYY" motif sequence
PRSYY

SEQ ID NO: 113 - DNA, 777 bp, Nicotiana tabacum
  source 1..777: mol_type = unassigned DNA
atggatacaagttctcgggacaagtcttttgctattgaccttaacacaaacccttcaccgcaaaacgtcaatagtattccgcatgagatgttggatgaagagttgatcaggatgaaagaggagaataagaagctaacgacaacgttaacaactttgtgtgagaactacaattacttgcaaactcacctaattgaattgctgcaaaaacacaactctgaggagcagaattccaaattattatctagaaaaagaaaggctgaagatgatcactgttgtgaaaattcagaaatcataattgaagaagcatcacctaagaagccaagggaaatcagaaccaacgtttcaactgtttgtgtaaaaactactccctccgatcaaagcgcagtggtgaaagatggatatcactggagaaaatatggtcaaaaagtcacaagagataatccttcacctagagcctactataagtgttcttttgcaccatcatgcccagtcaagaagaaggtgcaaagaagtgttgaagatccatcagttttagtagctacatatgagggggagcacaaccatcctctcccatcccaagctcaagtaacagtcccattaattaaccaaaatgttacaacaaatcctagttttctgaacaaattcatgcaagacattgacacaacttcgctacagcaagttttagtcgcacaaatggcgtcttccttgaccaagaaccctagtttcacagctgcagttgctgcagccatctccggaaaattctttgaatatgattaa

SEQ ID NO: 114 - protein, 258 aa, Nicotiana tabacum
MDTSSRDKSFAIDLNTNPSPQNVNSIPHEMLDEELIRMKEENKKLTTTLTTLCENYNYLQTHLIELLQKHNSEEQNSKLLSRKRKAEDDHCCENSEIIIEEASPKKPREIRTNVSTVCVKTTPSDQSAVVKDGYHWRKYGQKVTRDNPSPRAYYKCSFAPSCPVKKKVQRSVEDPSVLVATYEGEHNHPLPSQAQVTVPLINQNVTTNPSFLNKFMQDIDTTSLQQVLVAQMASSLTKNPSFTAAVAAAISGKFFEYD

SEQ ID NO: 115 - protein, 33 aa, unidentified
  REGION 1..33: note = Description of indentified:WRKY transcription factor zinc finger region sequence
  VARIANT 2..6: note = X is any known amino acid
  REGION 2..6: note = This region may encompass 4-5 residues
  VARIANT 2..6: note = One of these positions may be absent
  VARIANT 8..30: note = X is any known amino acid
  REGION 8..30: note = This region may encompass 22-23 residues
  VARIANT 8..30: note = One of these positions may be absent
  VARIANT 32: note = X is any known amino acid
CXXXXXCXXXXXXXXXXXXXXXXXXXXXXXHXH

SEQ ID NO: 116 - protein, 35 aa, unidentified
  REGION 1..35: note = Description of unidentified:WRKY transcription factor zinc finger region sequence
  VARIANT 2..8: note = X is any known amino acid
  VARIANT 10..32: note = X is any known amino acid
  VARIANT 34: note = X is any known amino acid
CXXXXXXXCXXXXXXXXXXXXXXXXXXXXXXXHXC

SEQ ID NO: 117 - protein, 4 aa, unidentified
  REGION 1..4: note = Description of Unknown:WRKY transcription factor "WRKY" motif sequence
WRKY

SEQ ID NO: 118 - DNA, 204 bp, synthetic construct
  misc_feature 1..204: note = Description of Artificial Sequence: Syntheticpolynucleotide
  source 1..204: mol_type = other DNA
cctcttcctggcataccagcagttgctaacatctatgtttttgttgttctattttgcacatttgtccactaataggtcctgtgttcttacacttcatcactatagtgtgtgtacagagaaacaggaccttttagtggacaaatgcctaaaatagggcaaaggatggtcacgtcttaatgttatattaatatgatggtcaggttt

SEQ ID NO: 119 - DNA, 204 bp, synthetic construct
  misc_feature 1..204: note = Description of Artificial Sequence: Syntheticpolynucleotide
  source 1..204: mol_type = other DNA
cctcttcctggcataccagcagttgctaacatctatgtttttgttgttctattttgcacatcatttgcgcggaaactacgatgttcttacacttcatcactatagtgtgtgtacagagaaatcgtagttaccgcgcaaatgatgcctaaaatagggcaaaggatggtcacgtcttaatgttatattaatatgatggtcaggttt

SEQ ID NO: 120 - DNA, 204 bp, synthetic construct
  misc_feature 1..204: note = Description of Artificial Sequence: Syntheticpolynucleotide
  source 1..204: mol_type = other DNA
cctcttcctggcataccagcagttgctaacatctatgtttttgttgttctattttgcacatacattcgtcttacggtccagtgttcttacacttcatcactatagtgtgtgtacagagaaactggaccgaaagacgaatgtatgcctaaaatagggcaaaggatggtcacgtcttaatgttatattaatatgatggtcaggttt

SEQ ID NO: 121 - DNA, 21 bp, synthetic construct
  misc_feature 1..21: note = Description of Artificial Sequence: Syntheticoligonucleotide
  source 1..21: mol_type = other DNA
tttgtccactaataggtcctg

SEQ ID NO: 122 - DNA, 21 bp, synthetic construct
  misc_feature 1..21: note = Description of Artificial Sequence: Syntheticoligonucleotide
  source 1..21: mol_type = other DNA
tcatttgcgcggaaactacga

SEQ ID NO: 123 - DNA, 21 bp, synthetic construct
  misc_feature 1..21: note = Description of Artificial Sequence: Syntheticoligonucleotide
  source 1..21: mol_type = other DNA
tacattcgtcttacggtccag

SEQ ID NO: 124 - DNA, 21 bp, synthetic construct
  misc_feature 1..21: note = Description of Artificial Sequence: Syntheticoligonucleotide
  source 1..21: mol_type = other DNA
caggaccttttagtggacaaa

SEQ ID NO: 125 - DNA, 21 bp, synthetic construct
  misc_feature 1..21: note = Description of Artificial Sequence: Syntheticoligonucleotide
  source 1..21: mol_type = other DNA
tcgtagttaccgcgcaaatga

SEQ ID NO: 126 - DNA, 21 bp, synthetic construct
  misc_feature 1..21: note = Description of Artificial Sequence: Syntheticoligonucleotide
  source 1..21: mol_type = other DNA
ctggaccgaaagacgaatgta

SEQ ID NO: 127 - protein, 34 aa, unidentified
  REGION 1..34: note = Description of Unknown:WRKY transcription factor "WRKY" motif sequence
  VARIANT 2..4: note = X is any known amino acid
  VARIANT 9..33: note = X is any known amino acid
  CROSSLNK 1..4: note = Aspartic acid bonded to tryptophan
  CROSSLNK 4..33: note = Tryptophan bonded to lysine
  SITE 4: note = Tryptophan triad bonded to aspatic acid and lysine
DXXXWRKYXXXXXXXXXXXXXXXXXXXXXXXXXK

(19) United States
(12) Patent Application Publication
(10) Pub. No.: US 2026/0327641 A1
(43) Pub. Date: Oct. 22, 2026

(54) COMPOSITIONS AND METHODS FOR PRODUCING TOBACCO PLANTS AND PRODUCTS HAVING REDUCED OR ELIMINATED SUCKERS

(71) Applicant: Altria Client Services LLC, Richmond, VA (US)

(72) Inventors: Raja S. PAYYAVULA, Henrico, VA (US); Yanxin SHEN, Henrico, VA (US); Chengalrayan KUDITHIPUDI, Midlothian, VA (US); Rajanikanth GOVINDARAJULU, Henrico, VA (US)

(73) Assignee: Altria Client Services LLC, Richmond, VA (US)

(21) Appl. No.: 19/656,529

(22) Filed: Apr. 23, 2026

Related U.S. Application Data
(63) Continuation of application No. 17/893,246, filed on Aug. 23, 2022, now Pat. No. 12,637,686.
Application No. 17/893,246 is a continuation of application No. 16/886,118, filed on May 28, 2020, now Pat. No. 11,447,790.

(51) Int. Cl. C12N 15/82

(52) U.S. Cl. CPC C12N 15/8262

(57) ABSTRACT

The present disclosure provides methods and compositions for controlling sucker growth in tobacco by altering the expression of different target genes.

### CROSS-REFERENCE TO RELATED APPLICATIONS

This application is a continuation of U.S. patent application Ser. No. 17/893,246, filed Aug. 23, 2022, which is a U.S. patent application Ser. No. 16/886,118, filed May 28, 2020, now U.S. Pat. No. 11,447,790, issued Sep. 20, 2022, which claims the benefit of U.S. Provisional Application No. 62/854,139, which was filed on May 29, 2019. The entire contents of these applications are incorporated by reference herein in their entireties.

### FIELD

The present disclosure provides methods and compositions for refining the expression of nucleic acids and proteins useful for the reduction or elimination of suckers in plants.

### INCORPORATION OF SEQUENCE LISTING

A sequence listing contained in the file named “P34703US02_SL.xml” which is 292,278 bytes (measured in operating system MS-Windows®), produced on Jun. 30, 2026, containing a total number of 127 sequences, starting from SEQ ID NO: 1 to SEQ ID NO: 127, is filed electronically herewith and incorporated by reference in its entirety.

### BACKGROUND

Tobacco is a plant species that exhibits exceptionally strong apical dominance. Molecular signals from the shoot apical meristem (SAM) mediate a hormonal signal that effectively inhibits axillary bud growth. Upon removal of the SAM (also known as “topping”), physiological and molecular changes occur, enabling the growth of new shoots (or “suckers”) from axillary meristems (buds). Sucker growth results in loss of yield and leaf quality. Suckers have been controlled by manual removal and through the application of chemicals. Maleic hydrazide and flumetralin are routinely used on topped plants to inhibit axillary bud growth (“suckering”). However, labor and chemical agents to control suckers are very expensive. Control of suckering in tobacco through conventional breeding, mutation breeding, and transgenic approaches have been a major objective for several decades but, to date, successful inhibition or elimination of suckering has not been achieved through these approaches. Recent molecular work has produced transgenic plants with reduced or eliminated suckers, but leaky expression of axillary bud-degrading genes can result in the death of seeds and embryos and prevents the production of successive generations of transgenic plants. Therefore, development of methods and compositions to prevent axillary bud-degrading genes from being expressed in non-desired tissues and/or organs would result in a reduction of the use of chemical agents and would reduce costs and labor associated with tobacco production.

### SUMMARY

In one aspect, this disclosure provides a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the modified tobacco plant comprises no suckers or reduced suckers after topping as compared to the control tobacco plant when grown under comparable growth conditions.

In one aspect, this disclosure provides a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety.

In one aspect, this disclosure provides a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, where the modified tobacco plant comprises no suckers or reduced suckers after topping as compared to a control tobacco plant when grown under comparable growth conditions.

In one aspect, this disclosure provides a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.

In one aspect, this disclosure provides a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, where the modified tobacco plant comprises no suckers or reduced suckers after topping as compared to a control tobacco plant when grown under comparable growth conditions.

In one aspect, this disclosure provides a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

In one aspect, this disclosure provides a tobacco product comprising cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the modified tobacco plant comprises no suckers or reduced suckers after topping as compared to the control tobacco plant when grown under comparable growth conditions.

In one aspect, this disclosure provides a tobacco product comprising cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety.

In one aspect, this disclosure provides a tobacco product comprising cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and where the modified tobacco plant comprises no suckers or reduced suckers after topping as compared to a control tobacco plant when grown under comparable growth conditions.

In one aspect, this disclosure provides a tobacco product comprising cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.

In one aspect, this disclosure provides a tobacco product comprising cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, where the modified tobacco plant comprises no suckers or reduced suckers after topping as compared to a control tobacco plant when grown under comparable growth conditions.

In one aspect, this disclosure provides a tobacco product comprising cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

In one aspect, this disclosure provides a method for producing a modified tobacco plant comprising: (a) inducing a mutation in at least one tobacco cell at a genomic locus encoding a polypeptide selected from the group consisting of a cyclin, a cyclin-dependent kinase (CDK), a CDK inhibitor, a MYB, and a WRKY transcription factor; (b) selecting at least one tobacco cell comprising the mutation from step (a); and (c) regenerating a modified tobacco plant from the at least one tobacco cell selected in step (b), where the modified tobacco plant comprises no suckers or reduced suckers after topping as compared to a control tobacco plant lacking the mutation when grown under comparable growth conditions.

In one aspect, this disclosure provides a method for producing a modified tobacco plant comprising: (a) introducing a recombinant DNA construct to at least one tobacco cell, where the recombinant DNA construct comprises a heterologous promoter operably linked to a nucleic acid encoding a polypeptide selected from the group consisting of a cyclin, a cyclin-dependent kinase (CDK), a CDK inhibitor, a MYB, and a WRKY transcription factor; (b) selecting at least one tobacco cell comprising the recombinant DNA construct; and (c) regenerating a modified tobacco plant from the at least one tobacco cell selected in step (b), where the modified tobacco plant comprises no suckers or reduced suckers after topping as compared to a control tobacco plant lacking the recombinant DNA construct when grown under comparable growth conditions.

In one aspect, this disclosure provides a method for producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor; (b) selecting at least one tobacco cell comprising the recombinant DNA construct; and (c) regenerating a modified tobacco plant from the at least one tobacco cell selected in step (b), where the modified tobacco plant comprises no suckers or reduced suckers after topping as compared to a control tobacco plant lacking the recombinant DNA construct when grown under comparable growth conditions.

In one aspect, this disclosure provides a method comprising preparing a tobacco product using cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the modified tobacco plant comprises no suckers or reduced suckers after topping as compared to a control tobacco plant when grown under comparable growth conditions.

In one aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and where the modified tobacco plant comprises no suckers or reduced suckers after topping as compared to a control tobacco plant when grown under comparable growth conditions.

In one aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and wherein said modified tobacco plant comprises no suckers or reduced suckers after topping as compared to a control tobacco plant when grown under comparable growth conditions.

In one aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, wherein said modified tobacco plant comprises no suckers or reduced suckers after topping as compared to a control tobacco plant when grown under comparable growth conditions.

In one aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.

In one aspect, this disclosure provides a method comprising preparing a tobacco product using cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, wherein said modified tobacco plant comprises no suckers or reduced suckers after topping as compared to a control tobacco plant when grown under comparable growth conditions.

In one aspect, this disclosure provides a method comprising preparing a tobacco product using cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

In one aspect, this disclosure provides a method comprising transforming a tobacco cell with a recombinant DNA construct comprising a heterologous promoter operably linked to a nucleic acid encoding a polypeptide selected from the group consisting of a cyclin, a cyclin-dependent kinase (CDK), a CDK inhibitor, a MYB, and a WRKY transcription factor.

In one aspect, this disclosure provides a method comprising transforming a tobacco cell with a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

In one aspect, this disclosure provides a method for producing a modified tobacco plant comprising: (a) crossing at least one tobacco plant of a first tobacco variety with at least one tobacco plant of a second tobacco variety, where the at least one tobacco plant of the first tobacco variety comprises a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the at least one tobacco plant comprises no suckers or reduced suckers after topping as compared to the control tobacco plant when grown under comparable growth conditions; and (b) selecting for progeny tobacco plants that exhibit no suckers or reduced suckers after topping compared to a control tobacco plant of the same cross grown under comparable growth conditions.

In one aspect, this disclosure provides a method for producing a modified tobacco plant comprising: (a) crossing at least one tobacco plant of a first tobacco variety with at least one tobacco plant of a second tobacco variety, where the at least one tobacco plant of the first tobacco variety comprises a recombinant DNA construct comprising a heterologous promoter operably linked to a nucleic acid encoding a polypeptide selected from the group consisting of a cyclin, a cyclin-dependent kinase (CDK), a CDK inhibitor, a MYB, and a WRKY transcription factor, where the at least one tobacco plant comprises no suckers or reduced suckers after topping as compared to a control tobacco plant when grown under comparable growth conditions; and (b) selecting for progeny tobacco plants that exhibit no suckers or reduced suckers after topping compared to a control tobacco plant of the same cross grown under comparable growth conditions.

In one aspect, this disclosure provides a method for producing a modified tobacco plant comprising: (a) crossing at least one tobacco plant of a first tobacco variety with at least one tobacco plant of a second tobacco variety, where the at least one tobacco plant of the first tobacco variety comprises a recombinant DNA construct comprising a heterologous promoter operably linked to a nucleic acid encoding at least one small RNA molecule capable of reducing the expression of an endogenous genomic locus that encodes a polypeptide selected from the group consisting of a cyclin, a cyclin-dependent kinase (CDK), a CDK inhibitor, a MYB, and a WRKY transcription factor, where the at least one tobacco plant comprises no suckers or reduced suckers after topping as compared to a control tobacco plant when grown under comparable growth conditions; and (b) selecting for progeny tobacco plants that exhibit no suckers or reduced suckers after topping compared to a control tobacco plant of the same cross grown under comparable growth conditions.

### BRIEF DESCRIPTION OF THE SEQUENCES

SEQ ID NOs: 1-44, 89-113, and 118-126 are nucleic acid sequences.

SEQ ID NOs: 45-88 and 114-117 are amino acid sequences.

Additional descriptions of the SEQ ID NOs provided herein can be found below in Table 1.

TABLE 1
Description of sequences

| SEQ ID NO | Sequence Type | Sequence Description |
|---|---|---|
| 1 | Nucleic acid | Cyclin-dependent kinase |
| 2 | Nucleic acid | Cyclin-dependent kinase |
| 3 | Nucleic acid | Cyclin-dependent kinase |
| 4 | Nucleic acid | Cyclin-dependent kinase |
| 5 | Nucleic acid | Cyclin-dependent kinase |
| | | inhibitor |
| 6 | Nucleic acid | Cyclin |
| 7 | Nucleic acid | Cyclin |
| 8 | Nucleic acid | Cyclin |
| 9 | Nucleic acid | Cyclin |
| 10 | Nucleic acid | Cyclin |
| 11 | Nucleic acid | Cyclin |
| 12 | Nucleic acid | Cyclin |
| 13 | Nucleic acid | Cyclin |
| 14 | Nucleic acid | Cyclin |
| 15 | Nucleic acid | Cyclin |
| 16 | Nucleic acid | Cyclin |
| 17 | Nucleic acid | Cyclin |
| 18 | Nucleic acid | Cyclin |
| 19 | Nucleic acid | Cyclin |
| 20 | Nucleic acid | Cyclin |
| 21 | Nucleic acid | Cyclin |
| 22 | Nucleic acid | Cyclin |
| 23 | Nucleic acid | Cyclin |
| 24 | Nucleic acid | Cyclin |
| 25 | Nucleic acid | Cyclin |
| 26 | Nucleic acid | Cyclin |
| 27 | Nucleic acid | Cyclin |
| 28 | Nucleic acid | Cyclin |
| 29 | Nucleic acid | Cyclin |
| 30 | Nucleic acid | Cyclin |
| 31 | Nucleic acid | Cyclin |
| 32 | Nucleic acid | Cyclin |
| 33 | Nucleic acid | MYB |
| 34 | Nucleic acid | MYB |
| 35 | Nucleic acid | MYB |
| 36 | Nucleic acid | MYB |
| 37 | Nucleic acid | MYB |
| 38 | Nucleic acid | MYB |
| 39 | Nucleic acid | MYB |
| 40 | Nucleic acid | MYB |
| 41 | Nucleic acid | MYB |
| 42 | Nucleic acid | MYB |
| 43 | Nucleic acid | MYB |
| 44 | Nucleic acid | MYB |
| 45 | Amino acid | Cyclin-dependent kinase |
| 46 | Amino acid | Cyclin-dependent kinase |
| 47 | Amino acid | Cyclin-dependent kinase |
| 48 | Amino acid | Cyclin-dependent kinase |
| 49 | Amino acid | Cyclin-dependent kinase |
| | | inhibitor |
| 50 | Amino acid | Cyclin |
| 51 | Amino acid | Cyclin |
| 52 | Amino acid | Cyclin |
| 53 | Amino acid | Cyclin |
| 54 | Amino acid | Cyclin |
| 55 | Amino acid | Cyclin |
| 56 | Amino acid | Cyclin |
| 57 | Amino acid | Cyclin |
| 58 | Amino acid | Cyclin |
| 59 | Amino acid | Cyclin |
| 60 | Amino acid | Cyclin |
| 61 | Amino acid | Cyclin |
| 62 | Amino acid | Cyclin |
| 63 | Amino acid | Cyclin |
| 64 | Amino acid | Cyclin |
| 65 | Amino acid | Cyclin |
| 66 | Amino acid | Cyclin |
| 67 | Amino acid | Cyclin |
| 68 | Amino acid | Cyclin |
| 69 | Amino acid | Cyclin |
| 70 | Amino acid | Cyclin |
| 71 | Amino acid | Cyclin |
| 72 | Amino acid | Cyclin |
| 73 | Amino acid | Cyclin |
| 74 | Amino acid | Cyclin |
| 75 | Amino acid | Cyclin |
| 76 | Amino acid | Cyclin |
| 77 | Amino acid | MYB |
| 78 | Amino acid | MYB |
| 79 | Amino acid | MYB |
| 80 | Amino acid | MYB |
| 81 | Amino acid | MYB |
| 82 | Amino acid | MYB |
| 83 | Amino acid | MYB |
| 84 | Amino acid | MYB |
| 85 | Amino acid | MYB |
| 86 | Amino acid | MYB |
| 87 | Amino acid | MYB |
| 88 | Amino acid | MYB |
| 89 | Nucleic acid | Promoter |
| 90 | Nucleic acid | Promoter |
| 91 | Nucleic acid | Promoter |
| 92 | Nucleic acid | Promoter |
| 93 | Nucleic acid | Promoter |
| 94 | Nucleic acid | Promoter |
| 95 | Nucleic acid | Promoter |
| 96 | Nucleic acid | Promoter |
| 97 | Nucleic acid | Promoter |
| 98 | Nucleic acid | Promoter |
| 99 | Nucleic acid | Promoter |
| 100 | Nucleic acid | Promoter |
| 101 | Nucleic acid | Promoter |
| 102 | Nucleic acid | Promoter |
| 103 | Nucleic acid | Promoter |
| 104 | Nucleic acid | Promoter |
| 105 | Nucleic acid | Promoter |
| 106 | Nucleic acid | Promoter |
| 107 | Nucleic acid | Promoter |
| 108 | Nucleic acid | Promoter |
| 109 | Nucleic acid | Promoter |
| 110 | Nucleic acid | WRKY core domain |
| 111 | Nucleic acid | WRKY core domain variant |
| 112 | Nucleic acid | WRKY domain motif |
| 113 | Nucleic acid | WRKY transcription factor |
| 114 | Amino acid | WRKY transcription factor |
| 115 | Amino acid | WRKY transcription factor |
| | | zinc finger region |
| 116 | Amino acid | WRKY transcription factor |
| | | zinc finger region |
| 117 | Amino acid | WRKY domain |
| 118 | Nucleic acid | Artificial miRNA precursor |
| 119 | Nucleic acid | Artificial miRNA precursor |
| 120 | Nucleic acid | Artificial miRNA precursor |
| 121 | Nucleic acid | Mature artificial miRNA |
| 122 | Nucleic acid | Mature artificial miRNA |
| 123 | Nucleic acid | Mature artificial miRNA |
| 124 | Nucleic acid | Artificial miRNA* |
| 125 | Nucleic acid | Artificial miRNA* |
| 126 | Nucleic acid | Artificial miRNA* |
| 127 | Nucleic acid | DWK salt bridge motif |

### BRIEF DESCRIPTION OF THE DRAWINGS

FIG. 1 is a Venn diagram depicting the overlap of differentially expressed genes in axillary buds between 4-hour, 24-hour, and 72-hour post-topping pairwise comparisons of water-treated and maleic hydrazine-treated tobacco plants. MH4 refers 4-hours after maleic hydrazide treatment; W4 refers to 4-hours after water treatment; MH24 refers to 24-hours after maleic hydrazide treatment; W24 refers to 24-hours after water treatment; MH72 refers to 72-hours after maleic hydrazide treatment; W72 refers to 72-hours after water treatment.

FIG. 2 depicts the collective mass of suckers from individual P1_2.4::MYB plants (20GH729; 20GH280; 20GH283; 20GH284; 20GH285; 20GH282; 20GH281; and 20GH182) one week after topping.

FIG. 3 depicts the collective mass of suckers from individual P1_2.4::CDKI plants (20GH290; 20GH294; 20GH287; 20GH293; 20GH295; 20GH288; 20GH291; 20GH292; 20GH296; and 20GH289) one week after topping.

FIG. 4 depicts the collective mass of suckers from individual plants (20GH304; 20GH311; 20GH302; 20GH303; 20GH308; 20GH314; 20GH310; 20GH301, 20GH307; 20GH312; 20GH313; 20GH305; 20GH306; and 20GH309) comprising the amiRNA-1 construct one week after topping.

FIG. 5 depicts the collective mass of suckers from individual plants (20GH320; 20GH325; 20GH317; 20GH319; 20GH321; 20GH324; 20GH316; 20GH318; 20GH322; 20GH326; 20GH315; and 20GH323) comprising the amiRNA-2 construct one week after topping.

FIG. 6 depicts the collective mass of suckers from individual plants (20GH328; 20GH333; 20GH331; 20GH332; 20GH329; 20GH327; 20GH334; and 20GH330) comprising the amiRNA-3 construct one week after topping.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof.

In an aspect, a modified tobacco plant provided herein comprises reduced suckering or no suckering after topping as compared to a control tobacco plant when grown under comparable conditions.

As used herein, “suckering” refers to the development and/or growth of axillary (or lateral) buds (“suckers”) from axillary meristems that grow between a leaf and the stalk. An axillary bud is an embryonic shoot that comprises an axillary meristem, surrounding leaf tissue, and surrounding stem tissue. See, for example, U.S. Patent Application Publication Nos. 2016/0281100; and 2017/0260535, which are incorporated by reference herein in their entireties.

As used herein, “topping” refers to the removal of the stalk apex, including the shoot apical meristem, flowers, and up to several adjacent leaves, when a plant is near maturity. Topping a tobacco plant results in the loss of apical dominance. Prior to topping, suckering is largely kept dormant by hormonal signals emanating from the shoot-apical meristem; topping removes the hormonal signals and can allow the outgrowth of suckers (“topping-induced suckering”). Provided suckering is sufficiently controlled, topping increases yield, increases value-per-acre, and results in desirable modifications to physical and chemical properties of tobacco leaves.

As used herein, “comparable growth conditions” refers to similar environmental conditions and/or agronomic practices for growing and making meaningful comparisons between two or more plant genotypes so that neither environmental conditions nor agronomic practices would contribute to, or explain, any differences observed between the two or more plant genotypes. Environmental conditions include, for example, light, temperature, water, humidity, and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp. 70-103. It is appreciated in the art that “comparable growth conditions” does not require identical growth conditions.

As used herein, “modified” refers to plants, seeds, plant parts, plant cells, and plant genomes that have been subjected to mutagenesis, genome editing, genetic transformation, or a combination thereof.

In an aspect, this disclosure provides a modified tobacco plant that comprise no suckers or reduced suckers after topping as compared to a control tobacco plant when grown under comparable conditions. As used herein, a “reduction” in the number of suckers, the size of suckers, and/or the impact suckers have on agronomic performance refers to a statistically significant reduction. As used herein, “statistically significant” refers to a p-value of less than 0.05 when using an appropriate measure of statistical significance (e.g., a one-tailed two sample t-test).

In one aspect, a modified plant or method provided herein requires reduced management for controlling suckering compared to a control plant when grown under comparable conditions. As used herein, “management” refers to manually removing suckers, application of chemicals (e.g., maleic hydrazide, flumetralin) to inhibit or remove suckers, or both. In one aspect, a modified plant or method provided herein requires reduced frequency of manual sucker removal, reduced frequency of chemical application, reduced quantities of chemical application, or a combination thereof, as compared to a control plant grown under comparable conditions. See, for example, Fisher et al. “Topping, Managing Suckers, and Using Ethephon,” pages 96-117 In: 2016 Flue-Cured Tobacco Information, North Carolina State University, which is herein incorporated by reference in its entirety.

In an aspect, a modified plant or method provided herein requires reduced frequency of maleic hydrazide application to reduce or eliminate suckers. In an aspect, a modified plant or method provided herein requires reduced quantities of maleic hydrazide application to reduce or eliminate suckers. In an aspect, a modified plant or method provided herein requires reduced frequency of flumetralin application to reduce or eliminate suckers. In an aspect, a modified plant or method provided herein requires reduced quantities of flumetralin application to reduce or eliminate suckers.

In one aspect, a modified plant or method provided herein requires manual removal of suckers 1% as frequently as a control plant when grown under comparable conditions. In one aspect, a modified plant or method provided herein requires manual removal of suckers 5% as frequently as a control plant when grown under comparable conditions. In one aspect, a modified plant or method provided herein requires manual removal of suckers 10% as frequently as a control plant when grown under comparable conditions. In one aspect, a modified plant or method provided herein requires manual removal of suckers 20% as frequently as a control plant when grown under comparable conditions. In one aspect, a modified plant or method provided herein requires manual removal of suckers 30% as frequently as a control plant when grown under comparable conditions. In one aspect, a modified plant or method provided herein requires manual removal of suckers 40% as frequently as a control plant when grown under comparable conditions. In one aspect, a modified plant or method provided herein requires manual removal of suckers 50% as frequently as a control plant when grown under comparable conditions. In one aspect, a modified plant or method provided herein requires manual removal of suckers 60% as frequently as a control plant when grown under comparable conditions. In one aspect, a modified plant or method provided herein requires manual removal of suckers 70% as frequently as a control plant when grown under comparable conditions. In one aspect, a modified plant or method provided herein requires manual removal of suckers 80% as frequently as a control plant when grown under comparable conditions. In one aspect, a modified plant or method provided herein requires manual removal of suckers 90% as frequently as a control plant when grown under comparable conditions. In one aspect, a modified plant or method provided herein requires manual removal of suckers 95% as frequently as a control plant when grown under comparable conditions.

In one aspect, a modified plant or method provided herein requires manual removal of suckers less than 1% as frequently as a control plant when grown under comparable conditions. In one aspect, a modified plant or method provided herein requires manual removal of suckers less than 5% as frequently as a control plant when grown under comparable conditions. In one aspect, a modified plant or method provided herein requires manual removal of suckers less than 10% as frequently as a control plant when grown under comparable conditions. In one aspect, a modified plant or method provided herein requires manual removal of suckers less than 20% as frequently as a control plant when grown under comparable conditions. In one aspect, a modified plant or method provided herein requires manual removal of suckers less than 30% as frequently as a control plant when grown under comparable conditions. In one aspect, a modified plant or method provided herein requires manual removal of suckers less than 40% as frequently as a control plant when grown under comparable conditions. In one aspect, a modified plant or method provided herein requires manual removal of suckers less than 50% as frequently as a control plant when grown under comparable conditions. In one aspect, a modified plant or method provided herein requires manual removal of suckers less than 60% as frequently as a control plant when grown under comparable conditions. In one aspect, a modified plant or method provided herein requires manual removal of suckers less than 70% as frequently as a control plant when grown under comparable conditions. In one aspect, a modified plant or method provided herein requires manual removal of suckers less than 80% as frequently as a control plant when grown under comparable conditions. In one aspect, a modified plant or method provided herein requires manual removal of suckers less than 90% as frequently as a control plant when grown under comparable conditions. In one aspect, a modified plant or method provided herein requires manual removal of suckers less than 95% as frequently as a control plant when grown under comparable conditions.

In one aspect, a modified plant provided herein requires manual removal of suckers between 10% and 95% as frequently as a control plant when grown under comparable conditions. In one aspect, a modified plant provided herein requires manual removal of suckers between 20% and 95% as frequently as a control plant when grown under comparable conditions. In one aspect, a modified plant provided herein requires manual removal of suckers between 30% and 95% as frequently as a control plant when grown under comparable conditions. In one aspect, a modified plant provided herein requires manual removal of suckers between 40% and 95% as frequently as a control plant when grown under comparable conditions. In one aspect, a modified plant provided herein requires manual removal of suckers between 50% and 95% as frequently as a control plant when grown under comparable conditions. In one aspect, a modified plant provided herein requires manual removal of suckers between 60% and 95% as frequently as a control plant when grown under comparable conditions. In one aspect, a modified plant provided herein requires manual removal of suckers between 70% and 95% as frequently as a control plant when grown under comparable conditions. In one aspect, a modified plant provided herein requires manual removal of suckers between 80% and 95% as frequently as a control plant when grown under comparable conditions. In one aspect, a modified plant provided herein requires manual removal of suckers between 90% and 95% as frequently as a control plant when grown under comparable conditions. In one aspect, a modified plant provided herein requires manual removal of suckers between 10% and 75% as frequently as a control plant when grown under comparable conditions. In one aspect, a modified plant provided herein requires manual removal of suckers between 10% and 50% as frequently as a control plant when grown under comparable conditions. In one aspect, a modified plant provided herein requires manual removal of suckers between 10% and 25% as frequently as a control plant when grown under comparable conditions. In one aspect, a modified plant provided herein requires manual removal of suckers between 50% and 75% as frequently as a control plant when grown under comparable conditions. In one aspect, a modified plant provided herein requires manual removal of suckers between 25% and 75% as frequently as a control plant when grown under comparable conditions. In one aspect, a modified plant provided herein requires manual removal of suckers between 25% and 50% as frequently as a control plant when grown under comparable conditions. In one aspect, a modified plant provided herein requires manual removal of suckers between 1% and 50% as frequently as a control plant when grown under comparable conditions. In one aspect, a modified plant provided herein requires manual removal of suckers between 1% and 25% as frequently as a control plant when grown under comparable conditions. In one aspect, a modified plant provided herein requires manual removal of suckers between 1% and 10% as frequently as a control plant when grown under comparable conditions.

In one aspect, a modified plant provided herein requires chemical application to control suckering 1% as frequently as a control plant when grown under comparable conditions. In one aspect, a modified plant provided herein requires chemical application to control suckering 5% as frequently as a control plant when grown under comparable conditions. In one aspect, a modified plant provided herein requires chemical application to control suckering 10% as frequently as a control plant when grown under comparable conditions. In one aspect, a modified plant provided herein requires chemical application to control suckering 20% as frequently as a control plant when grown under comparable conditions. In one aspect, a modified plant provided herein requires chemical application to control suckering 30% as frequently as a control plant when grown under comparable conditions. In one aspect, a modified plant provided herein requires chemical application to control suckering 40% as frequently as a control plant when grown under comparable conditions. In one aspect, a modified plant provided herein requires chemical application to control suckering 50% as frequently as a control plant when grown under comparable conditions. In one aspect, a modified plant provided herein requires chemical application to control suckering 60% as frequently as a control plant when grown under comparable conditions. In one aspect, a modified plant provided herein requires chemical application to control suckering 70% as frequently as a control plant when grown under comparable conditions. In one aspect, a modified plant provided herein requires chemical application to control suckering 80% as frequently as a control plant when grown under comparable conditions. In one aspect, a modified plant provided herein requires chemical application to control suckering 90% as frequently as a control plant when grown under comparable conditions. In one aspect, a modified plant provided herein requires chemical application to control suckering 95% as frequently as a control plant when grown under comparable conditions.

In one aspect, a modified plant provided herein requires chemical application to control suckering less than 1% as frequently as a control plant when grown under comparable conditions. In one aspect, a modified plant provided herein requires chemical application to control suckering less than 5% as frequently as a control plant when grown under comparable conditions. In one aspect, a modified plant provided herein requires chemical application to control suckering less than 10% as frequently as a control plant when grown under comparable conditions. In one aspect, a modified plant provided herein requires chemical application to control suckering less than 20% as frequently as a control plant when grown under comparable conditions. In one aspect, a modified plant provided herein requires chemical application to control suckering less than 30% as frequently as a control plant when grown under comparable conditions. In one aspect, a modified plant provided herein requires chemical application to control suckering less than 40% as frequently as a control plant when grown under comparable conditions. In one aspect, a modified plant provided herein requires chemical application to control suckering less than 50% as frequently as a control plant when grown under comparable conditions. In one aspect, a modified plant provided herein requires chemical application to control suckering less than 60% as frequently as a control plant when grown under comparable conditions. In one aspect, a modified plant provided herein requires chemical application to control suckering less than 70% as frequently as a control plant when grown under comparable conditions. In one aspect, a modified plant provided herein requires chemical application to control suckering less than 80% as frequently as a control plant when grown under comparable conditions. In one aspect, a modified plant provided herein requires chemical application to control suckering less than 90% as frequently as a control plant when grown under comparable conditions. In one aspect, a modified plant provided herein requires chemical application to control suckering less than 95% as frequently as a control plant when grown under comparable conditions.

In one aspect, a modified plant provided herein requires chemical application to control suckering between 10% and 95% as frequently as a control plant when grown under comparable conditions. In one aspect, a modified plant provided herein requires chemical application to control suckering between 20% and 95% as frequently as a control plant when grown under comparable conditions. In one aspect, a modified plant provided herein requires chemical application to control suckering between 30% and 95% as frequently as a control plant when grown under comparable conditions. In one aspect, a modified plant provided herein requires chemical application to control suckering between 40% and 95% as frequently as a control plant when grown under comparable conditions. In one aspect, a modified plant provided herein requires chemical application to control suckering between 50% and 95% as frequently as a control plant when grown under comparable conditions. In one aspect, a modified plant provided herein requires chemical application to control suckering between 60% and 95% as frequently as a control plant when grown under comparable conditions. In one aspect, a modified plant provided herein requires chemical application to control suckering between 70% and 95% as frequently as a control plant when grown under comparable conditions. In one aspect, a modified plant provided herein requires chemical application to control suckering between 80% and 95% as frequently as a control plant when grown under comparable conditions. In one aspect, a modified plant provided herein requires chemical application to control suckering between 90% and 95% as frequently as a control plant when grown under comparable conditions. In one aspect, a modified plant provided herein requires chemical application to control suckering between 10% and 75% as frequently as a control plant when grown under comparable conditions. In one aspect, a modified plant provided herein requires chemical application to control suckering between 10% and 50% as frequently as a control plant when grown under comparable conditions. In one aspect, a modified plant provided herein requires chemical application to control suckering between 10% and 25% as frequently as a control plant when grown under comparable conditions. In one aspect, a modified plant provided herein requires chemical application to control suckering between 50% and 75% as frequently as a control plant when grown under comparable conditions. In one aspect, a modified plant provided herein requires chemical application to control suckering between 25% and 50% as frequently as a control plant when grown under comparable conditions. In one aspect, a modified plant provided herein requires chemical application to control suckering between 25% and 75% as frequently as a control plant when grown under comparable conditions. In one aspect, a modified plant provided herein requires chemical application to control suckering between 1% and 50% as frequently as a control plant when grown under comparable conditions. In one aspect, a modified plant provided herein requires chemical application to control suckering between 1% and 25% as frequently as a control plant when grown under comparable conditions. In one aspect, a modified plant provided herein requires chemical application to control suckering between 1% and 10% as frequently as a control plant when grown under comparable conditions.

In one aspect, a modified plant provided herein requires a chemical spray volume of 1% of the volume used to control suckering of a control plant when grown under comparable conditions. In one aspect, a modified plant provided herein requires a chemical spray volume of 5% of the volume used to control suckering of a control plant when grown under comparable conditions. In one aspect, a modified plant provided herein requires a chemical spray volume of 10% of the volume used to control suckering of a control plant when grown under comparable conditions. In one aspect, a modified plant provided herein requires a chemical spray volume of 20% of the volume used to control suckering of a control plant when grown under comparable conditions. In one aspect, a modified plant provided herein requires a chemical spray volume of 30% of the volume used to control suckering of a control plant when grown under comparable conditions. In one aspect, a modified plant provided herein requires a chemical spray volume of 40% of the volume used to control suckering of a control plant when grown under comparable conditions. In one aspect, a modified plant provided herein requires a chemical spray volume of 50% of the volume used to control suckering of a control plant when grown under comparable conditions. In one aspect, a modified plant provided herein requires a chemical spray volume of 60% of the volume used to control suckering of a control plant when grown under comparable conditions. In one aspect, a modified plant provided herein requires a chemical spray volume of 70% of the volume used to control suckering of a control plant when grown under comparable conditions. In one aspect, a modified plant provided herein requires a chemical spray volume of 80% of the volume used to control suckering of a control plant when grown under comparable conditions. In one aspect, a modified plant provided herein requires a chemical spray volume of 90% of the volume used to control suckering of a control plant when grown under comparable conditions. In one aspect, a modified plant provided herein requires a chemical spray volume of 95% of the volume used to control suckering of a control plant when grown under comparable conditions.

In one aspect, a modified plant provided herein requires a chemical spray volume of less than 1% of the volume used to control suckering of a control plant when grown under comparable conditions. In one aspect, a modified plant provided herein requires a chemical spray volume of less than 5% of the volume used to control suckering of a control plant when grown under comparable conditions. In one aspect, a modified plant provided herein requires a chemical spray volume of less than 10% of the volume used to control suckering of a control plant when grown under comparable conditions. In one aspect, a modified plant provided herein requires a chemical spray volume of less than 20% of the volume used to control suckering of a control plant when grown under comparable conditions. In one aspect, a modified plant provided herein requires a chemical spray volume of less than 30% of the volume used to control suckering of a control plant when grown under comparable conditions. In one aspect, a modified plant provided herein requires a chemical spray volume of less than 40% of the volume used to control suckering of a control plant when grown under comparable conditions. In one aspect, a modified plant provided herein requires a chemical spray volume of less than 50% of the volume used to control suckering of a control plant when grown under comparable conditions. In one aspect, a modified plant provided herein requires a chemical spray volume of less than 60% of the volume used to control suckering of a control plant when grown under comparable conditions. In one aspect, a modified plant provided herein requires a chemical spray volume of less than 70% of the volume used to control suckering of a control plant when grown under comparable conditions. In one aspect, a modified plant provided herein requires a chemical spray volume of less than 80% of the volume used to control suckering of a control plant when grown under comparable conditions. In one aspect, a modified plant provided herein requires a chemical spray volume of less than 90% of the volume used to control suckering of a control plant when grown under comparable conditions. In one aspect, a modified plant provided herein requires a chemical spray volume of less than 95% of the volume used to control suckering of a control plant when grown under comparable conditions.

In one aspect, a modified plant provided herein requires a chemical spray volume between 10% and 95% less than a control plant when grown under comparable conditions. In one aspect, a modified plant provided herein requires a chemical spray volume between 20% and 95% less than a control plant when grown under comparable conditions. In one aspect, a modified plant provided herein requires a chemical spray volume between 30% and 95% less than a control plant when grown under comparable conditions. In one aspect, a modified plant provided herein requires a chemical spray volume between 40% and 95% less than a control plant when grown under comparable conditions. In one aspect, a modified plant provided herein requires a chemical spray volume between 50% and 95% less than a control plant when grown under comparable conditions. In one aspect, a modified plant provided herein requires a chemical spray volume between 60% and 95% less than a control plant when grown under comparable conditions. In one aspect, a modified plant provided herein requires a chemical spray volume between 70% and 95% less than a control plant when grown under comparable conditions. In one aspect, a modified plant provided herein requires a chemical spray volume between 80% and 95% less than a control plant when grown under comparable conditions. In one aspect, a modified plant provided herein requires a chemical spray volume between 90% and 95% less than a control plant when grown under comparable conditions. In one aspect, a modified plant provided herein requires a chemical spray volume between 10% and 75% less than a control plant when grown under comparable conditions. In one aspect, a modified plant provided herein requires a chemical spray volume between 10% and 50% less than a control plant when grown under comparable conditions. In one aspect, a modified plant provided herein requires a chemical spray volume between 10% and 25% less than a control plant when grown under comparable conditions. In one aspect, a modified plant provided herein requires a chemical spray volume between 25% and 50% less than a control plant when grown under comparable conditions. In one aspect, a modified plant provided herein requires a chemical spray volume between 25% and 75% less than a control plant when grown under comparable conditions. In one aspect, a modified plant provided herein requires a chemical spray volume between 50% and 75% less than a control plant when grown under comparable conditions. In one aspect, a modified plant provided herein requires a chemical spray volume between 1% and 50% less than a control plant when grown under comparable conditions. In one aspect, a modified plant provided herein requires a chemical spray volume between 1% and 25% less than a control plant when grown under comparable conditions. In one aspect, a modified plant provided herein requires a chemical spray volume between 1% and 10% less than a control plant when grown under comparable conditions.

In an aspect, reduced suckers comprises fewer total suckers, smaller average sucker size, or both, as compared to a control tobacco plant when grown under comparable conditions. In another aspect, smaller average sucker size comprises a measurement selected from the group consisting of reduced average mass, reduced average length, reduced average diameter, or any combination thereof, as compared to suckers of a control tobacco plant when grown under comparable growth conditions. In another aspect, smaller average sucker size comprises reduced average mass of suckers. In a further aspect, smaller average sucker size comprises reduced average length of suckers. In a still further aspect, smaller average sucker size comprises reduced average diameter of suckers.

In an aspect, a modified tobacco plant comprises fewer total suckers as compared to a control tobacco plant when grown under comparable conditions. In an aspect, a modified tobacco plant comprises a smaller average sucker size as compared to a control tobacco plant when grown under comparable conditions. In an aspect, a modified tobacco plant comprises a shorter average sucker length as compared to a control tobacco plant when grown under comparable conditions. In an aspect, a modified tobacco plant comprises a lower average sucker mass as compared to a control tobacco plant when grown under comparable conditions. In an aspect, a modified tobacco plant comprises a shorter average sucker diameter as compared to a control tobacco plant when grown under comparable conditions.

In an aspect, a modified tobacco plant comprises at least 1 fewer sucker as compared to a control tobacco plant when grown under comparable conditions. In an aspect, a modified tobacco plant comprises at least 2 fewer suckers as compared to a control tobacco plant when grown under comparable conditions. In an aspect, a modified tobacco plant comprises at least 3 fewer suckers as compared to a control tobacco plant when grown under comparable conditions. In an aspect, a modified tobacco plant comprises at least 4 fewer suckers as compared to a control tobacco plant when grown under comparable conditions. In an aspect, a modified tobacco plant comprises at least 5 fewer suckers as compared to a control tobacco plant when grown under comparable conditions. In an aspect, a modified tobacco plant comprises at least 7 fewer suckers as compared to a control tobacco plant when grown under comparable conditions. In an aspect, a modified tobacco plant comprises at least 10 fewer suckers as compared to a control tobacco plant when grown under comparable conditions. In an aspect, a modified tobacco plant comprises at least 15 fewer suckers as compared to a control tobacco plant when grown under comparable conditions. In an aspect, a modified tobacco plant comprises at least 20 fewer suckers as compared to a control tobacco plant when grown under comparable conditions. In an aspect, a modified tobacco plant comprises at least 25 fewer suckers as compared to a control tobacco plant when grown under comparable conditions. In an aspect, a modified tobacco plant comprises at least 30 fewer suckers as compared to a control tobacco plant when grown under comparable conditions.

In an aspect, a modified tobacco plant comprises at least 5% fewer suckers as compared to a control tobacco plant when grown under comparable conditions. In an aspect, a modified tobacco plant comprises at least 10% fewer suckers as compared to a control tobacco plant when grown under comparable conditions. In an aspect, a modified tobacco plant comprises at least 15% fewer suckers as compared to a control tobacco plant when grown under comparable conditions. In an aspect, a modified tobacco plant comprises at least 20% fewer suckers as compared to a control tobacco plant when grown under comparable conditions. In an aspect, a modified tobacco plant comprises at least 25% fewer suckers as compared to a control tobacco plant when grown under comparable conditions. In an aspect, a modified tobacco plant comprises at least 30% fewer suckers as compared to a control tobacco plant when grown under comparable conditions. In an aspect, a modified tobacco plant comprises at least 40% fewer suckers as compared to a control tobacco plant when grown under comparable conditions. In an aspect, a modified tobacco plant comprises at least 50% fewer suckers as compared to a control tobacco plant when grown under comparable conditions. In an aspect, a modified tobacco plant comprises at least 60% fewer suckers as compared to a control tobacco plant when grown under comparable conditions. In an aspect, a modified tobacco plant comprises at least 70% fewer suckers as compared to a control tobacco plant when grown under comparable conditions. In an aspect, a modified tobacco plant comprises at least 80% fewer suckers as compared to a control tobacco plant when grown under comparable conditions. In an aspect, a modified tobacco plant comprises at least 90% fewer suckers as compared to a control tobacco plant when grown under comparable conditions. In an aspect, a modified tobacco plant comprises at least 95% fewer suckers as compared to a control tobacco plant when grown under comparable conditions.

In an aspect, average sucker mass is measured using fresh sucker weight. In another aspect, average sucker mass is measured using dry sucker weight.

In an aspect, a modified tobacco plant comprises an average sucker mass at least 5% lower as compared to the average sucker mass of a control tobacco plant when grown under comparable conditions. In an aspect, a modified tobacco plant comprises an average sucker mass at least 10% lower as compared to the average sucker mass of a control tobacco plant when grown under comparable conditions. In an aspect, a modified tobacco plant comprises an average sucker mass at least 15% lower as compared to the average sucker mass of a control tobacco plant when grown under comparable conditions. In an aspect, a modified tobacco plant comprises an average sucker mass at least 20% lower as compared to the average sucker mass of a control tobacco plant when grown under comparable conditions. In an aspect, a modified tobacco plant comprises an average sucker mass at least 25% lower as compared to the average sucker mass of a control tobacco plant when grown under comparable conditions. In an aspect, a modified tobacco plant comprises an average sucker mass at least 30% lower as compared to the average sucker mass of a control tobacco plant when grown under comparable conditions. In an aspect, a modified tobacco plant comprises an average sucker mass at least 40% lower as compared to the average sucker mass of a control tobacco plant when grown under comparable conditions. In an aspect, a modified tobacco plant comprises an average sucker mass at least 50% lower as compared to the average sucker mass of a control tobacco plant when grown under comparable conditions. In an aspect, a modified tobacco plant comprises an average sucker mass at least 60% lower as compared to the average sucker mass of a control tobacco plant when grown under comparable conditions. In an aspect, a modified tobacco plant comprises an average sucker mass at least 70% lower as compared to the average sucker mass of a control tobacco plant when grown under comparable conditions. In an aspect, a modified tobacco plant comprises an average sucker mass at least 80% lower as compared to the average sucker mass of a control tobacco plant when grown under comparable conditions. In an aspect, a modified tobacco plant comprises an average sucker mass at least 90% lower as compared to the average sucker mass of a control tobacco plant when grown under comparable conditions. In an aspect, a modified tobacco plant comprises an average sucker mass at least 95% lower as compared to the average sucker mass of a control tobacco plant when grown under comparable conditions.

In an aspect, average sucker length is measured from the base of the sucker where it joins the main tobacco stem to the distal tip of the sucker stem.

In an aspect, a modified tobacco plant comprises an average sucker length at least 1 centimeter shorter as compared to the average sucker length of a control tobacco plant when grown under comparable conditions. In an aspect, a modified tobacco plant comprises an average sucker length at least 2 centimeters shorter as compared to the average sucker length of a control tobacco plant when grown under comparable conditions. In an aspect, a modified tobacco plant comprises an average sucker length at least 3 centimeters shorter as compared to the average sucker length of a control tobacco plant when grown under comparable conditions. In an aspect, a modified tobacco plant comprises an average sucker length at least 4 centimeters shorter as compared to the average sucker length of a control tobacco plant when grown under comparable conditions. In an aspect, a modified tobacco plant comprises an average sucker length at least 5 centimeters shorter as compared to the average sucker length of a control tobacco plant when grown under comparable conditions. In an aspect, a modified tobacco plant comprises an average sucker length at least 10 centimeters shorter as compared to the average sucker length of a control tobacco plant when grown under comparable conditions. In an aspect, a modified tobacco plant comprises an average sucker length at least 15 centimeters shorter as compared to the average sucker length of a control tobacco plant when grown under comparable conditions. In an aspect, a modified tobacco plant comprises an average sucker length at least 20 centimeters shorter as compared to the average sucker length of a control tobacco plant when grown under comparable conditions.

In an aspect, a modified tobacco plant comprises an average sucker length between 0.5 centimeters and 30 centimeters shorter as compared to the average sucker length of a control tobacco plant when grown under comparable conditions. In an aspect, a modified tobacco plant comprises an average sucker length between 0.5 centimeters and 25 centimeters shorter as compared to the average sucker length of a control tobacco plant when grown under comparable conditions. In an aspect, a modified tobacco plant comprises an average sucker length between 0.5 centimeters and 20 centimeters shorter as compared to the average sucker length of a control tobacco plant when grown under comparable conditions. In an aspect, a modified tobacco plant comprises an average sucker length between 0.5 centimeters and 15 centimeters shorter as compared to the average sucker length of a control tobacco plant when grown under comparable conditions. In an aspect, a modified tobacco plant comprises an average sucker length between 0.5 centimeters and 10 centimeters shorter as compared to the average sucker length of a control tobacco plant when grown under comparable conditions. In an aspect, a modified tobacco plant comprises an average sucker length between 0.5 centimeters and 5 centimeters shorter as compared to the average sucker length of a control tobacco plant when grown under comparable conditions. In an aspect, a modified tobacco plant comprises an average sucker length between 1 centimeter and 30 centimeters shorter as compared to the average sucker length of a control tobacco plant when grown under comparable conditions. In an aspect, a modified tobacco plant comprises an average sucker length between 1 centimeter and 20 centimeters shorter as compared to the average sucker length of a control tobacco plant when grown under comparable conditions. In an aspect, a modified tobacco plant comprises an average sucker length between 1 centimeter and 10 centimeters shorter as compared to the average sucker length of a control tobacco plant when grown under comparable conditions.

In an aspect, a modified tobacco plant comprises an average sucker length at least 5% shorter as compared to the average sucker length of a control tobacco plant when grown under comparable conditions. In an aspect, a modified tobacco plant comprises an average sucker length at least 10% shorter as compared to the average sucker length of a control tobacco plant when grown under comparable conditions. In an aspect, a modified tobacco plant comprises an average sucker length at least 15% shorter as compared to the average sucker length of a control tobacco plant when grown under comparable conditions. In an aspect, a modified tobacco plant comprises an average sucker length at least 20% shorter as compared to the average sucker length of a control tobacco plant when grown under comparable conditions. In an aspect, a modified tobacco plant comprises an average sucker length at least 25% shorter as compared to the average sucker length of a control tobacco plant when grown under comparable conditions. In an aspect, a modified tobacco plant comprises an average sucker length at least 30% shorter as compared to the average sucker length of a control tobacco plant when grown under comparable conditions. In an aspect, a modified tobacco plant comprises an average sucker length at least 40% shorter as compared to the average sucker length of a control tobacco plant when grown under comparable conditions. In an aspect, a modified tobacco plant comprises an average sucker length at least 50% shorter as compared to the average sucker length of a control tobacco plant when grown under comparable conditions. In an aspect, a modified tobacco plant comprises an average sucker length at least 60% shorter as compared to the average sucker length of a control tobacco plant when grown under comparable conditions. In an aspect, a modified tobacco plant comprises an average sucker length at least 70% shorter as compared to the average sucker length of a control tobacco plant when grown under comparable conditions. In an aspect, a modified tobacco plant comprises an average sucker length at least 80% shorter as compared to the average sucker length of a control tobacco plant when grown under comparable conditions. In an aspect, a modified tobacco plant comprises an average sucker length at least 90% shorter as compared to the average sucker length of a control tobacco plant when grown under comparable conditions. In an aspect, a modified tobacco plant comprises an average sucker length at least 95% shorter as compared to the average sucker length of a control tobacco plant when grown under comparable conditions.

In an aspect, sucker diameter is measured at the base of the sucker where it adjoins the main stem of the plant.

In an aspect, a modified tobacco plant comprises an average sucker diameter at least 5% shorter as compared to the average sucker diameter of a control tobacco plant when grown under comparable conditions. In an aspect, a modified tobacco plant comprises an average sucker diameter at least 10% shorter as compared to the average sucker diameter of a control tobacco plant when grown under comparable conditions. In an aspect, a modified tobacco plant comprises an average sucker diameter at least 15% shorter as compared to the average sucker diameter of a control tobacco plant when grown under comparable conditions. In an aspect, a modified tobacco plant comprises an average sucker diameter at least 20% shorter as compared to the average sucker diameter of a control tobacco plant when grown under comparable conditions. In an aspect, a modified tobacco plant comprises an average sucker diameter at least 25% shorter as compared to the average sucker diameter of a control tobacco plant when grown under comparable conditions. In an aspect, a modified tobacco plant comprises an average sucker diameter at least 30% shorter as compared to the average sucker diameter of a control tobacco plant when grown under comparable conditions. In an aspect, a modified tobacco plant comprises an average sucker diameter at least 40% shorter as compared to the average sucker diameter of a control tobacco plant when grown under comparable conditions. In an aspect, a modified tobacco plant comprises an average sucker diameter at least 50% shorter as compared to the average sucker diameter of a control tobacco plant when grown under comparable conditions. In an aspect, a modified tobacco plant comprises an average sucker diameter at least 60% shorter as compared to the average sucker diameter of a control tobacco plant when grown under comparable conditions. In an aspect, a modified tobacco plant comprises an average sucker diameter at least 70% shorter as compared to the average sucker diameter of a control tobacco plant when grown under comparable conditions. In an aspect, a modified tobacco plant comprises an average sucker diameter at least 80% shorter as compared to the average sucker diameter of a control tobacco plant when grown under comparable conditions. In an aspect, a modified tobacco plant comprises an average sucker diameter at least 90% shorter as compared to the average sucker diameter of a control tobacco plant when grown under comparable conditions. In an aspect, a modified tobacco plant comprises an average sucker diameter at least 95% shorter as compared to the average sucker diameter of a control tobacco plant when grown under comparable conditions.

In an aspect, a modified tobacco plant comprises an average sucker diameter at least 1 millimeter shorter as compared to the average sucker diameter of a control tobacco plant when grown under comparable conditions. In an aspect, a modified tobacco plant comprises an average sucker diameter at least 2 millimeters shorter as compared to the average sucker diameter of a control tobacco plant when grown under comparable conditions. In an aspect, a modified tobacco plant comprises an average sucker diameter at least 3 millimeters shorter as compared to the average sucker diameter of a control tobacco plant when grown under comparable conditions. In an aspect, a modified tobacco plant comprises an average sucker diameter at least 4 millimeters shorter as compared to the average sucker diameter of a control tobacco plant when grown under comparable conditions. In an aspect, a modified tobacco plant comprises an average sucker diameter at least 5 millimeters shorter as compared to the average sucker diameter of a control tobacco plant when grown under comparable conditions. In an aspect, a modified tobacco plant comprises an average sucker diameter at least 6 millimeters shorter as compared to the average sucker diameter of a control tobacco plant when grown under comparable conditions. In an aspect, a modified tobacco plant comprises an average sucker diameter at least 7 millimeters shorter as compared to the average sucker diameter of a control tobacco plant when grown under comparable conditions. In an aspect, a modified tobacco plant comprises an average sucker diameter at least 8 millimeters shorter as compared to the average sucker diameter of a control tobacco plant when grown under comparable conditions. In an aspect, a modified tobacco plant comprises an average sucker diameter at least 9 millimeters shorter as compared to the average sucker diameter of a control tobacco plant when grown under comparable conditions. In an aspect, a modified tobacco plant comprises an average sucker diameter at least 1 centimeter shorter as compared to the average sucker diameter of a control tobacco plant when grown under comparable conditions. In an aspect, a modified tobacco plant comprises an average sucker diameter at least 1.5 centimeters shorter as compared to the average sucker diameter of a control tobacco plant when grown under comparable conditions. In an aspect, a modified tobacco plant comprises an average sucker diameter at least 2 centimeters shorter as compared to the average sucker diameter of a control tobacco plant when grown under comparable conditions. In an aspect, a modified tobacco plant comprises an average sucker diameter at least 3 centimeters shorter as compared to the average sucker diameter of a control tobacco plant when grown under comparable conditions. In an aspect, a modified tobacco plant comprises an average sucker diameter at least 4 centimeters shorter as compared to the average sucker diameter of a control tobacco plant when grown under comparable conditions. In an aspect, a modified tobacco plant comprises an average sucker diameter at least 5 centimeters shorter as compared to the average sucker diameter of a control tobacco plant when grown under comparable conditions.

Any nucleic acid molecule, protein, or polypeptide provided herein is envisioned for use with any method provided herein. Any nucleic acid molecule, protein, or polypeptide provided herein is envisioned for use with any plant, plant part, cell, or seed provided herein. Any nucleic acid molecule, protein, or polypeptide provided herein is envisioned for use with any tobacco plant, tobacco plant part, tobacco cell, or tobacco seed provided herein.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded. A nucleic acid molecule can encode a polypeptide or a small RNA.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

In an aspect, an endogenous gene provided herein comprises a polynucleotide sequence at least 80% identical to a sequence selected from the group consisting of SEQ ID NOs: 1-44 and 113. In an aspect, an endogenous gene provided herein comprises a polynucleotide sequence at least 85% identical to a sequence selected from the group consisting of SEQ ID NOs: 1-44 and 113. In an aspect, an endogenous gene provided herein comprises a polynucleotide sequence at least 90% identical to a sequence selected from the group consisting of SEQ ID NOs: 1-44 and 113. In an aspect, an endogenous gene provided herein comprises a polynucleotide sequence at least 95% identical to a sequence selected from the group consisting of SEQ ID NOs: 1-44 and 113. In an aspect, an endogenous gene provided herein comprises a polynucleotide sequence at least 96% identical to a sequence selected from the group consisting of SEQ ID NOs: 1-44 and 113. In an aspect, an endogenous gene provided herein comprises a polynucleotide sequence at least 97% identical to a sequence selected from the group consisting of SEQ ID NOs: 1-44 and 113. In an aspect, an endogenous gene provided herein comprises a polynucleotide sequence at least 98% identical to a sequence selected from the group consisting of SEQ ID NOs: 1-44 and 113. In an aspect, an endogenous gene provided herein comprises a polynucleotide sequence at least 99% identical to a sequence selected from the group consisting of SEQ ID NOs: 1-44 and 113. In an aspect, an endogenous gene provided herein comprises a polynucleotide sequence 100% identical to a sequence selected from the group consisting of SEQ ID NOs: 1-44 and 113.

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

In an aspect, an endogenous gene provided herein encodes a polypeptide comprising an amino acid sequence at least 70% identical or similar to an amino acid sequence selected from the group consisting of SEQ ID NOs: 45-88 and 114. In an aspect, an endogenous gene provided herein encodes a polypeptide comprising an amino acid sequence at least 75% identical or similar to an amino acid sequence selected from the group consisting of SEQ ID NOs: 45-88 and 114. In an aspect, an endogenous gene provided herein encodes a polypeptide comprising an amino acid sequence at least 80% identical or similar to an amino acid sequence selected from the group consisting of SEQ ID NOs: 45-88 and 114. In an aspect, an endogenous gene provided herein encodes a polypeptide comprising an amino acid sequence at least 85% identical or similar to an amino acid sequence selected from the group consisting of SEQ ID NOs: 45-88 and 114. In an aspect, an endogenous gene provided herein encodes a polypeptide comprising an amino acid sequence at least 90% identical or similar to an amino acid sequence selected from the group consisting of SEQ ID NOs: 45-88 and 114. In an aspect, an endogenous gene provided herein encodes a polypeptide comprising an amino acid sequence at least 95% identical or similar to an amino acid sequence selected from the group consisting of SEQ ID NOs: 45-88 and 114. In an aspect, an endogenous gene provided herein encodes a polypeptide comprising an amino acid sequence at least 96% identical or similar to an amino acid sequence selected from the group consisting of SEQ ID NOs: 45-88 and 114. In an aspect, an endogenous gene provided herein encodes a polypeptide comprising an amino acid sequence at least 97% identical or similar to an amino acid sequence selected from the group consisting of SEQ ID NOs: 45-88 and 114. In an aspect, an endogenous gene provided herein encodes a polypeptide comprising an amino acid sequence at least 98% identical or similar to an amino acid sequence selected from the group consisting of SEQ ID NOs: 45-88 and 114. In an aspect, an endogenous gene provided herein encodes a polypeptide comprising an amino acid sequence at least 99% identical or similar to an amino acid sequence selected from the group consisting of SEQ ID NOs: 45-88 and 114. In an aspect, an endogenous gene provided herein encodes a polypeptide comprising an amino acid sequence 100% identical or similar to an amino acid sequence selected from the group consisting of SEQ ID NOs: 45-88 and 114.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31:3497-3500 (2003); Thompson et al., “Clustal W: Improving the sensitivity of progressive multiple sequence alignment through sequence weighting, position-specific gap penalties and weight matrix choice,” Nucleic Acids Research 22:4673-4680 (1994); Larkin M A et al., “Clustal W and Clustal X version 2.0,” Bioinformatics 23:2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

In one aspect, this disclosure provides a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the modified tobacco plant comprises no suckers or reduced suckers after topping as compared to the control tobacco plant when grown under comparable growth conditions.

In another aspect, this disclosure provides a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety.

In a further aspect, this disclosure provides a method for producing a modified tobacco plant comprising: (a) inducing a mutation in at least one tobacco cell at a genomic locus encoding a polypeptide selected from the group consisting of a cyclin, a cyclin-dependent kinase (CDK), a CDK inhibitor, a MYB, and a WRKY transcription factor; (b) selecting at least one tobacco cell comprising the mutation from step (a); and (c) regenerating a modified tobacco plant from the at least one tobacco cell selected in step (b), where the modified tobacco plant comprises no suckers or reduced suckers after topping as compared to a control tobacco plant lacking the mutation when grown under comparable growth conditions. In an aspect, this method further comprises (d) growing the modified tobacco plant regenerated in step (c). In another aspect, this method further comprises (e) crossing the modified tobacco plant grown in step (d) with a second tobacco plant; and (f) obtaining at least one seed from the crossing in step (e).

In an aspect, a modified tobacco plant, tobacco plant part, tobacco seed, tobacco cell, or tobacco genome provided herein comprises one or more mutations in an endogenous gene encoding a cyclin. In an aspect, a modified tobacco plant, tobacco plant part, tobacco seed, tobacco cell, or tobacco genome provided herein comprises one or more mutations in an endogenous gene encoding a cyclin-dependent kinase. In an aspect, a modified tobacco plant, tobacco plant part, tobacco seed, tobacco cell, or tobacco genome provided herein comprises one or more mutations in an endogenous gene encoding a cyclin-dependent kinase inhibitor. In an aspect, a modified tobacco plant, tobacco plant part, tobacco seed, tobacco cell, or tobacco genome provided herein comprises one or more mutations in an endogenous gene encoding a MYB. In an aspect, a modified tobacco plant, tobacco plant part, tobacco seed, tobacco cell, or tobacco genome provided herein comprises one or more mutations in an endogenous gene encoding a WRKY transcription factor.

Cyclins control the progression of cells through the cell cycle. They often play a role in the activation of CDKs. Cyclins are characterized as having a conserved cyclin box domain. Cyclin box domains typically comprise about 150 amino acids, which are organized into five alpha-helical regions. Cyclin box domains are important for binding other proteins, such as CDKs. See, for example, Noble et al., “The cyclin box fold: protein recognition in cell-cycle and transcription control,” Trends Biochem. Sci. 22:482-487 (1997); and Menges et al., “Genomic Organization and Evolutionary Conservation of Plant D-Type Cyclins,” Plant Physiology, 145:1558-1576 (2007), which are incorporated herein by reference in their entireties.

G₁ (Gap 1 phase) is the first part of interphase during the cell cycle in eukaryotic cells. Cells synthesize mRNA and proteins during G₁ phase, which ends with the transition into S phase. S phase (Synthesis phase) is the second part of interphase during the cell cycle, in which DNA is replicated. S phase ends with the transition into G₂ phase. G₂ (Gap 2 phase) typically involves rapid cellular growth as the cell prepares for transition to M phase (Mitosis phase). Cyclins and CDKs are important regulators at the checkpoints between the transition from G₁ to S phase and from G₂ to M phase, as well as during G₁ phase, S phase, G₂ phase, and M phase.

There are two main groups of cyclins: G₁/S cyclins, which are required for control of the cell cycle through the G₁/S transition, and G₂/M cyclins, which are required for control of the cell cycle through the G₂/M transition. If the required cyclins or CDKs are unable to function correctly, the cell cycle can arrest at the G₁/S transition or the G₂/M transition and prevent the formation of daughter cells. In an aspect, a cyclin provided herein is a G₁/S cyclin. In another aspect, a cyclin provided herein is a G₂/M cyclin.

In an aspect, this disclosure provides a polynucleotide that encodes a cyclin. In another aspect, this disclosure provides a cyclin.

In an aspect, this disclosure provides a mutated polynucleotide comprising a mutation in an endogenous gene, where the mutated polynucleotide encodes a mutated cyclin as compared to a cyclin encoded by the endogenous gene lacking the mutation. In an aspect, this disclosure provides a mutated polynucleotide comprising a mutation in an endogenous gene, where the mutated polynucleotide encodes a truncated cyclin as compared to a cyclin encoded by the endogenous gene lacking the mutation. In another aspect, this disclosure provides a non-naturally occurring truncated cyclin. In an aspect, this disclosure provides a polynucleotide encoding a mutated mRNA of an endogenous gene comprising a premature stop codon, where the mutated mRNA encodes a truncated cyclin as compared to an mRNA of the endogenous gene lacking the premature stop codon.

In an aspect, this disclosure provides a polynucleotide comprising a non-naturally occurring mutation in a sequence encoding a cyclin box domain. In an aspect, this disclosure provides a mutated polynucleotide comprising a mutation in an endogenous gene encoding a cyclin, where the mutated polynucleotide encodes a cyclin lacking a cyclin box domain. In another aspect, this disclosure provides a mutated polynucleotide comprising a mutation in an endogenous gene encoding a cyclin, where the mutated polynucleotide encodes a cyclin comprising a truncated cyclin box domain as compared to a cyclin encoded by the endogenous gene lacking the mutation. In an aspect, this disclosure provides a mutated polynucleotide comprising a mutation in an endogenous gene encoding a cyclin, where the mutated polynucleotide encodes a cyclin that is incapable of binding a CDK. In an aspect, this disclosure provides a mutated polynucleotide comprising a mutation in an endogenous gene encoding a cyclin, where the mutated polynucleotide encodes a cyclin that exhibits reduced binding affinity for at least one CDK as compared to a cyclin encoded by the endogenous gene lacking the mutation.

In an aspect, a mutated polynucleotide encodes a dominant negative allele of a cyclin. In another aspect, a mutated polynucleotide encodes a dominant positive allele of a cyclin. In a further aspect, a mutated polynucleotide encodes a constitutively active cyclin. In another aspect, a mutated polynucleotide encodes an inactive cyclin.

In an aspect, an endogenous gene encoding a cyclin comprises a polynucleotide sequence at least 80% identical to a sequence selected from the group consisting of SEQ ID NOs: 6-32. In an aspect, an endogenous gene encoding a cyclin comprises a polynucleotide sequence at least 85% identical to a sequence selected from the group consisting of SEQ ID NOs: 6-32. In an aspect, an endogenous gene encoding a cyclin comprises a polynucleotide sequence at least 90% identical to a sequence selected from the group consisting of SEQ ID NOs: 6-32. In an aspect, an endogenous gene encoding a cyclin comprises a polynucleotide sequence at least 95% identical to a sequence selected from the group consisting of SEQ ID NOs: 6-32. In an aspect, an endogenous gene encoding a cyclin comprises a polynucleotide sequence at least 96% identical to a sequence selected from the group consisting of SEQ ID NOs: 6-32. In an aspect, an endogenous gene encoding a cyclin comprises a polynucleotide sequence at least 97% identical to a sequence selected from the group consisting of SEQ ID NOs: 6-32. In an aspect, an endogenous gene encoding a cyclin comprises a polynucleotide sequence at least 98% identical to a sequence selected from the group consisting of SEQ ID NOs: 6-32. In an aspect, an endogenous gene encoding a cyclin comprises a polynucleotide sequence at least 99% identical to a sequence selected from the group consisting of SEQ ID NOs: 6-32. In an aspect, an endogenous gene encoding a cyclin comprises a polynucleotide sequence 100% identical to a sequence selected from the group consisting of SEQ ID NOs: 6-32.

In an aspect, an endogenous cyclin comprises an amino acid sequence at least 70% identical to a sequence selected from the group consisting of SEQ ID NOs: 50-76. In an aspect, an endogenous cyclin comprises an amino acid sequence at least 75% identical to a sequence selected from the group consisting of SEQ ID NOs: 50-76. In an aspect, an endogenous cyclin comprises an amino acid sequence at least 80% identical to a sequence selected from the group consisting of SEQ ID NOs: 50-76. In an aspect, an endogenous cyclin comprises an amino acid sequence at least 85% identical to a sequence selected from the group consisting of SEQ ID NOs: 50-76. In an aspect, an endogenous cyclin comprises an amino acid sequence at least 90% identical to a sequence selected from the group consisting of SEQ ID NOs: 50-76. In an aspect, an endogenous cyclin comprises an amino acid sequence at least 95% identical to a sequence selected from the group consisting of SEQ ID NOs: 50-76. In an aspect, an endogenous cyclin comprises an amino acid sequence at least 96% identical to a sequence selected from the group consisting of SEQ ID NOs: 50-76. In an aspect, an endogenous cyclin comprises an amino acid sequence at least 97% identical to a sequence selected from the group consisting of SEQ ID NOs: 50-76. In an aspect, an endogenous cyclin comprises an amino acid sequence at least 98% identical to a sequence selected from the group consisting of SEQ ID NOs: 50-76. In an aspect, an endogenous cyclin comprises an amino acid sequence at least 99% identical to a sequence selected from the group consisting of SEQ ID NOs: 50-76. In an aspect, a cyclin comprises an amino acid sequence 100% identical to a sequence selected from the group consisting of SEQ ID NOs: 50-76.

In an aspect, an endogenous cyclin comprises an amino acid sequence at least 70% similar to a sequence selected from the group consisting of SEQ ID NOs: 50-76. In an aspect, an endogenous cyclin comprises an amino acid sequence at least 75% similar to a sequence selected from the group consisting of SEQ ID NOs: 50-76. In an aspect, an endogenous cyclin comprises an amino acid sequence at least 80% similar to a sequence selected from the group consisting of SEQ ID NOs: 50-76. In an aspect, an endogenous cyclin comprises an amino acid sequence at least 85% similar to a sequence selected from the group consisting of SEQ ID NOs: 50-76. In an aspect, an endogenous cyclin comprises an amino acid sequence at least 90% similar to a sequence selected from the group consisting of SEQ ID NOs: 50-76. In an aspect, an endogenous cyclin comprises an amino acid sequence at least 95% similar to a sequence selected from the group consisting of SEQ ID NOs: 50-76. In an aspect, an endogenous cyclin comprises an amino acid sequence at least 96% similar to a sequence selected from the group consisting of SEQ ID NOs: 50-76. In an aspect, an endogenous cyclin comprises an amino acid sequence at least 97% similar to a sequence selected from the group consisting of SEQ ID NOs: 50-76. In an aspect, an endogenous cyclin comprises an amino acid sequence at least 98% similar to a sequence selected from the group consisting of SEQ ID NOs: 50-76. In an aspect, an endogenous cyclin comprises an amino acid sequence at least 99% similar to a sequence selected from the group consisting of SEQ ID NOs: 50-76. In an aspect, a cyclin comprises an amino acid sequence 100% similar to a sequence selected from the group consisting of SEQ ID NOs: 50-76.

Cyclin dependent kinases (CDKs) are a family of protein kinases that are involved in regulating the cell cycle, regulating transcription, and regulating mRNA processing. CDKs are recognized as belonging to the Enzyme Commission class 2.7.11.22. CDKs comprise a kinase domain, and they interact with, or bind to, cyclins. CDKs phosphorylate substrates on serine and threonine, so they can also be referred to as serine-threonine kinases. CDKs, in conjunction with cyclins, are required for cells to progress through the cell cycle. See, for example, Mironov et al., “Cyclin-Dependent Kinases and Cell Division in Plants—The Nexus,” Plant Cell, 11:509-521 (1999), which is herein incorporated by reference in its entirety.

In an aspect, this disclosure provides a polynucleotide that encodes a CDK. In another aspect, this disclosure provides a CDK.

In an aspect, this disclosure provides a mutated polynucleotide comprising a mutation in an endogenous gene, where the mutated polynucleotide encodes a mutated CDK as compared to a CDK encoded by the endogenous gene lacking the mutation. In an aspect, this disclosure provides a mutated polynucleotide comprising a mutation in an endogenous gene, where the mutated polynucleotide encodes a truncated CDK as compared to a CDK encoded by the endogenous gene lacking the mutation. In another aspect, this disclosure provides a non-naturally occurring truncated CDK. In an aspect, this disclosure provides a polynucleotide encoding a mutated mRNA of an endogenous gene comprising a premature stop codon, where the mutated mRNA encodes a truncated CDK as compared to an mRNA of the endogenous gene lacking the premature stop codon.

In an aspect, this disclosure provides a polynucleotide comprising a non-naturally occurring mutation in a sequence encoding a kinase domain. In an aspect, this disclosure provides a mutated polynucleotide comprising a mutation in an endogenous gene encoding a CDK, where the mutated polynucleotide encodes a CDK lacking a kinase domain. In another aspect, this disclosure provides a mutated polynucleotide comprising a mutation in an endogenous gene encoding a CDK, where the mutated polynucleotide encodes a CDK comprising a truncated kinase domain as compared to a CDK encoded by the endogenous gene lacking the mutation. In an aspect, this disclosure provides a mutated polynucleotide comprising a mutation in an endogenous gene encoding a CDK, where the mutated polynucleotide encodes a CDK that is incapable of binding a cyclin. In an aspect, this disclosure provides a mutated polynucleotide comprising a mutation in an endogenous gene encoding a CDK that exhibits reduced binding affinity for at least one cyclin as compared to a CDK encoded by the endogenous gene lacking the mutation.

In an aspect, a mutated polynucleotide encodes a dominant negative allele of a CDK. In another aspect, a mutated polynucleotide encodes a dominant positive allele of a CDK. In a further aspect, a mutated polynucleotide encodes a constitutively active CDK. In another aspect, a mutated polynucleotide encodes an inactive CDK.

In an aspect, an endogenous gene encoding a CDK comprises a polynucleotide sequence at least 80% identical to a sequence selected from the group consisting of SEQ ID NOs: 1-4. In an aspect, an endogenous gene encoding a CDK comprises a polynucleotide sequence at least 85% identical to a sequence selected from the group consisting of SEQ ID NOs: 1-4. In an aspect, an endogenous gene encoding a CDK comprises a polynucleotide sequence at least 90% identical to a sequence selected from the group consisting of SEQ ID NOs: 1-4. In an aspect, an endogenous gene encoding a CDK comprises a polynucleotide sequence at least 95% identical to a sequence selected from the group consisting of SEQ ID NOs: 1-4. In an aspect, an endogenous gene encoding a CDK comprises a polynucleotide sequence at least 96% identical to a sequence selected from the group consisting of SEQ ID NOs: 1-4. In an aspect, an endogenous gene encoding a CDK comprises a polynucleotide sequence at least 97% identical to a sequence selected from the group consisting of SEQ ID NOs: 1-4. In an aspect, an endogenous gene encoding a CDK comprises a polynucleotide sequence at least 98% identical to a sequence selected from the group consisting of SEQ ID NOs: 1-4. In an aspect, an endogenous gene encoding a CDK comprises a polynucleotide sequence at least 99% identical to a sequence selected from the group consisting of SEQ ID NOs: 1-4. In an aspect, an endogenous gene encoding a CDK comprises a polynucleotide sequence 100% identical to a sequence selected from the group consisting of SEQ ID NOs: 1-4.

In an aspect, an endogenous CDK comprises an amino acid sequence at least 70% identical to a sequence selected from the group consisting of SEQ ID NOs: 45-48. In an aspect, an endogenous CDK comprises an amino acid sequence at least 75% identical to a sequence selected from the group consisting of SEQ ID NOs: 45-48. In an aspect, an endogenous CDK comprises an amino acid sequence at least 80% identical to a sequence selected from the group consisting of SEQ ID NOs: 45-48. In an aspect, an endogenous CDK comprises an amino acid sequence at least 85% identical to a sequence selected from the group consisting of SEQ ID NOs: 45-48. In an aspect, an endogenous CDK comprises an amino acid sequence at least 90% identical to a sequence selected from the group consisting of SEQ ID NOs: 45-48. In an aspect, an endogenous CDK comprises an amino acid sequence at least 95% identical to a sequence selected from the group consisting of SEQ ID NOs: 45-48. In an aspect, an endogenous CDK comprises an amino acid sequence at least 96% identical to a sequence selected from the group consisting of SEQ ID NOs: 45-48. In an aspect, an endogenous CDK comprises an amino acid sequence at least 97% identical to a sequence selected from the group consisting of SEQ ID NOs: 45-48. In an aspect, an endogenous CDK comprises an amino acid sequence at least 98% identical to a sequence selected from the group consisting of SEQ ID NOs: 45-48. In an aspect, an endogenous CDK comprises an amino acid sequence at least 99% identical to a sequence selected from the group consisting of SEQ ID NOs: 45-48. In an aspect, a CDK comprises an amino acid sequence 100% identical to a sequence selected from the group consisting of SEQ ID NOs: 45-48.

In an aspect, an endogenous CDK comprises an amino acid sequence at least 70% similar to a sequence selected from the group consisting of SEQ ID NOs: 45-48. In an aspect, an endogenous CDK comprises an amino acid sequence at least 75% similar to a sequence selected from the group consisting of SEQ ID NOs: 45-48. In an aspect, an endogenous CDK comprises an amino acid sequence at least 80% similar to a sequence selected from the group consisting of SEQ ID NOs: 45-48. In an aspect, an endogenous CDK comprises an amino acid sequence at least 85% similar to a sequence selected from the group consisting of SEQ ID NOs: 45-48. In an aspect, an endogenous CDK comprises an amino acid sequence at least 90% similar to a sequence selected from the group consisting of SEQ ID NOs: 45-48. In an aspect, an endogenous CDK comprises an amino acid sequence at least 95% similar to a sequence selected from the group consisting of SEQ ID NOs: 45-48. In an aspect, an endogenous CDK comprises an amino acid sequence at least 96% similar to a sequence selected from the group consisting of SEQ ID NOs: 45-48. In an aspect, an endogenous CDK comprises an amino acid sequence at least 97% similar to a sequence selected from the group consisting of SEQ ID NOs: 45-48. In an aspect, an endogenous CDK comprises an amino acid sequence at least 98% similar to a sequence selected from the group consisting of SEQ ID NOs: 45-48. In an aspect, an endogenous CDK comprises an amino acid sequence at least 99% similar to a sequence selected from the group consisting of SEQ ID NOs: 45-48. In an aspect, a CDK comprises an amino acid sequence 100% similar to a sequence selected from the group consisting of SEQ ID NOs: 45-48.

Cyclin-dependent kinase inhibitors (CDKIs) are proteins that inhibit CDKs. CDKIs can bind to CDKs to prevent CDKs from binding to cyclins, thereby negatively regulating the cell cycle in eukaryotes. In plants, CDKIs are categorized into two families: KIP-RELATED PROTEINS (KRPs) and SIAMESE-RELATED PROTEINS (SMRs). See, for example, Kumar and Larkin, “Why do plants need so many cyclin-dependent kinase inhibitors?,” Plant Signaling & Behavior, 12: e1282021 (2017), which is herein incorporated by reference in its entirety.

In an aspect, this disclosure provides a polynucleotide that encodes a CDKI. In another aspect, this disclosure provides a CDKI. In an aspect, a CDKI provided herein is a KRP. In another aspect, a CDKI provided herein is an SMR.

In an aspect, this disclosure provides a mutated polynucleotide comprising a mutation in an endogenous gene, where the mutated polynucleotide encodes a mutated CDKI as compared to a CDKI encoded by the endogenous gene lacking the mutation. In an aspect, this disclosure provides a mutated polynucleotide comprising a mutation in an endogenous gene, where the mutated polynucleotide encodes a truncated CDKI as compared to a CDKI encoded by the endogenous gene lacking the mutation. In another aspect, this disclosure provides a non-naturally occurring truncated CDKI. In an aspect, this disclosure provides a polynucleotide encoding a mutated mRNA of an endogenous gene comprising a premature stop codon, where the mutated mRNA encodes a truncated CDKI as compared to an mRNA of the endogenous gene lacking the premature stop codon.

In an aspect, this disclosure provides a mutated polynucleotide comprising a mutation in an endogenous gene encoding a CDKI, where the mutated polynucleotide encodes a CDKI that is incapable of binding a CDK. In an aspect, this disclosure provides a mutated polynucleotide comprising a mutation in an endogenous gene encoding a CDKI, where the mutated polynucleotide encodes a CDKI that exhibits reduced binding affinity for at least one CDK as compared to a CDKI encoded by the endogenous gene lacking the mutation.

In an aspect, a mutated polynucleotide encodes a dominant negative allele of a CDKI. In another aspect, a mutated polynucleotide encodes a dominant positive allele of a CDKI. In a further aspect, a mutated polynucleotide encodes a constitutively active CDKI. In another aspect, a mutated polynucleotide encodes an inactive CDKI.

In an aspect, an endogenous gene encoding a CDKI comprises a polynucleotide sequence at least 80% identical to SEQ ID NO: 5. In an aspect, an endogenous gene encoding a CDKI comprises a polynucleotide sequence at least 85% identical to SEQ ID NO: 5. In an aspect, an endogenous gene encoding a CDKI comprises a polynucleotide sequence at least 90% identical to SEQ ID NO: 5. In an aspect, an endogenous gene encoding a CDKI comprises a polynucleotide sequence at least 95% identical to SEQ ID NO: 5. In an aspect, an endogenous gene encoding a CDKI comprises a polynucleotide sequence at least 96% identical to SEQ ID NO: 5. In an aspect, an endogenous gene encoding a CDKI comprises a polynucleotide sequence at least 97% identical to SEQ ID NO: 5. In an aspect, an endogenous gene encoding a CDKI comprises a polynucleotide sequence at least 98% identical to SEQ ID NO: 5. In an aspect, an endogenous gene encoding a CDKI comprises a polynucleotide sequence at least 99% identical to SEQ ID NO: 5. In an aspect, an endogenous gene encoding a CDKI comprises a polynucleotide sequence 100% identical to SEQ ID NO: 5.

In an aspect, an endogenous CDKI comprises an amino acid sequence at least 70% identical to SEQ ID NO: 49. In an aspect, an endogenous CDKI comprises an amino acid sequence at least 75% identical to SEQ ID NO: 49. In an aspect, an endogenous CDKI comprises an amino acid sequence at least 80% identical to SEQ ID NO: 49. In an aspect, an endogenous CDKI comprises an amino acid sequence at least 85% identical to SEQ ID NO: 49. In an aspect, an endogenous CDKI comprises an amino acid sequence at least 90% identical to SEQ ID NO: 49. In an aspect, an endogenous CDKI comprises an amino acid sequence at least 95% identical to SEQ ID NO: 49. In an aspect, an endogenous CDKI comprises an amino acid sequence at least 96% identical to SEQ ID NO: 49. In an aspect, an endogenous CDKI comprises an amino acid sequence at least 97% identical to SEQ ID NO: 49. In an aspect, an endogenous CDKI comprises an amino acid sequence at least 98% identical to SEQ ID NO: 49. In an aspect, an endogenous CDKI comprises an amino acid sequence at least 99% identical to SEQ ID NO: 49. In an aspect, a CDKI comprises an amino acid sequence 100% identical to SEQ ID NO: 49.

In an aspect, an endogenous CDKI comprises an amino acid sequence at least 70% similar to SEQ ID NO: 49. In an aspect, an endogenous CDKI comprises an amino acid sequence at least 75% similar to SEQ ID NO: 49. In an aspect, an endogenous CDKI comprises an amino acid sequence at least 80% similar to SEQ ID NO: 49. In an aspect, an endogenous CDKI comprises an amino acid sequence at least 85% similar to SEQ ID NO: 49. In an aspect, an endogenous CDKI comprises an amino acid sequence at least 90 similar identical to SEQ ID NO: 49. In an aspect, an endogenous CDKI comprises an amino acid sequence at least 95% similar to SEQ ID NO: 49. In an aspect, an endogenous CDKI comprises an amino acid sequence at least 96% similar to SEQ ID NO: 49. In an aspect, an endogenous CDKI comprises an amino acid sequence at least 97% similar to SEQ ID NO: 49. In an aspect, an endogenous CDKI comprises an amino acid sequence at least 98% similar to SEQ ID NO: 49. In an aspect, an endogenous CDKI comprises an amino acid sequence at least 99% similar to SEQ ID NO: 49. In an aspect, a CDKI comprises an amino acid sequence 100% similar to SEQ ID NO: 49.

MYB proteins (MYBs) are part of a family of transcription factors. In plants, MYBs comprise a conserved MYB DNA-binding domain. The MYB DNA-binding domain contains up to three imperfect repeats of about 55 amino acids each, in a helix-turn-helix structure. These repeats are termed R1, R2, and R3, and the R2/R3 repeats have been shown to bind directly to the major groove of DNA. A subfamily of plant-specific MYBs contains an R2R3-type MYB domain and are termed R2R3-type MYBs. Other subfamilies of MYBs include R1-type, 3R-type, and 4R-type MYBs. See, for example, Ambawat et al., “MYB transcription factor genes as regulators for plant responses: an overview,” Physiol. Mol. Biol. Plants, 19:307-321 (2013), which is herein incorporated by reference in its entirety.

In an aspect, a MYB provided herein is an R1-type MYB. In another aspect, a MYB provided herein is an R2R3-type MYB. In another aspect, a MYB provided herein is a 3R-type MYB. In another aspect, a MYB provided herein is a 4R-type MYB.

In an aspect, this disclosure provides a polynucleotide that encodes a MYB. In another aspect, this disclosure provides a MYB.

In an aspect, this disclosure provides a mutated polynucleotide comprising a mutation in an endogenous gene, where the mutated polynucleotide encodes a mutated MYB as compared to a MYB encoded by the endogenous gene lacking the mutation. In an aspect, this disclosure provides a mutated polynucleotide comprising a mutation in an endogenous gene, where the mutated polynucleotide encodes a truncated MYB as compared to a MYB encoded by the endogenous gene lacking the mutation. In another aspect, this disclosure provides a non-naturally occurring truncated MYB. In an aspect, this disclosure provides a polynucleotide encoding a mutated mRNA of an endogenous gene comprising a premature stop codon, where the mutated mRNA encodes a truncated MYB as compared to an mRNA of the endogenous gene lacking the premature stop codon.

In an aspect, this disclosure provides a polynucleotide comprising a non-naturally occurring mutation in a sequence encoding a MYB DNA-binding domain. In an aspect, this disclosure provides a mutated polynucleotide comprising a mutation in an endogenous gene encoding a MYB, where the mutated polynucleotide encodes a MYB lacking a MYB DNA-binding domain. In another aspect, this disclosure provides a mutated polynucleotide comprising a mutation in an endogenous gene encoding a MYB, where the mutated polynucleotide encodes a MYB comprising a truncated MYB DNA-binding domain as compared to a MYB encoded by the endogenous gene lacking the mutation.

In an aspect, this disclosure provides a mutated polynucleotide comprising a mutation in an endogenous gene encoding a MYB, where the mutated polynucleotide encodes a MYB that is incapable of binding DNA. In an aspect, this disclosure provides a mutated polynucleotide comprising a mutation in an endogenous gene encoding a MYB, where the mutated polynucleotide encodes a MYB that exhibits reduced binding affinity for DNA as compared to a MYB encoded by the endogenous gene lacking the mutation.

In another aspect, this disclosure provides a mutated polynucleotide comprising a mutation in an endogenous gene encoding a MYB, where the mutated polynucleotide encodes a MYB comprising a truncated R2R3-type MYB domain as compared to a MYB encoded by the endogenous gene lacking the mutation. In another aspect, this disclosure provides a mutated polynucleotide comprising a mutation in an endogenous gene encoding a MYB, where the mutated polynucleotide encodes a MYB comprising a truncated R1-type MYB domain as compared to a MYB encoded by the endogenous gene lacking the mutation. In another aspect, this disclosure provides a mutated polynucleotide comprising a mutation in an endogenous gene encoding a MYB, where the mutated polynucleotide encodes a MYB comprising a truncated 3R-type MYB domain as compared to a MYB encoded by the endogenous gene lacking the mutation. In another aspect, this disclosure provides a mutated polynucleotide comprising a mutation in an endogenous gene encoding a MYB, where the mutated polynucleotide encodes a MYB comprising a truncated 4R-type MYB domain as compared to a MYB encoded by the endogenous gene lacking the mutation.

In an aspect, a mutated polynucleotide encodes a dominant negative allele of a MYB. In another aspect, a mutated polynucleotide encodes a dominant positive allele of a MYB. In a further aspect, a mutated polynucleotide encodes a constitutively active MYB. In another aspect, a mutated polynucleotide encodes an inactive MYB.

In an aspect, an endogenous gene encoding a MYB comprises a polynucleotide sequence at least 80% identical to a sequence selected from the group consisting of SEQ ID NOs: 33-44. In an aspect, an endogenous gene encoding a MYB comprises a polynucleotide sequence at least 85% identical to a sequence selected from the group consisting of SEQ ID NOs: 33-44. In an aspect, an endogenous gene encoding a MYB comprises a polynucleotide sequence at least 90% identical to a sequence selected from the group consisting of SEQ ID NOs: 33-44. In an aspect, an endogenous gene encoding a MYB comprises a polynucleotide sequence at least 95% identical to a sequence selected from the group consisting of SEQ ID NOs: 33-44. In an aspect, an endogenous gene encoding a MYB comprises a polynucleotide sequence at least 96% identical to a sequence selected from the group consisting of SEQ ID NOs: 33-44. In an aspect, an endogenous gene encoding a MYB comprises a polynucleotide sequence at least 97% identical to a sequence selected from the group consisting of SEQ ID NOs: 33-44. In an aspect, an endogenous gene encoding a MYB comprises a polynucleotide sequence at least 98% identical to a sequence selected from the group consisting of SEQ ID NOs: 33-44. In an aspect, an endogenous gene encoding a MYB comprises a polynucleotide sequence at least 99% identical to a sequence selected from the group consisting of SEQ ID NOs: 33-44. In an aspect, an endogenous gene encoding a MYB comprises a polynucleotide sequence 100% identical to a sequence selected from the group consisting of SEQ ID NOs: 33-44.

In an aspect, an endogenous MYB comprises an amino acid sequence at least 70% identical to a sequence selected from the group consisting of SEQ ID NOs: 77-88. In an aspect, an endogenous MYB comprises an amino acid sequence at least 75% identical to a sequence selected from the group consisting of SEQ ID NOs: 77-88. In an aspect, an endogenous MYB comprises an amino acid sequence at least 80% identical to a sequence selected from the group consisting of SEQ ID NOs: 77-88. In an aspect, an endogenous MYB comprises an amino acid sequence at least 85% identical to a sequence selected from the group consisting of SEQ ID NOs: 77-88. In an aspect, an endogenous MYB comprises an amino acid sequence at least 90% identical to a sequence selected from the group consisting of SEQ ID NOs: 77-88. In an aspect, an endogenous MYB comprises an amino acid sequence at least 95% identical to a sequence selected from the group consisting of SEQ ID NOs: 77-88. In an aspect, an endogenous MYB comprises an amino acid sequence at least 96% identical to a sequence selected from the group consisting of SEQ ID NOs: 77-88. In an aspect, an endogenous MYB comprises an amino acid sequence at least 97% identical to a sequence selected from the group consisting of SEQ ID NOs: 77-88. In an aspect, an endogenous MYB comprises an amino acid sequence at least 98% identical to a sequence selected from the group consisting of SEQ ID NOs: 77-88. In an aspect, an endogenous MYB comprises an amino acid sequence at least 99% identical to a sequence selected from the group consisting of SEQ ID NOs: 77-88. In an aspect, a MYB comprises an amino acid sequence 100% identical to a sequence selected from the group consisting of SEQ ID NOs: 77-88.

In an aspect, an endogenous MYB comprises an amino acid sequence at least 70% similar to a sequence selected from the group consisting of SEQ ID NOs: 77-88. In an aspect, an endogenous MYB comprises an amino acid sequence at least 75% similar to a sequence selected from the group consisting of SEQ ID NOs: 77-88. In an aspect, an endogenous MYB comprises an amino acid sequence at least 80% similar to a sequence selected from the group consisting of SEQ ID NOs: 77-88. In an aspect, an endogenous MYB comprises an amino acid sequence at least 85% similar to a sequence selected from the group consisting of SEQ ID NOs: 77-88. In an aspect, an endogenous MYB comprises an amino acid sequence at least 90% similar to a sequence selected from the group consisting of SEQ ID NOs: 77-88. In an aspect, an endogenous MYB comprises an amino acid sequence at least 95% similar to a sequence selected from the group consisting of SEQ ID NOs: 77-88. In an aspect, an endogenous MYB comprises an amino acid sequence at least 96% similar to a sequence selected from the group consisting of SEQ ID NOs: 77-88. In an aspect, an endogenous MYB comprises an amino acid sequence at least 97% similar to a sequence selected from the group consisting of SEQ ID NOs: 77-88. In an aspect, an endogenous MYB comprises an amino acid sequence at least 98% similar to a sequence selected from the group consisting of SEQ ID NOs: 77-88. In an aspect, an endogenous MYB comprises an amino acid sequence at least 99% similar to a sequence selected from the group consisting of SEQ ID NOs: 77-88. In an aspect, a MYB comprises an amino acid sequence 100% similar to a sequence selected from the group consisting of SEQ ID NOs: 77-88.

WRKY transcription factors are one of the largest families of transcriptional regulators known in plants. A WRKY transcription factor is a protein that comprises a “WRKY domain.” A WRKY domain comprises about 60 to 70 amino acids, and is involved in DNA binding. The WRKY domain contains a highly conserved “WRKY core domain” having an amino acid motif of WRKYGQK (SEQ ID NO: 110). The conserved core of the WRKY domain can vary, and the amino acid motif WRKYGKK (SEQ ID NO: 111) is a commonly observed “WRKY core variant domain.” WRKY domains comprise a globular shape composed of five anti-parallel β-strands, and the conserved core is found on the second β-strand. The third β-strand also contains a highly conserved amino acid motif of PRSYY (SEQ ID NO: 112). In many WRKY transcription factors, the PR and SYY amino acids of the “PRSYY motif” (SEQ ID NO: 112) are encoded by different exons; the position of the intervening intron is highly conserved amongst WRKY transcription factors. WRKY domains also contain a “zinc finger region” comprising an amino acid motif of CX4-5CX22-23HXH (SEQ ID NO: 115) or CX7CX23HXC (SEQ ID NO: 116), where X can be any amino acid, C is a cysteine, and His a histidine. WRKY domains also contain a “DWK salt bridge” motif, where the conserved tryptophan (W) of the WRKY core (SEQ ID NO: 117) forms a triad with an aspartic acid (D) four amino acids upstream of the W, and a lysine (K) twenty-nine amino acids downstream of the W (SEQ ID NO: 127). It is believed that the DWK salt bridge is important for stabilizing the WRKY domain. See, for example, Rushton et al., “WRKY transcription factors,” Trends in Plant Science, 15:247-258 (2010), which is incorporated herein by reference in its entirety.

In an aspect, this disclosure provides a polynucleotide that encodes a WRKY transcription factor. In another aspect, this disclosure provides a WRKY transcription factor.

In an aspect, this disclosure provides a polynucleotide comprising a non-naturally occurring mutation, where the polynucleotide encodes a WRKY transcription factor. In an aspect, this disclosure provides a polynucleotide encoding a truncated WRKY transcription factor. In another aspect, this disclosure provides a non-naturally occurring truncated WRKY transcription factor. In an aspect, this disclosure provides a polynucleotide encoding an mRNA comprising a premature stop codon, where the mRNA encodes a truncated WRKY transcription factor.

In an aspect, this disclosure provides a mutated polynucleotide comprising a mutation in an endogenous gene, where the mutated polynucleotide encodes a mutated WRKY transcription factor as compared to a WRKY transcription factor encoded by the endogenous gene lacking the mutation. In an aspect, this disclosure provides a mutated polynucleotide comprising a mutation in an endogenous gene, where the mutated polynucleotide encodes a truncated WRKY transcription factor as compared to a WRKY transcription factor encoded by the endogenous gene lacking the mutation. In another aspect, this disclosure provides a non-naturally occurring truncated WRKY transcription factor. In an aspect, this disclosure provides a polynucleotide encoding a mutated mRNA of an endogenous gene comprising a premature stop codon, where the mutated mRNA encodes a truncated WRKY transcription factor as compared to an mRNA of the endogenous gene lacking the premature stop codon.

In an aspect, this disclosure provides a polynucleotide comprising a non-naturally occurring mutation in a sequence encoding a WRKY domain. In an aspect, this disclosure provides a mutated polynucleotide comprising a mutation in an endogenous gene encoding a WRKY transcription factor, where the mutated polynucleotide encodes a WRKY transcription factor lacking a WRKY domain. In another aspect, this disclosure provides a mutated polynucleotide comprising a mutation in an endogenous gene encoding a WRKY transcription factor, where the mutated polynucleotide encodes a WRKY transcription factor comprising a truncated WRKY domain as compared to a WRKY transcription factor encoded by the endogenous gene lacking the mutation.

In an aspect, this disclosure provides a polynucleotide comprising a non-naturally occurring mutation in a sequence encoding a WRKY core domain. In an aspect, this disclosure provides a mutated polynucleotide comprising a mutation in an endogenous gene encoding a WRKY transcription factor, where the mutated polynucleotide encodes a WRKY transcription factor lacking a WRKY core domain. In another aspect, this disclosure provides a mutated polynucleotide comprising a mutation in an endogenous gene encoding a WRKY transcription factor, where the mutated polynucleotide encodes a WRKY transcription factor comprising a truncated WRKY core domain as compared to a WRKY transcription factor encoded by the endogenous gene lacking the mutation.

In an aspect, this disclosure provides a polynucleotide comprising a non-naturally occurring mutation in a sequence encoding a WRKY core variant domain. In an aspect, this disclosure provides a mutated polynucleotide comprising a mutation in an endogenous gene encoding a WRKY transcription factor, where the mutated polynucleotide encodes a WRKY transcription factor lacking a WRKY core variant domain. In another aspect, this disclosure provides a mutated polynucleotide comprising a mutation in an endogenous gene encoding a WRKY transcription factor, where the mutated polynucleotide encodes a WRKY transcription factor comprising a truncated WRKY core variant domain as compared to a WRKY transcription factor encoded by the endogenous gene lacking the mutation.

In an aspect, this disclosure provides a polynucleotide comprising a non-naturally occurring mutation in a sequence encoding a PRSYY motif (SEQ ID NO: 112). In an aspect, this disclosure provides a mutated polynucleotide comprising a mutation in an endogenous gene encoding a WRKY transcription factor, where the mutated polynucleotide encodes a WRKY transcription factor lacking a PRSYY motif (SEQ ID NO: 112). In another aspect, this disclosure provides a mutated polynucleotide comprising a mutation in an endogenous gene encoding a WRKY transcription factor, where the mutated polynucleotide encodes a WRKY transcription factor comprising a truncated PRSYY motif (SEQ ID NO: 112) as compared to a WRKY transcription factor encoded by the endogenous gene lacking the mutation.

In an aspect, this disclosure provides a polynucleotide comprising a non-naturally occurring mutation in a sequence encoding a zinc finger region. In an aspect, this disclosure provides a mutated polynucleotide comprising a mutation in an endogenous gene encoding a WRKY transcription factor, where the mutated polynucleotide encodes a WRKY transcription factor lacking a zinc finger region. In another aspect, this disclosure provides a mutated polynucleotide comprising a mutation in an endogenous gene encoding a WRKY transcription factor, where the mutated polynucleotide encodes a WRKY transcription factor comprising a truncated zinc finger region as compared to a WRKY transcription factor encoded by the endogenous gene lacking the mutation.

In an aspect, this disclosure provides a polynucleotide comprising a non-naturally occurring mutation in a sequence encoding a DWK salt bridge motif. In an aspect, this disclosure provides a mutated polynucleotide comprising a mutation in an endogenous gene encoding a WRKY transcription factor, where the mutated polynucleotide encodes a WRKY transcription factor lacking a DWK salt bridge motif. In another aspect, this disclosure provides a mutated polynucleotide comprising a mutation in an endogenous gene encoding a WRKY transcription factor, where the mutated polynucleotide encodes a WRKY transcription factor comprising a truncated DWK salt bridge motif as compared to a WRKY transcription factor encoded by the endogenous gene lacking the mutation.

In an aspect, this disclosure provides a mutated polynucleotide comprising a mutation in an endogenous gene encoding a WRKY transcription factor, where the mutated polynucleotide encodes a WRKY transcription factor that is incapable of binding DNA. In an aspect, this disclosure provides a mutated polynucleotide comprising a mutation in an endogenous gene encoding a WRKY transcription factor, where the mutated polynucleotide encodes a WRKY transcription factor that exhibits reduced binding affinity for DNA as compared to a WRKY transcription factor encoded by the endogenous gene lacking the mutation.

In an aspect, a mutated polynucleotide encodes a dominant negative allele of a WRKY transcription factor. In another aspect, a mutated polynucleotide encodes a dominant positive allele of a WRKY transcription factor. In a further aspect, a mutated polynucleotide encodes a constitutively active WRKY transcription factor. In another aspect, a mutated polynucleotide encodes an inactive WRKY transcription factor.

In an aspect, an endogenous gene encoding a WRKY transcription factor comprises a polynucleotide sequence at least 80% identical to SEQ ID NO: 113. In an aspect, an endogenous gene encoding a WRKY transcription factor comprises a polynucleotide sequence at least 85% identical to SEQ ID NO: 113. In an aspect, an endogenous gene encoding a WRKY transcription factor comprises a polynucleotide sequence at least 90% identical to SEQ ID NO: 113. In an aspect, an endogenous gene encoding a WRKY transcription factor comprises a polynucleotide sequence at least 95% identical to SEQ ID NO: 113. In an aspect, an endogenous gene encoding a WRKY transcription factor comprises a polynucleotide sequence at least 96% identical to SEQ ID NO: 113. In an aspect, an endogenous gene encoding a WRKY transcription factor comprises a polynucleotide sequence at least 97% identical to SEQ ID NO: 113. In an aspect, an endogenous gene encoding a WRKY transcription factor comprises a polynucleotide sequence at least 98% identical to SEQ ID NO: 113. In an aspect, an endogenous gene encoding a WRKY transcription factor comprises a polynucleotide sequence at least 99% identical to SEQ ID NO: 113. In an aspect, an endogenous gene encoding a WRKY transcription factor comprises a polynucleotide sequence 100% identical to SEQ ID NO: 113.

In an aspect, an endogenous WRKY transcription factor comprises an amino acid sequence at least 70% identical to SEQ ID NO: 114. In an aspect, an endogenous WRKY transcription factor comprises an amino acid sequence at least 75% identical to SEQ ID NO: 114. In an aspect, an endogenous WRKY transcription factor comprises an amino acid sequence at least 80% identical to SEQ ID NO: 114. In an aspect, an endogenous WRKY transcription factor comprises an amino acid sequence at least 85% identical to SEQ ID NO: 114. In an aspect, an endogenous WRKY transcription factor comprises an amino acid sequence at least 90% identical to SEQ ID NO: 114. In an aspect, an endogenous WRKY transcription factor comprises an amino acid sequence at least 95% identical to SEQ ID NO: 114. In an aspect, an endogenous WRKY transcription factor comprises an amino acid sequence at least 96% identical to SEQ ID NO: 114. In an aspect, an endogenous WRKY transcription factor comprises an amino acid sequence at least 97% identical to SEQ ID NO: 114. In an aspect, an endogenous WRKY transcription factor comprises an amino acid sequence at least 98% identical to SEQ ID NO: 114. In an aspect, an endogenous WRKY transcription factor comprises an amino acid sequence at least 99% identical to SEQ ID NO: 114. In an aspect, a WRKY transcription factor comprises an amino acid sequence 100% identical to SEQ ID NO: 114.

In an aspect, an endogenous WRKY transcription factor comprises an amino acid sequence at least 70% similar to SEQ ID NO: 114. In an aspect, an endogenous WRKY transcription factor comprises an amino acid sequence at least 75% similar to SEQ ID NO: 114. In an aspect, an endogenous WRKY transcription factor comprises an amino acid sequence at least 80% similar to SEQ ID NO: 114. In an aspect, an endogenous WRKY transcription factor comprises an amino acid sequence at least 85% similar to SEQ ID NO: 114. In an aspect, an endogenous WRKY transcription factor comprises an amino acid sequence at least 90% similar to SEQ ID NO: 114. In an aspect, an endogenous WRKY transcription factor comprises an amino acid sequence at least 95% similar to SEQ ID NO: 114. In an aspect, an endogenous WRKY transcription factor comprises an amino acid sequence at least 96% similar to SEQ ID NO: 114. In an aspect, an endogenous WRKY transcription factor comprises an amino acid sequence at least 97% similar to SEQ ID NO: 114. In an aspect, an endogenous WRKY transcription factor comprises an amino acid sequence at least 98% similar to SEQ ID NO: 114. In an aspect, an endogenous WRKY transcription factor comprises an amino acid sequence at least 99% similar to SEQ ID NO: 114. In an aspect, a WRKY transcription factor comprises an amino acid sequence 100% similar to SEQ ID NO: 114.

In an aspect, a modified tobacco plant, tobacco plant part, tobacco seed, tobacco cell, or tobacco genome provided herein comprises one or more mutations. As used herein, a “mutation” refers to a non-naturally occurring alteration to DNA as compared to an endogenous reference DNA sequence. It will be appreciated that, when identifying a mutation, the endogenous reference DNA sequence should be from the same variety of tobacco. For example, if a modified tobacco plant comprising a mutation is from the variety TN90, then the endogenous reference sequence must be the endogenous TN90 sequence, not a homologous sequence from a different tobacco variety (e.g., K326). Similarly, if a modified tobacco cell comprising a mutation is a TN90 cell, then the endogenous reference sequence must be the endogenous TN90 sequence, not a homologous sequence from a tobacco cell from a different tobacco variety (e.g., K326).

In an aspect, a mutation provided herein creates a dominant allele of the mutated locus. Dominant alleles are alleles that mask the contribution of a second allele at the same locus. A dominant allele can be a “dominant negative allele” or a “dominant positive allele.” Dominant negative alleles, or antimorphs, are alleles that act in opposition to normal allelic function. A dominant negative allele typically does not function normally and either directly inhibits the activity of a wild-type protein (e.g., through dimerization) or inhibits the activity of a second protein that is required for the normal function of the wild-type protein (e.g., an activator or a downstream component of a pathway). For example, a dominant negative allele abrogates or reduces the normal function of an allele in a heterozygous or homozygous state. Dominant positive alleles can increase normal gene function (e.g., a hypermorph) or provide new functions for a gene (e.g., a neomorph). A semi-dominant allele occurs when penetrance of a linked phenotype in individuals heterozygous for the allele is less than that which is observed in individuals homozygous for the allele.

In an aspect, a mutation provided herein creates a dominant negative allele of the mutated locus. In another aspect, a mutation provided herein creates a dominant positive allele of a mutated locus.

As used herein, “inducing” a mutation refers to generating a mutation in a polynucleotide sequence via human intervention. Many suitable methods for inducing mutations in tobacco are known in the art. Non-limiting examples of such methods include use of chemical mutagens, use of radiation, and use of nucleases. In an aspect, inducing a mutation comprises the use of an agent selected from the group consisting of a chemical mutagen, irradiation, a transposon, Agrobacterium, and a nuclease.

In an aspect, inducing a mutation comprises the use of a chemical mutagen. In an aspect, a chemical mutagen comprises ethyl methanesulfonate (EMS).

In another aspect, inducing a mutation comprises the use of irradiation. In an aspect, irradiation comprises gamma rays, X-rays, or ionizing radiation. In another aspect, irradiation comprises the use of fast neutrons.

In an aspect, inducing a mutation comprises the use of a transposon. In another aspect, inducing a mutation comprises the use of Agrobacterium.

In a further aspect, inducing a mutation comprises the use of a nuclease. In an aspect, a nuclease is selected from the group consisting of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, and a Csm1 nuclease. In an aspect, inducing a mutation comprises the use of a CRISPR/Cas9 nuclease. In an aspect, inducing a mutation comprises the use of a CRISPR/Cpf1 nuclease. In an aspect, inducing a mutation comprises the use of a CRISPR/CasX nuclease. In an aspect, inducing a mutation comprises the use of a CRISPR/CasY nuclease. In an aspect, inducing a mutation comprises the use of a Csm1 nuclease.

Several types of mutations are known in the art. In an aspect, a mutation comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation comprises an inversion. An “inversion” refers to when a segment of a polynucleotide or amino acid sequence is reversed end-to-end. In an aspect, a mutation provided herein comprises a mutation selected from the group consisting of an insertion, a deletion, a substitution, and an inversion.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-untranslated region of an endogenous gene. In still another aspect, a mutation provided herein is positioned within a 3′-untranslated region of an endogenous gene. In yet another aspect, a mutation provided herein is positioned within a promoter of an endogenous gene.

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

In an aspect, a mutation in an endogenous gene results in a reduced level of expression as compared to the endogenous gene lacking the mutation. In another aspect, a mutation in an endogenous gene results in an increased level of expression as compared to the endogenous gene lacking the mutation. In a further aspect, a mutation in an endogenous gene results in a reduced level of activity by a protein or polypeptide encoded by the endogenous gene having the mutation as compared to a protein or polypeptide encoded by the endogenous gene lacking the mutation. In a further aspect, a mutation in an endogenous gene results in an increased level of activity by a protein or polypeptide encoded by the endogenous gene having the mutation as compared to a protein or polypeptide encoded by the endogenous gene lacking the mutation.

In an aspect, a mutation in a genomic locus results in a reduced level of expression as compared to the genomic locus lacking the mutation. In another aspect, a mutation in a genomic locus results in an increased level of expression as compared to the genomic locus lacking the mutation. In a further aspect, a mutation in a genomic locus results in a reduced level of activity by a protein or polypeptide encoded by the genomic locus having the mutation as compared to a protein or polypeptide encoded by the genomic locus lacking the mutation. In a further aspect, a mutation in a genomic locus results in an increased level of activity by a protein or polypeptide encoded by the genomic locus having the mutation as compared to a protein or polypeptide encoded by the genomic locus lacking the mutation.

Levels of gene expression are routinely investigated in the art. As non-limiting examples, gene expression can be measured using quantitative reverse transcriptase PCR (qRT-PCR), RNA sequencing, or Northern blots. In an aspect, gene expression is measured using qRT-PCR. In another aspect, gene expression is measured using a Northern blot. In another aspect, gene expression is measured using RNA sequencing.

Levels of protein activity are also routinely investigated in the art. For example, CDK activity can be measured using phosphorylation assays.

As used herein, the term “heterologous” refers to a combination of two or more DNA molecules or sequences, such as a promoter and an associated transcribable DNA sequence, coding sequence or gene, when such a combination is man-made and not normally found in nature.

In one aspect, the level of expression of an endogenous gene comprising a mutation is reduced by at least 1% as compared to the level of expression of the endogenous gene lacking the mutation. In one aspect, the level of expression of an endogenous gene comprising a mutation is reduced by at least 5% as compared to the level of expression of the endogenous gene lacking the mutation. In one aspect, the level of expression of an endogenous gene comprising a mutation is reduced by at least 10% as compared to the level of expression of the endogenous gene lacking the mutation. In one aspect, the level of expression of an endogenous gene comprising a mutation is reduced by at least 15% as compared to the level of expression of the endogenous gene lacking the mutation. In one aspect, the level of expression of an endogenous gene comprising a mutation is reduced by at least 20% as compared to the level of expression of the endogenous gene lacking the mutation. In one aspect, the level of expression of an endogenous gene comprising a mutation is reduced by at least 25% as compared to the level of expression of the endogenous gene lacking the mutation. In one aspect, the level of expression of an endogenous gene comprising a mutation is reduced by at least 50% as compared to the level of expression of the endogenous gene lacking the mutation. In one aspect, the level of expression of an endogenous gene comprising a mutation is reduced by at least 75% as compared to the level of expression of the endogenous gene lacking the mutation. In one aspect, the level of expression of an endogenous gene comprising a mutation is reduced by at least 90% as compared to the level of expression of the endogenous gene lacking the mutation. In one aspect, the level of expression of an endogenous gene comprising a mutation is reduced by at least 95% as compared to the level of expression of the endogenous gene lacking the mutation.

In one aspect, the level of expression of an endogenous gene comprising a mutation is reduced by between 1% and 99% as compared to the level of expression of the endogenous gene lacking the mutation. In one aspect, the level of expression of an endogenous gene comprising a mutation is reduced by between 1% and 90% as compared to the level of expression of the endogenous gene lacking the mutation. In one aspect, the level of expression of an endogenous gene comprising a mutation is reduced by between 1% and 75% as compared to the level of expression of the endogenous gene lacking the mutation. In one aspect, the level of expression of an endogenous gene comprising a mutation is reduced by between 1% and 50% as compared to the level of expression of the endogenous gene lacking the mutation. In one aspect, the level of expression of an endogenous gene comprising a mutation is reduced by between 1% and 25% as compared to the level of expression of the endogenous gene lacking the mutation. In one aspect, the level of expression of an endogenous gene comprising a mutation is reduced by between 1% and 10% as compared to the level of expression of the endogenous gene lacking the mutation. In one aspect, the level of expression of an endogenous gene comprising a mutation is reduced by between 75% and 99% as compared to the level of expression of the endogenous gene lacking the mutation. In one aspect, the level of expression of an endogenous gene comprising a mutation is reduced by between 50% and 99% as compared to the level of expression of the endogenous gene lacking the mutation. In one aspect, the level of expression of an endogenous gene comprising a mutation is reduced by between 25% and 99% as compared to the level of expression of the endogenous gene lacking the mutation. In one aspect, the level of expression of an endogenous gene comprising a mutation is reduced by between 10% and 99% as compared to the level of expression of the endogenous gene lacking the mutation. In one aspect, the level of expression of an endogenous gene comprising a mutation is reduced by between 50% and 75% as compared to the level of expression of the endogenous gene lacking the mutation. In one aspect, the level of expression of an endogenous gene comprising a mutation is reduced by between 25% and 75% as compared to the level of expression of the endogenous gene lacking the mutation. In one aspect, the level of expression of an endogenous gene comprising a mutation is reduced by between 10% and 50% as compared to the level of expression of the endogenous gene lacking the mutation.

In one aspect, the level of expression of an endogenous gene comprising a mutation is increased by at least 1% as compared to the level of expression of the endogenous gene lacking the mutation. In one aspect, the level of expression of an endogenous gene comprising a mutation is increased by at least 5% as compared to the level of expression of the endogenous gene lacking the mutation. In one aspect, the level of expression of an endogenous gene comprising a mutation is increased by at least 10% as compared to the level of expression of the endogenous gene lacking the mutation. In one aspect, the level of expression of an endogenous gene comprising a mutation is increased by at least 15% as compared to the level of expression of the endogenous gene lacking the mutation. In one aspect, the level of expression of an endogenous gene comprising a mutation is increased by at least 20% as compared to the level of expression of the endogenous gene lacking the mutation. In one aspect, the level of expression of an endogenous gene comprising a mutation is increased by at least 25% as compared to the level of expression of the endogenous gene lacking the mutation. In one aspect, the level of expression of an endogenous gene comprising a mutation is increased by at least 50% as compared to the level of expression of the endogenous gene lacking the mutation. In one aspect, the level of expression of an endogenous gene comprising a mutation is increased by at least 75% as compared to the level of expression of the endogenous gene lacking the mutation. In one aspect, the level of expression of an endogenous gene comprising a mutation is increased by at least 90% as compared to the level of expression of the endogenous gene lacking the mutation. In one aspect, the level of expression of an endogenous gene comprising a mutation is increased by at least 95% as compared to the level of expression of the endogenous gene lacking the mutation. In one aspect, the level of expression of an endogenous gene comprising a mutation is increased by at least 100% as compared to the level of expression of the endogenous gene lacking the mutation. In one aspect, the level of expression of an endogenous gene comprising a mutation is increased by at least 150% as compared to the level of expression of the endogenous gene lacking the mutation. In one aspect, the level of expression of an endogenous gene comprising a mutation is increased by at least 200% as compared to the level of expression of the endogenous gene lacking the mutation. In one aspect, the level of expression of an endogenous gene comprising a mutation is increased by at least 250% as compared to the level of expression of the endogenous gene lacking the mutation. In one aspect, the level of expression of an endogenous gene comprising a mutation is increased by at least 300% as compared to the level of expression of the endogenous gene lacking the mutation. In one aspect, the level of expression of an endogenous gene comprising a mutation is increased by at least 400% as compared to the level of expression of the endogenous gene lacking the mutation. In one aspect, the level of expression of an endogenous gene comprising a mutation is increased by at least 500% as compared to the level of expression of the endogenous gene lacking the mutation. In one aspect, the level of expression of an endogenous gene comprising a mutation is increased by at least 750% as compared to the level of expression of the endogenous gene lacking the mutation. In one aspect, the level of expression of an endogenous gene comprising a mutation is increased by at least 1000% as compared to the level of expression of the endogenous gene lacking the mutation.

In one aspect, the level of expression of an endogenous gene comprising a mutation is increased by between 1% and 1000% as compared to the level of expression of the endogenous gene lacking the mutation. In one aspect, the level of expression of an endogenous gene comprising a mutation is increased by between 1% and 750% as compared to the level of expression of the endogenous gene lacking the mutation. In one aspect, the level of expression of an endogenous gene comprising a mutation is increased by between 1% and 500% as compared to the level of expression of the endogenous gene lacking the mutation. In one aspect, the level of expression of an endogenous gene comprising a mutation is increased by between 1% and 400% as compared to the level of expression of the endogenous gene lacking the mutation. In one aspect, the level of expression of an endogenous gene comprising a mutation is increased by between 1% and 300% as compared to the level of expression of the endogenous gene lacking the mutation. In one aspect, the level of expression of an endogenous gene comprising a mutation is increased by between 1% and 200% as compared to the level of expression of the endogenous gene lacking the mutation. In one aspect, the level of expression of an endogenous gene comprising a mutation is increased by between 1% and 100% as compared to the level of expression of the endogenous gene lacking the mutation. In one aspect, the level of expression of an endogenous gene comprising a mutation is increased by between 1% and 75% as compared to the level of expression of the endogenous gene lacking the mutation. In one aspect, the level of expression of an endogenous gene comprising a mutation is increased by between 1% and 50% as compared to the level of expression of the endogenous gene lacking the mutation. In one aspect, the level of expression of an endogenous gene comprising a mutation is increased by between 1% and 25% as compared to the level of expression of the endogenous gene lacking the mutation. In one aspect, the level of expression of an endogenous gene comprising a mutation is increased by between 1% and 10% as compared to the level of expression of the endogenous gene lacking the mutation. In one aspect, the level of expression of an endogenous gene comprising a mutation is increased by between 100% and 1000% as compared to the level of expression of the endogenous gene lacking the mutation. In one aspect, the level of expression of an endogenous gene comprising a mutation is increased by between 100% and 750% as compared to the level of expression of the endogenous gene lacking the mutation. In one aspect, the level of expression of an endogenous gene comprising a mutation is increased by between 100% and 500% as compared to the level of expression of the endogenous gene lacking the mutation. In one aspect, the level of expression of an endogenous gene comprising a mutation is increased by between 100% and 250% as compared to the level of expression of the endogenous gene lacking the mutation. In one aspect, the level of expression of an endogenous gene comprising a mutation is increased by between 750% and 1000% as compared to the level of expression of the endogenous gene lacking the mutation. In one aspect, the level of expression of an endogenous gene comprising a mutation is increased by between 500% and 1000% as compared to the level of expression of the endogenous gene lacking the mutation. In one aspect, the level of expression of an endogenous gene comprising a mutation is increased by between 250% and 1000% as compared to the level of expression of the endogenous gene lacking the mutation. In one aspect, the level of expression of an endogenous gene comprising a mutation is increased by between 1% and 100% as compared to the level of expression of the endogenous gene lacking the mutation. In one aspect, the level of expression of an endogenous gene comprising a mutation is increased by between 50% and 100% as compared to the level of expression of the endogenous gene lacking the mutation.

As used herein, the term “endogenous gene” or “native gene” refers to a gene that originates within a tobacco genome. An “endogenous gene” is a gene that was not previously modified by human action. In an aspect, an endogenous gene is a nuclear gene. In another aspect, an endogenous gene is a mitochondrial gene. In a further aspect, an endogenous gene is a chloroplast gene.

As used herein, a “gene” refers to a polynucleotide that can produce a functional unit (e.g., without being limiting, for example, a protein, or a small RNA molecule). A gene can comprise a promoter, an enhancer sequence, a leader sequence, a transcriptional start site, a transcriptional stop site, a polyadenylation site, one or more exons, one or more introns, a 5′-untranslated region (UTR), a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a small RNA molecule or a precursor thereof. In another aspect, a gene encodes a protein or polypeptide.

As used herein, a “genomic locus” refers to a fixed position on a chromosome. In an aspect, a genomic locus comprises a polynucleotide encoding a gene. In an aspect, a genomic locus comprises a polynucleotide encoding an endogenous gene. In another aspect, a genomic locus comprises a polynucleotide encoding a transgene. In an aspect, a genomic locus can be transcribed from DNA to RNA. In an aspect, a genomic locus encodes a messenger RNA. In another aspect, a genomic locus encodes a small RNA molecule.

As commonly understood in the art, the term “promoter” refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

Promoters that drive expression in all or most tissues of the plant are referred to as “constitutive” promoters. Promoters that drive expression during certain periods or stages of development are referred to as “developmental” promoters. Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. As a non-limiting example, an “axillary bud-preferred promoter” causes relatively higher or preferential expression in axillary bud tissues, but can have lower levels of expression in other parts of a plant (e.g., roots, leaves, stem). Promoters that express within a specific tissue(s) of an organism, with little or no expression in other tissues, are referred to as “tissue-specific” promoters. As a non-limited example, an “axillary bud-specific promoter” drives expression in axillary bud tissues, with little to no detectable expression in other plant tissue types. An “inducible” promoter is a promoter that initiates transcription in response to an environmental stimulus such as heat, cold, drought, light, or other stimuli, such as wounding or chemical application.

In an aspect, a promoter provided herein is an axillary bud-specific promoter. In another aspect, a promoter provided herein is an axillary bud-preferred promoter. In an aspect, a promoter provided herein is a constitutive promoter. In another aspect, a promoter provided herein is an inducible promoter. In a further aspect, a promoter provided herein is a developmental promoter.

In an aspect, this disclosure provides a heterologous promoter. In another aspect, this disclosure provides a promoter that is operably linked to a heterologous polynucleotide. In another aspect, this disclosure provides a polynucleotide sequence that is operably linked to a heterologous promoter.

In an aspect, a heterologous promoter comprises an axillary bud-specific promoter. In another aspect, a heterologous promoter comprises an axillary bud-preferred promoter. In an aspect, a heterologous promoter comprises a constitutive promoter. In an aspect, a heterologous promoter comprises an inducible promoter. In an aspect, a heterologous promoter comprises a developmental promoter.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter provided herein is operably linked to a heterologous nucleic acid molecule.

In an aspect, a heterologous promoter comprises a polynucleotide sequence at least 90% identical to a polynucleotide selected from the group consisting of SEQ ID NOs: 89-109. In an aspect, a heterologous promoter comprises a polynucleotide sequence at least 91% identical to a polynucleotide selected from the group consisting of SEQ ID NOs: 89-109. In an aspect, a heterologous promoter comprises a polynucleotide sequence at least 92% identical to a polynucleotide selected from the group consisting of SEQ ID NOs: 89-109. In an aspect, a heterologous promoter comprises a polynucleotide sequence at least 93% identical to a polynucleotide selected from the group consisting of SEQ ID NOs: 89-109. In an aspect, a heterologous promoter comprises a polynucleotide sequence at least 94% identical to a polynucleotide selected from the group consisting of SEQ ID NOs: 89-109. In an aspect, a heterologous promoter comprises a polynucleotide sequence at least 95% identical to a polynucleotide selected from the group consisting of SEQ ID NOs: 89-109. In an aspect, a heterologous promoter comprises a polynucleotide sequence at least 96% identical to a polynucleotide selected from the group consisting of SEQ ID NOs: 89-109. In an aspect, a heterologous promoter comprises a polynucleotide sequence at least 97% identical to a polynucleotide selected from the group consisting of SEQ ID NOs: 89-109. In an aspect, a heterologous promoter comprises a polynucleotide sequence at least 98% identical to a polynucleotide selected from the group consisting of SEQ ID NOs: 89-109. In an aspect, a heterologous promoter comprises a polynucleotide sequence at least 99% identical to a polynucleotide selected from the group consisting of SEQ ID NOs: 89-109. In an aspect, a heterologous promoter comprises a polynucleotide sequence 100% identical to a polynucleotide selected from the group consisting of SEQ ID NOs: 89-109.

In an aspect, an axillary bud-preferred promoter comprises a polynucleotide sequence at least 90% identical to a polynucleotide selected from the group consisting of SEQ ID NOs: 89-109. In an aspect, an axillary bud-preferred promoter comprises a polynucleotide sequence at least 95% identical to a polynucleotide selected from the group consisting of SEQ ID NOs: 89-109. In an aspect, an axillary bud-preferred promoter comprises a polynucleotide sequence at least 96% identical to a polynucleotide selected from the group consisting of SEQ ID NOs: 89-109. In an aspect, an axillary bud-preferred promoter comprises a polynucleotide sequence at least 97% identical to a polynucleotide selected from the group consisting of SEQ ID NOs: 89-109. In an aspect, an axillary bud-preferred promoter comprises a polynucleotide sequence at least 98% identical to a polynucleotide selected from the group consisting of SEQ ID NOs: 89-109. In an aspect, an axillary bud-preferred promoter comprises a polynucleotide sequence at least 99% identical to a polynucleotide selected from the group consisting of SEQ ID NOs: 89-109. In an aspect, an axillary bud-preferred promoter comprises a polynucleotide sequence 100% identical to a polynucleotide selected from the group consisting of SEQ ID NOs: 89-109.

In an aspect, an axillary bud-specific promoter comprises a polynucleotide sequence at least 90% identical to a polynucleotide selected from the group consisting of SEQ ID NOs: 89-109. In an aspect, an axillary bud-specific promoter comprises a polynucleotide sequence at least 95% identical to a polynucleotide selected from the group consisting of SEQ ID NOs: 89-109. In an aspect, an axillary bud-specific promoter comprises a polynucleotide sequence at least 96% identical to a polynucleotide selected from the group consisting of SEQ ID NOs: 89-109. In an aspect, an axillary bud-specific promoter comprises a polynucleotide sequence at least 97% identical to a polynucleotide selected from the group consisting of SEQ ID NOs: 89-109. In an aspect, an axillary bud-specific promoter comprises a polynucleotide sequence at least 98% identical to a polynucleotide selected from the group consisting of SEQ ID NOs: 89-109. In an aspect, an axillary bud-specific promoter comprises a polynucleotide sequence at least 99% identical to a polynucleotide selected from the group consisting of SEQ ID NOs: 89-109. In an aspect, an axillary bud-specific promoter comprises a polynucleotide sequence 100% identical to a polynucleotide selected from the group consisting of SEQ ID NOs: 89-109.

In one aspect, this disclosure provides a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, where the modified tobacco plant comprises no suckers or reduced suckers after topping as compared to a control tobacco plant when grown under comparable growth conditions.

In another aspect, this disclosure provides a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.

In a further aspect, this disclosure provides a method for producing a modified tobacco plant comprising: (a) introducing a recombinant DNA construct to at least one tobacco cell, where the recombinant DNA construct comprises a heterologous promoter operably linked to a nucleic acid encoding a polypeptide selected from the group consisting of a cyclin, a cyclin-dependent kinase (CDK), a CDK inhibitor, a MYB, and a WRKY transcription factor; (b) selecting at least one tobacco cell comprising the recombinant DNA construct; and (c) regenerating a modified tobacco plant from the at least one tobacco cell selected in step (b), where the modified tobacco plant comprises no suckers or reduced suckers after topping as compared to a control tobacco plant lacking the recombinant DNA construct when grown under comparable growth conditions. In an aspect, this method further comprises (d) growing the modified tobacco plant regenerated in step (c). In another aspect, this method further comprises (e) crossing the modified tobacco plant grown in step (d) with a second tobacco plant; and (f) obtaining at least one seed from the crossing in step (e).

In an aspect, this disclosure provides a method comprising transforming a tobacco cell with a recombinant DNA construct comprising a heterologous promoter operably linked to a nucleic acid encoding a polypeptide selected from the group consisting of a cyclin, a cyclin-dependent kinase (CDK), a CDK inhibitor, a MYB, and a WRKY transcription factor. In in aspect, this method further comprises regenerating a modified tobacco plant from said tobacco cell, wherein said modified tobacco plant comprises said recombinant DNA construct.

In one aspect, this disclosure provides recombinant DNA constructs comprising polynucleotides provided herein. As used herein, the term “recombinant DNA construct” refers to a construct formed by laboratory methods of genetic recombination, such as molecular cloning. In an aspect, a recombinant DNA construct is synthetically produced. In another aspect, a recombinant DNA construct comprises a promoter operably linked to a heterologous polynucleotide sequence. In an aspect, a recombinant DNA construct comprises a polynucleotide sequence that encodes an amino acid sequence. In another aspect, a recombinant DNA construct comprises a polynucleotide sequence encoding a small RNA or a prescursor thereof. In an aspect, a recombinant DNA construct comprises a plasmid. In another aspect, a recombinant DNA construct comprises a vector.

As used herein, the terms “vector” or “plasmid” are used interchangeably and refer to a circular, double-stranded DNA molecule that is physically separate from chromosomal DNA. In one aspect, a plasmid or vector used herein is capable of replication in vivo. A “transformation vector,” as used herein, is a plasmid that is capable of transforming a plant cell. In an aspect, a plasmid provided herein is a bacterial plasmid. In another aspect, a plasmid provided herein is an Agrobacterium Ti plasmid or derived from an Agrobacterium Ti plasmid.

In an aspect, a vector provided herein comprises a promoter. In an aspect, a vector provided herein comprises an axillary bud-specific promoter. In an aspect, a vector provided herein comprises an axillary bud-preferred promoter. In another aspect, a vector provided herein comprises a small RNA. In another aspect, a vector provided herein comprises a small RNA precursor. In an aspect, a vector provided herein comprises an artificial miRNA. In another aspect, a vector provided herein comprises an artificial miRNA precursor. In another aspect, a vector provided herein comprises a sequence at least 80% identical to a sequence selected from the group consisting of SEQ ID NOs: 1-44 and 89-113, or fragments thereof. In another aspect, a vector provided herein comprises a sequence at least 85% identical to a sequence selected from the group consisting of SEQ ID NOs: 1-44 and 89-113, or fragments thereof. In another aspect, a vector provided herein comprises a sequence at least 90% identical to a sequence selected from the group consisting of SEQ ID NOs: 1-44 and 89-113, or fragments thereof. In another aspect, a vector provided herein comprises a sequence at least 95% identical to a sequence selected from the group consisting of SEQ ID NOs: 1-44 and 89-113, or fragments thereof. In another aspect, a vector provided herein comprises a sequence at least 96% identical to a sequence selected from the group consisting of SEQ ID NOs: 1-44 and 89-113, or fragments thereof. In another aspect, a vector provided herein comprises a sequence at least 97% identical to a sequence selected from the group consisting of SEQ ID NOs: 1-44 and 89-113, or fragments thereof. In another aspect, a vector provided herein comprises a sequence at least 98% identical to a sequence selected from the group consisting of SEQ ID NOs: 1-44 and 89-113, or fragments thereof. In another aspect, a vector provided herein comprises a sequence at least 99% identical to a sequence selected from the group consisting of SEQ ID NOs: 1-44 and 89-113, or fragments thereof. In another aspect, a vector provided herein comprises a sequence 100% identical to a sequence selected from the group consisting of SEQ ID NOs: 1-44 and 89-113, or fragments thereof.

As used herein, a “fragment” of a nucleic acid sequence or amino acid sequence comprises a contiguous segment of sequence comprising between 1% to 99.99% of the total length of a reference sequence. For example, if a nucleic acid sequence comprises 1000 nucleotides, a fragment of the nucleic acid sequence could comprise any number between 10 and 999 contiguous nucleotides of the reference sequence. This disclosure explicitly provides fragments of each of SEQ ID NOs: 1-114 where ever SEQ ID NOs 1-114 are referenced.

Numerous methods for introducing a recombinant DNA construct to a plant cell are known in the art, which can be used according to methods of the present application to produce a transgenic plant cell and plant. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, polyethylene glycol (PEG)-mediated transformation, etc., are also known in the art. Transgenic plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a tobacco cell with any of the nucleic acid molecules provided herein.

In an aspect, a method of providing a nucleic acid molecule to a tobacco cell comprises Agrobacterium-mediated transformation. In another aspect, a method of providing a nucleic acid molecule to a cell comprises PEG-mediated transformation. In another aspect, a method of providing a nucleic acid molecule to a cell comprises biolistic transformation. In another aspect, a method of providing a nucleic acid molecule to a cell comprises liposome-mediated transfection (lipofection). In another aspect, a method of providing a nucleic acid molecule to a cell comprises lentiviral transfection.

Lipofection is described in e.g., U.S. Pat. Nos. 5,049,386, 4,946,787; and 4,897,355) and lipofection reagents are sold commercially (e.g., Transfectam™, which is the cationic lipid dioctadecylamidoglycylspermine (DOGS), and Lipofectin™, which is a 1:1 (w/w) liposome formulation of the cationic lipid N-[1-(2,3-dioleyloxy) propyl]-n,n,n-trimethylammonium chloride (DOTMA) and dioleoyl phophotidylethanolamine (DOPE) in membrane-filtered water). Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration).

Any tobacco cell from which a fertile tobacco plant can be regenerated is contemplated as a useful recipient cell for practice of this disclosure. In an aspect, a recombinant DNA construct is introduced to a tobacco cell. In an aspect, a recombinant DNA construct is introduced to a tobacco protoplast cell. In another aspect, a recombinant DNA construct is introduced to a tobacco callus cell. In an aspect, a recombinant DNA construct is introduced to a tobacco cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference.

In one aspect, this disclosure provides a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, where the modified tobacco plant comprises no suckers or reduced suckers after topping as compared to a control tobacco plant when grown under comparable growth conditions.

In another aspect, this disclosure provides a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

In a further aspect, this disclosure provides a method for producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor; (b) selecting at least one tobacco cell comprising the recombinant DNA construct; and (c) regenerating a modified tobacco plant from the at least one tobacco cell selected in step (b), where the modified tobacco plant comprises no suckers or reduced suckers after topping as compared to a control tobacco plant lacking the recombinant DNA construct when grown under comparable growth conditions. In an aspect, this method further comprises (d) growing the modified tobacco plant regenerated in step (c). In another aspect, this method further comprises (e) crossing the modified tobacco plant grown in step (d) with a second tobacco plant; and (f) obtaining at least one seed from the crossing in step (e).

In another aspect, this disclosure provides a method comprising transforming a tobacco cell with a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor. In an aspect, this method further comprises regenerating a modified tobacco plant from said tobacco cell, wherein said modified tobacco plant comprises said recombinant DNA construct.

As used herein, the term “small RNA molecule” refers to any non-coding RNA molecule. In addition to providing small RNA molecules, this disclosure also provides a small RNA precursor molecule for each small RNA molecule.

In an aspect, a small RNA molecule comprises between 18 nucleotides and 30 nucleotides in length. In another aspect, a small RNA molecule comprises between 18 nucleotides and 24 nucleotides in length. In another aspect, a small RNA molecule comprises between 18 nucleotides and 22 nucleotides in length.

In another aspect, a small RNA molecule is 18 nucleotides in length. In another aspect, a small RNA molecule is 19 nucleotides in length. In another aspect, a small RNA molecule is 20 nucleotides in length. In another aspect, a small RNA molecule is 21 nucleotides in length. In another aspect, a small RNA molecule is 22 nucleotides in length. In another aspect, a small RNA molecule is 23 nucleotides in length. In another aspect, a small RNA molecule is 24 nucleotides in length. In another aspect, a small RNA molecule is 25 nucleotides in length. In another aspect, a small RNA molecule is 26 nucleotides in length. In another aspect, a small RNA molecule is 27 nucleotides in length. In another aspect, a small RNA molecule is 28 nucleotides in length.

In another aspect, a small RNA molecule is at least 18 nucleotides in length. In another aspect, a small RNA molecule is at least 19 nucleotides in length. In another aspect, a small RNA molecule is at least 20 nucleotides in length. In another aspect, a small RNA molecule is at least 21 nucleotides in length. In another aspect, a small RNA molecule is at least 22 nucleotides in length. In another aspect, a small RNA molecule is at least 23 nucleotides in length. In another aspect, a small RNA molecule is at least 24 nucleotides in length. In another aspect, a small RNA molecule is at least 25 nucleotides in length. In another aspect, a small RNA molecule is at least 26 nucleotides in length. In another aspect, a small RNA molecule is at least 27 nucleotides in length. In another aspect, a small RNA molecule is at least 28 nucleotides in length.

In an aspect, a small RNA molecule comprises between 18 nucleotides and 30 nucleotides. In another aspect, a small RNA molecule comprises between 18 nucleotides and 24 nucleotides. In another aspect, a small RNA molecule comprises between 18 nucleotides and 21 nucleotides. In another aspect, a small RNA molecule comprises between 21 nucleotides and 24 nucleotides. In another aspect, a small RNA molecule comprises between 21 nucleotides and 30 nucleotides.

In an aspect, a small RNA molecule provided herein is a microRNA (miRNA). In an aspect, a small RNA molecule provided herein is an artificial miRNA. In another aspect, a small RNA molecule provided herein is a small interfering RNA (siRNA). In another aspect, a small RNA molecule provided herein is a heterochromatic siRNA (hc-siRNA). In another aspect, a small RNA molecule provided herein is a Piwi-interacting RNA (piRNA). In an aspect, a small RNA molecule provided herein is a double-stranded RNA (dsRNA). In another aspect, a small RNA molecule provided herein is a hairpin double-stranded RNA (hp-dsRNA). In another aspect, a small RNA molecule provided herein is a trans-acting siRNA (ta-siRNA). In another aspect, a small RNA molecule provided herein is a naturally occurring antisense siRNA (nat-siRNA). In another aspect, a small RNA molecule provided herein is a Cas9-guide RNA (gRNA). In another aspect, a small RNA molecule provided herein is a Cpf1-gRNA. In another aspect, a small RNA molecule provided herein is a CasX-gRNA. In another aspect, a small RNA molecule provided herein is a Csm1-gRNA.

In an aspect, a small RNA molecule is selected from the group consisting of a dsRNA, a siRNA, a ta-siRNA, and a miRNA. In another aspect, a small RNA molecule is selected from the group consisting of a miRNA, an siRNA, a hc-siRNA, a piRNA, a dsRNA, a hp-dsRNA, a ta-siRNA, a nat-siRNA, a Cas9-gRNA, a Cpf1-gRNA, a CasX-gRNA, and a Csm1-gRNA.

In an aspect, a small RNA molecule provided herein comprises a polynucleotide sequence having at least 90% identity or complementarity with an endogenous mRNA. In an aspect, a small RNA molecule provided herein comprises a polynucleotide sequence having at least 91% identity or complementarity with an endogenous mRNA. In an aspect, a small RNA molecule provided herein comprises a polynucleotide sequence having at least 92% identity or complementarity with an endogenous mRNA. In an aspect, a small RNA molecule provided herein comprises a polynucleotide sequence having at least 93% identity or complementarity with an endogenous mRNA. In an aspect, a small RNA molecule provided herein comprises a polynucleotide sequence having at least 94% identity or complementarity with an endogenous mRNA. In an aspect, a small RNA molecule provided herein comprises a polynucleotide sequence having at least 95% identity or complementarity with an endogenous mRNA. In an aspect, a small RNA molecule provided herein comprises a polynucleotide sequence having at least 96% identity or complementarity with an endogenous mRNA. In an aspect, a small RNA molecule provided herein comprises a polynucleotide sequence having at least 97% identity or complementarity with an endogenous mRNA. In an aspect, a small RNA molecule provided herein comprises a polynucleotide sequence having at least 98% identity or complementarity with an endogenous mRNA. In an aspect, a small RNA molecule provided herein comprises a polynucleotide sequence having at least 99% identity or complementarity with an endogenous mRNA. In an aspect, a small RNA molecule provided herein comprises a polynucleotide sequence having 100% identity or complementarity with an endogenous mRNA.

In an aspect, a small RNA molecule comprises a polynucleotide sequence having at least 85% identity or complementarity with a polynucleotide sequence selected from the group consisting of SEQ ID NOs: 1-44 and 113. In an aspect, a small RNA molecule comprises a polynucleotide sequence having at least 90% identity or complementarity with a polynucleotide sequence selected from the group consisting of SEQ ID NOs: 1-44 and 113. In an aspect, a small RNA molecule comprises a polynucleotide sequence having at least 91% identity or complementarity with a polynucleotide sequence selected from the group consisting of SEQ ID NOs: 1-44 and 113. In an aspect, a small RNA molecule comprises a polynucleotide sequence having at least 92% identity or complementarity with a polynucleotide sequence selected from the group consisting of SEQ ID NOs: 1-44 and 113. In an aspect, a small RNA molecule comprises a polynucleotide sequence having at least 93% identity or complementarity with a polynucleotide sequence selected from the group consisting of SEQ ID NOs: 1-44 and 113. In an aspect, a small RNA molecule comprises a polynucleotide sequence having at least 94% identity or complementarity with a polynucleotide sequence selected from the group consisting of SEQ ID NOs: 1-44 and 113. In an aspect, a small RNA molecule comprises a polynucleotide sequence having at least 95% identity or complementarity with a polynucleotide sequence selected from the group consisting of SEQ ID NOs: 1-44 and 113. In an aspect, a small RNA molecule comprises a polynucleotide sequence having at least 96% identity or complementarity with a polynucleotide sequence selected from the group consisting of SEQ ID NOs: 1-44 and 113. In an aspect, a small RNA molecule comprises a polynucleotide sequence having at least 97% identity or complementarity with a polynucleotide sequence selected from the group consisting of SEQ ID NOs: 1-44 and 113. In an aspect, a small RNA molecule comprises a polynucleotide sequence having at least 98% identity or complementarity with a polynucleotide sequence selected from the group consisting of SEQ ID NOs: 1-44 and 113. In an aspect, a small RNA molecule comprises a polynucleotide sequence having at least 99% identity or complementarity with a polynucleotide sequence selected from the group consisting of SEQ ID NOs: 1-44 and 113. In an aspect, a small RNA molecule comprises a polynucleotide sequence having 100% identity or complementarity with a polynucleotide sequence selected from the group consisting of SEQ ID NOs: 1-44 and 113.

In an aspect, a small RNA molecule comprises a polynucleotide sequence identical to, or complementary to, at least 16 contiguous nucleotides of a polynucleotide sequence selected from the group consisting of SEQ ID NOs: 1-44 and 113. In an aspect, a small RNA molecule comprises a polynucleotide sequence identical to, or complementary to, at least 17 contiguous nucleotides of a polynucleotide sequence selected from the group consisting of SEQ ID NOs: 1-44 and 113. In an aspect, a small RNA molecule comprises a polynucleotide sequence identical to, or complementary to, at least 18 contiguous nucleotides of a polynucleotide sequence selected from the group consisting of SEQ ID NOs: 1-44 and 113. In an aspect, a small RNA molecule comprises a polynucleotide sequence identical to, or complementary to, at least 19 contiguous nucleotides of a polynucleotide sequence selected from the group consisting of SEQ ID NOs: 1-44 and 113. In an aspect, a small RNA molecule comprises a polynucleotide sequence identical to, or complementary to, at least 20 contiguous nucleotides of a polynucleotide sequence selected from the group consisting of SEQ ID NOs: 1-44 and 113. In an aspect, a small RNA molecule comprises a polynucleotide sequence identical to, or complementary to, at least 21 contiguous nucleotides of a polynucleotide sequence selected from the group consisting of SEQ ID NOs: 1-44 and 113. In an aspect, a small RNA molecule comprises a polynucleotide sequence identical to, or complementary to, at least 22 contiguous nucleotides of a polynucleotide sequence selected from the group consisting of SEQ ID NOs: 1-44 and 113. In an aspect, a small RNA molecule comprises a polynucleotide sequence identical to, or complementary to, at least 23 contiguous nucleotides of a polynucleotide sequence selected from the group consisting of SEQ ID NOs: 1-44 and 113. In an aspect, a small RNA molecule comprises a polynucleotide sequence identical to, or complementary to, at least 24 contiguous nucleotides of a polynucleotide sequence selected from the group consisting of SEQ ID NOs: 1-44 and 113. In an aspect, a small RNA molecule comprises a polynucleotide sequence identical to, or complementary to, at least 25 contiguous nucleotides of a polynucleotide sequence selected from the group consisting of SEQ ID NOs: 1-44 and 113. In an aspect, a small RNA molecule comprises a polynucleotide sequence identical to, or complementary to, at least 26 contiguous nucleotides of a polynucleotide sequence selected from the group consisting of SEQ ID NOs: 1-44 and 113. In an aspect, a small RNA molecule comprises a polynucleotide sequence identical to, or complementary to, at least 27 contiguous nucleotides of a polynucleotide sequence selected from the group consisting of SEQ ID NOs: 1-44 and 113. In an aspect, a small RNA molecule comprises a polynucleotide sequence identical to, or complementary to, at least 28 contiguous nucleotides of a polynucleotide sequence selected from the group consisting of SEQ ID NOs: 1-44 and 113. In an aspect, a small RNA molecule comprises a polynucleotide sequence identical to, or complementary to, at least 29 contiguous nucleotides of a polynucleotide sequence selected from the group consisting of SEQ ID NOs: 1-44 and 113. In an aspect, a small RNA molecule comprises a polynucleotide sequence identical to, or complementary to, at least 30 contiguous nucleotides of a polynucleotide sequence selected from the group consisting of SEQ ID NOs: 1-44 and 113.

In an aspect, this disclosure provides a small RNA capable of reducing the expression of a polynucleotide encoding a cyclin. In an aspect, this disclosure provides a small RNA capable of reducing the translation of a polynucleotide encoding a cyclin. In an aspect, this disclosure provides a small RNA capable of reducing the expression of a polynucleotide encoding a CDK. In an aspect, this disclosure provides a small RNA capable of reducing the translation of a polynucleotide encoding a CDK. In an aspect, this disclosure provides a small RNA capable of reducing the expression of a polynucleotide encoding a CDKI. In an aspect, this disclosure provides a small RNA capable of reducing the translation of a polynucleotide encoding a CDKI. In an aspect, this disclosure provides a small RNA capable of reducing the expression of a polynucleotide encoding a MYB. In an aspect, this disclosure provides a small RNA capable of reducing the translation of a polynucleotide encoding a MYB. In an aspect, this disclosure provides a small RNA capable of reducing the expression of a polynucleotide encoding a WRKY transcription factor. In an aspect, this disclosure provides a small RNA capable of reducing the translation of a polynucleotide encoding a WRKY transcription factor.

MicroRNAs (miRNAs) are non-protein coding RNAs, generally of between about 19 to about 25 nucleotides (commonly about 20-24 nucleotides in plants), that guide cleavage in trans of target transcripts, negatively regulating the expression of genes involved in various regulation and development pathways. In some cases, miRNAs serve to guide in-phase processing of siRNA primary transcripts.

Many microRNA genes (MIR genes) have been identified and made publicly available in a database (“miRBase”, available on line at microrna (dot) sanger (dot) ac (dot) uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). Transcription of MIR genes can be, at least in some cases, under promotional control of a MIR gene's own promoter. The primary transcript, termed a “pri-miRNA”, can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA.

Transgenic expression of miRNAs (whether a naturally occurring sequence or an artificial sequence) can be employed to regulate expression of the miRNA's target gene or genes. Inclusion of a miRNA recognition site in a transgenically expressed transcript is also useful in regulating expression of the transcript. Recognition sites of miRNAs have been validated in all regions of an mRNA, including the 5′ untranslated region, coding region, and 3′ untranslated region, indicating that the position of the miRNA target site relative to the coding sequence may not necessarily affect suppression.

Because miRNAs are important regulatory elements in eukaryotes, transgenic suppression of miRNAs is useful for manipulating biological pathways and responses. Various utilities of miRNAs, their precursors, their recognition sites, and their promoters are described in detail in U.S. Patent Application Publication 2006/0200878 A1, incorporated by reference herein. Non-limiting examples of these utilities include: (1) the expression of a native miRNA or miRNA precursor sequence to suppress a target gene; (2) the expression of an artificial miRNA or miRNA precursor sequence to suppress a target gene; (3) expression of a transgene with a miRNA recognition site, where the transgene is suppressed when the mature miRNA is expressed; (4) expression of a transgene driven by a miRNA promoter.

Designing an artificial miRNA sequence can be as simple as substituting sequence that is complementary to the intended target for nucleotides in the miRNA stem region of the miRNA precursor, as demonstrated by Zeng et al. (2002) Mol. Cell, 9:1327-1333. One non-limiting example of a general method for determining nucleotide changes in the native miRNA sequence to produce the engineered miRNA precursor includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402), for example, of both tobacco cDNA and genomic DNA databases, to identify target transcript orthologues and any potential matches to unrelated genes, thereby avoiding unintentional silencing of non-target sequences; (b) Analyzing the target gene for undesirable sequences (e.g., matches to sequences from non-target species), and score each potential 19-mer segment for GC content, Reynolds score (see Reynolds et al. (2004) Nature Biotechnol., 22:326-330), and functional asymmetry characterized by a negative difference in free energy (“.DELTA . . . DELTA.G” or “ΔΔG”) (see Khvorova et al. (2003) Cell, 114:209-216). Preferably 19-mers are selected that have all or most of the following characteristics: (1) a Reynolds score>4, (2) a GC content between about 40% to about 60%, (3) a negative ΔΔG, (4) a terminal adenosine, (5) lack of a consecutive run of 4 or more of the same nucleotide; (6) a location near the 3′ terminus of the target gene; (7) minimal differences from the miRNA precursor transcript. Positions at every third nucleotide in an siRNA have been reported to be especially important in influencing RNAi efficacy and an algorithm, “siExplorer” is publicly available at rna (dot) chem (dot) t (dot) u-tokyo (dot) ac (dot) jp/siexplorer.htm (see Katoh and Suzuki (2007) Nucleic Acids Res., 10.1093/nar/gkl1120); (c) Determining the reverse complement of the selected 19-mers to use in making a modified mature miRNA. The additional nucleotide at position 20 is preferably matched to the selected target sequence, and the nucleotide at position 21 is preferably chosen to either be unpaired to prevent spreading of silencing on the target transcript or paired to the target sequence to promote spreading of silencing on the target transcript; and (d) transforming the artificial miRNA into a plant.

In one aspect, an artificial miRNA provided herein is complementary to at least 18 contiguous nucleotides of a nucleic acid sequence selected from the group consisting of SEQ ID NOs: 1-44 and 113. In one aspect, an artificial miRNA provided herein is complementary to at least 19 contiguous nucleotides of a nucleic acid sequence selected from the group consisting of SEQ ID NOs: 1-44 and 113. In one aspect, an artificial miRNA provided herein is complementary to at least 20 contiguous nucleotides of a nucleic acid sequence selected from the group consisting of SEQ ID NOs: 1-44 and 113. In one aspect, an artificial miRNA provided herein is complementary to at least 21 contiguous nucleotides of a nucleic acid sequence selected from the group consisting of SEQ ID NOs: 1-44 and 113. In one aspect, an artificial miRNA provided herein is complementary to at least 22 contiguous nucleotides of a nucleic acid sequence selected from the group consisting of SEQ ID NOs: 1-44 and 113. In one aspect, an artificial miRNA provided herein is complementary to at least 23 contiguous nucleotides of a nucleic acid sequence selected from the group consisting of SEQ ID NOs: 1-44 and 113. In one aspect, an artificial miRNA provided herein is complementary to at least 24 contiguous nucleotides of a nucleic acid sequence selected from the group consisting of SEQ ID NOs: 1-44 and 113. In one aspect, an artificial miRNA provided herein is complementary to at least 25 contiguous nucleotides of a nucleic acid sequence selected from the group consisting of SEQ ID NOs: 1-44 and 113. In one aspect, an artificial miRNA provided herein is complementary to at least 26 contiguous nucleotides of a nucleic acid sequence selected from the group consisting of SEQ ID NOs: 1-44 and 113.

In one aspect, an artificial miRNA provided herein is at least 75% complementary to a nucleic acid sequence selected from the group consisting of SEQ ID NOs: 1-44 and 113. In one aspect, an artificial miRNA provided herein is at least 80% complementary to a nucleic acid sequence selected from the group consisting of SEQ ID NOs: 1-44 and 113. In one aspect, an artificial miRNA provided herein is at least 85% complementary to a nucleic acid sequence selected from the group consisting of SEQ ID NOs: 1-44 and 113. In one aspect, an artificial miRNA provided herein is at least 90% complementary to a nucleic acid sequence selected from the group consisting of SEQ ID NOs: 1-44 and 113. In one aspect, an artificial miRNA provided herein is at least 91% complementary to a nucleic acid sequence selected from the group consisting of SEQ ID NOs: 1-44 and 113. In one aspect, an artificial miRNA provided herein is at least 92% complementary to a nucleic acid sequence selected from the group consisting of SEQ ID NOs: 1-44 and 113. In one aspect, an artificial miRNA provided herein is at least 93% complementary to a nucleic acid sequence selected from the group consisting of SEQ ID NOs: 1-44 and 113. In one aspect, an artificial miRNA provided herein is at least 94% complementary to a nucleic acid sequence selected from the group consisting of SEQ ID NOs: 1-44 and 113. In one aspect, an artificial miRNA provided herein is at least 95% complementary to a nucleic acid sequence selected from the group consisting of SEQ ID NOs: 1-44 and 113. In one aspect, an artificial miRNA provided herein is at least 96% complementary to a nucleic acid sequence selected from the group consisting of SEQ ID NOs: 1-44 and 113. In one aspect, an artificial miRNA provided herein is at least 97% complementary to a nucleic acid sequence selected from the group consisting of SEQ ID NOs: 1-44 and 113. In one aspect, an artificial miRNA provided herein is at least 98% complementary to a nucleic acid sequence selected from the group consisting of SEQ ID NOs: 1-44 and 113. In one aspect, an artificial miRNA provided herein is at least 99% complementary to a nucleic acid sequence selected from the group consisting of SEQ ID NOs: 1-44 and 113. In one aspect, an artificial miRNA provided herein is 100% complementary to a nucleic acid sequence selected from the group consisting of SEQ ID NOs: 1-44 and 113.

In one aspect, an artificial miRNA provided herein reduces or eliminates RNA transcription or protein translation of a target gene.

In one aspect, an artificial miRNA, or a precursor thereof, is operably linked to an axillary bud-specific promoter. In another aspect, an artificial miRNA, or a precursor thereof, is operably linked to an axillary bud-preferred promoter. In another aspect, an artificial miRNA, or a precursor thereof, is operably linked to a promoter comprising a sequence selected from the group consisting of SEQ ID NOs: 89-109 and fragments thereof.

Tobacco is known in the art as a plant from the family Solanaceae. As used herein, a tobacco plant can be from any plant from the Nicotiana genus including, but not limited to Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572. In an aspect, a tobacco plant described here is a Nicotiana tabacum plant.

In one aspect, modified tobacco plants, seeds, cells, hybrids, varieties, or lines provided herein are essentially derived from or in the genetic background of BU 64, CC 101, CC 200, CC 13, CC 27, CC 33, CC 35, CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpão, GL 26H, GL 338, GL 350, GL 395, GL 600, GL 737, GL 939, GL 973, GF 157, GF 318, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY 907LC, KTY14×L8 LC, Little Crittenden, McNair 373, McNair 944, male sterile KY 14×L8, Narrow Leaf Madole, MS KY171, Narrow Leaf Madole (phph), MS Narrow Leaf Madole, MS TND950, PD 7302LC, PD 7305LC, PD 7309LC, PD 7312LC, PD 7318LC, PD 7319LC, MSTKS 2002, TKF 2002, TKF 6400, TKF 4028, TKF 4024, KT206LC, KT209LC, KT210LC, KT212LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H2O, Speight NF3, TI 1406, TI 1269, TN 86, TN86LC, TN 90, TN90LC, TN 97, TN97LC, TN D94, TN D950, a TR (Tom Rosson) Madole, VA 309, VA 359, or any commercial tobacco variety according to standard tobacco breeding techniques known in the art.

In an aspect, a modified tobacco plant is selected from the group consisting of a BU 64 plant, a CC 101 plant, a CC 200 plant, a CC 13 plant, a CC 27 plant, a CC 33 plant, a CC 35 plant, a CC 37 plant, a CC 65 plant, a CC 67 plant, a CC 301 plant, a CC 400 plant, a CC 500 plant, CC 600 plant, a CC 700 plant, a CC 800 plant, a CC 900 plant, a CC 1063 plant, a Coker 176 plant, a Coker 319 plant, a Coker 371 Gold plant, a Coker 48 plant, a CU 263 plant, a DF911 plant, a Galpao plant, a GL 26H plant, a GL 338 plant, a GL 350 plant, a GL 395 plant, a GL 600 plant, a GL 737 plant, a GL 939 plant, a GL 973 plant, a GF 157 plant, a GF 318 plant, an RJR 901 plant, an HB 04P plant, a K 149 plant, a K 326 plant, a K 346 plant, a K 358 plant, a K394 plant, a K 399 plant, a K 730 plant, an NC 196 plant, an NC 37NF plant, an NC 471 plant, an NC 55 plant, an NC 92 plant, an NC2326 plant, an NC 95 plant, an NC 925 plant, a PVH 1118 plant, a PVH 1452 plant, a PVH 2110 plant, a PVH 2254 plant, a PVH 2275 plant, a VA 116 plant, a VA 119 plant, a KDH 959 plant, a KT 200 plant, a KT204LC plant, a KY 10 plant, a KY 14 plant, a KY 160 plant, a KY 17 plant, a KY 171 plant, a KY 907 plant, a KY 907LC plant, a KTY14×L8 LC plant, a Little Crittenden plant, a McNair 373 plant, a McNair 944 plant, a male sterile KY 14×L8 plant, a Narrow Leaf Madole plant, a MS KY171 plant, a Narrow Leaf Madole (phph) plant, a MS Narrow Leaf Madole plant, a MS TND950 plant, a PD 7302LC plant, a PD 7305LC plant, a PD 7309LC plant, a PD 7312LC plant, a PD 7318LC plant, a PD 7319LC plant, a MSTKS 2002 plant, a TKF 2002 plant, a TKF 6400 plant, a TKF 4028 plant, a TKF 4024 plant, a KT206LC plant, a KT209LC plant, a KT210LC plant, a KT212LC plant, an NC 100 plant, an NC 102 plant, an NC 2000 plant, an NC 291 plant, an NC 297 plant, an NC 299 plant, an NC 3 plant, an NC 4 plant, an NC 5 plant, an NC 6 plant, an NC7 plant, an NC 606 plant, an NC 71 plant, an NC 72 plant, an NC 810 plant, an NC BH 129 plant, an NC 2002 plant, a Neal Smith Madole plant, an OXFORD 207 plant, a ‘Perique’ plant, a PVH03 plant, a PVH09 plant, a PVH19 plant, a PVH50 plant, a PVH51 plant, an R 610 plant, an R 630 plant, an R 7-11 plant, an R 7-12 plant, an RG 17 plant, an RG 81 plant, an RG H51 plant, an RGH 4 plant, an RGH 51 plant, an RS 1410 plant, a Speight 168 plant, a Speight 172 plant, a Speight 179 plant, a Speight 210 plant, a Speight 220 plant, a Speight 225 plant, a Speight 227 plant, a Speight 234 plant, a Speight G-28 plant, a Speight G-70 plant, a Speight H-6 plant, a Speight H2O plant, a Speight NF3 plant, a TI 1406 plant, a TI 1269 plant, a TN 86 plant, a TN86LC plant, a TN 90 plant, a TN90LC plant, a TN 97 plant, a TN97LC plant, a TN D94 plant, a TN D950 plant, a TR (Tom Rosson) Madole plant, a VA 309 plant, and a VA 359 plant.

In another aspect, a modified tobacco cell is selected from the group consisting of a BU 64 cell, a CC 101 cell, a CC 200 cell, a CC 13 cell, a CC 27 cell, a CC 33 cell, a CC 35 cell, a CC 37 cell, a CC 65 cell, a CC 67 cell, a CC 301 cell, a CC 400 cell, a CC 500 cell, CC 600 cell, a CC 700 cell, a CC 800 cell, a CC 900 cell, a CC 1063 cell, a Coker 176 cell, a Coker 319 cell, a Coker 371 Gold cell, a Coker 48 cell, a CU 263 cell, a DF911 cell, a Galpao cell, a GL 26H cell, a GL 338 cell, a GL 350 cell, a GL 395 cell, a GL 600 cell, a GL 737 cell, a GL 939 cell, a GL 973 cell, a GF 157 cell, a GF 318 cell, an RJR 901 cell, an HB 04P cell, a K 149 cell, a K 326 cell, a K 346 cell, a K 358 cell, a K394 cell, a K 399 cell, a K 730 cell, an NC 196 cell, an NC 37NF cell, an NC 471 cell, an NC 55 cell, an NC 92 cell, an NC2326 cell, an NC 95 cell, an NC 925 cell, a PVH 1118 cell, a PVH 1452 cell, a PVH 2110 cell, a PVH 2254 cell, a PVH 2275 cell, a VA 116 cell, a VA 119 cell, a KDH 959 cell, a KT 200 cell, a KT204LC cell, a KY 10 cell, a KY 14 cell, a KY 160 cell, a KY 17 cell, a KY 171 cell, a KY 907 cell, a KY 907LC cell, a KTY14×L8 LC cell, a Little Crittenden cell, a McNair 373 cell, a McNair 944 cell, a male sterile KY 14×L8 cell, a Narrow Leaf Madole cell, a MS KY171 cell, a Narrow Leaf Madole (phph) cell, a MS Narrow Leaf Madole cell, a MS TND950 cell, a PD 7302LC cell, a PD 7305LC cell, a PD 7309LC cell, a PD 7312LC cell, a PD 7318LC cell, a PD 7319LC cell, a MSTKS 2002 cell, a TKF 2002 cell, a TKF 6400 cell, a TKF 4028 cell, a TKF 4024 cell, a KT206LC cell, a KT209LC cell, a KT210LC cell, a KT212LC cell, an NC 100 cell, an NC 102 cell, an NC 2000 cell, an NC 291 cell, an NC 297 cell, an NC 299 cell, an NC 3 cell, an NC 4 cell, an NC 5 cell, an NC 6 cell, an NC7 cell, an NC 606 cell, an NC 71 cell, an NC 72 cell, an NC 810 cell, an NC BH 129 cell, an NC 2002 cell, a Neal Smith Madole cell, an OXFORD 207 cell, a ‘Perique’ cell, a PVH03 cell, a PVH09 cell, a PVH19 cell, a PVH50 cell, a PVH51 cell, an R 610 cell, an R 630 cell, an R 7-11 cell, an R 7-12 cell, an RG 17 cell, an RG 81 cell, an RG H51 cell, an RGH 4 cell, an RGH 51 cell, an RS 1410 cell, a Speight 168 cell, a Speight 172 cell, a Speight 179 cell, a Speight 210 cell, a Speight 220 cell, a Speight 225 cell, a Speight 227 cell, a Speight 234 cell, a Speight G-28 cell, a Speight G-70 cell, a Speight H-6 cell, a Speight H2O cell, a Speight NF3 cell, a TI 1406 cell, a TI 1269 cell, a TN 86 cell, a TN86LC cell, a TN 90 cell, a TN90LC cell, a TN 97 cell, a TN97LC cell, a TN D94 cell, a TN D950 cell, a TR (Tom Rosson) Madole cell, a VA 309 cell, and a VA 359 cell.

In another aspect, a modified tobacco seed is selected from the group consisting of a BU 64 seed, a CC 101 seed, a CC 200 seed, a CC 13 seed, a CC 27 seed, a CC 33 seed, a CC 35 seed, a CC 37 seed, a CC 65 seed, a CC 67 seed, a CC 301 seed, a CC 400 seed, a CC 500 seed, CC 600 seed, a CC 700 seed, a CC 800 seed, a CC 900 seed, a CC 1063 seed, a Coker 176 seed, a Coker 319 seed, a Coker 371 Gold seed, a Coker 48 seed, a CU 263 seed, a DF911 seed, a Galpao seed, a GL 26H seed, a GL 338 seed, a GL 350 seed, a GL 395 seed, a GL 600 seed, a GL 737 seed, a GL 939 seed, a GL 973 seed, a GF 157 seed, a GF 318 seed, an RJR 901 seed, an HB 04P seed, a K 149 seed, a K 326 seed, a K 346 seed, a K 358 seed, a K394 seed, a K 399 seed, a K 730 seed, an NC 196 seed, an NC 37NF seed, an NC 471 seed, an NC 55 seed, an NC 92 seed, an NC2326 seed, an NC 95 seed, an NC 925 seed, a PVH 1118 seed, a PVH 1452 seed, a PVH 2110 seed, a PVH 2254 seed, a PVH 2275 seed, a VA 116 seed, a VA 119 seed, a KDH 959 seed, a KT 200 seed, a KT204LC seed, a KY 10 seed, a KY 14 seed, a KY 160 seed, a KY 17 seed, a KY 171 seed, a KY 907 seed, a KY 907LC seed, a KTY14×L8 LC seed, a Little Crittenden seed, a McNair 373 seed, a McNair 944 seed, a male sterile KY 14×L8 seed, a Narrow Leaf Madole seed, a MS KY171 seed, a Narrow Leaf Madole (phph) seed, a MS Narrow Leaf Madole seed, a MS TND950 seed, a PD 7302LC seed, a PD 7305LC seed, a PD 7309LC seed, a PD 7312LC seed, a PD 7318LC seed, a PD 7319LC seed, a MSTKS 2002 seed, a TKF 2002 seed, a TKF 6400 seed, a TKF 4028 seed, a TKF 4024 seed, a KT206LC seed, a KT209LC seed, a KT210LC seed, a KT212LC seed, an NC 100 seed, an NC 102 seed, an NC 2000 seed, an NC 291 seed, an NC 297 seed, an NC 299 seed, an NC 3 seed, an NC 4 seed, an NC 5 seed, an NC 6 seed, an NC7 seed, an NC 606 seed, an NC 71 seed, an NC 72 seed, an NC 810 seed, an NC BH 129 seed, an NC 2002 seed, a Neal Smith Madole seed, an OXFORD 207 seed, a ‘Perique’ seed, a PVH03 seed, a PVH09 seed, a PVH19 seed, a PVH50 seed, a PVH51 seed, an R 610 seed, an R 630 seed, an R 7-11 seed, an R 7-12 seed, an RG 17 seed, an RG 81 seed, an RG H51 seed, an RGH 4 seed, an RGH 51 seed, an RS 1410 seed, a Speight 168 seed, a Speight 172 seed, a Speight 179 seed, a Speight 210 seed, a Speight 220 seed, a Speight 225 seed, a Speight 227 seed, a Speight 234 seed, a Speight G-28 seed, a Speight G-70 seed, a Speight H-6 seed, a Speight H2O seed, a Speight NF3 seed, a TI 1406 seed, a TI 1269 seed, a TN 86 seed, a TN86LC seed, a TN 90 seed, a TN90LC seed, a TN 97 seed, a TN97LC seed, a TN D94 seed, a TN D950 seed, a TR (Tom Rosson) Madole seed, a VA 309 seed, and a VA 359 seed.

As used herein, “tobacco plant” refers to a whole tobacco plant. A tobacco cell or tobacco tissue culture derived from a tobacco plant can comprise any tobacco plant parts or tobacco plant organs (e.g., leaves, stems, roots, etc.), tobacco plant tissues, tobacco seeds, tobacco plant cells, and/or progeny of the same. A progeny plant can be from any filial generation, e.g., F₁, F₂, F₃, F₄, F₅, F₆, F₇, etc. A tobacco plant cell is a biological cell of a tobacco plant, taken from a tobacco plant or derived through culture from a cell taken from a tobacco plant. As used herein, “seedling” refers to a tobacco plant that is equal to, or less than, 14 days post-germination.

In one aspect, a tobacco plant part provided herein includes, but is not limited to, a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In further aspects, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic plant cells. Somatic cells, contrary to germline cells, do not mediate plant reproduction.

Provided tobacco cells, tobacco tissues and tobacco organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue. In another aspect, this disclosure provides a tobacco plant chloroplast. In a further aspect, this disclosure provides an epidermal cell, a stomata cell, a trichome cell, a root hair, or a storage root.

In an aspect, this disclosure provides a tobacco protoplast cell. In another aspect, this disclosure provides a tobacco callus cell. In another aspect, this disclosure provides a tobacco seed cell. In another aspect, this disclosure provides a tobacco fruit cell. In another aspect, this disclosure provides a tobacco leaf cell. In another aspect, this disclosure provides a tobacco cotyledon cell. In another aspect, this disclosure provides a tobacco hypocotyl cell. In another aspect, this disclosure provides a tobacco meristem cell. In another aspect, this disclosure provides a tobacco embryo cell. In another aspect, this disclosure provides a tobacco root cell. In another aspect, this disclosure provides a tobacco shoot cell. In another aspect, this disclosure provides a tobacco stem cell. In another aspect, this disclosure provides a tobacco flower cell. In another aspect, this disclosure provides a tobacco inflorescence cell. In another aspect, this disclosure provides a tobacco stalk cell. In another aspect, this disclosure provides a tobacco pedicel cell. In another aspect, this disclosure provides a tobacco style cell. In another aspect, this disclosure provides a tobacco stigma cell. In another aspect, this disclosure provides a tobacco receptacle cell. In another aspect, this disclosure provides a tobacco petal cell. In another aspect, this disclosure provides a tobacco sepal cell. In another aspect, this disclosure provides a tobacco pollen cell. In another aspect, this disclosure provides a tobacco anther cell. In another aspect, this disclosure provides a tobacco filament cell. In another aspect, this disclosure provides a tobacco ovary cell. In another aspect, this disclosure provides a tobacco ovule cell. In another aspect, this disclosure provides a tobacco pericarp cell. In another aspect, this disclosure provides a tobacco phloem cell.

This disclosure provides modified tobacco plants, modified tobacco plant parts, modified tobacco seeds, and modified tobacco cells and methods of making the same. In an aspect, this disclosure provides a tobacco leaf of a modified tobacco plant. In another aspect, this disclosure provides a tobacco seed of a modified tobacco plant. In another aspect, this disclosure provides a tobacco stem of a modified tobacco plant. In a further aspect, this disclosure provides a tobacco plant part of a modified tobacco plant. In another aspect, this disclosure provides a tobacco cell of a modified tobacco plant. In a further aspect, this disclosure provides a dried tobacco leaf of a modified tobacco plant. In still a further aspect, this disclosure provides a cured tobacco leaf of a modified tobacco plant. In yet another aspect, this disclosure provides a fermented tobacco leaf of a modified tobacco plant.

In another aspect, this disclosure provides an alkaloid extracted from a modified tobacco plant. In another aspect, this disclosure provides nicotine extracted from a modified tobacco plant. In another aspect, this disclosure provides anatabine extracted from a modified tobacco plant. In another aspect, this disclosure provides anabasine extracted from a modified tobacco plant. In another aspect, this disclosure provides nornicotine extracted from a modified tobacco plant.

In an aspect, a modified tobacco plant, plant part, seed, cell, or genome is cisgenic. As used herein, “cisgenic” refers to genetic modification of a plant, plant cell, or plant genome in which all parts (e.g., promoter, donor nucleic acid, selection gene) have only plant origins (i.e., no non-plant origin parts are used). Cisgenic plants, plant cells, and plant genomes provided herein can lead to ready-to-use tobacco lines. In another aspect, a modified tobacco plant provided herein comprises no non-tobacco genetic material or sequences.

In an aspect, a modified plant comprises an increased leaf yield mass as compared to a control tobacco plant when grown under comparable conditions. In an aspect, leaf yield is selected from the group consisting of fresh leaf yield mass, dry leaf yield mass, and cured leaf yield mass.

In an aspect, a modified plant comprises at least 0.5% increased leaf yield mass as compared to a control tobacco plant when grown under comparable conditions. In an aspect, a modified plant comprises at least 0.5% increased fresh leaf yield mass as compared to a control tobacco plant when grown under comparable conditions. In an aspect, a modified plant comprises at least 0.5% increased dry leaf yield mass as compared to a control tobacco plant when grown under comparable conditions. In an aspect, a modified plant comprises at least 0.5% increased cured leaf yield mass as compared to a control tobacco plant when grown under comparable conditions. In an aspect, a modified plant comprises at least 1% increased leaf yield mass as compared to a control tobacco plant when grown under comparable conditions. In an aspect, a modified plant comprises at least 1% increased fresh leaf yield mass as compared to a control tobacco plant when grown under comparable conditions. In an aspect, a modified plant comprises at least 1% increased dry leaf yield mass as compared to a control tobacco plant when grown under comparable conditions. In an aspect, a modified plant comprises at least 1% increased cured leaf yield mass as compared to a control tobacco plant when grown under comparable conditions. In an aspect, a modified plant comprises at least 2% increased leaf yield mass as compared to a control tobacco plant when grown under comparable conditions. In an aspect, a modified plant comprises at least 2% increased fresh leaf yield mass as compared to a control tobacco plant when grown under comparable conditions. In an aspect, a modified plant comprises at least 2% increased dry leaf yield mass as compared to a control tobacco plant when grown under comparable conditions. In an aspect, a modified plant comprises at least 2% increased cured leaf yield mass as compared to a control tobacco plant when grown under comparable conditions. In an aspect, a modified plant comprises at least 3% increased leaf yield mass as compared to a control tobacco plant when grown under comparable conditions. In an aspect, a modified plant comprises at least 3% increased fresh leaf yield mass as compared to a control tobacco plant when grown under comparable conditions. In an aspect, a modified plant comprises at least 3% increased dry leaf yield mass as compared to a control tobacco plant when grown under comparable conditions. In an aspect, a modified plant comprises at least 3% increased cured leaf yield mass as compared to a control tobacco plant when grown under comparable conditions. In an aspect, a modified plant comprises at least 4% increased leaf yield mass as compared to a control tobacco plant when grown under comparable conditions. In an aspect, a modified plant comprises at least 4% increased fresh leaf yield mass as compared to a control tobacco plant when grown under comparable conditions. In an aspect, a modified plant comprises at least 4% increased dry leaf yield mass as compared to a control tobacco plant when grown under comparable conditions. In an aspect, a modified plant comprises at least 4% increased cured leaf yield mass as compared to a control tobacco plant when grown under comparable conditions. In an aspect, a modified plant comprises at least 5% increased leaf yield mass as compared to a control tobacco plant when grown under comparable conditions. In an aspect, a modified plant comprises at least 5% increased fresh leaf yield mass as compared to a control tobacco plant when grown under comparable conditions. In an aspect, a modified plant comprises at least 5% increased dry leaf yield mass as compared to a control tobacco plant when grown under comparable conditions. In an aspect, a modified plant comprises at least 5% increased cured leaf yield mass as compared to a control tobacco plant when grown under comparable conditions. In an aspect, a modified plant comprises at least 250% increased leaf yield mass as compared to a control tobacco plant when grown under comparable conditions. In an aspect, a modified plant comprises at least 250% increased fresh leaf yield mass as compared to a control tobacco plant when grown under comparable conditions. In an aspect, a modified plant comprises at least 250% increased dry leaf yield mass as compared to a control tobacco plant when grown under comparable conditions. In an aspect, a modified plant comprises at least 250% increased cured leaf yield mass as compared to a control tobacco plant when grown under comparable conditions. In an aspect, a modified plant comprises at least 25% increased leaf yield mass as compared to a control tobacco plant when grown under comparable conditions. In an aspect, a modified plant comprises at least 25% increased fresh leaf yield mass as compared to a control tobacco plant when grown under comparable conditions. In an aspect, a modified plant comprises at least 25% increased dry leaf yield mass as compared to a control tobacco plant when grown under comparable conditions. In an aspect, a modified plant comprises at least 25% increased cured leaf yield mass as compared to a control tobacco plant when grown under comparable conditions. In an aspect, a modified plant comprises at least 50% increased leaf yield mass as compared to a control tobacco plant when grown under comparable conditions. In an aspect, a modified plant comprises at least 50% increased fresh leaf yield mass as compared to a control tobacco plant when grown under comparable conditions. In an aspect, a modified plant comprises at least 50% increased dry leaf yield mass as compared to a control tobacco plant when grown under comparable conditions. In an aspect, a modified plant comprises at least 50% increased cured leaf yield mass as compared to a control tobacco plant when grown under comparable conditions. In an aspect, a modified plant comprises at least 75% increased leaf yield mass as compared to a control tobacco plant when grown under comparable conditions. In an aspect, a modified plant comprises at least 75% increased fresh leaf yield mass as compared to a control tobacco plant when grown under comparable conditions. In an aspect, a modified plant comprises at least 75% increased dry leaf yield mass as compared to a control tobacco plant when grown under comparable conditions. In an aspect, a modified plant comprises at least 75% increased cured leaf yield mass as compared to a control tobacco plant when grown under comparable conditions. In an aspect, a modified plant comprises at least 100% increased leaf yield mass as compared to a control tobacco plant when grown under comparable conditions. In an aspect, a modified plant comprises at least 100% increased fresh leaf yield mass as compared to a control tobacco plant when grown under comparable conditions. In an aspect, a modified plant comprises at least 100% increased dry leaf yield mass as compared to a control tobacco plant when grown under comparable conditions. In an aspect, a modified plant comprises at least 100% increased cured leaf yield mass as compared to a control tobacco plant when grown under comparable conditions.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation.

In an aspect, cured tobacco leaf provided herein is selected from the group consisting of air-cured tobacco leaf, fire-cured tobacco leaf, sun-cured tobacco leaf, and flue-cured tobacco leaf. In another aspect, cured tobacco material provided herein is selected from the group consisting of air-cured tobacco material, fire-cured tobacco material, sun-cured tobacco material, and flue-cured tobacco material. In an aspect, cured tobacco leaf is from a tobacco variety selected from the group consisting of a flue-cured variety, a bright variety, a Burley variety, a Virginia variety, a Maryland variety, a dark variety, an Oriental variety, and a Turkish variety. In another aspect, cured tobacco material is from a tobacco variety selected from the group consisting of a flue-cured variety, a bright variety, a Burley variety, a Virginia variety, a Maryland variety, a dark variety, an Oriental variety, and a Turkish variety.

Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In one aspect, the cured tobacco material of the present disclosure is flue-cured, sun-cured, air-cured, or fire-cured.

In one aspect, tobacco plants, seeds, plant parts, plant cells, and plant genomes provided herein are from a tobacco type selected from the group consisting of flue-cured tobacco, sun-cured tobacco, air-cured tobacco, dark air-cured tobacco, and dark fire-cured tobacco. In another aspect, tobacco plants, seeds, plant parts, plant cells, and plant genomes provided herein are from a tobacco type selected from the group consisting of Burley tobacco, Maryland tobacco, bright tobacco, Virginia tobacco, Oriental tobacco, Turkish tobacco, and Galpão tobacco.

In one aspect, a modified tobacco plant provided herein is of a tobacco variety selected from the group consisting of a flue-cured variety, a bright variety, a Burley variety, a Virginia variety, a Maryland variety, a Galpão variety, a dark variety, an Oriental variety, and a Turkish variety.

In one aspect, a modified tobacco plant provided herein is selected from the group consisting of a flue-cured variety tobacco plant, a bright variety tobacco plant, a Burley variety tobacco plant, a Virginia variety tobacco plant, a Maryland variety tobacco plant, a Galpão variety tobacco plant, a dark variety tobacco plant, an Oriental variety tobacco plant, and a Turkish variety tobacco plant. In one aspect, a modified tobacco cell provided herein is selected from the group consisting of a flue-cured variety tobacco cell, a bright variety tobacco cell, a Burley variety tobacco cell, a Virginia variety tobacco cell, a Maryland variety tobacco cell, a Galpão variety tobacco cell, a dark variety tobacco cell, an Oriental variety tobacco cell, and a Turkish variety tobacco cell. In one aspect, a modified tobacco plant part provided herein is selected from the group consisting of a flue-cured variety tobacco plant part, a bright variety tobacco plant part, a Burley variety tobacco plant part, a Virginia variety tobacco plant part, a Maryland variety tobacco plant part, a Galpão variety tobacco plant part, a dark variety tobacco plant part, an Oriental variety tobacco plant part, and a Turkish variety tobacco plant part. In one aspect, a modified tobacco seed provided herein is selected from the group consisting of a flue-cured variety tobacco seed, a bright variety tobacco seed, a Burley variety tobacco seed, a Virginia variety tobacco seed, a Maryland variety tobacco seed, a Galpão variety tobacco seed, a dark variety tobacco seed, an Oriental variety tobacco seed, and a Turkish variety tobacco seed.

As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an Ex F hybrid. The first and second hybrids can be further crossed to create the higher order hybrid (C×D)×(E×F) comprising genetic information from all four parent varieties. In an aspect, a modified tobacco plant provided herein is a hybrid tobacco plant. In another aspect, a modified tobacco seed provided herein is a hybrid tobacco seed.

As used herein, the term “crossing” refers to the deliberate mating of two plants. In an aspect, crossing comprises pollination and/or fertilization of a first tobacco plant by a second tobacco plant. The two tobacco plants being crossed can be distantly related, closely related, or identical. In an aspect, the two tobacco plants being crossed are both modified tobacco plants. In an aspect, the two tobacco plants being crossed are of the same tobacco variety. In an aspect, the two tobacco plants being crossed are of two different tobacco varieties. In an aspect, one of the two tobacco plants being crossed is male sterile. In an aspect, one of the two tobacco plants being crossed is female sterile. In an aspect, at least one of the two tobacco plants being crossed is a hybrid tobacco plant. In an aspect, at least one of the two tobacco plants being crossed is a modified tobacco plant.

In an aspect, this disclosure provides a method for producing a modified tobacco plant comprising: (a) crossing at least one tobacco plant of a first tobacco variety with at least one tobacco plant of a second tobacco variety, where the at least one tobacco plant of the first tobacco variety comprises a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the at least one tobacco plant comprises no suckers or reduced suckers after topping as compared to the control tobacco plant when grown under comparable growth conditions; and (b) selecting for progeny tobacco plants that exhibit no suckers or reduced suckers after topping compared to a control tobacco plant of the same cross grown under comparable growth conditions.

In another aspect, this disclosure provides a method for producing a modified tobacco plant comprising: (a) crossing at least one tobacco plant of a first tobacco variety with at least one tobacco plant of a second tobacco variety, where the at least one tobacco plant of the first tobacco variety comprises a recombinant DNA construct comprising a heterologous promoter operably linked to a nucleic acid encoding a polypeptide selected from the group consisting of a cyclin, a cyclin-dependent kinase (CDK), a CDK inhibitor, a MYB, and a WRKY transcription factor, where the at least one tobacco plant comprises no suckers or reduced suckers after topping as compared to a control tobacco plant when grown under comparable growth conditions; and (b) selecting for progeny tobacco plants that exhibit no suckers or reduced suckers after topping compared to a control tobacco plant of the same cross grown under comparable growth conditions.

In a further aspect, this disclosure provides a method for producing a modified tobacco plant comprising: (a) crossing at least one tobacco plant of a first tobacco variety with at least one tobacco plant of a second tobacco variety, where the at least one tobacco plant of said first tobacco variety comprises a recombinant DNA construct comprising a heterologous promoter operably linked to a nucleic acid encoding at least one small RNA molecule capable of reducing the expression of an endogenous genomic locus that encodes a polypeptide selected from the group consisting of a cyclin, a cyclin-dependent kinase (CDK), a CDK inhibitor, a MYB, and a WRKY transcription factor, where the at least one tobacco plant comprises no suckers or reduced suckers after topping as compared to a control tobacco plant when grown under comparable growth conditions; and (b) selecting for progeny tobacco plants that exhibit no suckers or reduced suckers after topping compared to a control tobacco plant of the same cross grown under comparable growth conditions.

In one aspect, a tobacco variety provided herein is male sterile. In another aspect, a tobacco variety provided herein is cytoplasmic male sterile (CMS). In an aspect, a modified tobacco plant provided herein is male sterile. In another aspect, a modified tobacco plant provided herein is cytoplasmic male sterile. Male sterile tobacco plants can be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y. 761 pp.

In another aspect, a tobacco variety provided herein is female sterile. As a non-limiting example, female sterile plants can be made by mutating the STIGI gene. See, for example, Goldman et al. 1994, EMBO Journal 13:2976-2984. In an aspect, a modified tobacco plant provided herein is female sterile.

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, modified tobacco plants or seeds provided herein are of a flue-cured tobacco variety selected from the group consisting of CC 13, CC 27, CC 33, CC35, CC 37, CC 65, CC 67, CC 700, GF 318, GL 338, GL 368, GL 939, K 346, K 399, K326, NC 102, NC 196, NC 291, NC 297, NC 299, NC 471, NC 55, NC 606, NC 71, NC 72, NC 92, PVH 1118, PVH 1452, PVH 2110, SPEIGHT 168, SPEIGHT 220, SPEIGHT 225, SPEIGHT 227, SPEIGHT 236, and any variety essentially derived from any one of the foregoing varieties. In another aspect, modified tobacco plants or seeds provided herein are in a flue-cured tobacco variety selected from the group consisting of Coker 48, Coker 176, Coker 371-Gold, Coker 319, Coker 347, GL 939, K 149, K326, K 340, K 346, K 358, K 394, K 399, K 730, NC 27NF, NC 37NF, NC 55, NC 60, NC 71, NC 72, NC 82, NC 95, NC 297, NC 606, NC 729, NC 2326, McNair 373, McNair 944, Ox 207, Ox 414 NF, Reams 126, Reams 713, Reams 744, RG 8, RG 11, RG 13, RG 17, RG 22, RG 81, RG H4, RG H51, Speight H-20, Speight G-28, Speight G-58, Speight G-70, Speight G-108, Speight G-111, Speight G-117, Speight 168, Speight 179, Speight NF-3, Va 116, Va 182, and any variety essentially derived from any one of the foregoing varieties. See WO 2004/041006 A1. In a further aspect, modified tobacco plants or seeds provided herein are in a flue-cured variety selected from the group consisting of K326, K346, and NC196.

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America. Maryland tobaccos are extremely fluffy, have good burning properties, low nicotine and a neutral aroma. Major Maryland growing countries include the United States of America and Italy.

In one aspect, modified tobacco plants or seeds provided herein are of a Burley tobacco variety selected from the group consisting of Clay 402, Clay 403, Clay 502, Ky 14, Ky 907, Ky 910, Ky 8959, NC 2, NC 3, NC 4, NC 5, NC 2000, TN 86, TN 90, TN 97, R 610, R 630, R 711, R 712, NCBH 129, HB4488PLC, PD 7319LC, Bu 21×Ky 10, HB04P, Ky 14×L 8, Kt 200, Newton 98, Pedigo 561, Pf561 and Va 509. In a further aspect, modified tobacco plants or seeds provided herein are in a Burley variety selected from the group consisting of TN 90, KT 209, KT 206, KT212, and HB 4488. In another aspect, modified tobacco plants or seeds provided herein are of a Maryland tobacco variety selected from the group consisting of Md 10, Md 40, Md 201, Md 609, Md 872 and Md 341.

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, modified tobacco plants or seeds provided herein are of a dark air-cured tobacco variety selected from the group consisting of Sumatra, Jatim, Dominican Cubano, Besuki, One sucker, Green River, Virginia sun-cured, and Paraguan Passado.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, modified tobacco plants or seeds provided herein are of a dark fire-cured tobacco variety selected from the group consisting of Narrow Leaf Madole, Improved Madole, Tom Rosson Madole, Newton's VH Madole, Little Crittenden, Green Wood, Little Wood, Small Stalk Black Mammoth, DT 508, DT 518, DT 592, KY 171, DF 911, DF 485, TN D94, TN D950, VA 309, and VA 359.

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, modified tobacco plants or seeds provided herein are of an Oriental tobacco variety selected from the group consisting of Izmir, Katerini, Samsun, Basma, Krumovgrad, Trabzon, Thesalian, Tasova, Sinop, Izmit, Hendek, Edirne, Semdinli, Adiyanman, Yayladag, Iskenderun, Duzce, Macedonian, Mavra, Prilep, Bafra, Bursa, Bucak, Bitlis, Balikesir, and any variety essentially derived from any one of the foregoing varieties.

The tobacco plants, plant parts, and tobacco material provided herein can be used in any tobacco product. As used herein, “tobacco product” is defined as any product made or derived from tobacco that is intended for human use or consumption. In an aspect, a tobacco product provided herein comprises cured components from a tobacco plant provided herein. In another aspect, a tobacco product provided herein comprises cured tobacco leaves from a tobacco plant provided herein. In an aspect, this disclosure provides a tobacco product comprising cured tobacco material from any tobacco plant provided herein.

In an aspect, this disclosure provides a tobacco product comprising cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety, and where the modified tobacco plant comprises no suckers or reduced suckers after topping as compared to the control tobacco plant when grown under comparable growth conditions. In another aspect, this disclosure provides a tobacco product comprising cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, where the mutation is not present in the endogenous gene in a control tobacco plant of the same variety.

In an aspect, this disclosure provides a method comprising preparing a tobacco product using cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety, and wherein said modified tobacco plant comprises no suckers or reduced suckers after topping as compared to a control tobacco plant when grown under comparable growth conditions. In another aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and wherein said modified tobacco plant comprises no suckers or reduced suckers after topping as compared to a control tobacco plant when grown under comparable growth conditions.

In an aspect, this disclosure provides a tobacco product comprising cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and where the modified tobacco plant comprises no suckers or reduced suckers after topping as compared to a control tobacco plant when grown under comparable growth conditions. In another aspect, this disclosure provides a tobacco product comprising cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.

In an aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, wherein said modified tobacco plant comprises no suckers or reduced suckers after topping as compared to a control tobacco plant when grown under comparable growth conditions. In another aspect, this disclosure provides a method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.

In an aspect, this disclosure provides a tobacco product comprising cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, where the modified tobacco plant comprises no suckers or reduced suckers after topping as compared to a control tobacco plant when grown under comparable growth conditions. In another aspect, this disclosure provides a tobacco product comprising cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

In an aspect, this disclosure provides a method comprising preparing a tobacco product using cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, wherein said modified tobacco plant comprises no suckers or reduced suckers after topping as compared to a control tobacco plant when grown under comparable growth conditions. In another aspect, this disclosure provides a method comprising preparing a tobacco product using cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.

Alkaloid compounds can be extracted, or isolated, from any tobacco plant or plant part provided herein. In an aspect, a tobacco product provided herein comprises an alkaloid extracted from a modified tobacco plant or part thereof. In another aspect, a tobacco product provided herein comprises nicotine extracted from a tobacco plant or tobacco plant part. In another aspect, a tobacco product provided herein comprises anatabine extracted from a tobacco plant or tobacco plant part. In another aspect, a tobacco product provided herein comprises anabasine extracted from a tobacco plant or tobacco plant part. In an aspect, a tobacco product provided herein comprises nornicotine extracted from a tobacco plant or plant part. In an aspect, a tobacco product provided herein comprises an alkaloid extracted from a modified tobacco plant, where the alkaloid is selected from the group consisting of nicotine, nornicotine, anatabine, and anabasine.

Alkaloid compounds extracted from tobacco plants or tobacco plant parts provided herein can be used to produce compositions suitable for use with non-combustible products. Exemplary non-combustible products include electronic cigarettes (“e-cigarettes”), electronic smoking articles, e-vapor products, aerosolized vapor products, and heated tobacco products. In an aspect, a non-combustible product provided herein comprises an alkaloid extracted from a tobacco plant or tobacco plant part provided herein. In an aspect, a non-combustible product provided herein comprises nicotine extracted from a tobacco plant or tobacco plant part provided herein. In an aspect, a non-combustible product provided herein comprises anabasine extracted from a tobacco plant or tobacco plant part provided herein. In an aspect, a non-combustible product provided herein comprises anatabine extracted from a tobacco plant or tobacco plant part provided herein. In an aspect, a non-combustible product provided herein comprises nornicotine extracted from a tobacco plant or tobacco plant part provided herein.

In one aspect, a non-combustible product provided herein is an e-cigarette. In another aspect, a non-combustible product provided herein is an electronic smoking article. In another aspect, a non-combustible product provided herein is an aerosolized vapor product. In another aspect, a non-combustible product provided herein is a heated tobacco product. In another aspect, a non-combustible product provided herein is an e-vapor product.

Tobacco products provided herein include, without limitation, cigarette products (e.g., cigarettes, bidi cigarettes, kreteks), cigar products (e.g., cigars, cigar wrapping tobacco, cigarillos), pipe tobacco products, products derived from tobacco, tobacco-derived nicotine products, smokeless tobacco products (e.g., moist snuff, dry snuff, chewing tobacco, moist smokeless tobacco, fine cut chewing tobacco, long cut chewing tobacco, pouched chewing tobacco, snus), films, chewables (e.g., gum), lozenges, dissolving strips, tabs, tablets, shaped parts, gels, consumable units, insoluble matrices, hollow shapes, reconstituted tobacco, expanded tobacco, and the like. See, for example, U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In one aspect, this disclosure provides nicotine derived from and a method of producing nicotine from a modified tobacco plant provided herein for use in a product.

In one aspect, a method provided herein comprises preparing a tobacco product using a cured tobacco leaf from a modified tobacco plant provided herein.

As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars. In an aspect, a tobacco product provided herein comprises reconstituted tobacco derived from a modified tobacco plant.

As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokeable material includes cured tobacco within a tobacco blend. In an aspect, a tobacco product of the present disclosure is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, and pouched chewing tobacco product. In another aspect, a tobacco product of the present disclosure is selected from the group consisting of a gum, a tablet, a lozenge, and a dissolving strip.

In an aspect, a tobacco product provided herein comprises cured tobacco material from a modified tobacco plant. In another aspect, a tobacco product provided herein comprises cured tobacco leaf material from a modified tobacco plant. In another aspect, a tobacco product provided herein comprises cured tobacco stem material from a modified tobacco plant.

In another aspect, a tobacco product of the present disclosure is a smokeless tobacco product. Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat treated smokeless tobacco. Dry snuff is finely ground tobacco that is placed in the mouth or used nasally. In a further aspect, a tobacco product of the present disclosure is selected from the group consisting of loose leaf chewing tobacco, plug chewing tobacco, moist snuff, and nasal snuff.

The present disclosure further provides a method manufacturing a tobacco product comprising tobacco material from modified tobacco plants provided herein. In one aspect, methods provided herein comprise conditioning aged tobacco material made from modified tobacco plants provided herein to increase its moisture content from between about 12.5% and about 13.5% to about 21%, blending the conditioned tobacco material to produce a desirable blend. In one aspect, the method of manufacturing a tobacco product provided herein further comprises casing or flavoring the blend. Generally, during the casing process, casing or sauce materials are added to blends to enhance their quality by balancing the chemical composition and to develop certain desired flavor characteristics. Further details for the casing process can be found in Tobacco Production, Chemistry and Technology, Edited by L. Davis and M. Nielsen, Blackwell Science, 1999.

This disclosure provides tobacco material from modified tobacco plants or parts thereof. Tobacco material obtained from tobacco plants, cells, lines, varieties, or hybrids of the present disclosure can be used to make tobacco products. In an aspect, tobacco material comprises leaf material. In an aspect, tobacco material comprises stem material. In an aspect, tobacco material comprises fresh tobacco material. In another aspect, tobacco material comprises dried tobacco material. In a further aspect, tobacco material comprises cured tobacco material. In still another aspect, tobacco material comprises fermented tobacco material. In an aspect, tobacco material provided herein can be used in any tobacco product provided herein. In an aspect, cured tobacco material provided herein comprises air-cured tobacco material. In another aspect, cured tobacco material provided herein comprises fire-cured tobacco material. In another aspect, cured tobacco material provided herein comprises sun-cured tobacco material. In another aspect, cured tobacco material provided herein comprises flue-cured tobacco material. In another aspect, cured tobacco material provided herein is selected from the group consisting of air-cured tobacco material, fire-cured tobacco material, sun-cured tobacco material, and flue-cured tobacco material.

Tobacco material provided herein can be also processed using methods including, but not limited to, heat treatment (e.g., cooking, toasting), flavoring, enzyme treatment, expansion and/or curing. Both fermented and non-fermented tobaccos can be processed using these techniques. Examples of suitable processed tobaccos include dark air-cured, dark fire cured, burley, flue cured, and cigar filler or wrapper, as well as the products from the whole leaf stemming operation. In one aspect, tobacco fibers include up to 70% dark tobacco on a fresh weight basis. For example, tobacco can be conditioned by heating, sweating and/or pasteurizing steps as described in U.S. Publication Nos. 2004/0118422 or 2005/0178398.

Tobacco material provided herein can be subject to fermentation. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. In addition to modifying the aroma of the leaf, fermentation can change either or both the color and texture of a leaf. Also during the fermentation process, evolution gases can be produced, oxygen can be taken up, the pH can change, and the amount of water retained can change. See, for example, U.S. Publication No. 2005/0178398 and Tso (1999, Chapter 1 in Tobacco, Production, Chemistry and Technology, Davis & Nielsen, eds., Blackwell Publishing, Oxford). Cured, or cured and fermented tobacco can be further processed (e.g., cut, expanded, blended, milled or comminuted) prior to incorporation into the oral product. The tobacco, in some cases, is long cut fermented cured moist tobacco having an oven volatiles content of between 48 and 50 weight percent prior to mixing with a copolymer and, optionally, flavorants and other additives.

In one aspect, tobacco material provided herein can be processed to a desired size. In certain aspects, tobacco fibers can be processed to have an average fiber size of less than 200 micrometers. In one aspect, tobacco fibers are between 75 and 125 micrometers. In another aspect, tobacco fibers are processed to have a size of 75 micrometers or less. In one aspect, tobacco fibers include long cut tobacco, which can be cut or shredded into widths of about 10 cuts/inch up to about 110 cuts/inch and lengths of about 0.1 inches up to about 1 inch. Double cut tobacco fibers can have a range of particle sizes such that about 70% of the double cut tobacco fibers falls between the mesh sizes of −20 mesh and 80 mesh.

Tobacco material provided herein can be processed to have a total oven volatiles content of about 10% by weight or greater; about 20% by weight or greater; about 40% by weight or greater; about 15% by weight to about 25% by weight; about 20% by weight to about 30% by weight; about 30% by weight to about 50% by weight; about 45% by weight to about 65% by weight; or about 50% by weight to about 60% by weight. Those of skill in the art will appreciate that “moist” tobacco typically refers to tobacco that has an oven volatiles content of between about 40% by weight and about 60% by weight (e.g., about 45% by weight to about 55% by weight, or about 50% by weight). As used herein, “oven volatiles” are determined by calculating the percentage of weight loss for a sample after drying the sample in a pre-warmed forced draft oven at 110 °C for 3.25 hours. An oral product can have a different overall oven volatiles content than the oven volatiles content of the tobacco fibers used to make the oral product. The processing steps described herein can reduce or increase the oven volatiles content.

The following exemplary, non-limiting embodiments are envisioned:
1. A modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety, and wherein said modified tobacco plant comprises no suckers or reduced suckers after topping as compared to said control tobacco plant when grown under comparable growth conditions.
2. A modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety.
3. A modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, wherein said modified tobacco plant comprises no suckers or reduced suckers after topping as compared to a control tobacco plant when grown under comparable growth conditions.
4. A modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.
5. A modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, wherein said modified tobacco plant comprises no suckers or reduced suckers after topping as compared to a control tobacco plant when grown under comparable growth conditions.
6. A modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.
7. The modified tobacco plant of embodiment 1 or 2, wherein said mutation comprises a mutation selected from the group consisting of an insertion, a deletion, a substitution, and an inversion.
8. The modified tobacco plant of any one of embodiments 1, 2, or 7, wherein said mutation comprises a null mutation.
9. The modified tobacco plant of any one of embodiments 1, 2, 7, or 8, wherein said mutation results in a premature stop codon in an mRNA transcript of said endogenous gene.
10. The modified tobacco plant of any one of embodiments 1, 2, or 7-9, wherein said mutation results in a truncation of said polypeptide.
11. The modified tobacco plant of any one of embodiments 1, 2, or 7-10, wherein said mutation is positioned within an exon of said endogenous gene.
12. The modified tobacco plant of any one of embodiments 1, 2, or 7-10, wherein said mutation is positioned within an intron of said endogenous gene.
13. The modified tobacco plant of any one of embodiments 1, 2, or 7, wherein said mutation is positioned within a 5′-untranslated region (UTR) or a 3′-UTR of said endogenous gene.
14. The modified tobacco plant of any one of embodiments 1, 2, or 7, wherein said mutation is positioned within a promoter of said endogenous gene.
15. The modified tobacco plant of any one of embodiments 1, 2, or 7-14, wherein said endogenous gene comprises a polynucleotide sequence at least 80% identical to a polynucleotide sequence selected from the group consisting of SEQ ID NOs: 1-44 and 113.
16. The modified tobacco plant of any one of embodiments 1, 2, or 7-15, wherein said endogenous gene encodes a polypeptide comprising an amino acid sequence at least 80% identical or similar to an amino acid sequence selected from the group consisting of SEQ ID NOs: 45-88 and 114.
17. The modified tobacco plant of any one of embodiments 1, 2, or 7-16, wherein said mutation results in reduced level of expression of said endogenous gene as compared to said control tobacco plant.
18. The modified tobacco plant of any one of embodiments 1, 2, or 7-16, wherein said mutation results in increased level of expression of said endogenous gene as compared to said control tobacco plant.
19. The modified tobacco plant of any one of embodiments 1, 2, or 7-16, wherein said mutation results in reduced level of activity of said polypeptide as compared to said control tobacco plant.
20. The modified tobacco plant of any one of embodiments 1, 2, or 7-16, wherein said mutation results in increased level of activity of said polypeptide as compared to said control tobacco plant.
21. The modified tobacco plant of any one of embodiments 3-6, wherein said heterologous promoter comprises an axillary meristem-specific promoter.
22. The modified tobacco plant of any one of embodiments 3-6, wherein said heterologous promoter comprises an axillary meristem-preferred promoter.
23. The modified tobacco plant of any one of embodiments 3-6, wherein said heterologous promoter comprises a polynucleotide sequence at least 90% identical to a polynucleotide sequence selected from the group consisting of SEQ ID NOs: 89-109.
24 The modified tobacco plant of any one of embodiments 3-6, wherein said small RNA molecule is selected from the group consisting of a double-stranded RNA, a small interfering RNA (siRNA), a trans-acting siRNA, and a microRNA.
25. The modified tobacco plant of embodiments 5 or 6, wherein said small RNA molecule comprises between 18 nucleotides and 30 nucleotides.
26. The modified tobacco plant of embodiments 5 or 6, wherein said small RNA molecule comprises a polynucleotide sequence having at least 90% identity or complementarity with said endogenous mRNA.
27. The modified tobacco plant of embodiments 5 or 6, wherein said small RNA molecule comprises between 18 nucleotides and 30 nucleotides.
28. The modified tobacco plant of embodiments 5 or 6, wherein said small RNA molecule comprises a polynucleotide sequence having at least 90% identity or complementarity with a polynucleotide sequence selected from the group consisting of SEQ ID NOs: 1-44 and 113.
29. The modified tobacco plant of embodiments 5 or 6, wherein said small RNA molecule comprises a polynucleotide sequence identical to, or complementary to, at least 18 contiguous nucleotides of a polynucleotide sequence selected from the group consisting of SEQ ID NOs: 1-44 and 113.
30 The modified tobacco plant of any one of embodiments 1-29, wherein said polypeptide is a cyclin.
31. The modified tobacco plant of any one of embodiments 1-29, wherein said polypeptide is a CDK.
32. The modified tobacco plant of any one of embodiments 1-29, wherein said polypeptide is a CDK inhibitor.
33. The modified tobacco plant of any one of embodiments 1-29, wherein said polypeptide is a MYB.
34 The modified tobacco plant of any one of embodiments 1-29, wherein said polypeptide is a WRKY transcription factor.
35. The modified tobacco plant of embodiment 30, wherein said cyclin is encoded by a polynucleotide sequence at least 80% identical to SEQ ID NOs: 6-32.
36 The modified tobacco plant of embodiment 30, wherein said cyclin comprises an amino acid sequence at least 80% identical or similar to an amino acid sequence selected from the group consisting of SEQ ID NOs: 50-76.
37. The modified tobacco plant of embodiment 31, wherein said CDK is encoded by a polynucleotide sequence at least 80% identical to SEQ ID NOs: 1-4.
38 The modified tobacco plant of embodiment 31, wherein said CDK comprises an amino acid sequence at least 80% identical or similar to an amino acid sequence at least 80% identical or similar to an amino acid sequence selected from the group consisting of SEQ ID NOs: 45-48.
39. The modified tobacco plant of embodiment 32, wherein said CDK inhibitor is encoded by a polynucleotide sequence at least 80% identical to SEQ ID NO: 5.
40. The modified tobacco plant of embodiment 32, wherein said CDK inhibitor comprises an amino acid sequence at least 80% identical or similar to an amino acid sequence selected from the group consisting of SEQ ID NO: 49.
41. The modified tobacco plant of embodiment 33, wherein said MYB is encoded by a polynucleotide sequence at least 80% identical to SEQ ID NOs: 33-44.
42. The modified tobacco plant of embodiment 33, wherein said MYB comprises an amino acid sequence at least 80% identical or similar to an amino acid sequence at least 80% identical or similar to an amino acid sequence selected from the group consisting of SEQ ID NOs: 77-88.
43. The modified tobacco plant of embodiment 34, wherein said WRKY transcription factor is encoded by a polynucleotide sequence at least 80% identical to SEQ ID NO: 113.
44. The modified tobacco plant of embodiment 34, wherein said WRKY transcription factor comprises an amino acid sequence at least 80% identical or similar to an amino acid sequence at least 80% identical or similar to SEQ ID NO: 114.
45. The modified tobacco plant of any one of embodiments 1-44, wherein said modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.
46. The modified tobacco plant of any one of embodiments 1-45, wherein said modified tobacco plant is selected from the group consisting of a BU 64 plant, a CC 101 plant, a CC 200 plant, a CC 13 plant, a CC 27 plant, a CC 33 plant, a CC 35 plant, a CC 37 plant, a CC 65 plant, a CC 67 plant, a CC 301 plant, a CC 400 plant, a CC 500 plant, CC 600 plant, a CC 700 plant, a CC 800 plant, a CC 900 plant, a CC 1063 plant, a Coker 176 plant, a Coker 319 plant, a Coker 371 Gold plant, a Coker 48 plant, a CU 263 plant, a DF911 plant, a Galpao plant, a GL 26H plant, a GL 338 plant, a GL 350 plant, a GL 395 plant, a GL 600 plant, a GL 737 plant, a GL 939 plant, a GL 973 plant, a GF 157 plant, a GF 318 plant, an RJR 901 plant, an HB 04P plant, a K 149 plant, a K 326 plant, a K 346 plant, a K 358 plant, a K394 plant, a K 399 plant, a K 730 plant, an NC 196 plant, an NC 37NF plant, an NC 471 plant, an NC 55 plant, an NC 92 plant, an NC2326 plant, an NC 95 plant, an NC 925 plant, a PVH 1118 plant, a PVH 1452 plant, a PVH 2110 plant, a PVH 2254 plant, a PVH 2275 plant, a VA 116 plant, a VA 119 plant, a KDH 959 plant, a KT 200 plant, a KT204LC plant, a KY 10 plant, a KY 14 plant, a KY 160 plant, a KY 17 plant, a KY 171 plant, a KY 907 plant, a KY 907LC plant, a KTY14×L8 LC plant, a Little Crittenden plant, a McNair 373 plant, a McNair 944 plant, a male sterile KY 14×L8 plant, a Narrow Leaf Madole plant, a MS KY171 plant, a Narrow Leaf Madole (phph) plant, a MS Narrow Leaf Madole plant, a MS TND950 plant, a PD 7302LC plant, a PD 7305LC plant, a PD 7309LC plant, a PD 7312LC plant, a PD 7318LC plant, a PD 7319LC plant, a MSTKS 2002 plant, a TKF 2002 plant, a TKF 6400 plant, a TKF 4028 plant, a TKF 4024 plant, a KT206LC plant, a KT209LC plant, a KT210LC plant, a KT212LC plant, an NC 100 plant, an NC 102 plant, an NC 2000 plant, an NC 291 plant, an NC 297 plant, an NC 299 plant, an NC 3 plant, an NC 4 plant, an NC 5 plant, an NC 6 plant, an NC7 plant, an NC 606 plant, an NC 71 plant, an NC 72 plant, an NC 810 plant, an NC BH 129 plant, an NC 2002 plant, a Neal Smith Madole plant, an OXFORD 207 plant, a ‘Perique’ plant, a PVH03 plant, a PVH09 plant, a PVH19 plant, a PVH50 plant, a PVH51 plant, an R 610 plant, an R 630 plant, an R 7-11 plant, an R 7-12 plant, an RG 17 plant, an RG 81 plant, an RG H51 plant, an RGH 4 plant, an RGH 51 plant, an RS 1410 plant, a Speight 168 plant, a Speight 172 plant, a Speight 179 plant, a Speight 210 plant, a Speight 220 plant, a Speight 225 plant, a Speight 227 plant, a Speight 234 plant, a Speight G-28 plant, a Speight G-70 plant, a Speight H-6 plant, a Speight H2O plant, a Speight NF3 plant, a TI 1406 plant, a TI 1269 plant, a TN 86 plant, a TN86LC plant, a TN 90 plant, a TN90LC plant, a TN 97 plant, a TN97LC plant, a TN D94 plant, a TN D950 plant, a TR (Tom Rosson) Madole plant, a VA 309 plant, and a VA 359 plant.
47. The modified tobacco plant of any one of embodiments 1-46, wherein said modified tobacco plant is a hybrid.
48. The modified tobacco plant of any one of embodiments 1-47, wherein said modified tobacco plant is male sterile or cytoplasmically male sterile.
49. The modified tobacco plant of any one of embodiments 1-47, wherein said modified tobacco plant is female sterile.
50. The modified tobacco plant of any one of embodiments 1, 3 or, wherein said reduced suckers comprises fewer total suckers, smaller average sucker size, or both, as compared to suckers of said control tobacco plant when grown under comparable growth conditions.
51. The modified tobacco plant of embodiment 50, wherein said smaller average sucker size comprises a measurement selected from the group consisting of reduced average mass, reduced average length, reduced average diameter, or any combination thereof, as compared to suckers of said control tobacco plant when grown under comparable growth conditions.
52. The modified tobacco plant of any one of embodiments 1-51, wherein said modified plant has increased leaf yield mass as compared to said control tobacco plant when grown under comparable growth conditions.
53. The modified tobacco plant of embodiment 52, wherein said increased leaf yield mass comprises an increase of at least 0.5%.
54. A tobacco leaf of the modified tobacco plant of any one of embodiments 1-53.
55. A tobacco seed of the modified tobacco plant of any one of c embodiments 1-53.
56. The tobacco leaf of embodiment 54, wherein said tobacco leaf is a cured tobacco leaf.
57. The tobacco leaf of embodiment 56, wherein said cured tobacco leaf is selected from the group consisting of is air-cured tobacco leaf, fire-cured tobacco leaf, sun-cured tobacco leaf, and flue-cured tobacco leaf.
58. A tobacco product comprising an alkaloid extracted from the modified tobacco plant, or a part thereof, of any one of embodiments 1-53.
59. The tobacco product of embodiment 58, wherein said alkaloid is selected from the group consisting of nicotine, nornicotine, anatabine, and anabasine.
60 The tobacco product of embodiment 58 or 59, wherein said tobacco product is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, and pouched chewing tobacco product.
61. The tobacco product of embodiment 58 or 59, wherein said tobacco product is selected from the group consisting of a gum, a tablet, a lozenge, and a dissolving strip.
62. The tobacco product of embodiment 58 or 59, wherein said tobacco product is a non-combustible product.
63. The tobacco product of embodiment 62, wherein said non-combustible product is selected from the group consisting of an electronic cigarette, an electronic smoking article, an aerosolized vapor product, and a heated tobacco product.
64. A tobacco product comprising cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety, and wherein said modified tobacco plant comprises no suckers or reduced suckers after topping as compared to said control tobacco plant when grown under comparable growth conditions.
65. A tobacco product comprising cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety.
66. A tobacco product comprising cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and wherein said modified tobacco plant comprises no suckers or reduced suckers after topping as compared to a control tobacco plant when grown under comparable growth conditions.
67. A tobacco product comprising cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.
68. A tobacco product comprising cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, wherein said modified tobacco plant comprises no suckers or reduced suckers after topping as compared to a control tobacco plant when grown under comparable growth conditions.
69. A tobacco product comprising cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.
70. The tobacco product of any one of embodiments 64-69, wherein said tobacco product is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, and a pouched chewing tobacco product.
71. The tobacco product of any one of embodiments 64-69, wherein said cured tobacco material is from a tobacco variety selected from the group consisting of a flue-cured variety, a bright variety, a Burley variety, a Virginia variety, a Maryland variety, a dark variety, an Oriental variety, and a Turkish variety.
72. The tobacco product of any one of embodiments 64-69, wherein said cured tobacco material is selected from the group consisting of air-cured tobacco material, fire-cured tobacco material, sun-cured tobacco material, and flue-cured tobacco material.
73. The tobacco product of any one of embodiments 64-69, wherein said cured tobacco material comprises cured tobacco leaf material.
74. The tobacco product of any one of embodiments 64-69, wherein said cured tobacco material comprises cured tobacco stem material.
75. A method for producing a modified tobacco plant comprising:
   (a) inducing a mutation in at least one tobacco cell at a genomic locus encoding a polypeptide selected from the group consisting of a cyclin, a cyclin-dependent kinase (CDK), a CDK inhibitor, a MYB, and a WRKY transcription factor;
   (b) selecting at least one tobacco cell comprising said mutation from step (a); and
   (c) regenerating a modified tobacco plant from said at least one tobacco cell selected in step (b), wherein said modified tobacco plant comprises no suckers or reduced suckers after topping as compared to a control tobacco plant lacking said mutation when grown under comparable growth conditions.
76. A method for producing a modified tobacco plant comprising:
   (a) introducing a recombinant DNA construct to at least one tobacco cell, wherein said recombinant DNA construct comprises a heterologous promoter operably linked to a nucleic acid encoding a polypeptide selected from the group consisting of a cyclin, a cyclin-dependent kinase (CDK), a CDK inhibitor, a MYB, and a WRKY transcription factor;
   (b) selecting at least one tobacco cell comprising said recombinant DNA construct; and
   (c) regenerating a modified tobacco plant from said at least one tobacco cell selected in step (b), wherein said modified tobacco plant comprises no suckers or reduced suckers after topping as compared to a control tobacco plant lacking said recombinant DNA construct when grown under comparable growth conditions.
77. A method for producing a modified tobacco plant comprising:
   (a) introducing a recombinant DNA construct to at least one tobacco cell, wherein said recombinant DNA construct comprises a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor;
   (b) selecting at least one tobacco cell comprising said recombinant DNA construct; and
   (c) regenerating a modified tobacco plant from said at least one tobacco cell selected in step (b), wherein said modified tobacco plant comprises no suckers or reduced suckers after topping as compared to a control tobacco plant lacking said recombinant DNA construct when grown under comparable growth conditions.
78. The method of embodiment 75, wherein said inducing comprises the use of an agent selected from the group consisting of: a chemical mutagen, irradiation, a transposon, Agrobacterium, and a nuclease.
79. The method of embodiment 78, wherein said nuclease is selected from the group consisting of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, a Csm1 nuclease, or any combination thereof.
80. The method of embodiment 78, wherein said chemical mutagen comprises ethyl methanesulfonate.
81 The method of embodiment 78, wherein said irradiation comprises gamma rays, X-rays, or ionizing radiation.
82 The method of embodiment 77, wherein said small RNA molecule is selected from the group consisting of a double-stranded RNA, a small interfering RNA (siRNA), a trans-acting siRNA, and a microRNA.
83. The method of embodiment 77 or 82, wherein said small RNA molecule comprises at least 18 nucleotides.
84. The method of any one of embodiments 77, 82, or 83, wherein said small RNA molecule comprises at least 90% identity or complementarity with said endogenous mRNA.
85. The method of any one of embodiments 77, 82, or 84, wherein said small RNA molecule comprises between 18 nucleotides and 30 nucleotides.
86. The method of any one of embodiments 77 or 82-85, wherein said small RNA molecule comprises at least 90% identity or complementarity with a polynucleotide sequence selected from the group consisting of SEQ ID NOs: 1-44 and 113.
87. The method of any one of embodiments 77 or 82-86, wherein said endogenous mRNA comprises a sequence identical to, or complementary to, at least 18 contiguous nucleotides of a polynucleotide sequence selected from the group consisting of SEQ ID NOs: 1-44 and 113.
88. The method of any one of embodiments 75-87, wherein said at least one tobacco cell is a tobacco protoplast cell.
89. The method of any one of embodiments 75-87, wherein said at least one tobacco cell is a tobacco callus cell.
90. The method of any one of embodiments 75-87, wherein said at least one tobacco cell is selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.
91. The method of any one of embodiments 75-77, wherein said method further comprises:
   (d) growing said modified tobacco plant regenerated in step (c).
92. The method of embodiment 91, wherein said method further comprises:
   (e) crossing said modified tobacco plant grown in step (d) with a second tobacco plant; and
   (f) obtaining at least one seed from said crossing in step (e).
93. The method of embodiments 76 or 77, wherein said heterologous promoter comprises an axillary meristem-specific promoter.
94. The modified tobacco plant of embodiment 93, wherein said heterologous promoter comprises a polynucleotide sequence at least 90% identical to a polynucleotide sequence selected from the group consisting of SEQ ID NOs: 89-109.
95. The method of embodiment 75, wherein said genomic locus comprises a polynucleotide sequence at least 80% identical to a polynucleotide sequence selected from the group consisting of SEQ ID NOs: 1-44 and 113.
96. The method of embodiment 75, wherein said genomic locus encodes a polypeptide comprising an amino acid sequence at least 80% identical or similar to an amino acid sequence selected from the group consisting of SEQ ID NOs: 45-88 and 114.
97. The method of embodiment 75, wherein said mutation results in reduced expression of said genomic locus as compared to said control tobacco plant.
98 The method of embodiment 75, wherein said mutation results in increased expression of said genomic locus as compared to said control tobacco plant.
99 The method of embodiment 75, wherein said mutation results in reduced activity of said polypeptide as compared to said control tobacco plant.
100. The method of embodiment 75, wherein said mutation results in increased activity of said polypeptide as compared to said control tobacco plant.
101. The method of any one of embodiments 75-100, wherein said polypeptide is a cyclin.
102. The method of any one of embodiments 75-100, wherein said polypeptide is a CDK.
103. The method of any one of embodiments 75-100, wherein said polypeptide is a CDK inhibitor.
104. The method of any one of embodiments 75-100, wherein said polypeptide is a MYB.
105. The method of any one of embodiments 75-100, wherein said polypeptide is a WRKY transcription factor.
106. The method of embodiment 101, wherein said cyclin is encoded by a polynucleotide sequence at least 80% identical to SEQ ID NOs: 6-32.
107. The method of embodiment 101, wherein said cyclin comprises an amino acid sequence at least 80% identical or similar to an amino acid sequence selected from the group consisting of SEQ ID NOs: 50-76.
108. The method of embodiment 102, wherein said CDK is encoded by a polynucleotide sequence at least 80% identical to SEQ ID NOs: 1-4.
109. The method of embodiment 102, wherein said CDK comprises an amino acid sequence at least 80% identical or similar to an amino acid sequence selected from the group consisting of SEQ ID NOs: 45-48.
110. The method of embodiment 103, wherein said CDK inhibitor is encoded by a polynucleotide sequence at least 80% identical to SEQ ID NO: 5.
111. The method of embodiment 103, wherein said CDK inhibitor comprises an amino acid sequence at least 80% identical or similar to an amino acid sequence selected from the group consisting of SEQ ID NO: 49.
112. The method of embodiment 104, wherein said MYB is encoded by a polynucleotide sequence at least 80% identical to SEQ ID NOs: 33-44.
113. The method of embodiment 104, wherein said MYB comprises an amino acid sequence at least 80% identical or similar to an amino acid sequence selected from the group consisting of SEQ ID NOs: 77-88.
114. The method of embodiment 105, wherein said WRKY transcription factor is encoded by a polynucleotide sequence at least 80% identical to SEQ ID NO: 113.
115. The method of embodiment 105, wherein said WRKY transcription factor comprises an amino acid sequence at least 80% identical or similar to SEQ ID NO: 114.
116. The method of any one of embodiments 75-114, wherein said modified tobacco plant is of a tobacco variety selected from the group consisting of a flue-cured variety, a bright variety, a Burley variety, a Virginia variety, a Maryland variety, a dark variety, an Oriental variety, and a Turkish variety.
117. The method of any one of embodiments 75-114, wherein said modified tobacco plant is selected from the group consisting of a BU 64 plant, a CC 101 plant, a CC 200 plant, a CC 13 plant, a CC 27 plant, a CC 33 plant, a CC 35 plant, a CC 37 plant, a CC 65 plant, a CC 67 plant, a CC 301 plant, a CC 400 plant, a CC 500 plant, CC 600 plant, a CC 700 plant, a CC 800 plant, a CC 900 plant, a CC 1063 plant, a Coker 176 plant, a Coker 319 plant, a Coker 371 Gold plant, a Coker 48 plant, a CU 263 plant, a DF911 plant, a Galpao plant, a GL 26H plant, a GL 338 plant, a GL 350 plant, a GL 395 plant, a GL 600 plant, a GL 737 plant, a GL 939 plant, a GL 973 plant, a GF 157 plant, a GF 318 plant, an RJR 901 plant, an HB 04P plant, a K 149 plant, a K 326 plant, a K 346 plant, a K 358 plant, a K394 plant, a K 399 plant, a K 730 plant, an NC 196 plant, an NC 37NF plant, an NC 471 plant, an NC 55 plant, an NC 92 plant, an NC2326 plant, an NC 95 plant, an NC 925 plant, a PVH 1118 plant, a PVH 1452 plant, a PVH 2110 plant, a PVH 2254 plant, a PVH 2275 plant, a VA 116 plant, a VA 119 plant, a KDH 959 plant, a KT 200 plant, a KT204LC plant, a KY 10 plant, a KY 14 plant, a KY 160 plant, a KY 17 plant, a KY 171 plant, a KY 907 plant, a KY 907LC plant, a KTY14×L8 LC plant, a Little Crittenden plant, a McNair 373 plant, a McNair 944 plant, a male sterile KY 14×L8 plant, a Narrow Leaf Madole plant, a MS KY171 plant, a Narrow Leaf Madole (phph) plant, a MS Narrow Leaf Madole plant, a MS TND950 plant, a PD 7302LC plant, a PD 7305LC plant, a PD 7309LC plant, a PD 7312LC plant, a PD 7318LC plant, a PD 7319LC plant, a MSTKS 2002 plant, a TKF 2002 plant, a TKF 6400 plant, a TKF 4028 plant, a TKF 4024 plant, a KT206LC plant, a KT209LC plant, a KT210LC plant, a KT212LC plant, an NC 100 plant, an NC 102 plant, an NC 2000 plant, an NC 291 plant, an NC 297 plant, an NC 299 plant, an NC 3 plant, an NC 4 plant, an NC 5 plant, an NC 6 plant, an NC7 plant, an NC 606 plant, an NC 71 plant, an NC 72 plant, an NC 810 plant, an NC BH 129 plant, an NC 2002 plant, a Neal Smith Madole plant, an OXFORD 207 plant, a ‘Perique’ plant, a PVH03 plant, a PVH09 plant, a PVH19 plant, a PVH50 plant, a PVH51 plant, an R 610 plant, an R 630 plant, an R 7-11 plant, an R 7-12 plant, an RG 17 plant, an RG 81 plant, an RG H51 plant, an RGH 4 plant, an RGH 51 plant, an RS 1410 plant, a Speight 168 plant, a Speight 172 plant, a Speight 179 plant, a Speight 210 plant, a Speight 220 plant, a Speight 225 plant, a Speight 227 plant, a Speight 234 plant, a Speight G-28 plant, a Speight G-70 plant, a Speight H-6 plant, a Speight H2O plant, a Speight NF3 plant, a TI 1406 plant, a TI 1269 plant, a TN 86 plant, a TN86LC plant, a TN 90 plant, a TN90LC plant, a TN 97 plant, a TN97LC plant, a TN D94 plant, a TN D950 plant, a TR (Tom Rosson) Madole plant, a VA 309 plant, and a VA 359 plant.
118. The method of any one of embodiments 75-117, wherein said modified tobacco plant is a hybrid.
119. The method of any one of embodiments 75-118, wherein said modified tobacco plant is male sterile or cytoplasmically male sterile.
120. The method of any one of embodiments 75-118, wherein said modified tobacco plant is female sterile.
121. The method of any one of embodiments 75-120, wherein said reduced suckers comprises fewer total suckers, smaller suckers, or both, as compared to suckers of said control tobacco plant when grown under comparable growth conditions.
122. The method of embodiment 121, wherein said smaller suckers comprise reduced average mass, reduced average length, reduced average diameter, or any combination thereof, as compared to suckers of said control tobacco plant when grown under comparable growth conditions.
123. The method of any one of embodiments 75-122, wherein said modified plant has increased leaf yield mass as compared to said control tobacco plant when grown under comparable growth conditions.
124. The method of embodiment 123, wherein said increased leaf yield mass comprises an increase of at least 0.5%.
125. A method comprising preparing a tobacco product using cured tobacco material from a modified tobacco plant comprising a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety, and wherein said modified tobacco plant comprises no suckers or reduced suckers after topping as compared to a control tobacco plant when grown under comparable growth conditions.
126. A method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, and wherein said modified tobacco plant comprises no suckers or reduced suckers after topping as compared to a control tobacco plant when grown under comparable growth conditions.
127. A method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter, wherein said modified tobacco plant comprises no suckers or reduced suckers after topping as compared to a control tobacco plant when grown under comparable growth conditions.
128. A method comprising preparing a tobacco product using cured tobacco material from a modified tobacco plant comprising a polynucleotide sequence encoding a polypeptide selected from the group consisting of a cyclin, a cyclin-dependent kinase (CDK), a CDK inhibitor, a MYB, and a WRKY transcription factor operably linked to a heterologous promoter.
129. A method comprising preparing a tobacco product using cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor, wherein said modified tobacco plant comprises no suckers or reduced suckers after topping as compared to a control tobacco plant when grown under comparable growth conditions.
130. A method comprising preparing a tobacco product using cured tobacco material from a modified tobacco plant comprising a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.
131. The method of any one of embodiments 125-130, wherein said tobacco product is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.
132. The method of any one of embodiments 125-131, wherein said cured tobacco material is from a tobacco variety selected from the group consisting of a flue-cured variety, a bright variety, a Burley variety, a Virginia variety, a Maryland variety, a dark variety, an Oriental variety, and a Turkish variety.
133. The method of any one of embodiments 125-132, wherein said cured tobacco material is selected from the group consisting of air-cured tobacco material, fire-cured tobacco material, sun-cured tobacco material, and flue-cured tobacco material.
134. The method of any one of embodiments 125-133, wherein said cured tobacco material comprises cured tobacco leaf material.
135. The method of any one of embodiments 125-133, wherein said cured tobacco material comprises cured tobacco stem material.
136. A method comprising transforming a tobacco cell with a recombinant DNA construct comprising a heterologous promoter operably linked to a nucleic acid encoding a polypeptide selected from the group consisting of a cyclin, a cyclin-dependent kinase (CDK), a CDK inhibitor, a MYB, and a WRKY transcription factor.
137. A method comprising transforming a tobacco cell with a recombinant DNA construct comprising a heterologous promoter operably linked to a nucleic acid encoding at least one small RNA molecule capable of reducing the expression of an endogenous mRNA that encodes a polypeptide selected from the group consisting of a cyclin, a cyclin-dependent kinase (CDK), a CDK inhibitor, a MYB, and a WRKY transcription factor.
138. The method of embodiments 136 or 137, wherein said method further comprises regenerating a modified tobacco plant from said tobacco cell, wherein said modified tobacco plant comprises said recombinant DNA construct.
139. The method of embodiment 138, wherein said modified tobacco plant comprises no or reduced suckers after topping as compared to a control tobacco plant lacking said recombinant DNA construct when grown under comparable conditions.
140. A method for producing a modified tobacco plant comprising:
   (a) crossing at least one tobacco plant of a first tobacco variety with at least one tobacco plant of a second tobacco variety, wherein said at least one tobacco plant of said first tobacco variety comprises a mutation in an endogenous gene encoding a polypeptide selected from the group consisting of a cyclin, a cyclin-dependent kinase (CDK), a CDK inhibitor, a MYB, and a WRKY transcription factor, wherein said mutation is not present in said endogenous gene in a control tobacco plant of the same variety, and wherein said at least one tobacco plant comprises no suckers or reduced suckers after topping as compared to said control tobacco plant when grown under comparable growth conditions; and
   (b) selecting for progeny tobacco plants that exhibit no suckers or reduced suckers after topping compared to a control tobacco plant of the same cross grown under comparable growth conditions.
141. A method for producing a modified tobacco plant comprising:
   (a) crossing at least one tobacco plant of a first tobacco variety with at least one tobacco plant of a second tobacco variety, wherein said at least one tobacco plant of said first tobacco variety comprises a recombinant DNA construct comprising a heterologous promoter operably linked to a nucleic acid encoding a polypeptide selected from the group consisting of a cyclin, a cyclin-dependent kinase (CDK), a CDK inhibitor, a MYB, and a WRKY transcription factor, wherein said at least one tobacco plant comprises no suckers or reduced suckers after topping as compared to a control tobacco plant when grown under comparable growth conditions; and
   (b) selecting for progeny tobacco plants that exhibit no suckers or reduced suckers after topping compared to a control tobacco plant of the same cross grown under comparable growth conditions.
142. A method for producing a modified tobacco plant comprising:
   (a) crossing at least one tobacco plant of a first tobacco variety with at least one tobacco plant of a second tobacco variety, wherein said at least one tobacco plant of said first tobacco variety comprises a recombinant DNA construct comprising a heterologous promoter operably linked to a nucleic acid encoding at least one small RNA molecule capable of reducing the expression of an endogenous genomic locus that encodes a polypeptide selected from the group consisting of a cyclin, a cyclin-dependent kinase (CDK), a CDK inhibitor, a MYB, and a WRKY transcription factor, wherein said at least one tobacco plant comprises no suckers or reduced suckers after topping as compared to a control tobacco plant when grown under comparable growth conditions; and
   (b) selecting for progeny tobacco plants that exhibit no suckers or reduced suckers after topping compared to a control tobacco plant of the same cross grown under comparable growth conditions.

Having now generally described the disclosure, the same will be more readily understood through reference to the following examples that are provided by way of illustration, and are not intended to be limiting of the present disclosure, unless specified.

### EXAMPLES

### Example 1. Identification of Differentially Expressed Genes

Two hundred ten TN90 tobacco plants are grown to layby stage (eight to ten fully expanded leaves). At this stage, axillary buds are collected from 30 plants to form three replicates, each containing the pooled axillary buds from ten plants. The remaining 180 plants are topped. Ninety of the plants are sprayed with water, and ninety of the plants are sprayed with maleic hydrazide (MH) per the manufacturer's dosing instructions (Arysta LifeScience Royal MH-30® XTRA). Axillary buds from water-treated (control) and MH-treated plants are collected four hours after spraying, 24 hours after spraying, and 72 hours after spraying. See Table 2. RNA is extracted from each collection and sequenced using Hiseq (Illumina).

TABLE 2
Experimental groups

| | | Number of |
|---|---|---|
| Treatment | Description | replicates |
| Control | Un-topped plants; no water or | 3 |
| | maleic hydrazide (MH) treatment | |
| 4 hour-water | Plants topped and treated with water; | 3 |
| | axillary bud samples were collected | |
| | 4 hours after treatment. | |
| 4 hour-MH | Plants topped and treated with MH; | 3 |
| | axillary bud samples were collected | |
| | 4 hours after treatment. | |
| 24 hour-water | Plants topped and treated with water; | 3 |
| | axillary bud samples were collected | |
| | 24 hours after treatment. | |
| 24 hour-MH | Plants topped and treated with MH; | 3 |
| | axillary bud samples were collected | |
| | 24 hours after treatment. | |
| 72 hour-water | Plants topped and treated with water; | 3 |
| | axillary bud samples were collected | |
| | 72 hours after treatment. | |
| 72 hour-MH | Plants topped and treated with MH; | 3 |
| | axillary bud samples were collected | |
| | 72 hours after treatment. | |
| | Total samples | 21 |

The RNA sequences are mapped to a proprietary tobacco reference genome using CLC Genomics Workbench (version 11.0.1; QIAGEN) using default parameters. A read count matrix including all samples was generated by aggregating the raw counts of the mapped reads for a given gene in each sample against a total of 98,751 genes using the Expression Browser Tool in CLC Genomics Workbench. The read count matrix was subjected to differential gene expression analysis using the R package edgeR (version 3.0.8). See Robinson et al. “edgeR: a Bioconductor package for differential expression analysis of digital gene expression data,” Bioinformatics, 26:139-140 (2010).

Genes with no expression or ubiquitously low expression were filtered out of the read count matrix in order to improve sensitivity of differential gene expression detection. Only genes having read counts per million reads of >1 in at least three RNA sequencing libraries were retained. These filters result in a filtered read count matrix containing 39,980 expressed genes.

The filtered read count matrix was normalized for compositional bias between libraries using the trimmed means of M (TMM) values method. See, for example, Robinson and Oshlack, “A scaling normalization method for differential expression analysis of RNA-seq data,” Genome Biology, 11: R25 (2010). Differentially expressed genes were detected between three pairwise comparisons: control samples at 4 hours, 24 hours, and 72 hours post-topping vs. MH-treated samples at 4 hours, 24 hours, and 72 hours post-topping, respectively. Genes having a p-value of ≤0.05 and an absolute value of logz fold changes of ≥1 or ≤1 are considered to be differentially expressed. In total, over 6300 genes are differentially expressed between the control and MH-treated samples. Gene expression values (reads per kilobase of transcript per million mapped reads (RPKM)) from the differentially expressed genes are used to perform downstream analysis and identify candidate genes.

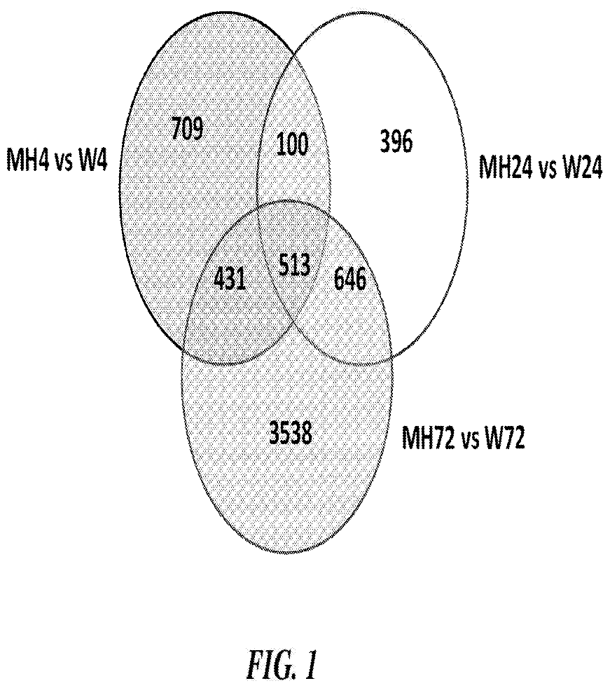

Seven hundred nine differentially expressed genes are identified only in the 4-hour post-topping pairwise comparison. Three hundred ninety-six differentially expressed genes are identified only in the 24-hour post-topping pairwise comparison. Three thousand five hundred thirty-eight differentially expressed genes are identified only in the 72-hour post-topping pairwise comparison. One hundred differentially expressed genes are identified in both the 4-hour and 24-hour post-topping pairwise comparisons. Four hundred thirty-one differentially expressed genes are identified in both the 4-hour and 72-hour pairwise comparisons. Six hundred forty-six differentially expressed genes are identified in both the 24-hour and 72-hour pairwise comparisons. Five hundred thirteen differentially expressed genes are identified in all three pairwise comparisons. FIG. 1 provides a Venn diagram depicting the overlap of differentially expressed genes between the different comparisons.

Differentially expressed genes that are predicted to play a role in cell proliferation (e.g., cyclins, cyclin-dependent kinases, MYBs, WRKY transcription factors) are identified as candidate genes.

### Example 2. Transformation and Regeneration of Modified Tobacco Plants

An expression vector is used as a backbone to generate multiple transformation vectors comprising recombinant DNA constructs (See Examples 5-6). The expression vector contains an axillary meristem-preferred promoter (e.g., SEQ ID NOs: 89-109), a NOS terminator, and a cassette comprising a kanamycin selection marker (NPT II) operably linked to an Actin2 promoter and a NOS terminator. Nucleic acid vectors comprising transgenes of interest are introduced into tobacco leaf discs via Agrobacterium transformation. See, for example, Mayo et al., 2006, Nat Protoc. 1:1105-11 and Horsch et al., 1985, Science 227:1229-1231.

Narrow Leaf Madole (NLM) tobacco plants are grown in Magenta™ GA-7 boxes (77 mm×77 mm×97 mm clear polycarbonate boxes with polypropylene lids) and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed and the Agrobacterium tumefaciens cell pellet is re-suspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog with B5 vitamins liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (1/2 MS medium) and discs are placed upside down in contact with filter paper overlaid on the co-cultivation TOM medium (MS medium with 20 g/L sucrose; 1 mg/L indole-3-acetic acid; and 2.5 mg/L 6-benzyl aminopurine (BAP)). The Petri plate is sealed with parafilm prior to incubation in dim light (60-80 mE/ms) with 18 hours on, 6 hours off photoperiods at 24 degrees Celsius for three days. After incubation, leaf discs are transferred to regeneration/selection TOM K medium Petri plates (TOM medium plus 300 mg/L kanamycin). Leaf discs are sub-cultured bi-weekly to fresh TOM K medium in dim light with 18 hours on, 6 hours off photoperiods at 24 degrees Celsius until shoots become excisable. Shoots from leaves are removed with forceps and inserted in MS basal medium with 100 mg/L kanamycin. Shoots on MS basal medium with 100 mg/L kanamycin are incubated at 24 degrees Celsius with 18 hours on, 6 hours off photoperiods with high intensity lighting (6080 mE/ms) to induce rooting.

### Example 3. Phenotypic Screening

When plantlets regenerated in Example 2 containing both shoots and roots grow large enough (e.g., reach approximately half the height of a Magenta™ GA-7 box), they are transferred to soil. Established seedlings are transferred to a greenhouse for further analysis and to set seed. Evaluation of suckering phenotypes is conducted by growing modified plants (T0, T1, T2, or later generations) and control plants to layby stage. Control plants are either NLM plants that have not been transformed or NLM plants that have been transformed with an empty p45-2-7 vector. Plants that have reached layby stage are manually topped (the shoot apical meristem and surrounding tissue is removed), and axillary bud growth is evaluated at specific time points after topping. Observations are typically performed at the time of topping (i.e., 0 hours), 24 hours (i.e., 1 day) after topping, 7-8 days after topping (i.e., one week), and/or 14-15 days (i.e., two weeks) after topping. Observations comprise qualitatively examining the presence or absence of axillary bud growth and overall plant appearance. Observations also comprise quantitatively measuring the fresh weight of all axillary buds at a specific time point after topping and/or measuring the length of all axillary bud outgrowths at a specific time point after topping.

### Example 4. Development of Modified Tobacco Plants Via Induced Mutation

Mutations are produced in CDK, CDK inhibitor, cyclin, MYB, and WRKY genes by specifically editing SEQ ID NOs: 1-44 and 113. Tobacco protoplasts are transfected using polyethylene glycol (PEG) with plasmids encoding a CRISPR protein or a CRISPR protein and specific guide RNA (gRNA) targeting individual genes at desired positions.

Transfected protoplasts are then immobilized in 1% agarose beads and subjected to tissue culture. When calli grow to approximately 1 millimeter in diameter, they are spread on TOM2 plates. Calli are screened for mutations (e.g., insertions or deletions (indels)) at the target positions using fragment analysis. Candidates, showing size shifts compared to wildtype control, are selected for further culture and the consequent shoots are tested by fragment analysis again to confirm the presence of mutations.

Modified tobacco plants (TO generation) comprising the targeted mutations and control tobacco plants lacking the mutations are grown to the layby stage as described in Example 2. Then, plants are topped to remove the shoot apical meristem and modified and control tobacco plants are phenotypically evaluated as described in Example 3.

### Example 5. Development of Modified Tobacco Plants Via Targeted Expression

Targeted over-expression of genes comprising SEQ ID NOs: 1-44 and 113 in axillary bud tissues can be used to reduce or eliminate sucker outgrowth in tobacco when operably linked to an axillary bud-preferred promoter (e.g. SEQ ID NOs: 83-109).

Six separate transformation vectors comprising SEQ ID NOs: 5, 33, or 36 driven by a promoter comprising SEQ ID NOs: 89 or 93 are constructed as described above in Example 2.

Modified tobacco plants (TO generation) comprising one the transformation vectors and control tobacco plants are grown to the layby stage as described in Example 2. Then, plants are topped to remove the shoot apical meristem and modified and control tobacco plants are phenotypically evaluated as described in Example 3.

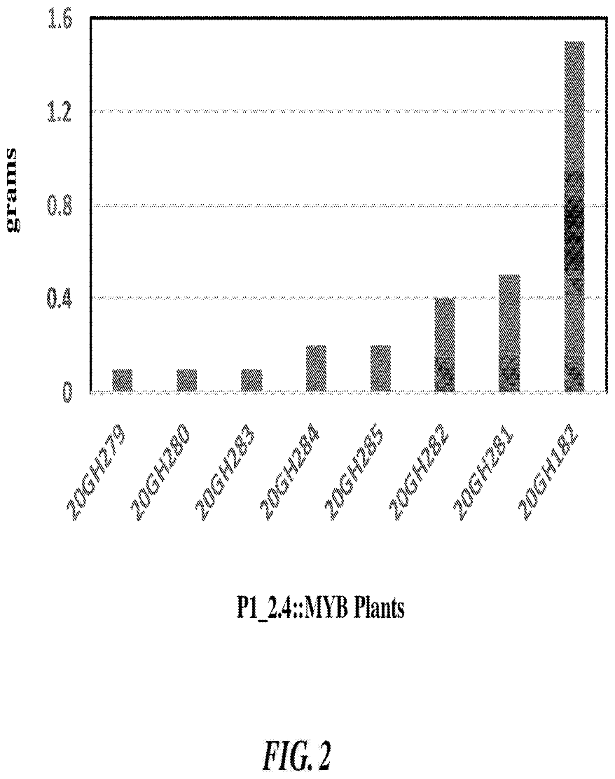

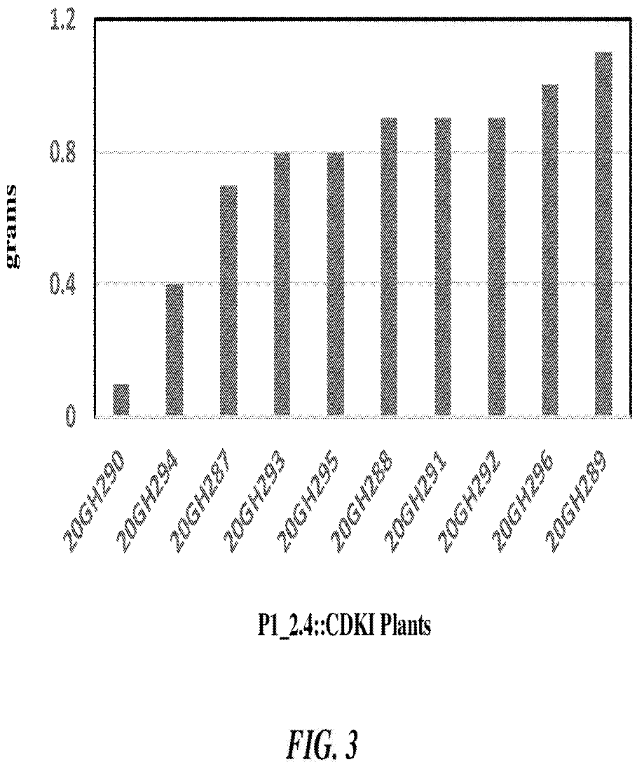

Additional transformation vectors comprising SEQ ID NO: 109 driving the expression of SEQ ID NO: 36 (P1_2.4::MYB) and SEQ ID NO: 109 driving the expression of SEQ ID NO: 5 (P1_2.4::CDKI) are also constructed and transformed into tobacco as described above in Example 2. Resulting modified tobacco plants are grown and phenotypically screened as described above in Example 3. FIG. 2 depicts the collective mass of suckers from individual P1_2.4::MYB plants one week after topping. FIG. 3 depicts the collective mass of suckers from individual P1_2.4::CDKI plants one week after topping.

### Example 6. Development of Modified Tobacco Plants Via Small RNA Molecules

Targeted suppression of genes comprising SEQ ID NOs: 1-44 and 113 in axillary bud tissues can be used to reduce or eliminate sucker outgrowth in tobacco. Transformation vectors comprising an axillary bud-preferred promoter (e.g., SEQ ID NOs: 89-109) driving the expression of an artificial miRNA designed to reduce the transcription or translation of SEQ ID NOs: 1-44 or 113 are created by modifying the Nt-miR6147 pre-miRNA and inserting the modified pre-miRNA into the transformation vector described in Example 2.

Additional transformation vectors are constructed to generate artificial miRNAs capable of suppressing the expression of CDKs in axillary buds. Each artificial miRNA construct is capable of suppressing two CDK genes. See Table 3.

TABLE 3
Artificial miRNA vectors.

| | Pro- | First | Second | Artificial | Artificial | |
|---|---|---|---|---|---|---|
| | moter | Targeted | Targeted | miRNA | Mature | Artificial |
| | SEQ | CDK | CDK | precursor | miRNA | miRNA* |
| Vector | ID | SEQ ID | SEQ ID | SEQ ID | SEQ ID | SEQ ID |
| Name | NO. | NO | NO | NO | NO | NO |
| amiRNA-1 | 109 | 1 | 4 | 118 | 121 | 124 |
| amiRNA-2 | 109 | 2 | 3 | 119 | 122 | 125 |
| amiRNA-3 | 109 | 2 | 3 | 120 | 123 | 126 |

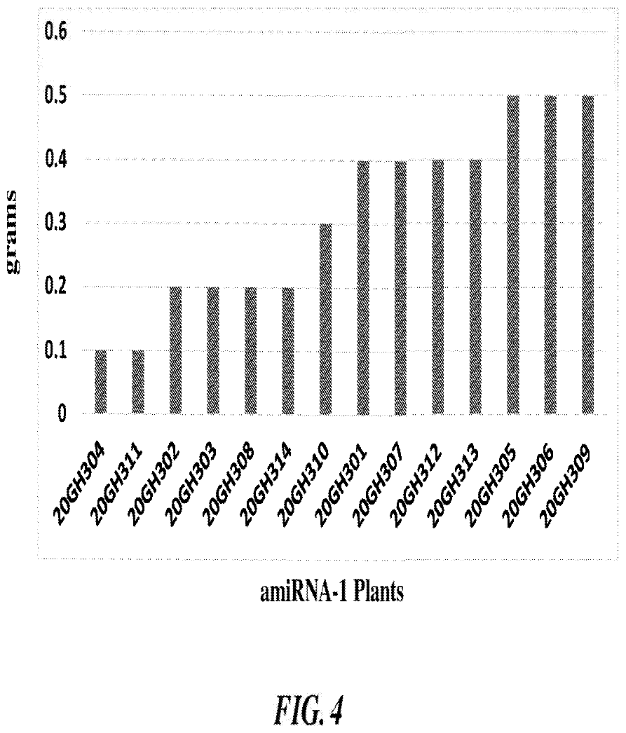

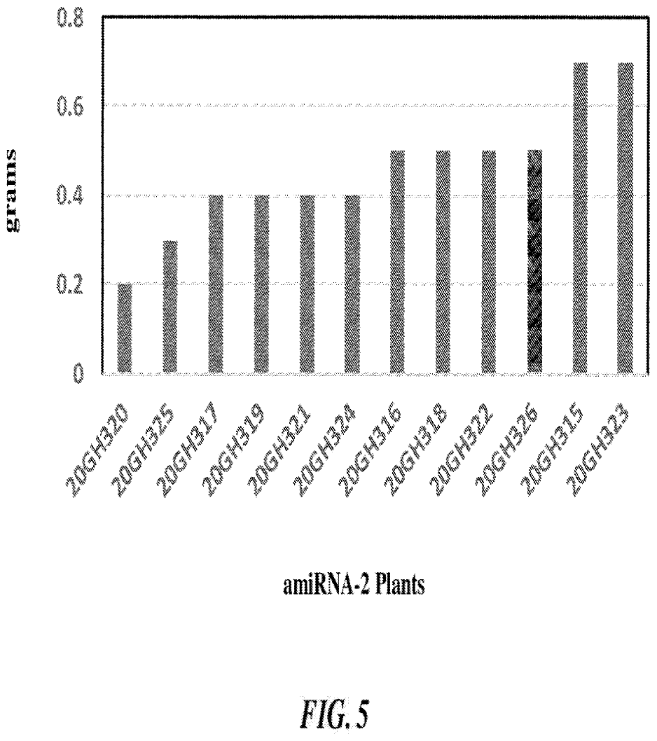

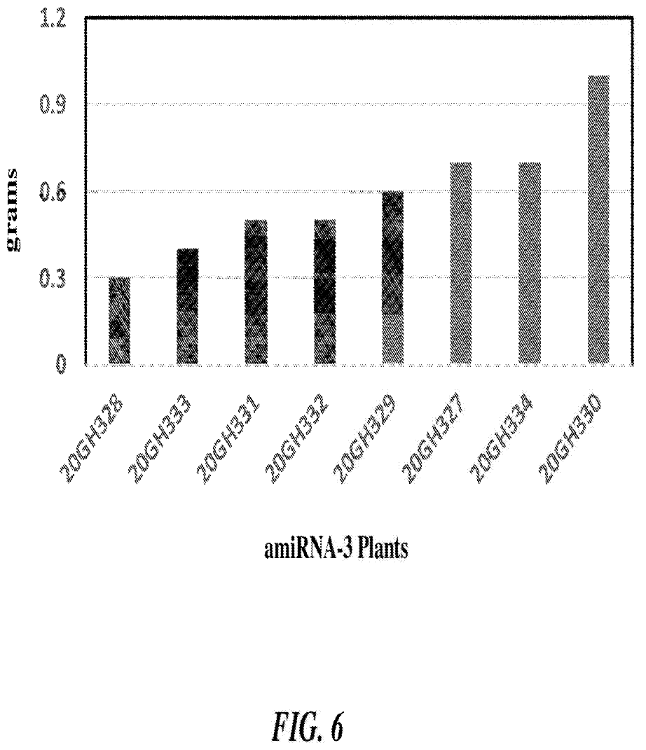

Modified tobacco plants (TO generation) comprising the transformation vectors and control tobacco plants are grown to the layby stage as described in Example 3. Then, plants are topped to remove the shoot apical meristem and modified and control tobacco plants are phenotypically evaluated as described in Example 4. FIGS. 4, 5, and 6 depict the collective mass of suckers from individual plants comprising amiRNA-1, amiRNA-2, and amiRNA-3, respectively, one week after topping.

1.-142. (canceled)

143. A method comprising:
(a) introducing a recombinant DNA construct to at least one tobacco cell, wherein the recombinant DNA construct comprises a polynucleotide encoding a cyclin-dependent kinase inhibitor (CDKI) polypeptide comprising an amino acid sequence at least 95% identical to SEQ ID NO: 49 operably linked to a heterologous axillary bud-preferred promoter;
(b) selecting at least one tobacco cell comprising the recombinant DNA construct; and
(c) regenerating a modified tobacco plant from the at least one tobacco cell selected in step (b).

144. The method of claim 143, wherein the modified tobacco plant comprises reduced suckers after topping as compared to a control tobacco plant when grown under comparable conditions.

145. The method of claim 143, wherein the amino acid sequence is at least 98% identical to SEQ ID NO: 49.

146. The method of claim 143, wherein the amino acid sequence is 100% identical to SEQ ID NO: 49.

147. The method of claim 143, wherein the polynucleotide is at least 95% identical to SEQ ID NO: 5.

148. The method of claim 143, wherein the polynucleotide is at least 98% identical to SEQ ID NO: 5.

149. The method of claim 143, wherein the polynucleotide is 100% identical to SEQ ID NO: 5.

150. The method of claim 143, wherein the heterologous axillary bud-preferred promoter comprises a nucleic acid sequence at least 98% identical to a nucleic acid sequence selected from the group consisting of SEQ ID NOs: 89 to 96, 98, 100, 102, 104, 106, 108, and 109.

151. The method of claim 143, wherein the heterologous axillary bud-preferred promoter comprises a nucleic acid sequence selected from the group consisting of SEQ ID NOs: 89 to 96, 98, 100, 102, 104, 106, 108, and 109.

152. The method of claim 143, wherein the modified tobacco plant is from a tobacco variety selected from the group consisting of a flue-cured variety, a bright variety, a Burley variety, a Virginia variety, a Maryland variety, a dark variety, an Oriental variety, and a Turkish variety.

153. A method comprising:
(a) crossing at least one tobacco plant of a first tobacco variety with at least one tobacco plant of a second tobacco variety, wherein the at least one tobacco plant of a first tobacco variety comprises a recombinant DNA construct comprising a polynucleotide encoding a cyclin-dependent kinase inhibitor (CDKI) polypeptide comprising an amino acid sequence at least 95% identical to SEQ ID NO: 49 operably linked to a heterologous axillary bud-preferred promoter; and
(b) selecting at least one progeny tobacco plant from the crossing of step (a), wherein the at least one progeny tobacco plant comprises the recombinant DNA construct.

154. The method of claim 153, wherein the at least one progeny tobacco plant exhibits reduced suckers after topping as compared to a control tobacco plant that lacks the recombinant DNA construct when grown under comparable conditions.

155. The method of claim 153, wherein the amino acid sequence is at least 98% identical to SEQ ID NO: 49.

156. The method of claim 153, wherein the amino acid sequence is 100% identical to SEQ ID NO: 49.

157. The method of claim 153, wherein the polynucleotide is at least 95% identical to SEQ ID NO: 5.

158. The method of claim 153, wherein the polynucleotide is at least 98% identical to SEQ ID NO: 5.

159. The method of claim 153, wherein the polynucleotide is 100% identical to SEQ ID NO: 5.

160. The method of claim 153, wherein the heterologous axillary bud-preferred promoter comprises a nucleic acid sequence at least 98% identical to a nucleic acid sequence selected from the group consisting of SEQ ID NOs: 89 to 96, 98, 100, 102, 104, 106, 108, and 109.

161. The method of claim 153, wherein the heterologous axillary bud-preferred promoter comprises a nucleic acid sequence selected from the group consisting of SEQ ID NOs: 89 to 96, 98, 100, 102, 104, 106, 108, and 109.

162. The method of claim 153, wherein: (i) the first tobacco variety is selected from the group consisting of a flue-cured variety, a bright variety, a Burley variety, a Virginia variety, a Maryland variety, a dark variety, an Oriental variety, and a Turkish variety; and (ii) the second tobacco variety is selected from the group consisting of a flue-cured variety, a bright variety, a Burley variety, a Virginia variety, a Maryland variety, a dark variety, an Oriental variety, and a Turkish variety.
